SEQUENCE LISTING (181 sequences)

SEQ ID NO: 1 - protein, 10 aa, synthetic construct
GFRFNPYAMN

SEQ ID NO: 2 - protein, 19 aa, synthetic construct
RIRSKSNNYETYYADSVKD

SEQ ID NO: 3 - protein, 9 aa, synthetic construct
YSDLYVLDY

SEQ ID NO: 4 - protein, 16 aa, synthetic construct
RSSKSLLHSNGNTFLY

SEQ ID NO: 5 - protein, 7 aa, synthetic construct
RMSNLAS

SEQ ID NO: 6 - protein, 9 aa, synthetic construct
MQHLEYPFT

SEQ ID NO: 7 - protein, 120 aa, synthetic construct
EVQLVEFGGGLVQPKGSLKLSCVVSGFRFNPYAMNWVRQAPGKGLEWLGRIRSKSNNYETYYADSVKDRFIISRDDSQKMVYLQMNNVKTEDTAMYYCVGYSDLYVLDYWGQGTSVTVSS

SEQ ID NO: 8 - protein, 112 aa, synthetic construct
DIVMTQAAPSVPVTPGESVSISCRSSKSLLHSNGNTFLYWFLQRPGQSPQLLIYRMSNLASGVPHRFSGSGSGTAFTLRISRVEAEDVGVYYCMQHLEYPFTFGSGTKLEIK

SEQ ID NO: 9 - protein, 450 aa, synthetic construct
EVQLVEFGGGLVQPKGSLKLSCVVSGFRFNPYAMNWVRQAPGKGLEWLGRIRSKSNNYETYYADSVKDRFIISRDDSQKMVYLQMNNVKTEDTAMYYCVGYSDLYVLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DIVMTQAAPSVPVTPGESVSISCRSSKSLLHSNGNTFLYWFLQRPGQSPQLLIYRMSNLASGVPHRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
GYTFTDYEMH

SEQ ID NO: 12 - protein, 17 aa, synthetic construct
AIDPKTGSTAYNQKFKG

SEQ ID NO: 13 - protein, 6 aa, synthetic construct
LRRFVY

SEQ ID NO: 14 - protein, 16 aa, synthetic construct
RSSKSLLHSNGNTFLY

SEQ ID NO: 15 - protein, 7 aa, synthetic construct
RVSNRFS

SEQ ID NO: 16 - protein, 9 aa, synthetic construct
SQSTHVPFT

SEQ ID NO: 17 - protein, 115 aa, synthetic construct
QVQLQQSGAELVRPGASVTLSCKASGYTFTDYEMHWVKQTPVHGLEWIGAIDPKTGSTAYNQKFKGKAIMTADKSSSTAYMELRSLTSEDSVVYYCTGLRRFVYWGQGTPVTVSA

SEQ ID NO: 18 - protein, 112 aa, synthetic construct
DVVMTQTPLSLPVSLGDQASISCRSRQSLVHSSGYTYLHWYLQKPGQSPKLLIYRVSNRFSGVPDRFSGSGSGTDFTLKISRVEAEDLGVYFCSQSTHVPFTFGSGTKLEIK

SEQ ID NO: 19 - protein, 445 aa, synthetic construct
QVQLQQSGAELVRPGASVTLSCKASGYTFTDYEMHWVKQTPVHGLEWIGAIDPKTGSTAYNQKFKGKAIMTADKSSSTAYMELRSLTSEDSVVYYCTGLRRFVYWGQGTPVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, synthetic construct
DVVMTQTPLSLPVSLGDQASISCRSRQSLVHSSGYTYLHWYLQKPGQSPKLLIYRVSNRFSGVPDRFSGSGSGTDFTLKISRVEAEDLGVYFCSQSTHVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 10 aa, synthetic construct
GYTFTDSEMH

SEQ ID NO: 22 - protein, 17 aa, synthetic construct
AIQPETGGTAYNQKFKA

SEQ ID NO: 23 - protein, 7 aa, synthetic construct
RRRNFDY

SEQ ID NO: 24 - protein, 16 aa, synthetic construct
RSSQSLFHSSGNTYLH

SEQ ID NO: 25 - protein, 7 aa, synthetic construct
KVSNRFS

SEQ ID NO: 26 - protein, 9 aa, synthetic construct
SQSTHVPFT

SEQ ID NO: 27 - protein, 116 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSS

SEQ ID NO: 28 - protein, 112 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIK

SEQ ID NO: 29 - protein, 445 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 10 aa, synthetic construct
GFTFSSYAMH

SEQ ID NO: 32 - protein, 17 aa, synthetic construct
IISYDGSNKYYADSVKG

SEQ ID NO: 33 - protein, 10 aa, synthetic construct
VPNWRGAFDI

SEQ ID NO: 34 - protein, 11 aa, synthetic construct
RASQSIGSHLH

SEQ ID NO: 35 - protein, 7 aa, synthetic construct
YASQSFS

SEQ ID NO: 36 - protein, 9 aa, synthetic construct
HQSYSLPLT

SEQ ID NO: 37 - protein, 119 aa, synthetic construct
QVQLVESGGGVVQPGRSLRLSCAASGFTFSSYAMHWVRQAPGKGLEWVGIISYDGSNKYYADSVKGRFTISRDNSKNTLYLQMNSLRAEDTALYSCARVPNWRGAFDIWGQGTMVTVSS

SEQ ID NO: 38 - protein, 107 aa, synthetic construct
EIVLTQSPDFQSVTPKEKVTITCRASQSIGSHLHWYQQKPDQSPKLLIKYASQSFSGVPSRFSGSGSGTDFSLTINSLETEDAATYFCHQSYSLPLTFGGGTKVEIK

SEQ ID NO: 39 - protein, 10 aa, synthetic construct
GFTFSNYNMI

SEQ ID NO: 40 - protein, 17 aa, synthetic construct
YISSSRSIISYADSVKG

SEQ ID NO: 41 - protein, 8 aa, synthetic construct
RWKGVFDY

SEQ ID NO: 42 - protein, 11 aa, synthetic construct
RASQGISSWLA

SEQ ID NO: 43 - protein, 7 aa, synthetic construct
AASSLQS

SEQ ID NO: 44 - protein, 9 aa, synthetic construct
QQYNSYPPT

SEQ ID NO: 45 - protein, 117 aa, synthetic construct
QVQLVESGGGLVQPGGSLRLSCAASGFTFSNYNMIWVRQTPGKGLEWISYISSSRSIISYADSVKGRFTISRDNAKNSLYLQMNSLRDEDTAVYYCARRWKGVFDYWGQGTLVTVSS

SEQ ID NO: 46 - protein, 107 aa, synthetic construct
DIVMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKVEIK

SEQ ID NO: 47 - protein, 10 aa, synthetic construct
GFRFGPYAMN

SEQ ID NO: 48 - protein, 19 aa, synthetic construct
RIRSKSNNYETYYADSVKD

SEQ ID NO: 49 - protein, 9 aa, synthetic construct
YSDLYVLDY

SEQ ID NO: 50 - protein, 16 aa, synthetic construct
RSSKSLLHSQGNTFLY

SEQ ID NO: 51 - protein, 7 aa, synthetic construct
RMSNLAS

SEQ ID NO: 52 - protein, 9 aa, synthetic construct
MQHLEYPFT

SEQ ID NO: 53 - protein, 120 aa, synthetic construct
EVQLVESGGGLVKPGGSLRLSCAASGFRFGPYAMNWVRQAPGKGLEWVGRIRSKSNNYETYYADSVKDRFTISRDDSKNTLYLQMNSLKTEDTAVYYCVGYSDLYVLDYWGQGTLVTVSS

SEQ ID NO: 54 - protein, 112 aa, synthetic construct
DIVMTQTPPSLPVNPGEPASISCRSSKSLLHSQGNTFLYWYLQKPGQSPQLLIYRMSNLASGVPDRFSGSGSGSDFTLKISWVEAEDVGVYYCMQHLEYPFTFGPGTKVDIK

SEQ ID NO: 55 - protein, 449 aa, synthetic construct
EVQLVESGGGLVKPGGSLRLSCAASGFRFGPYAMNWVRQAPGKGLEWVGRIRSKSNNYETYYADSVKDRFTISRDDSKNTLYLQMNSLKTEDTAVYYCVGYSDLYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 219 aa, synthetic construct
DIVMTQTPPSLPVNPGEPASISCRSSKSLLHSQGNTFLYWYLQKPGQSPQLLIYRMSNLASGVPDRFSGSGSGSDFTLKISWVEAEDVGVYYCMQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 5 aa, synthetic construct
NSGMH

SEQ ID NO: 58 - protein, 17 aa, synthetic construct
VIWYDGSKRYYADSVKG

SEQ ID NO: 59 - protein, 4 aa, synthetic construct
NDDY

SEQ ID NO: 60 - protein, 11 aa, synthetic construct
RASQSVSSYLA

SEQ ID NO: 61 - protein, 7 aa, synthetic construct
DASNRAT

SEQ ID NO: 62 - protein, 9 aa, synthetic construct
QQSSNWPRT

SEQ ID NO: 63 - protein, 113 aa, synthetic construct [sequence omitted]

SEQ ID NO: 64 - protein, 107 aa, synthetic construct [sequence omitted]

SEQ ID NO: 65 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLLSKADYEKHKVYACEVTHQGLSSPVTKSFNRGEC

SEQ ID NO: 67 - protein, 5 aa, synthetic construct
NYYMY

SEQ ID NO: 68 - protein, 17 aa, synthetic construct
GINPSNGGTNFNEKFKN

SEQ ID NO: 69 - protein, 11 aa, synthetic construct
RDYRFDMGFDY

SEQ ID NO: 70 - protein, 15 aa, synthetic construct
RASKGVSTSGYSYLH

SEQ ID NO: 71 - protein, 7 aa, synthetic construct
LASYLES

SEQ ID NO: 72 - protein, 9 aa, synthetic construct
QHSRDLPLT

SEQ ID NO: 73 - protein, 120 aa, synthetic construct [sequence omitted]

SEQ ID NO: 74 - protein, 111 aa, synthetic construct [sequence omitted]

SEQ ID NO: 75 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 8 aa, synthetic construct
GFTFSNFG

SEQ ID NO: 78 - protein, 8 aa, synthetic construct
ISGGGRDT

SEQ ID NO: 79 - protein, 10 aa, synthetic construct
VKWGNIYFDY

SEQ ID NO: 80 - protein, 6 aa, synthetic construct
LSINTF

SEQ ID NO: 81 - skipped

SEQ ID NO: 82 - protein, 9 aa, synthetic construct
QQSSNTPFT

SEQ ID NO: 83 - protein, 117 aa, synthetic construct [sequence omitted]

SEQ ID NO: 84 - protein, 107 aa, synthetic construct [sequence omitted]

SEQ ID NO: 85 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 5 aa, synthetic construct
TYWMH

SEQ ID NO: 88 - protein, 17 aa, synthetic construct
NIYPGTGGSNFDEKFKN

SEQ ID NO: 89 - protein, 8 aa, synthetic construct
WTTGTGAY

SEQ ID NO: 90 - protein, 17 aa, synthetic construct
KSSQSLLDSGNQKNFLT

SEQ ID NO: 91 - protein, 7 aa, synthetic construct
WASTRES

SEQ ID NO: 92 - protein, 9 aa, synthetic construct
QNDYSYPYT

SEQ ID NO: 93 - protein, 117 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 94 - protein, 113 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 95 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 5 aa, synthetic construct
DYYWN

SEQ ID NO: 98 - protein, 16 aa, synthetic construct
EINHRGSTNSNPSLKS

SEQ ID NO: 99 - protein, 12 aa, synthetic construct
GYSDYEYNWFDP

SEQ ID NO: 100 - protein, 11 aa, synthetic construct
RASQSISSYLA

SEQ ID NO: 101 - protein, 7 aa, synthetic construct
DASNRAT

SEQ ID NO: 102 - protein, 9 aa, synthetic construct
QQRSNWPLT

SEQ ID NO: 103 - protein, 120 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 104 - protein, 107 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 105 - protein, 447 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 214 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 8 aa, synthetic construct
GFTFSSYG

SEQ ID NO: 108 - protein, 8 aa, synthetic construct
IWYDGSNK

SEQ ID NO: 109 - protein, 16 aa, synthetic construct
ASVATSGDFDYYGMDV

SEQ ID NO: 110 - protein, 6 aa, synthetic construct
QRISTY

SEQ ID NO: 111 - skipped

SEQ ID NO: 112 - protein, 9 aa, synthetic construct
QQRSNWPLT

SEQ ID NO: 113 - protein, 123 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 114 - protein, 107 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 115 - protein, 449 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 214 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 5 aa, synthetic construct
NYGMN

SEQ ID NO: 118 - protein, 17 aa, synthetic construct
WINTDTGEPTYADDFKG

SEQ ID NO: 119 - protein, 16 aa, synthetic construct
NPPYYYGTNNAEAMDY

SEQ ID NO: 120 - protein, 11 aa, synthetic construct
SSSQDISNYLN

SEQ ID NO: 121 - protein, 7 aa, synthetic construct
YTSTLHL

SEQ ID NO: 122 - protein, 9 aa, synthetic construct
QQYYNLPWT

SEQ ID NO: 123 - protein, 125 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 124 - protein, 107 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 125 - protein, 125 aa, synthetic construct
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 126 - protein, 107 aa, synthetic construct
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 127 - protein, 451 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 128 - protein, 214 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 451 aa, synthetic construct
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 130 - protein, 214 aa, synthetic construct
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 5 aa, synthetic construct
DYNVD

SEQ ID NO: 132 - protein, 17 aa, synthetic construct
DINPNDGGTIYAQKFQE

SEQ ID NO: 133 - protein, 10 aa, synthetic construct
NYRWFGAMDH

SEQ ID NO: 134 - protein, 15 aa, synthetic construct
KASQSLDYEGDSDMN

SEQ ID NO: 135 - protein, 7 aa, synthetic construct
GASNLES

SEQ ID NO: 136 - protein, 9 aa, synthetic construct
QQSTEDPRT

SEQ ID NO: 137 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 138 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 139 - protein, 446 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 218 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 5 aa, synthetic construct
SYTMH

SEQ ID NO: 142 - protein, 17 aa, synthetic construct
FISYDGNNKYYADSVKG

SEQ ID NO: 143 - protein, 9 aa, synthetic construct
TGWLGPFDY

SEQ ID NO: 144 - protein, 12 aa, synthetic construct
RASQSVGSSYLA

SEQ ID NO: 145 - protein, 7 aa, synthetic construct
GAFSRAT

SEQ ID NO: 146 - protein, 9 aa, synthetic construct
QQYGSSPWT

SEQ ID NO: 147 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 148 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 149 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 447 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 146 aa, synthetic construct
CRTQLYGYNLCPYGGGSSGGSISSGLLSGRSDNHGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 153 - protein, 146 aa, synthetic construct
CRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 154 - protein, 146 aa, synthetic construct
CRTQLYGYNLCPYGGGSSGGSISSGLLSGRSANPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 155 - protein, 259 aa, synthetic construct
QGQSGSCRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 8 aa, synthetic construct
GFTFSSYG

SEQ ID NO: 157 - protein, 10 aa, synthetic construct
AVIWYDGSNK

SEQ ID NO: 158 - protein, 18 aa, synthetic construct
ARDPRGATLYYYYYGMDV

SEQ ID NO: 159 - protein, 6 aa, synthetic construct
QSINSY

SEQ ID NO: 160 - skipped

SEQ ID NO: 161 - protein, 9 aa, synthetic construct
QQYYSTPFT

SEQ ID NO: 162 - protein, 125 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 163 - protein, 108 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKR

SEQ ID NO: 164 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 166 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 107 aa, synthetic construct [sequence omitted]

SEQ ID NO: 168 - protein, 6 aa, synthetic construct
QGQSGS

SEQ ID NO: 169 - protein, 13 aa, synthetic construct
CRTQLYGYNLCPY

SEQ ID NO: 170 - protein, 13 aa, synthetic construct
ISSGLLSGRSDNH

SEQ ID NO: 171 - protein, 13 aa, synthetic construct
ISSGLLSGRSDNP

SEQ ID NO: 172 - protein, 13 aa, synthetic construct
ISSGLLSGRSANP

SEQ ID NO: 173 - protein, 189 aa, synthetic construct [sequence omitted]

SEQ ID NO: 174 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 175 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 176 - protein, 355 aa, synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 177 - protein, 525 aa, synthetic construct
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 178 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 179 - skipped

SEQ ID NO: 180 - protein, 7 aa, synthetic construct
YYYPDIF

SEQ ID NO: 181 - protein, 11 aa, synthetic construct
VTDYYYPDIFS

(19) United States
(12) Patent Application Publication
(10) Pub. No.: US 2025/0215087 A1
(43) Pub. Date: Jul. 3, 2025

(54) COMBINATION THERAPY OF KRAS INHIBITOR AND TREG DEPLETING AGENT

(71) Applicant: Bristol-Myers Squibb Company, Princeton, NJ (US)

(72) Inventors: David Julian Harry DOWNWARD, London (GB); Panayiotis ANASTASIOU, London (GB); Megan COLE, London (GB); Matthew John MEYER, Princeton, NJ (US); Febe VAN MALDEGEM, London (GB); Nicholas Stuart WILSON, Princeton, NJ (US)

(73) Assignee: Bristol-Myers Squibb Company, Princeton, NJ (US)

(21) Appl. No.: 19/006,029

(22) Filed: Dec. 30, 2024

Related U.S. Application Data
(60) Provisional application No. 63/616,357, filed on Dec. 29, 2023.

(51) Int. Cl. C07K 16/28; A61K 39/00; A61K 45/06; A61P 35/00; A61P 37/04; G01N 33/50

(52) U.S. Cl. CPC C07K 16/2818; A61K 45/06; A61P 35/00; A61P 37/04; C07K 16/2866; G01N 33/505; A61K 2039/505; C07K 2317/565

(57) ABSTRACT

In some aspects, the present disclosure is directed to a method of treating a tumor in a subject in need thereof comprising administering a KRAS inhibitor and a regulatory T cell (Treg)-depleting agent to the subject. In some aspects, the present disclosure is further directed to methods of reducing the number of Treg cells (Tregs) in a tumor environment (TME) in a subject who receives a therapy with a KRAS inhibitor comprising administering a Treg-depleting agent to the subject. In some aspects, the present disclosure is further directed to methods of treating a tumor in a subject who is identified as having an increased number of Tregs in a TME, or as having a spatial cellular community comprising Tregs in a TME.

### CROSS-REFERENCE TO EARLIER FILED APPLICATIONS

This application claims priority to and the benefit of U.S. Provisional Application No. 63/616,357, filed Dec. 29, 2023, the contents of which are incorporated herein by reference in its entirety.

### REFERENCE TO SEQUENCE LISTING SUBMITTED ELECTRONICALLY VIA EFS-WEB

The content of the electronically submitted sequence listing (Name: 3338_3330001_Sequencelisting_ST26.xml, Size: 172,036 bytes; and Date of Creation: Dec. 30, 2024) is incorporated herein by reference in its entirety.

### FIELD OF THE DISCLOSURE

The present disclosure inter alia provides methods of treating a tumor in a subject in need thereof comprising administering a KRAS inhibitor and a regulatory T cell (Treg)-depleting agent to the subject. The present disclosure further provides methods of reducing the number of Treg cells (Tregs) in a tumor environment (TME) in a subject who receives a therapy with a KRAS inhibitor comprising administering a Treg-depleting agent to the subject. The present disclosure further provides methods of treating a tumor in a subject who is identified as having an increased number of Tregs in a TME, or as having a spatial cellular community comprising Tregs in a TME. In some aspects of the methods of the present disclosure, the subject a human.

### BACKGROUND OF THE DISCLOSURE

Recent years have seen a transformation in the treatment of cancers, such as non-small cell lung cancer (NSCLC), with the introduction of immune checkpoint blockade, which has increased survival rates of patients with a previously poor prognosis. Despite this, only a subset of patients responds and many responders acquire resistance over time (Borghaei et al. (2015) N. Eng. J. Med. 373:1627-1639; Gettinger et al. (2018) J. Thorac. Oncol. 13:831-839). In 2021, a further breakthrough occurred when the KRAS inhibitor sotorasib (also known as AMG 510, LUMAKRAS®, or LUMYKRAS®; see formula (I)) was approved for the treatment of locally advanced or metastatic KRAS-G12C mutant NSCLC. This followed successful clinical trials where 80% of patients achieved temporary disease control following sotorasib treatment. However, despite a modest improvement in progression free survival, sotorasib gave no improvement in overall survival compared to docetaxel (de Langen et al. (2023) Lancet 401:733-746), demonstrating its limitations for use as a monotherapy. Following the approval of sotorasib, adagrasib (also known as MRTX849 or KRAZATI®; see formula (II)), another KRAS-G12C inhibitor, was clinically approved.

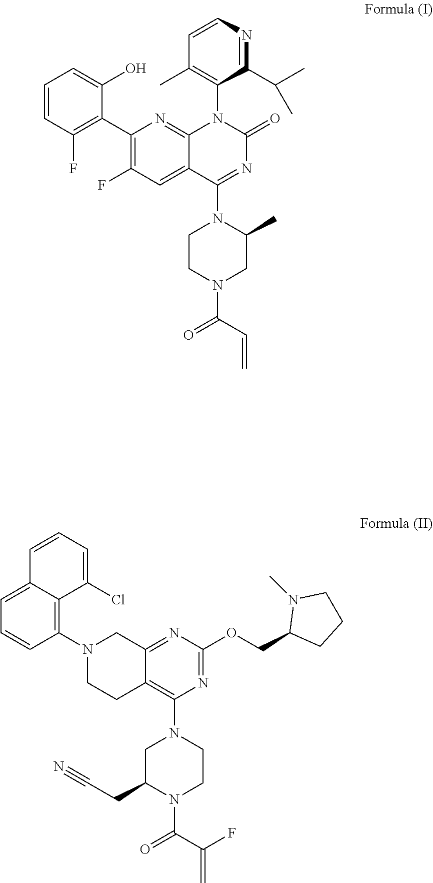

Previous analysis of mice bearing immune-cold orthotopic Lewis lung (3LL) NSCLC tumors had established that following seven consecutive days of treatment with the KRAS-G12C inhibitor MRTX1257 (see formula (III)), tumors did not regress although tumor growth was inhibited. This indicated that KRAS-G12C inhibitors alone are not sufficient to cause tumor regression in the 3LL tumor model (van Maldegem et al. (2021) Nat. Commun. 12:5906). Also a combination of this KRAS-G12C inhibitor with anti-PD-1 or anti-PD-L1 therapy (even when further combined with anti-LAG-3 therapy) did not increase responses in this tumor model (Mugarza et al. (2022) Sci. Adv. 8: eabm8780). This is also reflective of the failure to see a beneficial effect on response of combined KRAS inhibition and PD-1 blockade in the clinical setting, either due to combination toxicities or lack of efficacy (Chour et al. (2023) J. Thorac. Oncol. 18 (10): 1408-1415). Chour et al. reported that “[s]equential anti-PD-(L)1 and sotorasib therapy are associated with a significantly increased risk of severe sotorasib-related hepatotoxicity and severe non-liver AEs [adverse events].” See Abstract of Chour et al. (2023).

There is clearly an unmet need for more effective therapeutic combinations with KRAS-G12C inhibitors to improve T cell-mediated anti-tumoral immune responses, and the invention claimed herein addresses this need by disclosing combinations of KRAS inhibitors with Treg-depleting agents for enhanced anti-tumor activity.

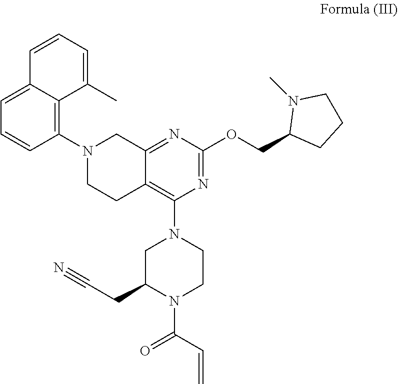 [SMILES: C=CC(=O)N1CCN(c2nc(OC[C@@H]3CCCN3C)nc3c2CCN(c2cccc4cccc(C)c24)C3)C[C@@H]1CC#N]

### BRIEF SUMMARY OF THE DISCLOSURE

In some aspects, the present disclosure is directed to a method of treating a tumor in a subject in need thereof comprising administering a KRAS inhibitor and a regulatory T cell (“Treg”)-depleting agent to the subject. In some aspects, the subject is a human. In some aspects, the KRAS inhibitor is a KRAS-G12C inhibitor (such as adagrasib, sotorasib, or MRTX1257). In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or 4A19 (imzokitug)), or an antigen binding portion thereof.

In some aspects, the present disclosure is also directed to a method of treating a tumor in a subject in need thereof comprising: administering a KRAS inhibitor; measuring the number of regulatory T cells (“Tregs”) in the tumor microenvironment (“TME); and administering a “Treg”-depleting agent to the subject.

In some aspects, the present disclosure is directed to a method of treating a tumor in a subject in need thereof comprising administering a Treg-depleting agent to the subject, wherein the subject is identified as having an increased number of Tregs in the TME after a therapy with a KRAS inhibitor.

In some aspects, the present disclosure is also directed to a method of treating a tumor in a subject in need thereof comprising administering a KRAS inhibitor and a Treg-depleting agent to the subject.

In some aspects, the present disclosure is directed to a method of reducing the number of Tregs in the TME of a tumor in a subject who is treated with a KRAS inhibitor comprising administering a Treg-depleting agent to the subject.

In some aspects, the present disclosure is directed to a method of identifying a subject eligible for a Treg-depleting agent therapy after a KRAS inhibitor therapy comprising measuring the number of Tregs in a TME in the subject.

In some aspects, the present disclosure is directed to a method of predicting the responsiveness of a subject having a tumor to an anti-tumor therapy, the method comprising determining, or ordering the determination of, the number of Treg cells in a TME, wherein the number of Treg cells is predictive of the responsiveness of the subject to the anti-tumor therapy.

In some aspects, the present disclosure is directed to a method of treating a tumor in a subject in need thereof comprising: administering a KRAS inhibitor; measuring the number of Tregs in the tumor microenvironment (“TME); and administering a Treg-depleting agent and a KRAS inhibitor to the subject, wherein the subject is identified as having an increased number of Tregs in the TME after a therapy with a KRAS inhibitor.

In some aspects, the present disclosure is further directed to a method of reducing the number of Tregs in the TME of a tumor in a subject who receives a therapy with a KRAS inhibitor comprising administering a Treg-depleting agent to the subject. In some aspects, the subject is a human. In some aspects, the KRAS inhibitor is a KRAS-G12C inhibitor (such as adagrasib, sotorasib, or MRTX1257). In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of treating a tumor in a subject in need thereof comprising administering a Treg-depleting agent to the subject, wherein the subject is identified as having an increased number of Tregs in the TME after a therapy with a KRAS inhibitor. In some aspects, the subject is a human. In some aspects, the KRAS inhibitor is a KRAS-G12C inhibitor (such as adagrasib, sotorasib, or MRTX1257). In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of identifying a subject with an increased number of Tregs in a TME in the subject after a therapy with a KRAS inhibitor comprising measuring the number of Tregs in a spatial cellular community in the TME. In some aspects, the subject is a human. In some aspects, the KRAS inhibitor is a KRAS-G12C inhibitor (such as adagrasib, sotorasib, or MRTX1257).

In some aspects, the present disclosure is further directed to a method of treating a tumor in a subject in need thereof comprising administering a Treg-depleting agent to the subject identified by the methods as described herein as having an increased number of Tregs in the TME after a therapy with a KRAS inhibitor. In some aspects, the subject is a human. In some aspects, the KRAS inhibitor is a KRAS-G12C inhibitor (such as adagrasib, sotorasib, or MRTX1257). In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of predicting the responsiveness of a subject having a tumor to an anti-tumor therapy, the method comprising determining, or ordering the determination of, the number of Tregs in the TME, wherein the number of Tregs is predictive of the responsiveness of the subject to the anti-tumor therapy. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a programmed cell death protein 1 (PD-1) axis-blocking agent, such as an anti-PD-1 or an anti-PD-L1 antibody.

In some aspects, the present disclosure is further directed to a method of selecting a subject (which might be a human) having a tumor for an anti-tumor therapy (which might comprise a KRAS inhibitor, such as a KRAS-G12C inhibitor, for example, adagrasib, sotorasib, or MRTX1257, and/or a PD-1 axis-blocking agent, such as an anti-PD-1 or an anti-PD-L1 antibody), the method comprising:
determining, or ordering the determination of, the number of Tregs in the TME, wherein the number of Tregs is predictive of the responsiveness of the subject to the anti-tumor therapy; and
selecting the subject for the anti-tumor therapy on the basis that the subject is predicted to be responsive to the anti-tumor therapy.

In some aspects, the present disclosure is further directed to a method of treating a tumor in a subject in need thereof comprising administering an anti-tumor therapy to the subject, wherein the subject is identified as not having an increased number of Tregs in the TME prior to administering the anti-tumor therapy to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of treating a tumor in a subject in need thereof comprising administering an anti-tumor therapy and a Treg-depleting agent to the subject, wherein the subject is identified as having an increased number of Tregs in the TME prior to administering the anti-tumor therapy and the Treg-depleting agent to the subject. In some aspects, the subject is a human. In some aspects, the anti-tumor therapy comprises a KRAS inhibitor (such as adagrasib, sotorasib, or MRTX1257). In some aspects, the anti-tumor therapy comprises a PD-1 axis-blocking agent, such as an anti-PD-1 or an anti-PD-L1 antibody. In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of treating a tumor in a subject in need thereof comprising administering an anti-tumor therapy to the subject, wherein the subject is identified as a subject that will be responsive to the anti-tumor therapy by determining the number of Tregs in the TME prior to administering the anti-tumor therapy to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of treating a tumor in a subject in need thereof comprising administering an anti-tumor therapy and a Treg-depleting agent to the subject, wherein the subject is identified as a subject that will not be responsive to the anti-tumor therapy by determining the number of Tregs in the TME prior to administering the anti-tumor therapy and the Treg-depleting agent to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody. In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of predicting the responsiveness of a subject having a tumor to an anti-tumor therapy, e.g., a KRAS inhibitor therapy, a PD-1 axis-blocking therapy, or the combination thereof, the method comprising determining, or ordering the determination of, the absence of a spatial cellular community comprising CD4⁺ T cells, CD8⁺ T cells, Tregs, and DCs in the TME, wherein the absence of the spatial cellular community is predictive that the subject will be responsive to the anti-tumor therapy. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of selecting a subject (which might be a human) having a tumor for an anti-tumor therapy (which might comprise a KRAS inhibitor, such as a KRAS-G12C inhibitor, for example, adagrasib, sotorasib, or MRTX1257, and/or a PD-1 axis-blocking agent, such as an anti-PD-1 or an anti-PD-L1 antibody), the method comprising:
determining, or ordering the determination of, the absence of a spatial cellular community comprising CD4⁺ T cells, CD8⁺ T cells, Tregs, and DCs in the TME; and
selecting the subject for the anti-tumor therapy if the spatial cellular community is absent in the TME.

In some aspects, the present disclosure is further directed to a method of treating a tumor in a subject in need thereof comprising administering an anti-tumor therapy to the subject, wherein the subject is identified as not having a spatial cellular community comprising CD4⁺ T cells, CD8⁺ T cells, Tregs, and DCs in the TME prior to administering the anti-tumor therapy to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of treating a tumor in a subject in need thereof comprising administering an anti-tumor therapy and a Treg-depleting agent to the subject, wherein the subject is identified as having a spatial cellular community comprising CD4⁺ T cells, CD8⁺ T cells, Tregs, and DCs in the TME prior to administering the anti-tumor therapy to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody. In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method of predicting the responsiveness of a subject having a tumor to an anti-tumor therapy, the method comprising determining, or ordering the determination of, the number of spatial cellular communities comprising CD4⁺ T cells, CD8⁺ T cells, Tregs, and DCs in the TME, wherein the number of the spatial cellular communities is predictive of the responsiveness of the subject to the anti-tumor therapy. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of selecting a subject (which might be a human) having a tumor for an anti-tumor therapy (which might comprise a KRAS inhibitor, such as a KRAS-G12C inhibitor, for example, adagrasib, sotorasib, or MRTX1257, and/or a PD-1 axis-blocking agent, such as an anti-PD-1 or an anti-PD-L1 antibody), the method comprising:
determining, or ordering the determination of, the number of spatial cellular communities comprising CD4⁺ T cells, CD8⁺ T cells, Tregs, and DCs in the TME, wherein the number of the spatial cellular communities is predictive of the responsiveness of the subject to the anti-tumor therapy; and
selecting the subject for the anti-tumor therapy on the basis that the subject is predicted to be responsive to the anti-tumor therapy.

In some aspects, the present disclosure is further directed to a method of treating a tumor in a subject in need thereof comprising administering an anti-tumor therapy to the subject, wherein the subject is identified as a subject that will be responsive to the anti-tumor therapy by determining the number of spatial cellular communities comprising CD4⁺ T cells, CD8⁺ T cells, Tregs, and DCs in the TME prior to administering the anti-tumor therapy to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody.

In some aspects, the present disclosure is further directed to a method of treating a tumor in a subject in need thereof comprising administering an anti-tumor therapy and a Treg-depleting agent to the subject, wherein the subject is identified as a subject that will not be responsive to the anti-tumor therapy by determining the number of spatial cellular communities comprising CD4⁺ T cells, CD8⁺ T cells, Tregs, and DCs in the TME prior to administering the anti-tumor therapy and the Treg-depleting agent to the subject. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the anti-tumor therapy comprises a PD-1 axis-blocking agent, such as an anti-PD-1 or an anti-PD-L1 antibody. In some aspects, the Treg-depleting agent comprises an anti-CTLA-4 antibody (such as tremelimumab or ipilimumab), or an antigen binding portion thereof. In some aspects, the Treg-depleting agent comprises an anti-CCR8 antibody (such as anti-CCR8-antibody 14S15, 14S15h, or imzokitug), or an antigen binding portion thereof.

In some aspects, the present disclosure is further directed to a method for identifying a subject having a tumor who is not suitable for treatment with a KRAS inhibitor and a PD-1 axis-blocking agent comprising determining that the subject possesses spatial cellular communities in the TME, wherein the spatial cellular communities are rich in Tregs. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the PD-1 axis-blocking agent, is an anti-PD-1 or an anti-PD-L1 antibody.

In some aspects, the present disclosure is further directed to a method for identifying a subject having a tumor who is not suitable for treatment with a KRAS inhibitor and a PD-1 axis-blocking agent comprising determining that the subject possesses spatial cellular communities in the TME, wherein the spatial cellular communities comprise about 0.5% to about 2% Tregs. In some aspects, the subject is a human. In some aspects, the anti-tumor therapy comprises a KRAS inhibitor (such as a KRAS-G12C inhibitor, for example, adagrasib, sotorasib, or MRTX1257). In some aspects, the PD-1 axis-blocking agent, is an anti-PD-1 or an anti-PD-L1 antibody.

In the present disclosure, the 3LL immune evasive orthotopic lung tumor model, in which effector immune cells are excluded from the tumor, was used to seek for more effective therapeutic combinations with KRAS inhibition. Imaging mass cytometry (IMC) was applied to analyse the make-up of these tumors in situ. IMC is particularly useful for studying the tumor microenvironment (TME) due to its ability to capture up to 40 markers simultaneously. Spatial information remains intact, meaning that cell phenotypes can be analysed in the context of their spatial neighbors (Giesen et al. (2014) Nat. Methods 11:417-422). Obtaining spatial information was shown to be very valuable when studying the TME, as it provides insight into the cellular interactions dictating local immune activation or suppression, as mechanisms of immune response or resistance to treatment.

The present disclosure inter alia provides data on the identification of neighborhood communities through single cell spatial analysis to investigate which cellular interaction patterns may restrain anti-tumoral immune responses in the 3LL tumor model. A community resembling a T cell activation hub was identified, where regulatory T cell (Treg) interactions are thought to play a key role in dampening anti-tumoral immune responses following KRAS-G12C inhibition (see, in particular, Examples 1 to 6). In parallel, cellular community analysis of treatment-naïve human lung adenocarcinoma patient samples from the RUBICON TRACERx cohort suggested that similar local Treg control is restraining immune responses in a subset of patients (see, in particular, Example 7). When exploring the effect of combining a KRAS-G12C inhibitor with a Treg-depleting agent (in particular, with an anti-CTLA-4 or an anti-CCR8 antibody) in the in vivo orthotopic setting, markedly improved responses were observed (see, in particular, Examples 8 and 9).

The findings of the present disclosure open up not only the perspective of combining KRAS inhibitors (such as in particular KRAS-G12C inhibitors) with Treg-depleting agents (such as in particular anti-CTLA-4 or anti-CCR8 antibodies) to improve durable response rates, but also the potential to use Treg numbers, as well as the presence and/or numbers of the spatial cellular communities described in the present disclosure as selective biomarkers to predict the outcome of clinical treatments comprising KRAS inhibitors (such as in particular combination therapies of KRAS inhibitors with anti-PD-(L)1). Such a selective biomarker would be highly valuable as selective therapy would be possible, avoiding an unnecessary increase of toxicity risks in patients receiving the therapy who are, however, likely to not respond.

Moreover, as the spatial cellular communities as described herein have not only been observed in the context of KRAS inhibitor treatment (see, for example, Example 7, in which samples from naïve human patients were analyzed), the presence and/or number of such communities also have the potential to be predictive for the benefit a patient might observe from therapies that do not necessarily comprise a KRAS inhibitor (such as in particular anti-PD-(L)1 and anti-CTLA-4 combination therapies).

### BRIEF DESCRIPTION OF THE DRAWINGS

FIGS. 1A-1B show the antigens bound by panels of 27 antibodies for dataset 1 (FIG. 1A) and 21 antibodies for dataset 2 (FIG. 1B) that identify multiple cell types across lymphocyte, myeloid, tumor and stromal compartments, as well as immune checkpoint markers and cell phenotypic markers including for detection of proliferation and apoptosis. Additional antigens bound by antibodies for the dataset 2 panel (FIG. 1B) include LAG-3 for identifying exhausted T cells, MHC-I, and CXCL9, a chemokine associated with T cell attraction.

FIG. 2 shows dimensionality reduction using tSNE (t-distributed Stochastic Neighbor Embedding) of 62 communities generated with a k-input value of 250 and 47 communities generated with a k-input of 350 into Rphenograph using dataset 1, where tSNE analysis was run based on the proportion of each cell type contributing to each community. The analysis revealed very similar patterns of community phenotypes.

FIG. 3 shows tSNE plot of the 18 communities generated for dataset 1 and 18 communities generated for dataset 2 following Rphenograph clustering based on the proportion of only the cell types shared across both datasets within the neighborhood of each cell. A separate community clustering analysis on both datasets, based on shared cell types only, demonstrated that the method to generate communities was stable to altered input data.

FIG. 4 shows percentage distribution of all 18 communities across normal, interface and tumor domains of the tissue for dataset 1.

FIG. 5 shows percentage distribution of each community across Vehicle (left) and MRTX1257 (right) treatment groups. Bars ordered by increasing percentage distribution in Vehicle setting.

FIGS. 6A-6B show Pearson correlation calculation on the proportion of each cell type pair within each community *=p<0.05, **=p<0.01, ***=p<0.001. Cell types clustered based on correlation value (FIG. 6A). Pearson correlation calculation of the proportion of Dendritic cells CD103 and T cells CD8⁺ in each of the 18 communities for dataset 1 (FIG. 6B).

FIGS. 7A-7B shows Pearson correlation calculation on the proportion of each cell type pair within each ROI. *=p<0.05, **=p<0.01, ***=p<0.001. Cell types clustered based on correlation value (FIG. 7A). Pearson correlation calculation of the proportion of Dendritic cells CD103 and T cells CD8⁺ in each of the 12 ROIs for dataset 1 (FIG. 7B).

FIGS. 8A-8B show number of CD8⁺ T cells assigned to each community, with bars coloured by distribution of those cells across Vehicle (bottom) and MRTX1257 (top) treatment groups for dataset 1 (FIG. 8A) and dataset 2 (FIG. 8B).

FIG. 9 shows percentage distribution of all CD8⁺ T cells in dataset 1 and 2 across T/NA, T/M1, T/DC, T/M2_1 and T/M2_2 communities and all ‘other’ communities.

FIGS. 10A-10B show distribution of all cells assigned to T/NA, T/M1, T/DC, T/M2_1 and T/M2_2 communities across Vehicle and MRTX1257 treatment groups, relative to the proportion of each treatment group across the whole cohort size for dataset 1 (left) and dataset 2 (right; FIG. 10A). Percentage of all CD8⁺ T cells found in the top 5 communities, coloured by their distribution across each of the top 5 communities in Vehicle (left) and MRTX1257 (right) treatment groups, for dataset 1 (left) and dataset 2 (right; FIG. 10B).

FIG. 11 shows cell count of each of the top 5 communities relative to the cross section through the tissue, where 0 represents the centre point of the tumor.

FIGS. 12A-12B show mean expression of PD-L1 (FIG. 12A) and MHC-II (FIG. 12B) on macrophages type 1 and type 2 for dataset 1 (left) and dataset 2 (right) in T/NA (left), T/M1 (center left), T/DC (center), T/M2_1 (center right) and T/M2_2 (right) communities for Vehicle and MRTX1257 treatment groups, values were log 2 scaled.

FIGS. 13A-13D show mean expression of PD-L1 (FIG. 13A) and CD86 (FIG. 13B) on dendritic cells and CD103⁺ dendritic cells for in T/NA (left), T/M1 (center left), T/DC (center), T/M2_1 (center right) and T/M2_2 (right) communities and Vehicle and MRTX1257 treatment groups for dataset 1 only (values were log 2 scaled). Mean expression of PD-L1 on dendritic cells and dendritic cells CD103 in communities A-E for Vehicle and MRTX1257 treatment groups, values were log 2 scaled (FIG. 13C). Mean expression of MHC-II on dendritic cells and dendritic cells CD103 for dataset 1 (left) and dataset 2 (right) in T/NA (left), T/M1 (center left), T/DC (center), T/M2_1 (center right) and T/M2_2 (right) communities for Vehicle and MRTX1257 treatment groups, values were log 2 scaled (FIG. 13D).

FIG. 14 show mean expression of CXCL9 on dendritic cells, CD103⁺ dendritic cells, macrophages type 1 and macrophages type 2 combined for communities A-E in Vehicle and MRTX-treated groups in dataset 2, values were log 2 scaled. Centre line shows median expression for each treatment group.

FIGS. 15A-15D show mean expression of Ki67 (FIG. 15A) and cleaved-caspase 3 (c-casp3; FIG. 15B) on tumor cells in T/DC (left), T/M2_1 (center) and T/M2_2 (right) communities following treatment with MRTX1257 for dataset 1, values were log 2 scaled. Mean expression of Ki67 (FIG. 15C) and cleaved-caspase 3 (c-casp3; FIG. 15D) on tumor cells in T/DC (left), T/M2_1 (center) and T/M2_2 (right) communities following treatment with MRTX1257 for dataset 2, values were log 2 scaled.

FIGS. 16A-16D show mean expression of PD-1 on CD8⁺ T cells in communities T/NA, T/M1, T/DC, T/M2_1 and T/M2_2 in Vehicle and MRTX1257-treated groups for dataset 1, values were log 2 scaled. Centre line shows median expression for each treatment group (FIG. 16A). Mean expression of PD-1 on CD8⁺ T cells for dataset 2 (FIG. 16B), CD4⁺ T cells for dataset 1 (left) and dataset 2 (right; FIG. 16C) and regulatory T cells for dataset 1 (left) and dataset 2 (right; FIG. 16D) in T/NA, T/M1, T/DC, T/M2_1 and T/M2_2 communities for Vehicle and MRTX-treated groups.

FIGS. 17A-17B shows mean expression of LAG-3 on T cells CD4⁺, T cells CD8⁺ and T reg cells in T/NA (left), T/M1 (center left), T/DC (center), T/M2_1 (center right) and T/M2_2 (right) communities for Vehicle and MRTX1257 treatment groups, values were log 2 scaled (FIG. 17A). Percentage of PD-1⁺ CD8⁺ T cells that are positive for LAG-3 expression (based on a mean expression threshold of 0.5), across T/NA, T/M1, T/DC, T/M2_1 and T/M2_2 communities following MRTX treatment (FIG. 17B).

FIGS. 18A-18C show minimum distance of dendritic cells and CD103⁺ dendritic cells that have ‘low’ or ‘high’ CXCL9 expression (based on a threshold of 0.5) to various T cells: PD-1⁺ CD8⁺ T cells within 800 pixels in the T/DC community from dataset 2, distance values were log 2 scaled (FIG. 18A); to PD-1⁺ CD4⁺ T cells (FIG. 18B); and PD-1⁺ regulatory T cells (FIG. 18C).

FIGS. 19A-19B show mean expression of Ki67 on CD4⁺, CD8⁺ and regulatory T cells within the T/DC community for Vehicle and MRTX1257 treatment groups from dataset 2, values were log 2 scaled (FIG. 19A). Number of times a c-casp3⁺ tumor cell is found in the 15-pixel neighborhood of a CD8⁺ T cell within the T/DC community, compared across Vehicle and MRTX1257 treatment groups for dataset 2, averaged per ROI. Count is relative to the proportion of tumor cells that were c-casp3⁺ in Vehicle vs MRTX1257 treatment groups. Each dot represents the value of one ROI (FIG. 19B).

FIGS. 20A-20B show number of times a c-casp3⁺ tumor cell is found in the 15-pixel neighborhood of a CD8⁺ T cells within T/DC, T/M2_1 or T/M2_2 communities following MRTX1257 treatment for dataset 1 (left) and dataset 2 (right). Values averaged across ROIs (FIG. 20A). Number of times a c-casp3⁺ tumor cell is found in the 15-pixel neighborhood of a CD4⁺ T cell (left) and regulatory T cell (right), within the T/DC community, compared across Vehicle and MRTX1257 treatment groups for dataset 2. Count is relative to the proportion of tumor cells that were c-casp3⁺ in Vehicle vs MRTX1257 treatment groups and averaged across ROIs (FIG. 20B).

FIGS. 21A-21E show log 2 fold changes in enrichment from neighbourhood analysis for CD8⁺ T cells in the T/DC community following treatment with MRTX1257. Filled circles represent images from which enrichment value was statistically significant compared to randomization of the spatial arrangements following treatment with MRTX1257 for dataset 2 (FIG. 21A). Log 2 fold changes in enrichment from neighbourhood analysis for CD8⁺ T cells in T/NA (FIG. 21B), T/M1 (FIG. 21C), T/M2_1 (FIG. 21D) and T/M2_2 (FIG. 21E) communities, following treatment with MRTX1257. Filled circles represent images from which enrichment value was statistically significant compared to randomization of the spatial arrangements within all top 5 communities following treatment with MRTX1257.

FIG. 22 shows count of regulatory T cells in the top 5 communities, split by Vehicle and MRTX1257 treatment groups for dataset 1 (left) and dataset 2 (right): T/NA (top), T/M1 (first from the top), T/DC (Second from the top); T/M2_1 (third from the top); and T/M2_2 (bottom).

FIG. 23 shows percentage of CD8⁺ T cells within the ‘Tregs’ and ‘No Tregs’ neighborhoods of T/DC community in MRTX1257 treatment group, averaged across ROIs.

FIG. 24 shows log 2 fold changes in enrichment from neighbourhood analysis for CD8⁺ T cells in ‘Tregs’ (top) and ‘No Tregs’ (bottom) neighborhoods within the T/DC community following treatment with MRTX1257. Filled circles represent images from which enrichment value was statistically significant compared to randomization of the spatial arrangements within the T/DC community following treatment with MRTX1257 for dataset 2.

FIG. 25 shows number of times a c-casp3⁺ tumor cell is found in the 15-pixel neighborhood of a CD8⁺ T cell within the T/DC community, compared across ‘Tregs’ and ‘No Tregs’ neighborhoods in dataset 2, averaged per ROI. Count is relative to the proportion of tumor cells that were c-casp3⁺ in ‘Treg’ vs ‘No Treg’ groups.

FIGS. 26A-26B shows log 2 fold changes in enrichment from neighbourhood analysis for CD4⁺ T cells in ‘Tregs’ (top) and ‘No Tregs’ (bottom) neighborhoods within T/DC community following treatment with MRTX1257. Filled circles represent images from which enrichment value was statistically significant compared to randomization of the spatial arrangements within T/DC community following treatment with MRTX1257 for dataset 2 (FIG. 26A). Number of times a c-casp3⁺ tumor cell is found in the 15-pixel neighborhood of a CD4⁺ T cell within T/DC community, compared across ‘Tregs’ and ‘No Tregs’ neighborhoods in dataset 2, averaged per ROI following MRTX1257 treatment. Count is relative to the proportion of tumor cells that were c-casp3⁺ in ‘Treg’ vs ‘No Treg’ groups (FIG. 26B).

FIGS. 27A-27B shows log 2 fold changes in enrichment from neighbourhood analysis for CD8⁺ T cells (FIG. 27A) and CD4⁺ T cells (FIG. 27B) in ‘Tregs’ (top) and ‘No Tregs’ (bottom) neighborhoods within T/M2_2 community following treatment with MRTX1257. Filled circles represent images from which enrichment value was statistically significant compared to randomization of the spatial arrangements within the T/M2_2 community following treatment with MRTX1257 for dataset 2.

FIG. 28 shows proportion of LUAD tumor cores (right panel) and LUSC tumor cores (left panel) that contain at least 25 cells/mm² of Treg communities (p1_C6, p1_C7, p1_C16, p1_C17, p1_C23, p1_C27) and p2_C1: Tumor border communities. 68 LUAD tumor cores from 40 patients and 47 LUSC tumor cores from 22 patients). Per-image median distance between cells of a community and their nearest tumor cell cluster. No co-occurrence is shown in the top from the middle bar, and co-occurrence is shown in the bottom from the middle bar.

FIGS. 29A-29B shows schematic outline of in vivo survival experiment in mice (FIG. 29A). Kaplan-Meier analysis of the overall survival of mice using the 3 Lewis Lung carcinoma model under Vehicle, anti-PD-1+ anti-CTLA-4, anti-PD-1+ anti-CCR8, MRTX+ anti-PD-1, MRTX+ anti-PD-1+ anti-CTLA-4, and MRTX+ anti-PD-1+ anti-CCR8 treatment groups (FIG. 29B).

FIGS. 30A-30D show tumor volume changes after 1 week of treatment for vehicle (left), anti-PD-1+ anti-CTLA-4 (first from the left), MRTX+ anti-PD-1 (second from the left), and MRTX+ anti-PD-1+ anti-CTLA4 (right) (FIG. 30A), after 2 weeks of treatment for MRTX+ anti-PD-1 (left), and MRTX+ anti-PD-1+ anti-CTLA4 (right) (FIG. 30B), and after 3 weeks of treatment for MRTX+ anti-PD-1 (left), and MRTX+ anti-PD-1+ anti-CTLA4 (right) (FIG. 30D) as measured by μCT scanning. Also shown are the differences between the first and second week as measured by μCT scanning for MRTX+ anti-PD-1 (left), and MRTX+ anti-PD-1+ anti-CTLA4 (right) (FIG. 30C).

FIGS. 31A-31F show percentage of all CD45⁺ cells identified as T regulatory cells (gated as CD45⁺ CD3⁺ CD4⁺ Foxp3⁺) measured by flow cytometry in the tumor (each dot represents a mouse, one-way ANOVA) (FIG. 31A). Percentage of all CD8⁺ T cells identified as effector memory CD8⁺ T cells (Tem) measured by flow cytometry in the tumor (each dot represents a mouse, one-way ANOVA) (FIG. 31B). Percentage of all CD45⁺ cells identified as CD8⁺ T cells measured by flow cytometry in the tumor (each dot represents a mouse, one-way ANOVA) (FIG. 31C). Percentage of all CD8⁺ T cells that are identified as PD-1⁺ measured by flow cytometry in the tumor (each dot represents a mouse, one-way ANOVA) (FIG. 31D). PD-1 signal from all CD8⁺ T cells measured by flow cytometry in the tumor (each dot represents a mouse, one-way ANOVA) (FIG. 31E). Percentage of all CD8⁺ T cells identified as LAG-3⁺ measured by flow cytometry in the tumor (each dot represents a mouse, one-way ANOVA) (FIG. 31F).

FIGS. 32A-32E show percentage of all CD45⁺ cells identified as T regulatory cells (gated as CD45⁺ CD3⁺ CD4⁺ Foxp3⁺) measured by flow cytometry in the tumor (each dot represents a mouse, one-way ANOVA; FIG. 32A). Percentage of all CD45⁺ cells identified as CD8⁺ T cells (gated as CD45⁺ CD3⁺ CD8⁺) (FIG. 32B) and CD4⁺ T cells (FIG. 32C), respectively, measured by flow cytometry (each dot represents a mouse, one-way ANOVA). Percentage of CD8⁺ T cells that are PD-1⁺ (FIG. 32D) and CD39⁺ (FIG. 32E), respectively, measured by flow cytometry in the tumor.

FIGS. 33A-33B show distribution of CD8⁺ T cells in the tumor that are TCF1⁺ Naïve CD8 (bottom), PD-1⁺ TCF1⁺ stem cell like CD8 (first from the bottom), PD-1⁺ TIM3^{low}CD39^{low} transitory effector CD8 (second from the bottom) and PD-1⁺ TIM3^{high}CD39^{high} exhausted CD8 (third from the bottom) and other (top) across treatment groups (FIG. 33A). Percentage of DC1s in the tumor that are CD86⁺ measured by flow cytometry (FIG. 33B).

FIG. 34 shows representative flow cytometry contour plots for CD4⁺ FOXP3⁺ Treg cells in different treatment groups.

FIGS. 35A-35B shows percentage of all CD45⁺ cells identified as regulatory T cells (gated as CD45⁺ CD3⁺ CD4⁺ Foxp3⁺) measured by flow cytometry in the tumor draining lymph nodes (each dot represents a mouse, one-way ANOVA) (FIG. 35A). Histogram of CTLA-4 expression on FOXP3⁺ Tregs following MRTX treatment in the tumor (right peak) or tumor draining lymph node (left peak) (FIG. 35B).

FIG. 36 shows the percentage of CD4⁺ T cells, CD8⁺ T cells and Treg cells that are PD-1⁺, measured by flow cytometry in the lymph nodes (upper panel), and the percentage of CD4⁺ T cells, CD8⁺ T cells and Treg cells that are Ki67″, measured by flow cytometry in the lymph nodes (lower panel).

### DETAILED DESCRIPTION OF THE DISCLOSURE

In order that the present disclosure can be more readily understood, certain terms are defined throughout in the present application.

In addition, various aspects of the present disclosure are described in further detail in the sections titled “Method of treating a tumor comprising administering a KRAS inhibitor and a Treg-depleting agent”, “Kits”, and “Further methods of the present disclosure” below. As will be acknowledged by a person of ordinary skill in the art to which this disclosure belongs, all of the various aspects described herein can be combined in any and all variations.

In general, the headings provided herein are not to be understood as limiting the various aspects of the disclosure. Accordingly, the terms defined herein are more fully defined by reference to the specification in its entirety.

All publications, patents, and patent applications mentioned in this specification are herein incorporated by reference to the same extent as if each individual publication, patent, or patent application was specifically and individually indicated to be incorporated by reference.

### I. Definitions

As used in this application, except as otherwise expressly provided herein, each of the following terms shall have the meaning set forth below. Additional definitions are set forth throughout the specification. Unless defined otherwise in this application, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

As used herein, the terms “a,” “an,” and “the” mean “one or more” and include the plural unless the context is inappropriate.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred, with the proviso that if the nominal value is a percentage (%), the value cannot exceed 100%. Thus, for example, the term “about 99%” refers to “89.1% to 100%.”

As used herein, the expression “and/or” in connection with two or more recited objects includes individually each of the recited objects and the various combinations of two or more of the recited objects, unless otherwise understood from the context and use. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

Units, prefixes, and symbols are denoted in their Système International de Unites (SI) accepted form.

As described herein, numeric ranges (such as concentration ranges, percentage ranges, or ratio ranges) are inclusive of the values defining the range. Further, any numeric range is to be understood to include the value of any integer within the recited range and, when appropriate, fractions thereof (such as one-tenth and one-hundredth of an integer), unless otherwise indicated.

Unless otherwise indicated, the nucleotide sequences disclosed in the specification are written left to right in 5′ to 3′ orientation and the amino acid sequences are written left to right in amino (N) to carboxy (C) orientation.

“Administering,” “administer” or “administration” refers to the physical introduction of a therapeutic agent to a subject, using any of the various methods and delivery systems known to those skilled in the art. Routes of administration include parenteral and non-parenteral administration. Parenteral administration modes (i.e., modes of administration other than enteral and topical administration) include, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Non-parenteral administration modes include, without limitation, topical, epidermal or mucosal administration, for example, intranasally, orally, vaginally, rectally, sublingually or topically. In some aspects, the antibodies of the present disclosure (such as the anti-CCR8, anti-CTLA-4, anti-PD-1, and/or anti-PD-L1 antibodies described herein) are administered via parenteral administration mode (such as by injection or infusion). In some aspects, the antibodies of the present disclosure (such as the anti-CCR8, anti-CTLA-4, anti-PD-1, and/or anti-PD-L1 antibodies described herein) are administered by intravenous, intraperitoneal, intramuscular, subcutaneous, or spinal administration. In some aspects, the antibodies of the present disclosure (such as the anti-CCR8, anti-CTLA-4, anti-PD-1, and/or anti-PD-L1 antibodies described herein) are administered by intravenous administration. In some aspects, the antibodies of the present disclosure (such as the anti-CCR8, anti-CTLA-4, anti-PD-1, and/or anti-PD-L1 antibodies described herein) are administered by subcutaneous administration. The antibodies of the present disclosure may be administered by injection or infusion. In some aspects, the KRAS inhibitors of the present disclosure (such as the KRAS G12C inhibitors, for example, adagrasib, sotorasib, or MRTX1257) are administered orally, optionally in the form of a tablet. Administering can be performed once or a plurality of times (i.e., more than once) over a treatment period (which might comprise one or more treatment cycles). In some aspects, administration occurs once daily over a treatment period. In some aspects, administration occurs twice daily over a treatment period. In some aspects, administration occurs every week, every two weeks, every three weeks, or every four weeks over a treatment period. Administering may also include prescribing a medication, even if the medication is actually delivered by another medical professional, or by the patient himself or herself. Dosing and administration can be performed for any number of cycles of treatment throughout a treatment period, from one, two, three, four cycles, etc., up to continuous treatment (repeating the dosing until no longer necessary, disease recurrence, or unacceptable toxicity is reached). For a combination therapy, one cycle comprises at least one dose of each therapeutic of the combination therapy (such as a KRAS inhibitor and a Treg-depleting agent).

“Regulatory T cells” are abbreviated herein as “Tregs” or as “Treg cells.” Consequently, a “regulatory T cell” is abbreviated herein as “Treg” or as “Treg cell.” Tregs are a subpopulation of T cells that modulate the immune system, maintain tolerance to self-antigens, and/or prevent autoimmune disease. In addition to expressing the CD4 molecule, CD4⁺ Tregs are characterized by high expression of the transcription factor forkhead box P3 (Foxp3). In general, the term “Treg” or “Tregs” as used herein refers to a CD4⁺ FOXP3⁺ Treg or to CD4⁺ FOXP3⁺ Tregs, respectively. The Treg may further be characterized as a CD4⁺ CD45⁺ FOXP3⁺ Treg, a CD4⁺ CD25⁺ FOXP3⁺ Treg, a CD45⁺ CD4⁺ CD25⁺ FOXP3⁺ Treg, a CD4⁺ CD3⁺ FOXP3⁺ Treg, or a CD45⁺ CD4⁺ CD3⁺ FOXP3⁺ Treg. Tregs suppress the immune system via multiple mechanisms including downregulating the induction and proliferation of effector T cells, secretion of chemokines and inhibitory cytokines, and suppression of dendritic cell maturation and function. Thus, Tregs not only have the potential to protect against autoimmunity in maintaining self-tolerance, but also impede effective tumor immunity by inhibiting tumor-specific T cell responses and promoting tumor growth. In some aspects, the Treg is a tumor-infiltrating Treg. In some aspects, the Treg expresses CCR8. In some aspects, the Treg is a tumor-infiltrating Treg that expresses CCR8. In some aspects, the Treg expresses CTLA-4. In some aspects, the Treg is a tumor-infiltrating Treg that expresses CTLA-4.

The term “Treg-depleting agent” refers to an agent that targets Tregs for functional inhibition and/or clearance. Tregs express receptors which can be targeted to reduce the activity and/or frequency of Tregs and subsequently enhance anti-tumor immunity. A Treg-depleting agent may exhibit one or more of the following effects: reduce the frequency, reduce the function, block mobilization, enable phagocytosis, enable polarization, and potentiate differentiation of Tregs to non-Tregs. Examples of Treg-depleting agents are such that are capable of specifically binding to C-C motif chemokine receptor 8 (CCR8), cytotoxic T-lymphocyte associated protein 4 (CTLA-4), C-C motif chemokine receptor 4 (CCR4), CD25, T cell immunoglobulin and mucin domain-containing protein 3 (TIM-3), V-domain Ig suppressor of T cell activation (VISTA), glucocorticoid-Induced TNFR-Related protein (GITR), 4-1BB (also known as TNFRSF9 or CD137), OX-40, CD27, inducible T cell co-stimulator (ICOS), CD15s (sialyl Lewis x), Mucosa-associated lymphoid tissue lymphoma translocation protein 1 (MALT1), tumor necrosis factor receptor 2 (TNFR2), TGF-β receptor, and/or T cell immunoreceptor with Ig and ITIM domains (TIGIT). In some aspects, the Treg-depleting agent binds to human CCR8, CTLA-4, CCR4, CD25, TIM-3, VISTA, GITR, 4-1BB, OX-40, CD27, ICOS, CD15s (sialyl Lewis x), MALT1, TNFR2, TGF-receptor, and/or TIGIT. In some aspects, the Treg-depleting agent is capable of specifically binding to CCR8 and/or CTLA-4. In some aspects, the Treg-depleting agent is capable of specifically binding to human CCR8 and/or CTLA-4. In some aspects, the Treg-depleting agent is capable of specifically binding to CCR8. In some aspects, the Treg-depleting agent is capable of specifically binding to human CCR8. In some aspects, the Treg-depleting agent comprises an anti-CCR8 antibody. In some aspects, the Treg-depleting agent comprises an anti-CTLA-4 antibody. In some aspects, the Treg-depleting agent comprises an anti-human-CCR8 antibody. In some aspects, the Treg-depleting agent comprises an anti-human-CTLA-4 antibody. In some aspects, the Treg-depleting agent specifically depletes Tregs infiltrating into tumor tissues (by functional inhibition and/or clearance of Tregs) but does not affect tumor-reactive effector T cells and/or peripheral Tregs.

A Treg-depleting agent that is capable of specifically binding to a target, such as a protein on the surface of a Treg, for example, CCR8 and/or CTLA-4, binds to said target with high affinity, reflected by a dissociation constant (K_{D}) of 1 μM to 10 pM or lower. In general, any K_{D} greater than about 100 μM is considered to indicate nonspecific binding. As used herein, a Treg-depleting agent that “specifically binds” to a protein refers to a Treg-depleting agent that binds to the protein and substantially identical proteins with high affinity, which means having a K_{D} of about 100 nM or lower, about 10 nM or lower, about 5 nM or lower, or between about 5 nM and 0.1 nM or lower. A protein is “substantially identical” to a given protein if it exhibits a high degree of sequence identity to the given protein, for example, if it exhibits at least 80%, at least 90%, at least 95%, at least 97%, or at least 99% sequence identity to the sequence of the given protein. The “K_{D}” refers to the dissociation constant for a particular Treg-depleting agent-target interaction, which is obtained from the ratio of k_{off} to kₒₙ (i.e., k_{off}/kₒₙ) and is expressed as a molar concentration (e.g., nM). The term “kₒₙ” refers to the association rate or “on rate” for the association of a Treg-depleting agent and its target, whereas the term “k_{off}” refers to the dissociation rate for the Treg-depleting agent-target complex. K_{D} values for Treg-depleting agents can be determined using methods well established in the art, such as surface plasmon resonance (SPR), kinetic exclusion assay (KinExA®; Sapidyne Instruments, Boise, ID), or bio-layer interferometry (BLI; ForteBio, Fremont, CA). An exemplary SPR method uses a BIACORE® biosensor system (GE Healthcare, Chicago, IL).

Regarding general information on Tregs and their role in cancer, as well as Treg-depleting agents, reference is also made, for example, to Chen et al. (2022) Int. Immunopharmacol. 104:108469; Huppert et al. (2022) Cell Mol. Immunol. 19 (1): 33-45; Tanaka et al. (2019) Eur. J. Immunol. 49:1140-1146; and Iglesias-Escudero et al. (2023) Mol. Cancer 22:26; all of which herewith incorporated by reference.

The term “monotherapy” refers to a single type of treatment such as, for example, the administration to a patient of a single drug (such as a KRAS inhibitor), or the use of radiation therapy or surgery alone, to treat a disease or condition. The administration of a drug by itself does not constitute monotherapy if in the same course of treatment (i.e., in the same treatment period) it is preceded or followed by another type of treatment for the disease or condition, such as the administration of an additional drug.

In contrast, “combination therapy” refers to a treatment modality that combines at least two types of therapy such as, for example, the administration to a patient of two or more drugs (such as a KRAS inhibitor and a Treg-depleting agent), or the administration of a drug plus radiation therapy or surgery, to treat a disease or condition. These two or more treatments can, but need not be administered concurrently to the patient but are part of the same course of treatment (i.e., of the same treatment period). In some aspects, the different therapies are administered concurrently. In some aspects, the different therapies are administered sequentially. If the combination therapy comprises two or more drugs (such as a KRAS inhibitor and a Treg-depleting agent), the two or more drugs can, but need not be administered as a mixture. In some aspects, the two or more drugs are administered separately. In some aspects, the two or more drugs are administered as a mixture. The dose and dosing interval of a first component of the combination therapy is usually based on the dose and dosing interval of a second or further component of the combination therapy, to elicit an overall therapeutic benefit. Although combination therapy is most convenient when dosing schedules are the same or multiples of one another (e.g. Q4W and Q8W), it also encompasses administration on different days if dosing intervals do not align for any given cycle.

An “antibody” refers to an immunoglobulin which is capable of specifically binding to an antigen. An antibody comprises at least two heavy chains and two light chains interconnected by disulfide bonds. In some aspects, the antibody comprises two and no more than two heavy chains and/or two and no more than two light chains. Each heavy chain comprises a heavy chain variable domain (abbreviated herein as VH), and a heavy chain constant region. The heavy chain constant region comprises three heavy chain constant domains, CH1, CH2 and CH3. A heavy chain can have the C-terminal lysine or not. In some aspects, the antibody of the present disclosure comprises the C-terminal lysine. In some aspects, the antibody of the present disclosure does not comprise the C-terminal lysine. The amino acid sequence of the IgG1 heavy chain constant region is given in SEQ ID NO: 166 (Table 1). In some aspects, the antibody comprises a heavy chain comprising the IgG1 heavy chain constant region as set forth in SEQ ID NO: 166. Each light chain comprises a light chain variable domain (abbreviated herein as VL) and a light chain constant region. The light chain constant region comprises one light chain constant domain, CL. The amino acid sequence of the kappa light chain constant region is given in SEQ ID NO: 167 (Table 1). In some aspects, the antibody comprises a light chain comprising the kappa light chain constant region as set forth in SEQ ID NO: 167. The VH and VL regions can be further subdivided into regions of hypervariability, termed complementarity determining regions (CDRs), interspersed with regions that are more conserved between antibodies, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain CDRs are abbreviated herein as CDRH1, CDRH2, and CDRH3. The light chain CDRs are abbreviated herein as CDRL1, CDRL2, and CDRL3. The variable regions of the heavy and light chains comprise a binding domain that interacts with an antigen. A variety of methods have been used to delineate the CDR domains within an Ab, including the Kabat, Chothia, AbM, contact, and IMGT definitions. The constant regions of the antibodies can mediate the binding of the immunoglobulin to host tissues or factors, including various cells of the immune system (e.g., effector cells) and the first component (C1q) of the classical complement system. In some aspects, wherein the antibody is an activatable antibody, the VL may further comprise a masking moiety, a cleavable moiety, a spacer element and optionally other sequence elements as described herein.

TABLE 1
Sequences of IgG1 constant region
and kappa constant region.

| IgG1 | ASTKGPSVFPLAPSSKSTSGGTAALGCLVK |
|---|---|
| constant | DYFPEPVTVSWNSGALTSGVHTFPAVLQSS |
| region | GLYSLSSVVTVPSSSLGTQTYICNVNHKPS |
| (SEQ ID | NTKVDKRVEPKSCDKTHTCPPCPAPELLGG |
| NO: 166) | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPG |
| Kappa | RTVAAPSVFIFPPSDEQLKSGTASVVCLLN |
| constant | NFYPREAKVQWKVDNALQSGNSQESVTEQD |
| region | SKDSTYSLSSTLTLSKADYEKHKVYACEVT |
| (SEQ ID NO: | HQGLSSPVTKSFNRGEC |
| 167) | |

In some aspects, the heavy chains and/or the light chains of an antibody are (substantially) identical. In some aspects, the heavy chains and/or the light chains of an antibody are distinct (as in, for example, bispecific antibodies). In some aspects, the antibody comprises two and no more than two (substantially) identical heavy chains and two and no more than two (substantially) identical light chains. Antibody chains are substantially identical (but not entirely identical) if, for example, they differ due to post-translational modifications, such as C-terminal cleavage of lysine residues, alternative glycosylation patterns, etc.

An antibody that is capable of specifically binding to an antigen (e.g., CTLA-4 or CCR8) is referred to herein as an anti-antigen antibody (e.g. an “anti-CTLA-4 antibody” or an “anti-CCR8 antibody”). The antigen may be from human origin (e.g. an “anti-human-CTLA-4 antibody” or an “anti-human-CCR8 antibody”). As will be acknowledged by a skilled person in the art, the antibody applied in the methods of the present disclosure, in which the subject is defined to be a human, is an antibody that specifically binds to (at least) the human antigen. Such antibodies may or may not bind to the corresponding antigen from other species (such as mouse). For example, an anti-CTLA-4 antibody specifically binds to CTLA-4, an anti-CCR8 antibody specifically binds to CCR8, an anti-PD-1 antibody binds specifically to PD-1, an anti-PD-L1 antibody binds specifically to PD-L1, and an anti-LAG-3 antibody binds specifically to LAG-3.

An antibody that is capable of specifically binding to an antigen binds to said antigen with high affinity, reflected by a dissociation constant (K_{D}) of 1 μM to 10 pM or lower. In general, any K_{D} greater than about 100 μM is considered to indicate nonspecific binding. As used herein, an antibody that “specifically binds” to an antigen refers to an antibody that binds to the antigen and substantially identical antigens with high affinity, which means having a K_{D} of about 100 nM or lower, about 10 nM or lower, about 5 nM or lower, or between about 5 nM and 0.1 nM or lower. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. The “K_{D}” refers to the dissociation constant for a particular antibody-antigen interaction, which is obtained from the ratio of k_{off} to kₒₙ (i.e., k_{off}/kₒₙ) and is expressed as a molar concentration (e.g., nM). The term “kₒₙ” refers to the association rate or “on rate” for the association of an antibody and its antigen, whereas the term “k_{off}” refers to the dissociation rate for the Ab-antigen complex. K_{D} values for antibodies can be determined using methods well established in the art, such as surface plasmon resonance (SPR), kinetic exclusion assay (KinExA®; Sapidyne Instruments, Boise, ID), or bio-layer interferometry (BLI; ForteBio, Fremont, CA). An exemplary SPR method uses a BIACORE® biosensor system (GE Healthcare, Chicago, IL).

The antibody may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class or subclass (e.g., IgM or IgG1) that is encoded by the heavy chain constant region genes. In some aspects, the antibody comprises a heavy chain constant region which is of a human IgG1 isotype. In some aspects, the antibody comprises a heavy chain constant region which is of a human IgG3 isotype. Human IgG1 and IgG3 Ab isotypes are able to mediate antibody-dependent cellular cytotoxicity (ADCC) through binding to activating Fcγ receptors, particularly the CD16 (FcγRIIIa) receptor expressed by human NK cells and monocytes. Additionally, therapeutic IgG1 Abs have long-term stability in blood mediated via binding to the neonatal Fc receptor (FcRn).

The term “antibody” includes both naturally occurring and non-naturally occurring antibodies, including allotypic variants; monoclonal and polyclonal antibodies; chimeric and humanized antibodies; human or non-human antibodies; wholly synthetic antibodies; and single chain antibodies. The term “antibody” also includes monospecific antibodies, bispecific antibodies, and multispecific antibodies. As used herein, a “bispecific antibody” is an antibody that is capable of specifically binding two antigens, wherein the first and second antigen are different. As used herein, a “multispecific” antibody is capable of specifically binding more than one antigen, e.g., at least two (i.e., a “bispecific” antibody), at least three (i.e., a “trispecific” antibody), at least four, at least five, or at least six antigens. In some aspects, the antibody is a monospecific antibody. In some aspects, the antibody is a bispecific antibody. In some aspects, the antibody is a monoclonal antibody. In some aspects, the antibody is a chimeric antibody. In some aspects, the antibody is a humanized antibody. In some aspects, the antibody is a human antibody. In some aspects, the antibody is a human IgG1 antibody. In some aspects, the antibody is a nonfucosylated antibody. In some aspects, the antibody is an activatable antibody. An activatable antibody may also be referred to as a “probody.” In some aspects, the antibody is an isolated antibody.

The term “monoclonal antibody” refers to a preparation of antibody molecules of single molecular composition, i.e., antibody molecules whose primary amino acid sequences are identical or essentially identical, and which exhibit a single binding specificity and affinity for a particular epitope. Monoclonal antibodies may be produced by hybridoma, recombinant means, transgenic animals or other techniques known to those skilled in the art.

A “human antibody” refers to an antibody having variable regions in which both the framework and CDR regions are derived from human germline immunoglobulin sequences. Furthermore, the constant region also is derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo). However, in contrast to a “humanized antibody,” a “human antibody,” as used herein, does not encompass antibodies in which CDR sequences derived from the germline of another mammalian species, such as a mouse, have been grafted onto human framework sequences.

A “humanized antibody” refers to an antibody in which some, most, or all of the amino acids outside the CDRs of a non-human antibody are replaced with corresponding amino acids derived from human immunoglobulins. A “humanized antibody” retains an antigenic specificity similar to that of the original antibody. In some aspects, the humanized antibody refers to an antibody in which all of the amino acids outside the CDRs of a non-human antibody are replaced with corresponding amino acids derived from human immunoglobulins.

A “chimeric antibody” refers to an antibody in which the variable antibody regions are derived from one species and the constant antibody regions are derived from another species, such as an antibody in which the variable regions are derived from a mouse antibody and the constant regions are derived from a human antibody.

An “activatable antibody” refers to a modified form of an antibody that binds to targets of therapeutic interest wherein the antibody comprises structural modifications that inhibit binding to the target until cleaved by proteases more prevalent and/or active in the tumor microenvironment than in peripheral tissue. Thus, activatable antibodies can reduce peripheral toxicity at a given dose, allowing higher (and thus potentially more efficacious) doses for any given level of toxicity, as the antibody is selectively cleaved (and thereby activated) at the tumor site. In some aspects, the activatable antibody comprises a light chain variable domain that further comprises a masking moiety and a cleavable moiety at the amino-terminus, as disclosed, for example, in WO 2018/085555 or in Table 2. If the activatable antibody comprises two light chain variable domains that further comprise a masking moiety and a cleavable moiety at the amino-terminus, the activatable antibody in vivo can exist as a mixture of inactive/uncleaved, mono-cleaved, and dual-cleaved forms. In some aspects, the activatable antibody comprises a light chain variable domain that further comprises a spacer, a masking moiety, and a cleavable moiety at the amino-terminus, as disclosed, for example, in WO 2018/085555 or in Table 2.

TABLE 2
Spacer, masking moiety, and cleavable
moieties suitable for the activatable
antibodies of the present disclosure.

| | |
|---|---|
| Spacer | QGQSGS |
| (SEQ ID NO: 168) | |
| Masking moiety YV39 | CRTQLYGYNLCPY |
| (SEQ ID NO: 169) | |
| Cleavable moiety 2001 | ISSGLLSGRSDNH |
| (SEQ ID NO: 170) | |
| Cleavable moiety 2011 | ISSGLLSGRSDNP |
| (SEQ ID NO: 171) | |
| Cleavable moiety 2012 | ISSGLLSGRSANP |
| (SEQ ID NO: 172) | |

A “nonfucosylated antibody” is an antibody that does not comprise a core fucose residue on the N-linked glycan at position N297 (EU numbering convention; see the EU index as in Kabat et al. (1991) Sequences of Proteins of Immunological Interest, National Institutes of Health, Bethesda, MD). The absence of core fucose residues strongly enhances antibody-dependent cellular cytotoxicity (ADCC) via improved binding of IgG to activating FcγRIIIA (CD16) without altering antigen binding or complement-dependent cytotoxicity (CDC). Natsume et al. (2009) Drug Des. Devel. Ther. 3:7. Binding of a nonfucosylated antibody to Tregs facilitates engagement of the FcγR on NK cells and myelomonocytic Teffs. This FcγR engagement drives NK cell activation leading to enhanced Treg killing by ADCC. There is convincing evidence that afucosylated tumor-specific antibodies translate into enhanced therapeutic activity in mouse models in vivo. See Nimmerjahn & Ravetch (2005) Science 310:1510; Mossner et al. (2010) Blood 115:4393. In some aspects, the antibody comprises an IgG1 heavy chain constant region that does not comprise a core fucose residue on the N-linked glycan at position N297.

Modification of antibody glycosylation can be accomplished by, for example, expressing the antibody in a host cell with altered glycosylation machinery. Cells with altered glycosylation machinery have been described in the art and can be used as host cells in which to express antibodies with altered glycosylation. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87 (15): 614), such that antibodies expressed in these cell lines lack fucose on their carbohydrates. As another example, EP1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 2003/035835 describes a variant CHO cell line, Lec13, with reduced ability to attach fucose to Asn (297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta (1,4)-N-acetylglucosaminyltransferase III (GnTIII)) such that antibodies expressed in the engineered cell lines exhibit increased bisecting GlcNac structures which results in increased Fcγ receptor binding, and thus enhanced ADCC activity, of the antibodies. See also Umaña et al. (1999) Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein. U.S. Pat. No. 8,163,551; WO 2009/135181.

Nonfucosylated antibodies exhibit greatly increased biological activity due to enhanced Fcγ receptor binding, when compared with fucosylated antibodies. Thus, antibody preparations that contain not only nonfucosylated antibodies but that also contain a certain portion of fucosylated antibodies are suitable for the methods of the present disclosure. Residual levels of fucosylated heavy chains will not significantly reduce the biological activity, such as ADCC, of an antibody preparation that comprises mostly nonfucosylated heavy chains. Nonfucosylated antibodies may exhibit ten-fold higher affinity for CD16, and up to 30- to 100-fold enhancement of ADCC activity, so even a small increase in the proportion of nonfucosylated antibodies may drastically increase the ADCC activity of a preparation. In some aspects, an antibody preparation (for example, comprising an anti-CCR8 or an anti-CTLA-4 antibody) that can be administered to the subject in the method of the present disclosure comprises greater than about 95% nonfucosylated antibody heavy chains, such as about 100%. In some aspects, the antibody preparation comprises less than or equal to about 95% heavy chains lacking fucose, e.g. antibody preparations in which between about 80 and about 95% of heavy chains lack fucose, such as between about 85% and about 95%, and between about 90% and 95%. In some aspects, the antibody preparation comprises about 5%, about 10%, about 20%, about 30%, about 40%, about 50%, about 60%, about 70%, about 80%, about 90%, about 95%, or about 100% of heavy chains lacking fucose.

The term “nonfucosylated antibody,” as used herein, thus refers also to a multiplicity of antibodies (such as antibodies in an antibody preparation), of which greater than 95% of the heavy chains are nonfucosylated. In some aspects, 100% of the antibody heavy chains are nonfucosylated.

In some aspects, nonfucosylated antibodies and/or antibody preparations comprising nonfucosylated antibodies are produced in cells lacking an enzyme essential to fucosylation, such as FUT8 (e.g. U.S. Pat. No. 7,214,775), or in cells in which an exogenous enzyme partially depletes the pool of metabolic precursors for fucosylation (e.g. U.S. Pat. No. 8,642,292), or in cells cultured in the presence of a small molecule inhibitor of an enzyme involved in fucosylation (e.g. WO 2009/135181).

The level of fucosylation in an antibody preparation may be determined by any method known in the art, including but not limited to gel electrophoresis, liquid chromatography, and mass spectrometry. In some aspects, the level of fucosylation in an antibody preparation is determined by hydrophilic interaction chromatography (or hydrophilic interaction liquid chromatography, HILIC). To determine the level of fucosylation of an antibody preparation, samples are denatured, treated with PNGase F to cleave N-linked glycans, and analyzed for fucose content. LC/MS determination of full-length antibody chains is an alternative method to detect the level of fucosylation of an antibody preparation, but mass spectroscopy is inherently less quantitative.

An exemplary method for analyzing the fucosylation of an antibody is described in the following. Antibodies are first denatured using urea and then reduced using DTT (dithiothreitol). Samples are then digested overnight at 37° C. with PNGase F to remove N-linked glycans. Released glycans are collected, filtered, dried, and derivatized with 2-aminobenzoic acid (2-AA) or 2-aminobenzamide (2-AB). The resulting labeled glycans are then resolved on a HILIC column and the eluted fractions are quantified by fluorescence, and dried. The fractions are then treated with exoglycosidases, such as a (1-2,3,4,6) fucosidase (BKF), which releases core a (1,6)-linked fucose residues. Untreated samples and BKF-treated samples are then analyzed by liquid chromatography. Glycans comprising a (1,6)-linked fucose residues exhibit altered elution after BKF treatment, whereas nonfucosylated glycans are unchanged. The oligosaccharide composition is also confirmed by mass spectrometry. See, e.g., Zhu et al. (2014) MAbs 6:1474. Percent nonfucosylation is calculated as one hundred times the molar ratio of (glycans lacking a fucose a1,6-linked to the first GlcNac residue at the N-linked glycan at N297 (EU numbering) of the antibody heavy chain) to (the total of all glycans at that location (glycans lacking fucose and those having a1,6-linked fucose)

As outlined above, as nonfucosylated antibodies exhibit greatly increased biological activity, in some aspects, the antibody may also be a hypofucosylated antibody. A “hypofucosylated antibody” refers to a multiplicity of antibodies (such as antibodies in an antibody preparation), of which 95% or less of the heavy chains lack fucose. In some aspects, between 80% and 95% of the heavy chains lack fucose. In some aspects, between 90% and 95% of the heavy chains lack fucose. In some aspects, between 80% and 85% of the heavy chains lack fucose.

An “antigen binding portion” of an antibody refers to any portion of an antibody less than the whole antibody (i.e., the full-length antibody) that retains the ability to specifically bind to the antigen bound by the whole antibody. Examples of antigen binding portions encompassed within the term “antigen binding portion” of an antibody, e.g., an anti-CTLA-4 antibody, an anti-CCR8-antibody, an anti-PD-1 antibody, an anti-PD-L1 antibody, or an anti-LAG-3 antibody described herein, include (i) a Fab fragment (fragment from papain cleavage) or a similar monovalent fragment consisting of the VL, VH, LC and CHI domains; (ii) a F(ab′)₂ fragment (fragment from pepsin cleavage) or a similar bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a single domain antibody (dAb) fragment (Ward et al. (1989) Nature 341:544-546), which consists of a VH domain; (vi) an isolated complementarity determining region (CDR), (vii) a combination of two or more isolated CDRs which can optionally be joined by a synthetic linker, (viii) a bi-single domain antibody which consists of two VH domains linked by a hinge (dual-affinity re-targeting antibodies (DARTs)); and (ix) a dual variable domain immunoglobulin. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Antigen binding portions of antibodies can be produced by recombinant DNA techniques, or by enzymatic or chemical cleavage of intact immunoglobulins.

Within the meaning of the present disclosure, when reference is made to an antibody heavy chain or light chain, or an antibody VH or VL, or CDRs of an antibody, the phrase “comprising the amino acid sequence (as) set forth in,” can be used interchangeably with the phrase “comprising consecutively linked amino acids having the sequence (as) set forth in.” For example, the expression “a VH comprising a CDRH1 comprising the amino acid sequence as set forth in SEQ ID NO: 1” can be used interchangeably with the expression “a VH comprising a CDRH1 comprising consecutively linked amino acids having the sequence as set forth in SEQ ID NO: 1.”

The term “tumor” refers to benign (non-cancerous) or malignant (cancerous) tumors, including pre-cancerous lesions. In some aspects, the tumor is a malignant tumor. A malignant tumor is the result of unregulated cancer cell division and/or growth. The term “tumor” includes solid and liquid tumors. Liquid tumors may also be referred to as hematological malignancies, including all types of leukemias, lymphomas, and myeolomas. In some aspects, the tumor is a solid tumor. A solid tumor refers to any mass of tissue that results from excessive cell growth or proliferation. A cancerous solid tumor is the result of cancer cells forming a lump or growth. In some aspects, the (malignant) tumor is metastatic. In some aspects, the (malignant) tumor is refractory. In some aspects, the (malignant) tumor is relapsed. In some aspects, the (malignant) tumor is (locally) advanced. In aspects of the present disclosure, wherein the tumor is described to be a certain type of cancer (such as a tumor being a lung cancer), reference is made to a tumor which is derived from that certain type of cancer. In some aspects, the tumor carries one or more mutations in KRAS, such as G12C and/or a G12D mutations. In some aspects, the tumor carries a G12C mutation. In some aspects, the tumor is a lung cancer. In some aspects, the tumor is a NSCLC.

An “inhibitor,” “blocking agent,” and “antagonist” generally includes, without limitation, any molecule capable of blocking, reducing, or otherwise limiting an interaction or activity of a target molecule (e.g., LAG-3, PD-1, or PD-L1). In some aspects, the antagonist is an antibody or antigen binding portion thereof. In some aspects, the antagonist is a small molecule.

“KRAS,” as used herein, refers to the Kirsten rat sarcoma virus oncogene. KRAS, as used herein, may refer to the genetic locus (the gene) or the gene product, depending on the context. Generally, when referring to the genetic locus, “KRAS” is used italicized (“KRAS”). As will be acknowledged by a person skilled in the art, in aspects of the present disclosure, wherein the subject is a human, the term “KRAS” (such as, in “KRAS” inhibitor) refers to human KRAS. The canonical human KRAS amino acid sequence is set forth in SEQ ID NO: 173 (Table 3). The KRAS gene is a member of the rat sarcoma viral oncogene family (RAS), which includes two other isoforms in humans: the Harvey and neuroblastoma rat sarcoma viral oncogenes (HRAS, NRAS). KRAS proteins function as a finely regulated molecular switch that controls multiple signalling cascades by cycling between activated and inactivated conformations. KRAS proteins can be activated by growth factors, chemokines, Ca²⁺ or receptor tyrosine kinase (RTK). Activated KRAS protein can in turn activate multiple signalling pathways, including the rapidly accelerated fibrosarcoma (RAF)-mitogen-activated protein kinase (MEK)-extracellular regulated protein kinases (ERK) signalling pathway (RAF/MEK/ERK signalling pathway), phosphoinositide 3-kinase (PI3K)-protein kinase B (AKT)-mammalian target of rapamycin (mTOR) signalling pathway (PI3K/AKT/mTOR signaling pathway signalling pathway), and other signalling pathways, revealing a wide range of KRAS communications with multiple signalling pathways.

TABLE 3
Canonical human KRAS sequence
(SEQ ID NO: 173; Uniprot ID: P01116-1).

MTEYKLVVVGAGGVGKSALTIQLIQNHFVDEYDPTIEDSYRKQVV
IDGETCLLDILDTAGQEEYSAMRDQYMRTGEGFLCVFAINNTKSF
EDIHHYREQIKRVKDSEDVPMVLVGNKCDLPSRTVDTKQAQDLAR
SYGIPFIETSAKTRORVEDAFYTLVREIRQYRLKKISKEEKTPGC
VKIKKCIIM

KRAS (such as human KRAS) may have one or more mutations (e.g. amino acid substitutions, deletions, insertions, frame-shifts, nonsense or gene amplification) compared to the wild-type KRAS sequence, wherein the one or more mutations are associated with cancer (i.e., the KRAS may be a “mutant KRAS”). In some aspects, the KRAS (such as human KRAS) comprises one amino acid substitution compared to the wild-type KRAS sequence (such as the canconical human KRAS sequence described herein). A “mutant KRAS” does not encompass sporadic mutations in KRAS that are not known to be associated with cancer but may be found in individual subjects. Exemplary mutations in KRAS that are associated with cancers, such as NSCLC, include, but are not limited to, G12C, G12C, G12V, G12D, G12A, G12S, G12R, G12F, G13C, G13D, Q61L, Q61H, Q61K, A146T, and L19F. The most common mutations in NSCLC are G12C, G12V and G12D. Karachaliou et al. (2013) Clin. Lung Cancer 14:205. In some aspects, the KRAS mutation is G12C.

The presence or absence of mutations in KRAS can be detected by any method known in the art. An exemplary method for determination of the presence or absence of mutations in KRAS in tumors of patients is described in the following: a sample of tumor tissue is obtained, e.g. by resection or biopsy, or optionally a sample of peripheral blood is obtained (to detect circulating tumor cells). DNA can be extracted from fresh tissue, formalin-fixed paraffin-embedded (FFPE) tissue blocks or frozen tissue. Sequence analysis can be performed by any method known in the art, but would typically involve hybridization or a polymerase chain reaction (PCR)-based method, and may employ next generation sequencing technology. Exemplary methods include PCR-based sequencing, high resolution melting analysis (HRMA), amplification refractory mutation system (ARMS) and cleavage amplification polymorphism sequence-tagged sites (PCR-RFLP). See, e.g., Tan & Du (2012) World J. Gastroenterol. 18:5171. In some aspects, the cancer comprises a KRAS carrying a G12C, G12V and/or G12D mutation. In some aspects, the cancer comprises a KRAS carrying a G12C.

A “KRAS inhibitor,” as used herein, is capable of specifically binding to a KRAS (such as a mutant KRAS, in particular a human mutant KRAS) and thereby inhibiting the activity of said KRAS (such as said mutant KRAS, in particular said human mutant KRAS). As will be acknowledged by a person skilled in the art, in aspects of the methods of the present disclosure, wherein the subject is a human, the KRAS inhibitor specifically binds to a human KRAS (such as a human mutant KRAS). The KRAS inhibitor may bind covalently or non-covalently to KRAS. In some aspects, the KRAS inhibitor binds covalently to KRAS. For example, the KRAS inhibitor may inhibit a KRAS having a mutation at position G12 of the canconical human KRAS sequence. In some aspects, the KRAS inhibitor is an irreversible inhibitor of KRAS. In some aspects, the KRAS inhibitor is a KRAS G12C inhibitor (i.e., an inhibitor capable of specifically binding to and inhibiting KRAS having a G12C mutation). In some aspects, the KRAS inhibitor is a small molecule. In some aspects, the KRAS inhibitor is sotorasib. In some aspects, the KRAS inhibitor is adagrasib. In some aspects, the KRAS inhibitor is MRTX-1257. Further information on KRAS mutations and KRAS inhibitors can be obtained, for example, in Huang et al., 2021, Signal Transduction and Targeted Therapy 6:368, herewith incorporated by reference in its entirety.

A “subject” includes a human or a non-human animal. The non-human animal may be a nonhuman primate, a sheep, a dog, a rabbit, or a rodent such as a mouse, a rat or a guinea pig. In some aspects, the subject is a mammal such as a nonhuman primate, sheep, dog, cat, rabbit, human, ferret or rodent. In some aspects, the subject is a human. The terms “subject” and “patient” are used interchangeably herein.

“Treatment,” “treating,” or “therapy” of a subject refers to any type of intervention or process performed on, or administration of an active agent to, the subject (such as a human) with the objective of reversing, alleviating, ameliorating, inhibiting, slowing down or preventing the onset, progression, development, severity or recurrence of a symptom, complication, condition or biochemical indicia associated with a disease or enhancing overall survival. In some aspects, treating a tumor refers to slowing down the growth of the tumor. In some aspects, treating a tumor refers to preventing the progression of the growth of the tumor. In some aspects, treating a tumor refers to reducing the size of the tumor. In some aspects, the treatment expands the progression free survival (i.e., the time from randomization or initiation of treatment to the occurrence of disease progression or death) of the subject (such as a human) compared to the progression free survival without treatment. In some aspects, the treatment expands the life span of the subject (such as a human) compared to the life span without treatment.

In some aspects of the methods of the present disclosure, the KRAS inhibitor, the Treg-depleting agent, and/or the additional anti-tumor treatment are administered to the subject (such as a human) in a therapeutically effective amount (or at a therapeutically effective dose). A “therapeutically effective amount” or “therapeutically effective dose” of a therapeutic agent is any amount of the agent that, when used alone or in combination with another therapeutic agent, promotes improvement of biochemical indicia associated with a disease, disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), and/or a prevention or reduction of impairment or disability due to the disease affliction, or otherwise an amelioration of disease symptoms in the subject (such as a human). In addition, the terms “effective” and “effectiveness” with regard to a treatment includes both pharmacological effectiveness and physiological safety. Pharmacological effectiveness refers to the ability of the drug to promote disease regression, e.g., cancer regression, in the subject (such as a human). Physiological safety refers to an acceptable level of toxicity, or other adverse physiological effects at the cellular, organ and/or organism level (adverse effects) resulting from administration of the drug. The efficacy of a therapeutic agent can be evaluated using a variety of methods known to the skilled practitioner, such as in human subjects during clinical trials, in animal model systems predictive of efficacy in humans, or by assaying the activity of the agent in in vitro assays.

In some aspects, a therapeutically effective amount of an anti-tumor therapy promotes tumor regression, optionally to the point of eliminating the tumor. “Promoting tumor regression” means that administering a therapeutically effective amount of the anti-tumor therapy, alone or in combination with an additional anti-tumor treatment, results in a reduction in tumor growth or size, necrosis of the tumor, a decrease in severity of at least one disease symptom, an increase in frequency and duration of disease symptom-free periods, or a prevention of impairment or disability due to the disease affliction.

By way of example for the treatment of tumors, a therapeutically effective amount of an anti-tumor agent inhibits cell growth or tumor growth by at least about 20%, at least about 40%, at least about 60%, or at least about 80% relative to untreated subjects. The ability of an agent or treatment to inhibit tumor growth can be evaluated in an animal model system such as, for example, any of the Lewis Lung (3LL) carcinoma, CT26 colon adenocarcinoma, MC38 colon adenocarcinoma, SA1N fibrosarcoma, 4T1 mammary carcinoma, and MB49 bladder carcinoma mouse tumor models. Alternatively, tumor growth inhibition can be measured by evaluating the ability of the agent or treatment to inhibit cell growth in vitro using assays known to the skilled practitioner. In some aspects of the present disclosure, tumor regression can be observed and continue for a period of at least about 20 days, at least about 40 days, or at least about 60 days by administration of a therapeutically effective amount of an anti-tumor agent.

In some aspects of the methods of the present disclosure, the KRAS inhibitor, the Treg cell depleting agent, and/or the additional anti-tumor treatment are administered to the subject (such as a human) in a prophylactically effective amount (or at a prophylactically effective dose). A “prophylactically effective amount” or “prophylactically effective dose” of a therapeutic agent is any amount of the agent that, when administered alone or in combination with another therapeutic agent to a subject (such as a human) at risk of developing a disease or of suffering a recurrence of disease, inhibits the development or recurrence of the disease. “Inhibiting” the development or recurrence of a disease means either lessening the likelihood of the disease's development or recurrence, or preventing the development or recurrence of the disease entirely.

A subject that is “responsive” to a certain therapy, such as a therapy with a KRAS inhibitor, refers to a subject that gains a benefit from the therapy. In contrast thereto, a subject that is “not responsive” to a certain therapy refers to a subject that does not gain a benefit from the therapy.

Consequently, “predicting the responsiveness” of a subject to a certain therapy refers to predicting whether the subject is likely to gain a benefit from the therapy. A subject that is “predicted to be responsive” to a certain therapy, such as a therapy with a KRAS inhibitor, refers to a subject that is predicted to gain a benefit from the therapy. In contrast thereto, a subject that is “predicted to not be responsive” to a certain therapy refers to a subject that is predicted to not gain a benefit from the therapy.

The presence/absence/number of a biomarker in a subject, such as the number of Tregs in a TME and/or the absence of a spatial cellular community and/or the number of said spatial cellular community in a TME as described in the present disclosure, can be “predictive that a subject will be responsive” to a certain therapy (or, in other words, can be “predictive of the responsiveness” of a subject to a certain therapy). The presence/absence/number of the biomarker is indicative that the subject is likely to gain a benefit from the therapy or not, depending on the particular biomarker. For example, high levels of Tregs in a TME can be predictive that a subject will not be responsive to therapy with a KRAS inhibitor.

Gaining a benefit from the therapy may comprise one or more of the following: reversing, alleviating, ameliorating, inhibiting, slowing down or preventing the onset, progression, development, severity or recurrence of a symptom, complication, condition or biochemical indicia associated with a disease or enhancing overall survival. In some aspects, the benefit comprises slowing down the growth of the tumor. In some aspects, the benefit comprises preventing the progression of the growth of the tumor. In some aspects, the benefit comprises reducing the size of the tumor. In some aspects, the benefit comprises expanding the progression free survival (i.e., the time from randomization or initiation of treatment to the occurrence of disease progression or death) of the subject compared to the progression free survival without treatment. In some aspects, the benefit comprises expanding the life span of the subject compared to the life span without treatment.

“Ordering the determination of,” or “ordering the comparison of,” as used herein, refers to the act of instructing, ordering or directing that the determination or comparison is performed by others. Such instructing, ordering or directing might be performed, for example, by a physician, or another medical professional under the direction of a physician, and might involve ordering the determination or comparison to be performed at a commercial laboratory or in a medical facility laboratory. The physician who is responsible for instructing, ordering or directing such determination or comparison would typically also be the one to prescribe and/or administer an anti-tumor therapy (such as, a KRAS inhibitor and a Treg-depleting agent) to a subject (such as a human).

A subject is “scheduled to be administered an anti-tumor therapy” or “selected for an anti-tumor therapy” by any definitive step taken in the treatment protocol for the subject (which might be a human) that dictates the future administration of the anti-tumor therapy. Such scheduling or selecting may include, but is not limited to, noting in a patient's medical record, including a digital medical record, the suitability of that patient for administration of the anti-tumor therapy (such as a KRAS inhibitor therapy), or entering into the medical record a diagnosis that carries with it assignment of the patient to the patient subset that can be administered the anti-tumor therapy (such as a KRAS inhibitor therapy). Scheduling or selecting might also comprise the presecription of the anti-tumor therapy (such as a KRAS therapy). “Scheduling” and “selecting,” as used herein, does not encompass purely mental steps, and instead requires an individual, such as a physician, to take an objectively verifiable action as outlined above.

A “tumor microenvironment,” or “TME,” is an ecosystem in the vicinity of tumor cells, including, but not limited to, blood vessels, immune cells, fibroblasts, signaling molecules, and the extracellular matrix (ECM). Mutual interaction between cancer cells and the different components of the TME support its growth and invasion in healthy tissues which generally correlates with tumor resistance to current treatments and poor prognosis. Tumors can influence the TME by releasing extracellular signals, promoting tumor angiogenesis and inducing peripheral immune tolerance, while the immune cells in the TME can affect the growth and evolution of cancerous cells.

The TME may be analyzed by imaging mass cytometry (IMC), a method to explore spatial cellular patterns in the TME as described herein. IMC is particularly useful to study the TME due to its ability to capture up to 40 markers simultaneously (multiplexing method). IMC enables the analysis of the make-up of a tumor tissue in situ. With IMC, spatial cellular information in the tissue remains intact, meaning that cell phenotypes can be analyzed in the context of their spatial neighbors. Obtaining spatial information is particularly useful when studying the TME, as it provides insights into the cellular interactions dictating local immune activation or suppression, as mechanism of immune response or resistance to treatment. An exemplary way to conduct IMC is described herein, in particular, in the Materials and Methods sections to Examples 1-6 and/or Example 7 below. In some aspects, IMC (including sample preparation, staining, imaging, and cell typing) is essentially carried out as described in the Materials and Methods sections to Examples 1-6 and/or Example 7 below. In general, IMC is conducted using a tissue slice sample, such as a tumor tissue slice.

Alternatively, or in addition, the TME may be analyzed by flow cytometry, such as fluorescence activated cell sorting (FACS). In some aspects, the TME is analyzed by FACS as described in the Materials and Methods section to Example 9 below.

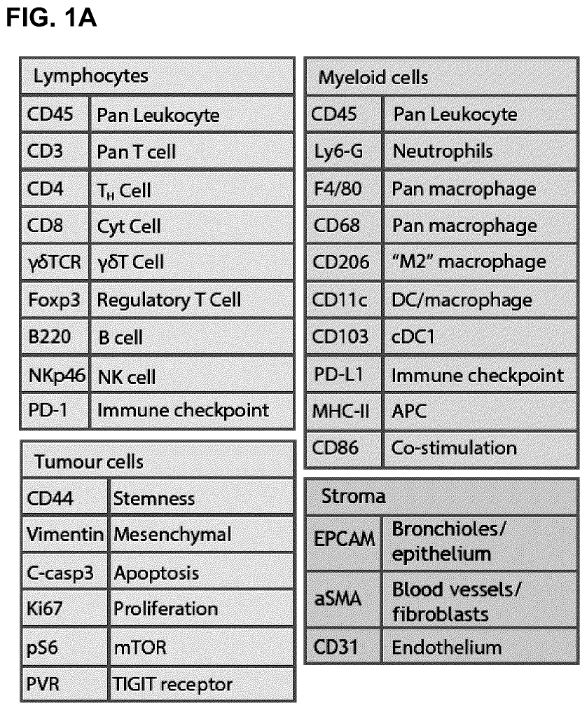

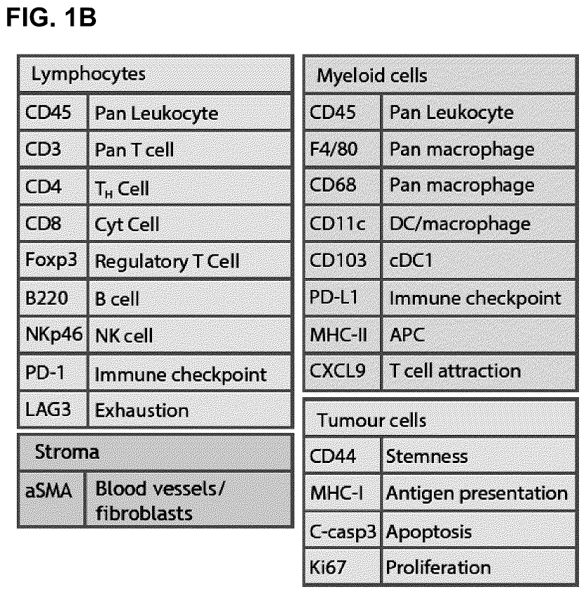

As used herein, a “spatial cellular community” (which might also be referred to as “spatial community,” “cell community,” or “community” in short) refers to a number of cells gathered together in a tissue, such as a tumor tissue or a TME. The cell density of a spatial cellular community can be calculated by taking the number of cells assigned to a spatial cellular community divided by the total tissue area (cells/mm²). In some aspects, the cell density of a spatial cellular community is at least 20, at least 25, at least 30, at least 35, or at least 40 cells/mm². In some aspects, the cell density of a spatial cellular community is at least 45, at least 50, at least 55, at least 60, or at least 65 cells/mm². In some aspects, the cell density of a spatial cellular cumminty is at least 25 cells/mm². A spatial cellular community comprises one or more cell “neighborhoods.” A cell neighborhood includes a certain cell of interest (such as a Treg) and its neighboring cells. A cell is considered to be in the “neighborhood” of another cell or “neighboring” another cell, if the cell is located within a certain radius of the cell boundary of the other cell of interest. In some aspects, a cell is considered to be in the “neighborhood” of another cell or “neighboring” another cell or “interacting” with another cell, if the cell is located within about 15 μm (pixels) of the cell boundary of the other cell of interest. A 15-pixel radius depicts the average size of a cell, and therefore, cells that are located within about 15 μm (pixels) of the cell boundary of the other cell of interest are those “up to one cell away.” As a result of using a fixed radius to define cells as neighboring another cell, cell neighborhoods are not equal in cell number, but instead reflect the local density surrounding each cell. Whether a cell is in the neighborhood of another cell is typically determined using IMC for analyzing a (tumor) tissue slice. To identify cell types in a tissue, antibody panels such as the antibody panels shown in FIG. 1A and FIG. 1B can be applied. In some aspects, the spatial cellular community is identified by IMC, optionally by analyzing a tissue slice. Unless the context indicates otherwise, the spatial cellular community is a spatial cellular community in a TME of a tumor. In some aspects, the spatial cellular community is rich in T cells, such as CD8⁺ T cells. In some aspects, the spatial cellular community is rich in Tregs, e.g., the spatial cellular community comprises about 0.5% to about 2% Tregs (wherein 100% refer to all cells in the spatial cellular community). In some aspects, the spatial cellular community comprises CD4⁺ T cells, CD8⁺ T cells, Tregs, and dendritic cells (DCs). In some aspects, at least 50% of the Tregs in the spatial cellular community are Tregs in the neighborhood of CD8⁺ T cells in the spatial cellular community. In some aspects, at least 60%, 70%, 80%, 90%, or 95% of the Tregs in the spatial cellular community are Tregs in the neighborhood of CD8⁺ T cells in the spatial cellular community. In some aspects, the Tregs in the neighborhood of CD8⁺ T cells inhibit said CD8⁺ T cells (and thereby dampen the immune response). In some aspects, the spatial cellular community comprises tumor cells, such as casp3⁺ tumor cells. Consequently, even if reference is made to a spatial cellular community in a tumor microenvironment, this does not exclude that the community also comprises (even a significant number of) tumor cells. In some aspects, the spatial cellular community is located peritumorally (i.e., around the tumor). In some aspects, the spatial cellular community is located intratumorally.

As used herein, the term “programmed cell death protein 1 axis-blocking agent” or “PD-1 axis-blocking agent,” refers to an agent that is capable of selectively blocking the PD-1 axis. The term encompasses the following antagonists: a PD-L1 antagonist, a PD-1 antagonist, and a PD-L2 antagonist. In some aspects, the PD-1 axis-blocking agent is a PD-1 antagonist or a PD-L1 antagonist. In some aspects, the PD-1 axis-blocking agent comprises an antibody, or an antigen binding portion thereof. In some aspects, the PD-1 axis-blocking agent comprises an anti-PD-L1 antibody, an anti-PD-1 antibody, or an antigen binding portion of any of the foregoing. In some aspects, the PD-1 axis-blocking agent comprises an anti-PD-1 antibody, or an antigen binding portion thereof. In some aspects, the PD-1 axis-blocking agent comprises an anti-PD-L1 antibody, or an antigen binding portion thereof. In some aspects, wherein the subject is a human, the PD-1 axis-blocking agent is a human PD-1 axis-blocking agent (i.e., an agent capable of selectively blocking human PD-1, human PD-L1, and/or human PD-L2).

“Programmed cell death protein 1” or “PD-1” refers to an immunoinhibitory receptor belonging to the CD28 family that is expressed predominantly on previously activated T cells in vivo, and binds to two ligands, PD-L1 and PD-L2. In some aspects, the PD-1 is human PD-1. In some aspects, the PD-1 is a variant, an isoform, or a species homolog of human PD-1, or an analog having at least one common epitope with human PD-1. The amino acid sequence of human PD-1 is set forth in SEQ ID NO: 174 (Table 4).

TABLE 4
Sequence of human PD-1
(SEQ ID NO: 174; Genbank Accession No. U64863).

MQIPQAPWPVVWAVLQLGWRPGWFLDSPDRPWNPPTFSPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL

“Programmed Death Ligand-1” (PD-L1) is one of two cell surface glycoprotein ligands for PD-1 (the other being PD-L2) that downregulate T cell activation and cytokine secretion upon binding to PD-1. In some aspects, the PD-L1 is human PD-L1. In some aspects, the PD-L1 is a variant, an isoform, or a species homolog of human PD-L1, or an analog having at least one common epitope with human PD-L1. The amino acid sequence of human PD-L1 is set forth in SEQ ID NO: 175 (Table 5).

TABLE 5
Sequence of human PD-L1
(SEQ ID NO: 175; Genbank Accession No. Q9NZQ7).

MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTTNEIFYCTFRRLDPEENHTAELV
IPELPLAHPPNERTHLVILGAILLCLGVALTFIFRLRKGRMMDVK
KCGIQDTNSKKQSDTHLEET

“C-C Motif Chemokine Receptor 8” (“CCR8”; also known in the art as, for example, CY6, TER1, CCR-8, CKRL1, CDw198, CMKBR8, GPRCY6, CMKBRL2, or CC-CKR-8) is a seven-transmembrane GPCR; this GPCR has been shown to be expressed primarily on intratumoral FOXP3^{high} Tregs. In some aspects, the CCR8 is human CCR8. In some aspects, the CCR8 is a variant, an isoform, or a species homolog of human CCR8, such as mouse CCR8, or an analog having at least one common epitope with human CCR8. The complete human CCR8 and mouse CCR8 amino acid sequences can be found under Genbank Accession Nos. AAI07160.1 and NP_031746.1, respectively. The amino acid sequence of human CCR8 (SEQ ID NO: 176) is also shown in Table 6.

TABLE 6
Human CCR8 amino acid sequence
(SEQ ID NO: 176; Genbank Accession
No. AAI07160.1).
Amino acid residues 15-21 are
highlighted (bold and underlined).

MDYTLDLSVT TVTDYYYPDI FSSPCDAELI
QTNGKLLLAV FYCLLFVFSL LGNSLVILVL
VVCKKLRSIT DVYLLNLALS DLLFVFSFPF
QTYYLLDQWV FGTVMCKVVS GFYYIGFYSS
MFFITLMSVD RYLAVVHAVY ALKVRTIRMG
TTLCLAVWLT AIMATIPLLV FYQVASEDGV
LQCYSFYNQQ TLKWKIFTNF KMNILGLLIP
FTIFMFCYIK ILHQLKRCQN HNKTKAIRLV
LIVVIASLLF WVPFNVVLFL TSLHSMHILD
GCSISQQLTY ATHVTEIISF THCCVNPVIY
AFVGEKFKKH LSEIFQKSCS QIFNYLGRQM
PRESCEKSSS CQQHSSRSSS VDYIL

“Lymphocyte activation gene 3” or “LAG-3” is an immune checkpoint receptor protein found on the cell surface of effector T cells and regulatory T cells (Tregs) and functions to control T cell response, activation and growth. In some aspects, the LAG-3 is human LAG-3. In some aspects, the LAG-3 is a variant, an isoform, or a species homolog of human LAG-3, such as mouse LAG-3, or an analog having at least one common epitope with human LAG-3. The amino acid sequence of human LAG-3 is set forth in SEQ ID NO: 177 (Table 7).

TABLE 7
Sequence of human LAG-3 (SEQ ID NO: 177;
Genebank accession No. NP_002277).

MWEAQFLGLLFLQPLWVAPVKPLQPGAEVPVVWAQEGAPAQLPCS
PTIPLQDLSLLRRAGVTWQHQPDSGPPAAAPGHPLAPGPHPAAPS
SWGPRPRRYTVLSVGPGGLRSGRLPLQPRVOLDERGRORGDFSLW
LRPARRADAGEYRAAVHLRDRALSCRLRLRLGQASMTASPPGSLR
ASDWVILNCSFSRPDRPASVHWFRNRGQGRVPVRESPHHHLAESF
LFLPQVSPMDSGPWGCILTYRDGFNVSIMYNLTVLGLEPPTPLTV
YAGAGSRVGLPCRLPAGVGTRSFLTAKWTPPGGGPDLLVTGDNGD
FTLRLEDVSQAQAGTYTCHIHLQEQQLNATVTLAIITVTPKSFGS
PGSLGKLLCEVTPVSGQERFVWSSLDTPSQRSFSGPWLEAQEAQL
LSQPWQCQLYQGERLLGAAVYFTELSSPGAQRSGRAPGALPAGHL
LLFLILGVLSLLLLVTGAFGFHLWRRQWRPRRFSALEQGIHPPQA
QSKIEELEQEPEPEPEPEPEPEPEPEPEQL

“Cytotoxic T-lymphocyte associated protein 4” or “CTLA-4” also known as CD152 (cluster of differentiation 152), is a protein receptor that functions as an immune checkpoint and downregulates immune responses. In some aspects, the CTLA-4 is human CTLA-4. In some aspects, the CTLA-4 is a variant, an isoform, or a species homolog of human CTLA-4, such as mouse CTLA-4, or an analog having at least one common epitope with human CTLA-4. The amino acid sequence of human CTLA-4 (SEQ ID NO: 178) is shown in Table 8.

TABLE 8
Human CTLA-4 amino acid sequence
(SEQ ID NO: 178; Genebank accession
No. NP_005205.2).

MACLGFQRHKAQLNLATRTWPCTLLFFLLFIPVFCKAMHVAQPAV
VLASSRGIASFVCEYASPGKATEVRVTVLRQADSQVTEVCAATYM
MGNELTFLDDSICTGTSSGNQVNLTIQGLRAMDTGLYICKVELMY
PPPYYLGIGNGTQIYVIDPEPCPDSDFLLWILAAVSSGLFFYSFL
LTAVSLSKMLKKRSPLTTGVYVKMPPTEPECEKQFQPYFIPIN

### II. Method of Treating a Tumor Comprising Administering a KRAS Inhibitor and a Treg-Depleting Agent

As was found herein, depletion of Treg cells in addition to administrating a KRAS inhibitor led to profound reduction in tumor growth, longer survival and enhanced, and in some cases sustained, anti-tumor immunity (see, in particular, Examples 8 and 9 of the present disclosure).

In some aspects, the present disclosure is thus directed to a method of treating a tumor in a subject in need thereof comprising administering a KRAS inhibitor and a regulatory T cell (“Treg”)-depleting agent to the subject. In some aspects, the method further comprises administering to the subject an additional anti-tumor treatment. The subject may be a human.

In some aspects, the present disclosure is also directed to a method of treating a tumor in a subject in need thereof comprising administering only a KRAS inhibitor and a Treg-depleting agent as anti-tumor agents to the subject (i.e., the method does not comprise administration of an additional anti-tumor treatment to the subject). In some of the aspects described in the present paragraph, the KRAS inhibitor is a KRAS G12C inhibitor (such as sotorasib, adagrasib, or MRTX-1257) and the Treg-depleting agent comprises an anti-CCR8 antibody (such as 14S15, 14S15h, or imzokitug), or an antigen binding portion thereof. In some of the aspects described in the present paragraph, the KRAS inhibitor is a KRAS G12C inhibitor (such as sotorasib, adagrasib, or MRTX-1257) and the Treg-depleting agent comprises anti-CTLA-4 antibody (such as tremelimumab or ipilimumab), or an antigen binding portion thereof. The subject may be a human. The tumor may be a lung cancer, such as NSCLC. The NSCLC in turn may be a lung adenocarcinoma or a squamous cell carcinoma.

In some aspects, the present disclosure is also directed to a method of treating a tumor in a subject in need thereof comprising administering a means for inhibiting KRAS and a means for depleting Treg cells in the subject. In some aspects, the method further comprises administering to the subject an additional means for anti-tumor treatment.

In some aspects, the present disclosure is also directed to a method of treating a tumor in a subject in need thereof comprising administering only a means for inhibiting KRAS and a means for depleting Treg cells to the subject (i.e., the method does not comprise administration of an additional anti-tumor treatment to the subject).

In some aspects, the KRAS inhibitor and the Treg-depleting agent are administered separately. Separate administration means that the KRAS inhibitor and the Treg-depleting agent are not provided in the same pharmaceutical composition, but in separate pharmaceutical compositions. For example, the KRAS inhibitor can be provided in the form of a tablet, whereas the Treg-depleting agent can be provided in the form of a liquid (such as a suspension or solution) that can be administered intravenously.

In some aspects, the KRAS inhibitor and the Treg-depleting agent are administered sequentially. Within the meaning of the present disclosure, a sequential administration refers to an administration of two or more agents more than about 2 hours apart within a treatment regimen. For example, one agent may be administered at Day 1, whereas the other agent may be administered at Day 3 of a treatment regimen. In some aspects, the KRAS inhibitor and the Treg-depleting agent are administered about 2.5 hours apart, about 3 hours apart, about 3.5 hours apart, about 4 hours apart, about 4.5 hours apart, about 5 hours apart, about 5.5 hours apart, or about 6 hours apart. In some aspects, the KRAS inhibitor and the Treg-depleting agent are administered about 12 hours apart, about 16 hours apart, about 20 hours apart, or about 24 hours apart. In some aspects, the KRAS inhibitor and the Treg-depleting agent are administered about 2 days apart, about 3 days apart, about 4 days apart, about 5 days apart, about 6 days apart, or about 7 days apart.

In some aspects, the KRAS inhibitor is administered prior to the Treg-depleting agent. In some aspects, the KRAS inhibitor is administered about 2.5 hours, about 3 hours, about 3.5 hours, about 4 hours, about 4.5 hours, about 5 hours, about 5.5 hours, or about 6 hours prior to the Treg-depleting agent. In some aspects, the KRAS inhibitor is administered about 12 hours, about 16 hours, about 20 hours, or about 24 hours prior to the Treg-depleting agent. In some aspects, the KRAS inhibitor is administered about 2 days, about 3 days, about 4 days, about 5 days, about 6 days, or about 7 days prior to the Treg-depleting agent.

In some aspects, the Treg-depleting agent is administered prior to the KRAS inhibitor. In some aspects, the Treg-depleting agent is administered about 2.5 hours, about 3 hours, about 3.5 hours, about 4 hours, about 4.5 hours, about 5 hours, about 5.5 hours, or about 6 hours prior to the KRAS inhibitor. In some aspects, the Treg-depleting agent is administered about 12 hours, about 16 hours, about 20 hours, or about 24 hours prior to the KRAS inhibitor. In some aspects, the Treg-depleting agent is administered about 2 days, about 3 days, about 4 days, about 5 days, about 6 days, or about 7 days prior to the KRAS inhibitor.

In some aspects, the KRAS inhibitor and the Treg-depleting agent are administered concurrently. Within the meaning of the present disclosure, a concurrent administration refers to an administration of two or more agents no more than about 2 hours apart within a treatment regimen. In some aspects, the KRAS inhibitor and the Treg-depleting agent are administered about 2 hours apart, about 1.5 hours apart, about 1 hour apart, or about 0.5 hours apart. In some aspects, the KRAS inhibitor and the Treg-depleting agent are administered less than about 2 hours apart, less than about 1.5 hours apart, less than about 1 hour apart, or less than about 0.5 hours apart. In some aspects, KRAS inhibitor and the Treg-depleting agent are administered about 0.4 hours apart, about 0.3 hours apart, about 0.2 hours apart, or about 0.1 hours apart. In some aspects, KRAS inhibitor and the Treg-depleting agent are administered less than about 0.4 hours apart, less than about 0.3 hours apart, less than about 0.2 hours apart, or less than about 0.1 hours apart.

In some aspects, the method further comprises determining, or ordering the determination of, the number of Tregs in the TME. In some aspects, the number of Tregs is determined prior to administering the KRAS inhibitor and/or the Treg-depleting agent. In some aspects, the number of Tregs is determined after administering the KRAS inhibitor and/or the Treg cell depleting agent. In some aspects, the Tregs are Tregs in the neighborhood of CD8⁺ T cells. In some aspects, the CD8⁺ T cells in the neighborhood of the Tregs are CD8⁺ T cells that are further neighboring DCs and CD4⁺ T cells. In some aspects, the number of Tregs is determined in a spatial cellular community in the TME comprising CD4⁺ T cells, CD8⁺ T cells, Tregs, and dendritic cells (DCs). As regards further aspects relating to the spatial cellular community, reference is, for example, also made to the section titled “Further methods of the present disclosure”. In some aspects, the number of Tregs is determined in a tumor tissue sample from the subject. In some aspects, the tumor tissue sample is a tumor tissue slice. In some aspects, the number of Tregs is determined by imaging mass cytometry (IMC).

In some aspects, administering the Treg-depleting agent reduces tumor growth compared to growth of the tumor prior to administering the Treg-depleting agent. In some aspects, administering the Treg-depleting agent reduces tumor volume compared to the volume of the tumor prior to administering the Treg-depleting agent. In some aspects, administering the Treg-depleting agent increases survival probability of the subject compared to the probability of survival prior to administering the Treg-depleting agent. In some aspects, administering the Treg-depleting agent enhances anti-tumor immunity compared to the anti-tumor immunity prior to administering the Treg-depleting agent. In some aspects, enhancing the anti-tumor immunity comprises increasing CD8⁺ T cell infiltration compared to the CD8⁺ T cell infiltration prior to administering the Treg-depleting agent. In some aspects, enhancing the anti-tumor immunity comprises increasing the number of activated CD8⁺ T cells compared to the number of activated CD8⁺ T cells prior to administering the Treg-depleting agent. In some aspects, administering the Treg-depleting agent enhances the systemic immune response compared to the systemic immune response prior to administering the Treg-depleting agent.

Further aspects regarding the KRAS inhibitor (see, in particular, section titled “KRAS inhibitor”), the Treg-depleting agent (see, in particular, section titled “Treg-depleting agent”), the additional anti-tumor treatment (see, in particular, section titled “Additional anti-tumor treatment”), the tumor (see, in particular, section titled “Tumor”), and the subject (see, in particular, section titled “Subject”), are described in the below sections. In addition, some aspects of the method of treating a tumor of the present disclosure are described in the following.

In some aspects, the subject is a human subject, the KRAS inhibitor is a KRAS G12C inhibitor, e.g., adagrasib, sotorasib, or MRTX-1257, the tumor is a lung cancer (e.g., NSCLC (such as a lung adenocarcinoma or a squamous cell carcinoma), and the Treg-depleting agent comprises an anti-CCR8 antibody (such as 14S15, 14S15h, or imzokitug), or an antigen binding portion thereof. In some of the aspects described in this paragraph, the anti-CCR8 antibody is a monoclonal antibody and optionally a nonfucosylated and/or activatable antibody. In some of the aspects described in this paragraph, the method further comprises administering to the subject an additional anti-tumor treatment, which optionally is an anti-PD-1 or an anti-PD-L1 antibody. The anti-PD-1 antibody may be nivolumab or pembrolizumab. The anti-PD-L1 antibody may be atezolizumab, durvalumab, or avelumab.

In some aspects, the subject is a human subject, the KRAS inhibitor is a KRAS G12C inhibitor (e.g., adagrasib, sotorasib, or MRTX-1257), the tumor is a lung cancer (e.g., NSCLC (such as a lung adenocarcinoma or a squamous cell carcinoma)), and the Treg-depleting agent comprises an anti-CTLA-4 antibody (such as tremelimumab or ipilimumab), or an antigen binding portion thereof. In some of the aspects described in this paragraph, the anti-CTLA-4 antibody is a monoclonal antibody and optionally a nonfucosylated and/or activatable antibody. In some of the aspects described in this paragraph, the method further comprises administering to the subject an additional anti-tumor treatment, which optionally is an anti-PD-1 or an anti-PD-L1 antibody. The anti-PD-1 antibody may be nivolumab or pembrolizumab. The anti-PD-L1 antibody may be atezolizumab, durvalumab, or avelumab.

### 1. KRAS Inhibitor

In the following, the KRAS inhibitor suitable for the methods of the present disclosure is further described.

In some aspects, the KRAS inhibitor inhibits one or more of KRAS G12A, KRAS G12C, KRAS G12D, KRAS G12R, KRAS G12S, KRAS G12V, KRAS G12R, KRAS G12F, KRAS G13C, KRAS G13D, KRAS Q61L, KRAS Q61H, and KRAS Q61K. In some aspects, the KRAS inhibitor inhibits KRAS G12A, KRAS G12C, KRAS G12D, KRAS G12R, KRAS G12S, KRAS G12V, KRAS G12R, KRAS G12F, KRAS G13C, KRAS G13D, KRAS Q61L, KRAS Q61H, or KRAS Q61K.

There are currently many KRAS inhibiting compounds in early dose escalation studies, which are all useful for the methods of the present disclosure. Said KRAS inhibitors include G12D inhibitors, allele-specific G12D PROTACs, pan-RAS PROTACs, pan-RAS glue-like compounds, and RAS: PI3K interrupters (Herzberg, B. and Manji, G. (2023) Oncologist. 28:283-286). For example, quinazolines which inhibit KRAS G12A, G12C, G12D, G12R, G12S, G12V, G13D, and Q61H are disclosed in WO 2023/150284. Tetrahydropyridopyrimidines which inhibit KRAS G12A, G12C, G12D, G12R, G12S, G12V, G13D and Q61H are disclosed in WO 2022/133038. Azaquinazolines which inhibit KRAS G12A, G12C, G12D, G12R, G12S, G12V, G13D and Q61H are disclosed in WO 2022/132200. KRAS G12C inhibitors ARS-853 (Araxes Pharma) and ARS-1620 (Araxes Pharma) are disclosed in Hansen R, Peters U, Babbar A, et al. (2018) Nat Struct Mol Biol. 25 (6): 454-462. MRTX-1257 (Mirati Therapeutics) which inhibits KRAS G12C is disclosed in Marx, et al. (2020) Mol. Cancer Res. 18 (5_Supplement): B30. MRTX-EX185 (Mirati Therapeutics), which inhibits KRAS G12C is disclosed in Vasta, J. D., Peacock, D. M., Zheng, Q. et al. (2022) Nat. Chem. Biol. 18:596-604. MRTX-1133 (Mirati Therapeutics), which inhibits KRAS G12D is disclosed in WO 2023/059600 and WO 2023/059594. RMC-6291 (Revolution Medicines), RMC-6236 (Revolution Medicines), RMC-036 (Revolution Medicines), RMC-037 (Revolution Medicines), JAB-21822 (Jacobio Pharmaceuticals), JNJ-74699157 (Janssen), GFH925 (GenFleet), YL-15293 (Shanghai YingLi Pharmaceutical), and YL-17231 (Shanghai YingLi Pharmaceutical), which inhibit KRAS G12C are disclosed in WO 2023/009572. ASP2453 (Astellas), which inhibits KRAS G12C is disclosed in Nakayama, A., Nagashima, T., Nishizono, Y. et al. (2022) Br. J. Cancer 126:744-753. BBO-8520 (Bridge Bio), BI 1701963 (BI), and ERAS-3490 (Erasca), which inhibit KRAS G12C are disclosed in Herzberg, B. and Manji, G. (2023) Oncologist. 28:283-286. SY-5933 is disclosed in clinical trial NCT06006793. JDQ443 (Novartis) which inhibits KRAS G12C, is disclosed in WO 2021/120890.

In some aspects, the KRAS inhibitor is a KRAS inhibitor as described in any of the following international patent publications, which are herewith incorporated by reference in their entirety: WO/2023/150284, WO/2023/059600, WO/2023/059594, WO/2023/059596, WO/2023/059597, WO/2023/059598, WO/2023/039020, WO/2022/251193, WO/2022/221528, WO/2022/165142, WO/2022/133038, WO/2022/132200, WO/2022/098625, WO/2022/087270, WO/2022/066646, WO/2022/056307, WO/2022/031678, WO/2022/015375, WO/2021/173524, WO/2021/141628, WO/2021/061749, WO/2021/041671, WO/2020/146613, WO/2020/118066, WO/2020/101736, WO/2020/055760, WO/2020/055761, WO/2020/055755, WO/2020/055756, WO/2020/055758, WO/2020/047192, WO/2019/217307, WO/2019/099524, and WO/2017/201161.

In some aspects, the KRAS inhibitor is a small molecule.

In some aspects, the KRAS inhibitor inhibits one or more of KRAS G12C, KRAS G12D, and KRAS G12V. In some aspects, the KRAS inhibitor inhibits KRAS G12C, KRAS G12D, or KRAS G12V.

In some aspects, the KRAS inhibitor is a KRAS G12D inhibitor (i.e., the KRAS inhibitor inhibits KRAS carrying a G12D mutation). In some aspects, the KRAS inhibitor is a KRAS G12C inhibitor (i.e., the KRAS inhibitor inhibits KRAS carrying a G12C mutation).

In some aspects, the KRAS inhibitor is selected from ARS-853, ARS-1620, MRTX-1257, MRTX-EX185, MRTX-1133, ASP2453, RMC-6291, RMC-6236, RMC-036, RMC-037, BBO-8520, BI 1701963, ERAS-3490, JAB-21822, JNJ-74699157, GFH925, YL-15293, YL-17231, SY-5933, and JDQ443.

In some aspects, the KRAS inhibitor is ARS-853. In some aspects, the KRAS inhibitor is ARS-1620. In some aspects, the KRAS inhibitor is MRTX-1257. In some aspects, the KRAS inhibitor is MRTX-EX185. In some aspects, the KRAS inhibitor is MRTX-1133. In some aspects, the KRAS inhibitor is ASP2453. In some aspects, the KRAS inhibitor is RMC-6291. In some aspects, the KRAS inhibitor is RMC-6236. In some aspects, the KRAS inhibitor is RMC-036. In some aspects, the KRAS inhibitor is RMC-037. In some aspects, the KRAS inhibitor is BBO-8520. In some aspects, the KRAS inhibitor is BI 1701963. In some aspects, the KRAS inhibitor is ERAS-3490. In some aspects, the KRAS inhibitor is JAB-21822. In some aspects, the KRAS inhibitor is JNJ-74699157. In some aspects, the KRAS inhibitor is GFH925. In some aspects, the KRAS inhibitor is YL-15293. In some aspects, the KRAS inhibitor is YL-17231. In some aspects, the KRAS inhibitor is SY-5933. In some aspects, the KRAS inhibitor is JDQ443.

In some aspects, the KRAS inhibitor is a KRAS G12C inhibitor, wherein the KRAS G12C inhibitor is sotorasib, adagrasib, or MRTX-1257.

In some aspects, the KRAS inhibitor is sotorasib. See formula (I) above. Sotorasib and its preparation are disclosed, for example, in WO 2018/217651. Sotorasib is sold under the brand names LUMAKRAS® and LUMYKRAS®. Sotorasib forms an irreversible, covalent bond with the unique cysteine of KRAS-G12C, locking the protein in an inactive state that prevents downstream signaling without affecting wild-type, non-mutated KRAS.

In some aspects, the KRAS inhibitor is adagrasib. See formula (II) above. Adagrasib is described, for example, in Example 478 of PCT Application WO 2019/099524. Adagrasib is sold under the brand name KRAZATI®. It irreversibly binds to the KRAS G12C locking it in the inactive GDP bound state.

In some aspects, the KRAS inhibitor is MRTX-1257. See formula (III) above. MRTX-1257 covalently binds to the cysteine at position 12 of KRAS, locking it in the inactive conformation. MRTX-1257 is disclosed, for example, in Marx, et al. (2020) Mol. Cancer Res. 18 (5_Supplement): B30.

The KRAS inhibitor can be formulated by any method well known in the art and can be prepared for administration by any administration route known to a person of ordinary skill in the art. In some aspects, the KRAS inhibitor is administered by parenteral, intraperitoneal, intradermal, intracardiac, intraventricular, intracranial, intracerebrospinal, intrasynovial, intrathecal, intramuscular, intravitreous, intravenous, intra-arterial, oral, buccal, sublingual, transdermal, topical, intratracheal, intrarectal, subcutaneous, or topical administration.

In some aspects, the KRAS inhibitor is administered systemically.

In some aspects, the KRAS inhibitor is administered intravenously.

In some aspects, the KRAS inhibitor is administered intramuscularly.

In some aspects, the KRAS inhibitor is administered orally. In some aspects, the KRAS inhibitor is administered in form of a tablet.

In some aspects, the KRAS inhibitor is sotorasib, wherein sotorasib is administered orally, optionally in the form of a tablet. In some aspects, the KRAS inhibitor is adagrasib, wherein adagrasib is administered orally, optionally in the form of a tablet. In some aspects, the KRAS inhibitor is MRTX-1257, wherein MRTX-1257 is administered orally, optionally in the form of a tablet.

In some aspects, the KRAS inhibitor is administered once daily. In some aspects, the KRAS inhibitor is sotorasib and sotorasib is administered orally, once daily, optionally in the form of a tablet.

In some aspects, the KRAS inhibitor is administered more than once a day, such as twice daily or three times a day. In some aspects, the KRAS inhibitor is administered twice daily. In some aspects, the KRAS inhibitor is agagrasib and agagrasib is administered orally, twice daily, optionally in the form of a tablet.

In some aspects, the KRAS inhibitor is administered at a daily dose of about 500 mg to about 2000 mg. In some aspects, the KRAS inhibitor is administered at a daily dose of about 600 mg to about 1800 mg. In some aspects, the KRAS inhibitor is administered at a daily dose of about 800 mg to about 1500 mg. In some aspects, the KRAS inhibitor is administered at a daily dose of about 900 mg to about 1300 mg. In some aspects, the KRAS inhibitor is administered at a daily dose of about 900 mg to about 1000 mg. In some aspects, the KRAS inhibitor is administered at a daily dose of about 1100 mg to about 1300 mg.

In some aspects, the KRAS inhibitor is administered at a daily dose of about 500 mg, about 550 mg, about 600 mg, about 650 mg, about 700 mg, about 750 mg, about 800 mg, about 850 mg, about 900 mg, about 950 mg, about 960 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some aspects, the KRAS inhibitor is administered at a daily dose of about 960 mg (optionally, wherein the KRAS inhibitor is sotorasib, which is orally administered once daily in the form of a tablet). In some aspects, the KRAS inhibitor is administered at a daily dose of about 1200 mg (optionally, wherein the KRAS inhibitor is adagrasib, which is orally administered twice daily in the form of a tablet).

In some aspects, the KRAS inhibitor is a means for KRAS inhibition (wherein the means are described herein).

2. Treg-depleting agent

In the following, the Treg-depleting agent suitable for the methods of the present disclosure is further described.

In some aspects, the Treg-depleting agent is capable of specifically binding to CCR8, CTLA-4, CCR4, CD25, TIM-3, VISTA, GITR, 4-1BB, OX-40, CD27, ICOS, CD15s (sialyl Lewis x), MALT1, TNFR2, TGF-B receptor, and/or TIGIT. In some aspects, the Treg-depleting agent is capable of specifically binding to CCR8, CTLA-4, CCR4, and/or CD25.

In some aspects, the Treg-depleting agent is capable of specifically binding to human CCR8, human CTLA-4, human CCR4, human CD25, human TIM-3, human VISTA, human GITR, human 4-1BB, human OX-40, human CD27, human ICOS, human CD15s (sialyl Lewis x), human MALT1, human TNFR2, human TGF-β receptor, and/or human TIGIT.

In some aspects, the Treg-depleting agent is capable of specifically binding to (human) CCR8. In some aspects, the Treg-depleting agent is capable of specifically binding to (human) CTLA-4. In some aspects, the Treg-depleting agent is capable of specifically binding to (human) CCR4. In some aspects, the Treg-depleting agent is capable of specifically binding to (human) CD25. In some aspects, the Treg-depleting agent is capable of specifically binding to (human) TIM-3. In some aspects, the Treg-depleting agent is capable of specifically binding to (human) VISTA. In some aspects, the Treg-depleting agent is capable of specifically binding to (human) GITR. In some aspects, the Treg-depleting agent is capable of specifically binding to (human) 4-1BB. In some aspects, the Treg-depleting agent is capable of specifically binding to (human) OX-40. In some aspects, the Treg-depleting agent is capable of specifically binding to (human) CD27. In some aspects, the Treg-depleting agent is capable of specifically binding to (human) ICOS. In some aspects, the Treg-depleting agent is capable of specifically binding to (human) CD15s (sialyl Lewis x). In some aspects, the Treg-depleting agent is capable of specifically binding to (human) MALT1. In some aspects, the Treg-depleting agent is capable of specifically binding to (human) TNFR2. In some aspects, the Treg-depleting agent is capable of specifically binding to (human) TGF-B receptor. In some aspects, the Treg-depleting agent is capable of specifically binding to (human) TIGIT.

In some aspects, the Treg-depleting agent is capable of specifically binding to CCR8 and/or CTLA-4. In some aspects, the Treg-depleting agent is capable of specifically binding to human CCR8 and/or CTLA-4. In some aspects, the Treg-depleting agent is capable of specifically binding to CCR8. In some aspects, the Treg-depleting agent is capable of specifically binding to human CCR8.

In some aspects, the Treg-depleting agent is not a Treg-depleting agent that is capable of specifically binding to CTLA-4. In some aspects, the Treg-depleting agent is not an anti-CTLA-4 antibody, or an antigen binding portion thereof.

As will be acknowledged by a person of ordinary skill in the art, in aspects, wherein the subject in the methods of the present disclosure is a human, the Treg-depleting agent used in the methods of the present disclosure is capable of specifically binding to a human target (such as human CCR8 or human CTLA-4).

In some aspects, the Treg-depleting agent comprises a small molecule, a nucleic acid, a peptide, or a protein.

In some aspects, the Treg-depleting agent comprises a nucleic acid. In some aspects, the nucleic acid comprises a siRNA, an antisense oligonucleotide, an antigomir, or a miRNA. In some aspects, the antisense oligonucleotide targets FOXP3 in Treg cells.

In some aspects, the Treg-depleting agent comprises a small molecule. In some aspects, the small molecule is FLX475, galunisertib, LY3200882, vactosertib, gamabufotalin or zoledronic acid. In some aspects, the small molecule is FLX475.

In some aspects, the Treg-depleting agent comprises a peptide. In some aspects, the peptide is methionine encephalin.

In some aspects, the Treg-depleting agent comprises a protein. In some aspects, the protein is bintrafusp alfa or denileukin difitox. In some aspects, the protein comprises an antibody or an antigen binding portion thereof.

In some aspects, the Treg-depleting agent comprises an antibody or an antigen binding portion thereof. In some aspects, the antibody or the antigen binding portion thereof is capable of specifically binding to CCR8, CTLA-4, CCR4, CD25, TIM-3, VISTA, GITR, 4-1BB, OX-40, CD27, ICOS, CD15s (sialyl Lewis x), MALT1, TNFR2, TGF-β receptor, and/or TIGIT. In some aspects, the antibody or the antigen binding portion thereof is capable of specifically binding to human CCR8, CTLA-4, CCR4, CD25, TIM-3, VISTA, GITR, 4-1BB, OX-40, CD27, ICOS, CD15s (sialyl Lewis x), MALT1, TNFR2, TGF-β receptor, and/or TIGIT. In some aspects, the antibody or the antigen binding portion thereof is capable of specifically binding to (human) CCR8. In some aspects, the antibody or the antigen binding portion thereof is capable of specifically binding to (human) CTLA-4. In some aspects, the antibody or the antigen binding portion thereof is capable of specifically binding to (human) CCR4. In some aspects, the antibody or the antigen binding portion thereof is capable of specifically binding to (human) CD25. In some aspects, the antibody or the antigen binding portion thereof is capable of specifically binding to (human) TIM-3. In some aspects, the antibody or the antigen binding portion thereof is capable of specifically binding to (human) VISTA. In some aspects, the antibody or the antigen binding portion thereof is capable of specifically binding to (human) GITR. In some aspects, the antibody or the antigen binding portion thereof is capable of specifically binding to (human) 4-1BB. In some aspects, the antibody or the antigen binding portion thereof is capable of specifically binding to (human) OX-40. In some aspects, the antibody or the antigen binding portion thereof is capable of specifically binding to (human) CD27. In some aspects, the antibody or the antigen binding portion thereof is capable of specifically binding to (human) ICOS. In some aspects, the antibody or the antigen binding portion thereof is capable of specifically binding to (human) CD15s (sialyl Lewis x). In some aspects, the antibody or the antigen binding portion thereof is capable of specifically binding to (human) MALT1. In some aspects, the antibody or the antigen binding portion thereof is capable of specifically binding to (human) TNFR2. In some aspects, the antibody or the antigen binding portion thereof is capable of specifically binding to (human) TGF-β receptor. In some aspects, the antibody or the antigen binding portion thereof is capable of specifically binding to (human) TIGIT.

In some aspects, the antibody or the antigen binding portion thereof is capable of specifically binding to CCR8 and/or CTLA-4. In some aspects, the antibody or the antigen binding portion thereof is capable of specifically binding to human CCR8 and/or human CTLA-4. In some aspects, the antibody or the antigen binding portion thereof is capable of specifically binding to CCR8. In some aspects, the antibody or the antigen binding portion thereof is capable of specifically binding to human CCR8. In some aspects, the antibody or the antigen binding portion thereof for the Treg-depleting agent is capable of specifically binding to CTLA-4. In some aspects, the antibody or the antigen binding portion thereof for the Treg-depleting agent is capable of specifically binding to human CTLA-4.

In some aspects, the Treg-depleting agent comprises a bispecific antibody against CTLA-4 and OX40, such as ATOR-1015, or an antigen binding portion thereof.

In some aspects, the Treg-depleting agent comprises an antibody or an antigen binding portion thereof. In some aspects, the antibody is a monoclonal antibody. In some aspects, the antibody is a human antibody. In some aspects, the antibody is a humanized antibody. In some aspects, the antibody mediates ADCC, antibody-dependent cellular phagocytosis (“ADCP”), and/or CDC. In some aspects, the antibody comprises a heavy chain constant region, which is of a human IgG1 or IgG3 isotype, wherein the IgG1 or IgG3 isotypes are examples of antibodies that mediate ADCC, ADCP, and/or CDC. In some aspects, the antibody is a bispecific antibody. In some aspects, the antibody is an activatable antibody (wherein the activatable antibody might be a monoclonal antibody). In some aspects, the antibody is a nonfucosylated antibody (wherein the nonfucosylated antibody might be a monoclonal antibody). In some aspects, the antibody is a hypofucosylated antibody (wherein the nonfucosylated antibody might be a monoclonal antibody).

In some aspects, the antibody (such as an anti-CCR8 antibody) comprises a modified IgG1 heavy chain constant region, which contains a mutation, or a multiplicity of mutations, that mediate enhanced ADCC (via enhanced Fcγ receptor binding). In some aspects, the mutation or multiplicity of mutations is chosen from G236A; S239D; F243L; E333A; G236A/I332E; S239D/I332E; S267E/H268F; S267E/S324T; H268F/S324T; G236A/S239D/I332E; S239D/A330L/I332E; S267E/H268F/S324T; and G236A/S239D/A330L/I332E (all residue numbering per the EU numbering system). In some aspects, the antibody comprises a human IgG1 constant domain with the following mutations: G236A, S239D, A330L, and I332E (all residue numbering per the EU numbering system). In some aspects, the antibody comprises a human IgG1 constant domain with the following mutations: S239D, A330L, and I332E (all residue numbering per the EU numbering system).

In some aspects, wherein the Treg-depleting agent comprises an antibody, or an antigen binding portion thereof, the antibody or antigen binding portion thereof is administered by parenteral administration (systemically), such as intravenous administration. In some aspects, the antibody or antigen binding portion thereof is administered by intravenous infusion. In some aspects, the antibody or antigen binding portion thereof is administered by subcutaneous injection. In some aspects, the Treg-depleting agent is administered locally to the tumor tissue. In some aspects, the Treg-depleting agent is administered intra-tumorally or peri-tumorally.

In some aspects, wherein the Treg-depleting agent comprises an antibody, or an antigen binding portion thereof (such as an anti-CCR8 antibody, or antigen binding portion thereof), the antibody or antigen binding portion thereof is administered at about 0.01 to about 20 mg/kg, such as at about 0.1 to about 10 mg/kg, of the subject's body weight. In some aspects, the antibody or antigen binding portion thereof is administered at about 0.1, 0.3, 1, 2, 3, 5 or 10 mg/kg body weight. In some aspects, the antibody or antigen binding portion thereof is administered at about 0.1 to about 2,000 mg, or about 1 to about 1,000 mg, such as about 0.3, 1, 3, 5, 10, 30, 60, 100, 150, 200, 240, 300, 400, 500, 600, 800 or 1,000 mg.

In some aspects, wherein the Treg-depleting agent comprises an antibody, or an antigen binding portion thereof (such as an anti-CCR8 antibody, or antigen binding portion thereof), the antibody or antigen binding portion thereof is administered every week, every two weeks, every three weeks, every four weeks, every five weeks, or every six weeks. In some aspects, the antibody or antigen binding portion thereof is administered every week. In some aspects, the antibody or antigen binding portion thereof is administered every two weeks. In some aspects, the antibody or antigen binding portion thereof is administered every three weeks.

In some aspects, wherein the Treg-depleting agent comprises an antibody, or an antigen binding portion thereof (such as an anti-CCR8 antibody, or antigen binding portion thereof), the antibody or antigen binding portion thereof is administered once per week, once every 2 weeks, once every 3 weeks, once every 4 weeks, once a month, once every 3-6 months, or longer.

In some aspects, wherein the Treg-depleting agent comprises an antibody, or an antigen binding portion thereof, the antibody or antigen binding portion thereof is administered every 5 days, every 6 days, every 7 days, every 8 days, every 9 days, every 10 days, every 11 days, every 12 days, every 13 days, every 14 days, every 15 days, every 16 days, every 17 days, every 18 days, every 19 days, every 20 days, every 21 days, every 22 days, every 23 days, every 24 days, every 25 days, every 26 days, every 27 days, or every 28 days.

In some aspects, the Treg-depleting agent comprises an anti-TGF-β receptor antibody or an antigen binding portion thereof. In some aspects, the anti-TGF-β receptor antibody is DS-1055a or fresolimumab.

In some aspects, the Treg-depleting agent comprises an anti-CD25 antibody or an antigen binding portion thereof. In some aspects, the anti-CD25 antibody is daclizumab.

In some aspects, the Treg-depleting agent comprises an anti-4-1BB antibody or an antigen binding portion thereof. In some aspects, the anti-4-1BB antibody is urelumab, utomilumab, EU101, or YH004.

In some aspects, the Treg-depleting agent comprises an anti-CD27 antibody or an antigen binding portion thereof. In some aspects, the anti-CD27 antibody is varilumab, CDX-1127 or BMS-986215.

In some aspects, the Treg-depleting agent comprises an anti-CCR4 antibody or an antigen binding portion thereof. In some aspects, the anti-CCR4 antibody is mogamulizumab. Mogamulizumab, sold under the brand name POTELIGEO®, is a humanized, afucosylated monoclonal antibody targeting CC chemokine receptor 4 (CCR4). See, for example, Watson et al. (2019) J. Adv. Pract. Oncol. 10 (8): 883-888.

In the subsections below, further aspects regarding specific Treg-depleting agents of the present disclosure are described.

In some aspects, the Treg cell depleting agent is a means for depleting Treg cells (as described herein). In some aspects, the Treg cell depleting agent is a means for specifically binding to CCR8 (as described herein). In some aspects, the Treg cell depleting agent is a means for specifically binding to CTLA-4 (as described herein).

### a) Anti-CCR8 Antibody, or Antigen Binding Fragment Thereof

In some aspects, the Treg-depleting agent comprises an anti-CCR8 antibody (i.e., an antibody capable of specifically binding to CCR8), or an antigen binding portion thereof.

In some aspects, the Treg-depleting agent comprises an anti-CCR8 antibody. In some aspects, the Treg-depleting agent comprises a monoclonal anti-CCR8 antibody. In some aspects, the Treg-depleting agent comprises a human anti-CCR8 antibody. In some aspects, the Treg-depleting agent comprises a humanized anti-CCR8 antibody. In some aspects, the Treg-depleting agent comprises a chimeric anti-CCR8 antibody. In some aspects, the Treg-depleting agent comprises an anti-human-CCR8 antibody (i.e., the antibody specifically binds to human CCR8). The sequence of human CCR8 is given above in Table 6 (SEQ ID NO: 176). In some aspects, the Treg-depleting agent comprises a monospecific anti-CCR8 antibody. In some aspects, the Treg-depleting agent comprises a bispecific anti-CCR8 antibody.

In some aspects, the Treg-depleting agent comprises a human anti-human-CCR8 antibody. In some aspects, the Treg-depleting agent comprises a monoclonal human anti-human-CCR8 antibody.

In some aspects, the anti-CCR8 antibody comprises a heavy chain constant region which is of a human IgG1 isotype.

In some aspects, the anti-CCR8 antibody comprises a heavy chain constant region which is of a human IgG3 isotype.

In some aspects, the anti-CCR8 antibody is a hypofucosylated anti-CCR8 antibody.

In some aspects, the anti-CCR8 antibody is a nonfucosylated anti-CCR8 antibody.

In some aspects, the anti-CCR8 antibody is a nunfocusoylated anti-human-CCR8 antibody that comprises a heavy chain constant region which is of a human IgG1 isotype.

In some aspects, the anti-CCR8 antibody comprises a modified IgG1 heavy chain constant region which contains a mutation, or a multiplicity of mutations, that mediate enhanced ADCC (via enhanced Fcγ receptor binding). In some aspects, the mutation or multiplicity of mutations is chosen from G236A; S239D; F243L; E333A; G236A/I332E; S239D/I332E; S267E/H268F; S267E/S324T; H268F/S324T; G236A/S239D/I332E; S239D/A330L/I332E; S267E/H268F/S324T; and G236A/S239D/A330L/I332E (all residue numbering per the EU numbering system).

In some aspects, the anti-CCR8 antibody is a nonfucosylated anti-CCR8 antibody and comprises a modified IgG1 heavy chain constant region which contains a mutation, or a multiplicity of mutations, that mediate enhanced ADCC (via enhanced Fcγ receptor binding), optionally wherein the mutation or multiplicity of mutations is chosen from G236A; S239D; F243L; E333A; G236A/I332E; S239D/I332E; S267E/H268F; S267E/S324T; H268F/S324T; G236A/S239D/I332E; S239D/A330L/I332E; S267E/H268F/S324T; and G236A/S239D/A330L/I332E (all residue numbering per the EU numbering system).

In some aspects, the anti-CCR8 antibody is an activatable anti-CCR8 antibody.

In some aspects, the anti-CCR8 antibody or antigen binding portion thereof binds to human CCR8 with a K_{D} of 1 μM to 10 pM or lower. In some aspects, the anti-CCR8 antibody or antigen binding portion thereof binds to human CCR8 with a K_{D} of about 100 nM or lower, about 10 nM or lower, about 5 nM or lower, about 5 nM or lower, or about 0.1 nM or lower. K_{D} values for antibodies can be determined using methods well established in the art, such as surface plasmon resonance (SPR), kinetic exclusion assay (KinExA®; Sapidyne Instruments, Boise, ID), or bio-layer interferometry (BLI; ForteBio, Fremont, CA). An example of a SPR method uses a BIACORE® biosensor system (GE Healthcare, Chicago, IL).

Binding affinities for an anti-CCR8 antibody binding to, e.g., human CCR8 can also be determined by measuring the EC₅₀ for binding to CCR8-expressing cell lines (such as CHO, 293F, or Raji cell lines expressing CCR8), which is the concentration of the antibody that achieves half of the maximal binding. In some aspects, the anti-CCR8 antibody or the antigen binding portion thereof binds to CHO cells expressing human CCR8 with an EC₅₀ of: about 10 nM or lower; about 5 nM or lower; about 2 nM or lower; about 1.7 nM or lower; about 1 nM or lower; about 0.5 nM or lower; or about 0.1 nM or lower. In some aspects, the anti-CCR8 antibody or the antigen binding portion thereof binds to CHO cells expressing human CCR8 with an EC₅₀ of: between about 0.1 nM and about 10 nM; between about 0.1 nM and about 2 nM; between about 0.5 nM and about 5 nM; between about 1 nM and about 2 nM; or between about 0.5 nM and about 1 nM. In some aspects, the EC₅₀ is measured as described in Example 11 of WO 2021/194942, hereby incorporated by reference.

In some aspects, the anti-CCR8 antibody or antigen binding portion thereof specifically binds to activated human Tregs with an EC₅₀ of: about 50 nM or lower, about 14 nM or lower, about 10 nM or lower; about 5 nM or lower; about 2 nM or lower; about 0.5 nM or lower; about 0.3 nM or lower; about 0.1 nM or lower; or about 0.03 nM or lower. In some aspects, the anti-CCR8 antibody or antigen binding portion thereof specifically binds to activated human Tregs with an EC₅₀ of: about 1.7 nM, between about 0.03 nM and about 10 nM; between about 0.1 nM and about 5 nM; or between about 0.2 nM and about 2 nM. In some aspects, the EC₅₀ is measured as described in Example 11 of WO 2021/194942, hereby incorporated by reference.

In some aspects, the anti-CCR8 antibody or antigen binding portion thereof, mediates depletion of activated human Tregs with an EC₅₀, optionally as measured by an apoptosis assay as described in Example 19 of WO 2021/194942, of: about 500 pM or lower; about 100 pM or lower; about 30 pM or lower; about 15 pM or lower; about 5 pM or lower; about 1 pM or lower; about 13 pM; between about 1 pM and about 500 pM; between about 5 pM and about 100 pM; or between about 10 pM and about 50 pM.

In some aspects, the anti-CCR8 antibody or antigen binding portion thereof binds to an N-terminal peptide of human CCR8 (i.e., is an anti-human-CCR8 antibody or antigen binding portion thereof), wherein human CCR8 comprises a sulfated tyrosine-15 residue and/or a sulfated tyrosine-17 residue (e.g., a peptide of N-terminal CCR8 residues 1-35 sulfated at positions tyr-15 and tyr-17).

In some aspects, the anti-CCR8 antibody or antigen binding portion thereof binds to an N-terminal peptide of human CCR8 comprising sulfated tyrosine-15 and tyrosine-17 residues with a K_{D}, as measured by surface plasmon resonance (SPR), of: about 100 nM or lower; about 50 nM or lower; about 10 nM or lower; about 5 nM or lower; about 1.6 nM; about 1.0 nM or lower; about 0.5 nM or lower; about 0.1 nM or lower; between about 100 nM and about 0.1 nM; between about 50 nM and about 0.5 nM; between about 10 nM and about 1 nM; or between about 2 nM and about 1 nM. In some aspects, SPR is performed as described in Example 11 of WO 2021/194942, hereby incorporated by reference.

In some aspects, the anti-CCR8 antibody or antigen binding portion thereof binds to an N-terminal peptide of human CCR8 comprising a single sulfated residue, i.e., tyrosine-15, with a K_{D}, as measured by SPR, of: about 100 nM or lower; about 50 nM or lower; about 25 nM or lower; about 10 nM or lower; about 1.0 nM or lower; about 20 nM; between about 100 nM and about 1 nM; between about 50 nM and about 10 nM; or between about 30 nM and about 20 nM. In some aspects, SPR is performed as described in Example 11 of WO 2021/194942, hereby incorporated by reference.

In some aspects, the anti-CCR8 antibody or antigen binding portion thereof, inhibits binding of C-C Motif Chemokine Ligand 1 (CCL1) to CCR8 and inhibits CCR8/CCL1 signaling, optionally as measured by inhibition of calcium flux as described in Example 15 of WO 2021/194942, hereby incorporated by reference. In some aspects, the anti-CCR8 antibody or antigen binding portion thereof, inhibits CCR8/CCL1 signaling with an IC₅₀ of: about 10 nM or lower; about 5 nM or lower; about 1 nM or lower; about 0.5 nM or lower; about 0.1 nM or lower; about 0.01 nM or lower; between about 0.01 nM and about 10 nM; between about 0.05 nM and about 5 nM; between about 0.1 nM and about 1 nM; or about 0.46 nM.

In some aspects, the anti-CCR8 antibody or antigen binding portion thereof, mediates depletion of the CCR8-expressing cell with an EC₅₀, optionally as measured by a CD16 cross-linking assay as described in Example 17 of WO 2021/194942, of: about 100 pM or lower; about 30 pM or lower; about 10 pM or lower; about 3 pM or lower; about 1 pM or lower; about 0.5 pM or lower; about 0.1 pM or lower; about 0.05 pM or lower; about 0.7 pM; between about 0.05 pM and about 50 pM; between about 0.1 pM and about 10 nM; between about 0.3 nM and about 7 nM; or between about 0.6 nM and about 3 nM.

In some aspects, the anti-CCR8 antibody or antigen binding portion thereof, binds to an N-terminal peptide of human CCR8 (i.e., is an anti-human-CCR8 antibody or antigen binding portion thereof), wherein the epitope to which the anti-CCR8 antibody or antigen binding portion thereof binds to, comprises at least one amino acid within a peptide having the sequence Y₁₅Y₁₆Y₁₇P₁₈D₁₉I₂₀F₂₁ (SEQ ID NO: 180, see also Table 6, wherein the corresponding amino acids are highlighted). In some aspects, the epitope comprises 2, 3, 4, 5, 6, or all the amino acids within a peptide having the sequence of SEQ ID NO: 180. In some aspects, the epitope peptide comprises a sulfated tyrosine-17 residue and/or a sulfated tyrosine-15 residue. In some aspects, the anti-CCR8 antibody binds to an epitope located in the N-terminal domain of human CCR8 (optionally with a K_{D} of about 10 nM or lower), wherein the epitope comprises a peptide having the sequence Y₁₅Y₁₆Y₁₇P₁₈D₁₉I₂₀F₂₁ (SEQ ID NO: 180) and a sulfated tyrosine-15 residue and/or a sulfated tyrosine-17 residue.

In some aspects, the anti-CCR8 antibody or antigen binding portion thereof binds to an N-terminal peptide of human CCR8 (i.e., is an anti-human-CCR8 antibody or antigen binding portion thereof), wherein the epitope to which the anti-CCR8 antibody or antigen binding portion thereof binds to, comprises at least one amino acid within a peptide having the sequence V₁₂T₁₃D₁₄Y₁₅Y₁₆Y₁₇P₁₈D₁₉I₂₀F₂₁S₂₂ (SEQ ID NO: 181). In some aspects, the epitope comprises 2, 3, 4, 5, 6, 7, 8, 9, 10 or all the amino acids within a peptide having the sequence of SEQ ID NO: 181. In some aspects, the anti-CCR8 antibody or antigen binding portion thereof binds to an N-terminal peptide of human CCR8 (i.e., is an anti-human-CCR8 antibody or antigen binding portion thereof), wherein the epitope to which the anti-CCR8 antibody or antigen binding portion thereof binds to, comprises or consists of SEQ ID NO: 181. In some aspects, the epitope peptide comprises a sulfated tyrosine-17 residue and/or a sulfated tyrosine-15 residue. In some aspects, the anti-CCR8 antibody binds to an epitope located in the N-terminal domain of human CCR8 (optionally with a K_{D} of about 10 nM or lower), wherein the epitope comprises or consists of a peptide having the sequence V₁₂T₁₃D₁₄Y₁₅Y₁₆Y₁₇P₁₈D₁₉I₂₀F₂₁S₂₂ (SEQ ID NO: 181) and a sulfated tyrosine-15 residue and/or a sulfated tyrosine-17 residue.

Exemplary amino acid sequences of CDRH1, CDRH2, CDRH3, CDRL1, CDRL2, CDRL3, VH, VL, heavy chain, and light chain amino acid sequences of anti-CCR8 antibodies suitable for application in the methods of the present disclosure are described in Table 9.

TABLE 9
Exemplary CDRH1, CDRH2, CDRH3, CDRL1, CDRL2, CDRL3, VH, VL, heavy chain, and
light chain amino acid sequences of anti-CCR8 antibodies suitable for
application in the methods of the present disclosure.
Anti-CCR8 antibody 14S15

| | |
|---|---|
| CDRH1 (SEQ ID NO: 1) | GFRFNPYAMN |
| CDRH2 (SEQ ID NO: 2) | RIRSKSNNYE TYYADSVKD |
| CDRH3 (SEQ ID NO: 3) | YSDLYVLDY |
| CDRL1 (SEQ ID NO: 4) | RSSKSLLHSN GNTFLY |
| CDRL2 (SEQ ID NO: 5) | RMSNLAS |
| CDRL3 (SEQ ID NO: 6) | MQHLEYPFT |
| VH (SEQ ID NO: 7) | EVQLVEFGGG LVQPKGSLKL SCVVSGFRFN PYAMNWVRQA |
| | PGKGLEWLGR IRSKSNNYET YYADSVKDRF IISRDDSQKM |
| | VYLQMNNVKT EDTAMYYCVG YSDLYVLDYW GQGTSVTVSS |
| VL (SEQ ID NO: 8) | DIVMTQAAPS VPVTPGESVS ISCRSSKSLL HSNGNTFLYW |
| | FLQRPGQSPQ LLIYRMSNLA SGVPHRFSGS GSGTAFTLRI |
| | SRVEAEDVGV YYCMQHLEYP FTFGSGTKLE IK |
| Heavy chain | EVQLVEFGGG LVQPKGSLKL SCVVSGFRFN PYAMNWVRQA |
| (SEQ ID NO: 9) | PGKGLEWLGR IRSKSNNYET YYADSVKDRF IISRDDSQKM |
| | VYLQMNNVKT EDTAMYYCVG YSDLYVLDYW GQGTSVTVSS |
| | ASTKGPSVFP LAPSSKSTSG GTAALGCLVK DYFPEPVTVS |
| | WNSGALTSGV HTFPAVLQSS GLYSLSSVVT VPSSSLGTQT |
| | YICNVNHKPS NTKVDKRVEP KSCDKTHTCP PCPAPELLGG |
| | PSVFLFPPKP KDTLMISRTP EVTCVVVDVS HEDPEVKENW |
| | YVDGVEVHNA KTKPREEQYN STYRVVSVLT VLHQDWLNGK |
| | EYKCKVSNKA LPAPIEKTIS KAKGQPREPQ VYTLPPSREE |
| | MTKNQVSLTC LVKGFYPSDI AVEWESNGQP ENNYKTTPPV |
| | LDSDGSFFLY SKLTVDKSRW QQGNVFSCSV MHEALHNHYT |
| | QKSLSLSPGK |
| Light chain | DIVMTQAAPS VPVTPGESVS ISCRSSKSLL HSNGNTFLYW |
| (SEQ ID NO: 10) | FLQRPGQSPQ LLIYRMSNLA SGVPHRFSGS GSGTAFTLRI |
| | SRVEAEDVGV YYCMQHLEYP FTFGSGTKLE IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSFNRGEC |

Anti-CCR8 antibody 18Y12

| | |
|---|---|
| CDRH1 (SEQ ID NO: 11) | GYTFTDYEMH |
| CDRH2 (SEQ ID NO: 12) | AIDPKTGSTA YNQKFKG |
| CDRH3 (SEQ ID NO: 13) | LRRFVY |
| CDRL1 (SEQ ID NO: 14) | RSSKSLLHSN GNTFLY |
| CDRL2 (SEQ ID NO: 15) | RVSNRFS |
| CDRL3 (SEQ ID NO: 16) | SQSTHVPFT |
| VH (SEQ ID NO: 17) | QVQLQQSGAE LVRPGASVTL SCKASGYTFT DYEMHWVKQT |
| | PVHGLEWIGA IDPKTGSTAY NQKFKGKAIM TADKSSSTAY |
| | MELRSLTSED SVVYYCTGLR RFVYWGQGTP VTVSA |
| VL (SEQ ID NO: 18) | DVVMTQTPLS LPVSLGDQAS ISCRSRQSLV HSSGYTYLHW |
| | YLQKPGQSPK LLIYRVSNRF SGVPDRFSGS GSGTDFTLKI |
| | SRVEAEDLGV YFCSQSTHVP FTFGSGTKLE IK |
| Heavy chain | QVQLQQSGAE LVRPGASVTL SCKASGYTFT DYEMHWVKQT |
| (SEQ ID NO: 19) | PVHGLEWIGA IDPKTGSTAY NQKFKGKAIM TADKSSSTAY |
| | MELRSLTSED SVVYYCTGLR RFVYWGQGTP VTVSAASTKG |
| | PSVFPLAPSS KSTSGGTAAL GCLVKDYFPE PVTVSWNSGA |
| | LTSGVHTFPA VLQSSGLYSL SSVVTVPSSS LGTQTYICNV |
| | NHKPSNTKVD KRVEPKSCDK THTCPPCPAP ELLGGPSVFL |
| | FPPKPKDTLM ISRTPEVTCV VVDVSHEDPE VKFNWYVDGV |
| | EVHNAKTKPR EEQYNSTYRV VSVLTVLHQD WLNGKEYKCK |
| | VSNKALPAPI EKTISKAKGQ PREPQVYTLP PSREEMTKNQ |
| | VSLTCLVKGF YPSDIAVEWE SNGQPENNYK TTPPVLDSDG |
| | SFFLYSKLTV DKSRWQQGNV FSCSVMHEAL HNHYTQKSLS |
| | LSPGK |
| Light chain | DVVMTQTPLS LPVSLGDQAS ISCRSRQSLV HSSGYTYLHW |
| (SEQ ID NO: 20) | YLQKPGQSPK LLIYRVSNRF SGVPDRFSGS GSGTDFTLKI |
| | SRVEAEDLGV YFCSQSTHVP FTFGSGTKLE IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSFNRGEC |

Anti-CCR8 antibody 4A19 (imzokitug)

| | |
|---|---|
| CDRH1 (SEQ ID NO: 21) | GYTFTDSEMH |
| CDRH2 (SEQ ID NO: 22) | AIQPETGGTA YNQKFKA |
| CDRH3 (SEQ ID NO: 23) | RRRNFDY |
| CDRL1 (SEQ ID NO: 24) | RSSQSLFHSS GNTYLH |
| CDRL2 (SEQ ID NO: 25) | KVSNRFS |
| CDRL3 (SEQ ID NO: 26) | SQSTHVPFT |
| VH (SEQ ID NO: 27) | QVQLVQSGAE VKKPGASVKV SCKASGYTFT DSEMHWVRQA |
| | TGQGLEWMGA IQPETGGTAY NQKFKARVTM TRDTSISTAY |
| | MELSSLRSED TAVYYCARRR RNFDYWGQGT LVTVSS |
| VL (SEQ ID NO: 28) | DIVMTQTPLS LSVTPGQPAS ISCRSSQSLF HSSGNTYLHW |
| | YLQKPGQPPQ LLIYKVSNRF SGVPDRFSGS GSGTDFTLKI |
| | SRVEAEDVGV YYCSQSTHVP FTFGQGTKLE IK |
| Heavy chain | QVQLVQSGAE VKKPGASVKV SCKASGYTFT DSEMHWVRQA |
| (SEQ ID NO: 29) | TGQGLEWMGA IQPETGGTAY NQKFKARVTM TRDTSISTAY |
| | MELSSLRSED TAVYYCARRR RNFDYWGQGT LVTVSSASTK |
| | GPSVFPLAPS SKSTSGGTAA LGCLVKDYFP EPVTVSWNSG |
| | ALTSGVHTFP AVLQSSGLYS LSSVVTVPSS SLGTQTYICN |
| | VNHKPSNTKV DKRVEPKSCD KTHTCPPCPA PELLGGPSVF |
| | LFPPKPKDTL MISRTPEVTC VVVDVSHEDP EVKFNWYVDG |
| | VEVHNAKTKP REEQYNSTYR VVSVLTVLHQ DWLNGKEYKC |
| | KVSNKALPAP IEKTISKAKG QPREPQVYTL PPSREEMTKN |
| | QVSLTCLVKG FYPSDIAVEW ESNGQPENNY KTTPPVLDSD |
| | SLSPG VDKSRWQQGN VFSCSVMHEA LHNHYTQKSL |
| | GSFFLYSKLT |
| Light chain | DIVMTQTPLS LSVTPGQPAS ISCRSSQSLF HSSGNTYLHW |
| (SEQ ID NO: 30) | YLQKPGQPPQ LLIYKVSNRF SGVPDRFSGS GSGTDFTLKI |
| | SRVEAEDVGV YYCSQSTHVP FTFGQGTKLE IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSFNRGEC |

Anti-CCR8 antibody 10R3

| | |
|---|---|
| CDRH1 (SEQ ID NO: 31) | GFTFSSYAMH |
| CDRH2 (SEQ ID NO: 32) | IISYDGSNKY YADSVKG |
| CDRH3 (SEQ ID NO: 33) | VPNWRGAFDI |
| CDRL1 (SEQ ID NO: 34) | RASQSIGSHL H |
| CDRL2 (SEQ ID NO: 35) | YASQSFS |
| CDRL3 (SEQ ID NO: 36) | HQSYSLPLT |
| VH (SEQ ID NO: 37) | QVQLVESGGG VVQPGRSLRL SCAASGFTFS SYAMHWVRQA |
| | PGKGLEWVGI ISYDGSNKYY ADSVKGRFTI SRDNSKNTLY |
| | LQMNSLRAED TALYSCARVP NWRGAFDIWG QGTMVTVSS |
| VL (SEQ ID NO: 38) | EIVLTQSPDF QSVTPKEKVT ITCRASQSIG SHLHWYQQKP |
| | DQSPKLLIKY ASQSFSGVPS RFSGSGSGTD FSLTINSLET |
| | EDAATYFCHQ SYSLPLTFGG GTKVEIK |

Anti-CCR8 antibody 8D55

| | |
|---|---|
| CDRH1 (SEQ ID NO: 39) | GFTFSNYNMI |
| CDRH2 (SEQ ID NO: 40) | YISSSRSIIS YADSVKG |
| CDRH3 (SEQ ID NO: 41) | RWKGVFDY |
| CDRL1 (SEQ ID NO: 42) | RASQGISSWL A |
| CDRL2 (SEQ ID NO: 43) | AASSLQS |
| CDRL3 (SEQ ID NO: 44) | QQYNSYPPT |
| VH (SEQ ID NO: 45) | QVQLVESGGG LVQPGGSLRL SCAASGFTFS NYNMIWVRQT |
| | PGKGLEWISY ISSSRSIISY ADSVKGRFTI SRDNAKNSLY |
| | LQMNSLRDED TAVYYCARRW KGVFDYWGQG TLVTVSS |
| VL (SEQ ID NO: 46) | DIVMTQSPSS LSASVGDRVT ITCRASQGIS SWLAWYQQKP |
| | EKAPKSLIYA ASSLQSGVPS RFSGSGSGTD FTLTISSLQP |
| | EDFATYYCQQ YNSYPPTFGQ GTKVEIK |

Anti-CCR8 antibody 14S15h

| | |
|---|---|
| CDRH1 (SEQ ID NO: 47) | GFRFGPYAMN |
| CDRH2 (SEQ ID NO: 48) | RIRSKSNNYE TYYADSVKD |
| CDRH3 (SEQ ID NO: 49) | YSDLYVLDY |
| CDRL1 (SEQ ID NO: 50) | RSSKSLLHSQ GNTFLY |
| CDRL2 (SEQ ID NO: 51) | RMSNLAS |
| CDRL3 (SEQ ID NO: 52) | MQHLEYPFT |
| VH (SEQ ID NO: 53) | EVQLVESGGG LVKPGGSLRL SCAASGFRFG PYAMNWVRQA |
| | PGKGLEWVGR IRSKSNNYET YYADSVKDRF TISRDDSKNT |
| | LYLQMNSLKT EDTAVYYCVG YSDLYVLDYW GQGTLVTVSS |
| VL (SEQ ID NO: 54) | DIVMTQTPPS LPVNPGEPAS ISCRSSKSLL HSQGNTFLYW |
| | YLQKPGQSPQ LLIYRMSNLA SGVPDRFSGS GSGSDFTLKI |
| | SWVEAEDVGV YYCMQHLEYP FTFGPGTKVD IK |
| Heavy chain | EVQLVESGGG LVKPGGSLRL SCAASGFRFG PYAMNWVRQA |
| (SEQ ID NO: 55) | PGKGLEWVGR IRSKSNNYET YYADSVKDRF TISRDDSKNT |
| | LYLQMNSLKT EDTAVYYCVG YSDLYVLDYW GQGTLVTVSS |
| | ASTKGPSVFP LAPSSKSTSG GTAALGCLVK DYFPEPVTVS |
| | WNSGALTSGV HTFPAVLQSS GLYSLSSVVT VPSSSLGTQT |
| | YICNVNHKPS NTKVDKRVEP KSCDKTHTCP PCPAPELLGG |
| | PSVFLFPPKP KDTLMISRTP EVTCVVVDVS HEDPEVKFNW |
| | YVDGVEVHNA KTKPREEQYN STYRVVSVLT VLHQDWLNGK |
| | EYKCKVSNKA LPAPIEKTIS KAKGQPREPQ VYTLPPSREE |
| | MTKNQVSLTC LVKGFYPSDI AVEWESNGQP ENNYKTTPPV |
| | LDSDGSFFLY SKLTVDKSRW QQGNVFSCSV MHEALHNHYT |
| | QKSLSLSPG |
| Light chain | DIVMTQTPPS LPVNPGEPAS ISCRSSKSLL HSQGNTFLYW |
| (SEQ ID NO: 56) | YLQKPGQSPQ LLIYRMSNLA SGVPDRFSGS GSGSDFTLKI |
| | SWVEAEDVGV YYCMQHLEYP FTFGPGTKVD IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSFNRGEC |

In some aspects, the anti-CCR8 antibody or antigen binding portion thereof comprises a VH comprising a CDRH1 comprising (or consisting of) the amino acid sequence set forth in SEQ ID NO: 1, a CDRH2 comprising (or consisting of) the amino acid sequence set forth in SEQ ID NO: 2, and a CDRH3 comprising (or consisting of) the amino acid sequence set forth in SEQ ID NO: 3; and a VL comprising a CDRL1 comprising (or consisting of) the amino acid sequence set forth in SEQ ID NO: 4, a CDRL2 comprising (or consisting of) the amino acid sequence set forth in SEQ ID NO: 5, and a CDRL3 comprising (or consisting of) the amino acid sequence set forth in SEQ ID NO: 6. In some aspects, the anti-CCR8 antibody is a chimeric, humanized, or human anti-human-CCR8 antibody.

In some aspects, the anti-CCR8 antibody or antigen binding portion thereof comprises a VH comprising (or consisting of) the amino acid sequence set forth in SEQ ID NO: 7, and a VL comprising (or consisting of) the amino acid sequence set forth in SEQ ID NO: 8. In some aspects, the anti-CCR8 antibody comprises a heavy chain comprising (or consisting of) the amino acid sequence as set forth in SEQ ID NO: 9 and a light chain comprising (or consisting of) the amino acid sequence as set forth in SEQ ID NO: 10.

In some aspects, the anti-CCR8 antibody, or antigen binding portion thereof, comprises a VH comprising a CDRH1 comprising (or consisting of) the amino acid sequence set forth in SEQ ID NO: 11, a CDRH2 comprising (or consisting of) the amino acid sequence set forth in SEQ ID NO: 12, and a CDRH3 comprising (or consisting of) the amino acid sequence set forth in SEQ ID NO: 13; and a VL comprising a CDRL1 comprising (or consisting of) the amino acid sequence set forth in SEQ ID NO: 14, a CDRL2 comprising (or consisting of) the amino acid sequence set forth in SEQ ID NO: 15, and a CDRL3 comprising (or consisting of) the amino acid sequence set forth in SEQ ID NO: 16. In some aspects, the anti-CCR8 antibody is a chimeric, humanized, or human anti-human-CCR8 antibody.

In some aspects, the anti-CCR8 antibody, or antigen binding portion thereof, comprises a VH comprising (or consisting of) the amino acid sequence set forth in SEQ ID NO: 17, and a VL comprising (or consisting of) the amino acid sequence set forth in SEQ ID NO: 18. In some aspects, the anti-CCR8 antibody comprises a heavy chain comprising (or consisting of) the amino acid sequence as set forth in SEQ ID NO: 19 and a light chain comprising (or consisting of) the amino acid sequence as set forth in SEQ ID NO: 20.

In some aspects, the anti-CCR8 antibody, or antigen binding portion thereof, comprises a VH comprising a CDRH1 comprising (or consisting of) the amino acid sequence set forth in SEQ ID NO: 21, a CDRH2 comprising (or consisting of) the amino acid sequence set forth in SEQ ID NO: 22, and a CDRH3 comprising (or consisting of) the amino acid sequence set forth in SEQ ID NO: 23; and a VL comprising a CDRL1 comprising (or consisting of) the amino acid sequence set forth in SEQ ID NO: 24, a CDRL2 comprising (or consisting of) the amino acid sequence set forth in SEQ ID NO: 25, and a CDRL3 comprising (or consisting of) the amino acid sequence set forth in SEQ ID NO: 26. In some aspects, the anti-CCR8 antibody is a chimeric, humanized, or human anti-human-CCR8 antibody.

In some aspects, the anti-CCR8 antibody, or antigen binding portion thereof, comprises a VH comprising (or consisting of) the amino acid sequence set forth in SEQ ID NO: 27, and a VL comprising (or consisting of) the amino acid sequence set forth in SEQ ID NO: 28. In some aspects, the anti-CCR8 antibody comprises a heavy chain comprising (or consisting of) the amino acid sequence as set forth in SEQ ID NO: 29 and a light chain comprising (or consisting of) the amino acid sequence as set forth in SEQ ID NO: 30.

In some aspects, the anti-CCR8 antibody, or antigen binding portion thereof, comprises a VH comprising a CDRH1 comprising (or consisting of) the amino acid sequence set forth in SEQ ID NO: 31, a CDRH2 comprising (or consisting of) the amino acid sequence set forth in SEQ ID NO: 32, and a CDRH3 comprising (or consisting of) the amino acid sequence set forth in SEQ ID NO: 33; and a VL comprising a CDRL1 comprising (or consisting of) the amino acid sequence set forth in SEQ ID NO: 34, a CDRL2 comprising (or consisting of) the amino acid sequence set forth in SEQ ID NO: 35, and a CDRL3 comprising (or consisting of) the amino acid sequence set forth in SEQ ID NO: 36. In some aspects, the anti-CCR8 antibody is a chimeric, humanized, or human anti-human-CCR8 antibody.

In some aspects, the anti-CCR8 antibody, or antigen binding portion thereof, comprises a VH comprising (or consisting of) the amino acid sequence set forth in SEQ ID NO: 37, and a VL comprising (or consisting of) the amino acid sequence set forth in SEQ ID NO: 38.

In some aspects, the anti-CCR8 antibody, or antigen binding portion thereof, comprises a VH comprising a CDRH1 comprising (or consisting of) the amino acid sequence set forth in SEQ ID NO: 39, a CDRH2 comprising (or consisting of) the amino acid sequence set forth in SEQ ID NO: 40, and a CDRH3 comprising (or consisting of) the amino acid sequence set forth in SEQ ID NO: 41; and a VL comprising a CDRL1 comprising (or consisting of) the amino acid sequence set forth in SEQ ID NO: 42, a CDRL2 comprising (or consisting of) the amino acid sequence set forth in SEQ ID NO: 43, and a CDRL3 comprising (or consisting of) the amino acid sequence set forth in SEQ ID NO: 44. In some aspects, the anti-CCR8 antibody is a chimeric, humanized, or human anti-human-CCR8 antibody.

In some aspects, the anti-CCR8 antibody, or antigen binding portion thereof, comprises a VH comprising (or consisting of) the amino acid sequence set forth in SEQ ID NO: 45, and a VL comprising (or consisting of) the amino acid sequence set forth in SEQ ID NO: 46.

In some aspects, the anti-CCR8 antibody, or antigen binding portion thereof, comprises a VH comprising a CDRH1 comprising (or consisting of) the amino acid sequence set forth in SEQ ID NO: 47, a CDRH2 comprising (or consisting of) the amino acid sequence set forth in SEQ ID NO: 48, and a CDRH3 comprising (or consisting of) the amino acid sequence set forth in SEQ ID NO: 49; and a VL comprising a CDRL1 comprising (or consisting of) the amino acid sequence set forth in SEQ ID NO: 50, a CDRL2 comprising (or consisting of) the amino acid sequence set forth in SEQ ID NO: 51, and a CDRL3 comprising (or consisting of) the amino acid sequence set forth in SEQ ID NO: 52. In some aspects, the anti-CCR8 antibody is a chimeric, humanized, or human anti-human-CCR8 antibody.

In some aspects, the anti-CCR8 antibody, or antigen binding portion thereof, comprises a VH comprising (or consisting of) the amino acid sequence set forth in SEQ ID NO: 53, and a VL comprising (or consisting of) the amino acid sequence set forth in SEQ ID NO: 54.

In some aspects, the anti-CCR8 antibody, or antigen binding portion thereof, comprises a heavy chain comprising (or consisting of) the amino acid sequence as set forth in SEQ ID NO: 55 and a light chain comprising (or consisting of) the amino acid sequence as set forth in SEQ ID NO: 56.

In some aspects, the anti-CCR8 antibody, or antigen binding portion thereof, comprises a VH and/or VL comprising amino acid sequences that are highly similar or homologous to the amino acid sequences of any of the above anti-CCR8 antibodies and which retain the functional properties of these antibodies. In some aspects, the anti-CCR8 antibody, or antigen binding portion thereof, comprises a VH and/or VL comprising an amino acid sequence that is at least 80% identical to the amino acid sequence as set forth in SEQ ID NO: 27 and/or SEQ ID NO: 28, respectively. As used herein, the percent sequence identity between two amino acid sequences is a function of the number of identical positions shared by the sequences relative to the length of the sequences compared (i.e., % identity=number of identical positions/total number of positions being compared×100), taking into account the number of any gaps, and the length of each such gap, introduced to maximize the degree of sequence identity between the two sequences. The comparison of sequences and determination of percent identity between two sequences can be accomplished using mathematical algorithms that are well known to those of ordinary skill in the art. In some aspects, the anti-CCR8 antibody comprises a VH and/or VL comprising an amino acid sequence that is at least 85%, 90%, 95%, or 99% identical to the amino acid sequence as set forth in SEQ ID NO: 27 and/or SEQ ID NO: 28, respectively. In some aspects, wherein the anti-CCR8 antibody comprises a VH and/or VL comprising an amino acid sequence that is highly similar or homologous to the amino acid sequences of any of the anti-CCR8 antibodies as described above, the VH and/or VL amino acid sequences may have 100% identity to at least 1, 2, 3, 4, 5, or all 6 CDRs and at least 85%, 90%, 95%, or 99% identity to the relevant full VH and/or VL sequence of any of the anti-CCR8 antibodies as described above.

In some aspects, the Treg cell depleting agent comprises an anti-CCR8 antibody as described in WO 2021/194942. In some aspects, the Treg cell depleting agent comprises an antigen binding portion of an anti-CCR8 antibody as described in WO 2021/194942. WO 2021/194942 is herewith incorporated by reference in its entirety.

In some aspects, the anti-CCR8 antibody is BMS-986340 (imzokitug), GS-1811, S-531011, or HBM1022.

In some aspects, the anti-CCR8 antibody is a humanized anti-CCR8 antibody as described in Dépis et al., 2020 (Preclinical evaluation of JTX-1811, an anti-CCR8 antibody with enhanced ADCC activity, for preferential depletion of tumor-infiltrating regulatory T cells [abstract]. In: Proceedings of the Annual Meeting of the American Association for Cancer Research 2020; 2020 Apr. 27-28 and June 22-24. Philadelphia (PA): AACR; Cancer Res 80 (16 Suppl): Abstract nr 4532); WO 2020/138489; or Harbour BioMed, 2020 (Harbour BioMed presented its newly discovered anti-human CCR8 novel monoclonal antibodies at the 16th PEGS Boston 2020. Sep. 2, 2020 press release, available at https://www.harbourbiomed.com/news/126.html #(last accessed Dec. 11, 2023)).

In some aspects, the anti-CCR8 antibody or antigen binding portion thereof is described in any of the following (all of which herewith incorporated by reference in their entirety): WO 2007/044756, WO 2013/131010, WO 2017/198631, WO 2018/112033, WO 2019/157098, WO2023010054, WO2022216965, WO2022256563, WO2023208203, WO2021/152186, WO2021260206, WO2021260208, WO2021260209, WO2021260210, WO2023174396, WO2022241034, WO2021194942, WO2023116880, WO2022081718, WO2023288241, WO2022042690, WO2018112032, WO2023020621, WO2023208182, WO2021163064, WO2022078277, U.S. Pat. No. 10,087,259, WO2021178749, WO2022268192, WO2022003156, WO2022136647, WO2022136649, WO2022136650, WO2022117569, WO2022117572, CN110835374, WO2023137466, WO2023193732, WO2018181425, WO2020138489, WO2022004760, WO2021142002, WO2023098888, WO2024227083-A2, US2024141052-A1, WO2024120418-A1, US2024182588-A1, WO2024109657-A1, US2024165227-A1, WO2024094003-A1, WO2024088346-A1, WO2024077239-A1, WO2024062076-A1, WO-2024062072-A3, WO2024059909-A1, WO2024062019-A1, WO2024062082-A1, WO2024040216-A3, WO2024027823-A1, WO2024008110-A1, US2024352134-A1, US2024360228-A1, U.S. Pat. No. 11,932,696-B2, WO2024165468-A1, and WO2024052517-A3.

In some aspects, the anti-CCR8 antibody is a bispecific antibody. CN 110835374, for example, describes a bispecific anti-CCR8 antibody useful in the methods of the present disclosure.

In some aspects, the anti-CCR8 binds to the same epitope as any of the anti-CCR8 antibodies as described herein, such as antibody imzokitug, 14S15, or 14S15h. In some aspects, the antibody described in this paragraph is a monocloncal antibody. In some aspects, the antibody described in this paragraph is a chimeric, humanized, or human antibody.

In some aspects, the anti-CCR8 antibody is an antibody that cross-competes with any of the anti-CCR8 antibodies as described above, such as antibody imzokitug, 14S15, or 14S15h. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of the reference antibody by virtue of their binding to the same epitope region of CCR8. Cross-competing antibodies can be readily identified based on their ability to cross-compete with the anti-CCR8 antibodies described herein in standard CCR8 binding assays such as BIACORE® analysis, ELISA assays or flow cytometry. In some aspects, the Treg-depleting agent comprises an antigen binding portion of the anti-CCR8 antibody (such as an anti-human-CCR8 antibody) that cross-competes for the binding to CCR8 (such as human CCR8) with any anti-CCR8 antibody described herein, such as antibody imzokitug, 14S15, or 14S15h.

In some aspects, the Treg-depleting agent is a bispecific molecule comprising any of the anti-CCR8 antibodies or antigen binding portions thereof as described herein, linked to a binding domain that has a different binding specificity than the anti-CCR8 antibodies or antigen binding portions thereof.

In some aspects, the Treg cell depleting agent is an immunoconjugate comprising any of the anti-CCR8 antibodies or antigen binding portions thereof as described herein, linked to a cytolytic agent, such as a cytotoxin or a radioactive isotope.

In some aspects, the Treg cell depleting agent is a means for specifically binding to CCR8 (wherein the means are described herein).

### b) Anti-CTLA-4 Antibody, or Antigen Binding Fragment Thereof

In some aspects, the Treg-depleting agent comprises an anti-CTLA-4 antibody (i.e., an antibody capable of specifically binding to CTLA-4), or an antigen binding portion thereof.

In some aspects, the Treg-depleting agent comprises an anti-CTLA-4 antibody. In some aspects, the Treg-depleting agent comprises a monoclonal anti-CTLA-4 antibody. In some aspects, the Treg-depleting agent comprises a human anti-CTLA-4 antibody. In some aspects, the Treg-depleting agent comprises a humanized anti-CTLA-4 antibody. In some aspects, the Treg-depleting agent comprises a chimeric anti-CTLA-4 antibody. In some aspects, the Treg-depleting agent comprises an anti-human-CTLA-4 antibody (i.e., the antibody specifically binds to human CTLA-4). In some aspects, the Treg-depleting agent comprises a monospecific anti-CTLA-4 antibody. In some aspects, the Treg-depleting agent comprises is a bispecific anti-CTLA-4 antibody.

In some aspects, the Treg-depleting agent comprises is a human anti-human-CTLA-4 antibody. In some aspects, the Treg-depleting agent comprises a monoclonal human anti-human-CTLA-4 antibody.

In some aspects, the anti-CTLA-4 antibody comprises a heavy chain constant region which is of a human IgG1 isotype.

In some aspects, the anti-CTLA-4 antibody comprises a heavy chain constant region which is of a human IgG3 isotype.

In some aspects, the anti-CTLA-4 antibody is a hypofucosylated anti-CTLA-4 antibody.

In some aspects, the anti-CTLA-4 antibody is a nonfucosylated anti-CTLA-4 antibody.

In some aspects, the anti-CTLA-4 antibody a nunfocusoylated anti-human-CTLA-4 antibody that comprises a heavy chain constant region which is of a human IgG1 isotype.

In some aspects, the anti-CTLA-4 antibody comprises a modified IgG1 heavy chain constant region which contains a mutation, or a multiplicity of mutations, that mediate enhanced ADCC (via enhanced Fcγ receptor binding). In some aspects, the mutation or multiplicity of mutations is chosen from G236A; S239D; F243L; E333A; G236A/I332E; S239D/I332E; S267E/H268F; S267E/S324T; H268F/S324T; G236A/S239D/I332E; S239D/A330L/I332E; S267E/H268F/S324T; and G236A/S239D/A330L/I332E (all residue numbering per the EU numbering system).

In some aspects, the anti-CTLA-4 antibody is a nonfucosylated anti-CTLA-4 antibody and comprises a modified IgG1 heavy chain constant region which contains a mutation, or a multiplicity of mutations, that mediate enhanced ADCC (via enhanced Fcγ receptor binding), optionally wherein the mutation or multiplicity of mutations is chosen from G236A; S239D; F243L; E333A; G236A/I332E; S239D/I332E; S267E/H268F; S267E/S324T; H268F/S324T; G236A/S239D/I332E; S239D/A330L/I332E; S267E/H268F/S324T; and G236A/S239D/A330L/I332E (all residue numbering per the EU numbering system).

In some aspects, the anti-CTLA-4 antibody is an activatable anti-CTLA-4 antibody.

Exemplary amino acid sequences of anti-CTLA-4 antibodies suitable for application in the methods of the present disclosure are described in Table 10.

TABLE 10
Exemplary CDRH1, CDRH2, CDRH3, CDRL1, CDRL2, CDRL3, VH, VL, heavy chain,
and light chain amino acid sequences of anti-CTLA-4 antibodies suitable
for application in the methods of the present disclosure. The masking
moiety in the activatable sequences is bolded, the cleavable moiety in
the activatable sequences is italicized and underlined, and the VL
section in the activatable sequences is underlined.
Ipilimumab

| | |
|---|---|
| CDRH1 (SEQ ID NO: 141) | SYTMH |
| CDRH2 (SEQ ID NO: 142) | FISYDGNNKYYADSVKG |
| CDRH3 (SEQ ID NO: 143) | TGWLGPFDY |
| CDRL1 (SEQ ID NO: 144) | RASQSVGSSYLA |
| CDRL2 (SEQ ID NO: 145) | GAFSRAT |
| CDRL3 (SEQ ID NO: 146) | QQYGSSPWT |
| VH (SEQ ID NO: 147) | QVQLVESGGGVVQPGRSLRLSCAASGFTFSSYTMHWVRQAPGKGLEWVTF |
| | ISYDGNNKYYADSVKGRFTISRDNSKNTLYLQMNSLRAEDTAIYYCARTG |
| | WLGPFDYWGQGTLVTVSS |
| VL (SEQ ID NO: 148) | EIVLTQSPGTLSLSPGERATLSCRASQSVGSSYLAWYQQKPGQAPRLLIY |
| | GAFSRATGIPDRFSGSGSGTDFTLTISRLEPEDFAVYYCQQYGSSPWTFG |
| | QGTKVEIK |
| Heavy chain | QVQLVESGGGVVQPGRSLRLSCAASGFTFSSYTMHWVRQAPGKGLEWVTF |
| (SEQ ID NO: 149) | ISYDGNNKYYADSVKGRFTISRDNSKNTLYLQMNSLRAEDTAIYYCARTG |
| | WLGPFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Heavy chain lacking C- | QVQLVESGGGVVQPGRSLRLSCAASGFTFSSYTMHWVRQAPGKGLEWVTF |
| terminal K | ISYDGNNKYYADSVKGRFTISRDNSKNTLYLQMNSLRAEDTAIYYCARTG |
| (SEQ ID NO: 150) | WLGPFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPG |
| Light chain | EIVLTQSPGTLSLSPGERATLSCRASQSVGSSYLAWYQQKPGQAPRLLIY |
| (SEQ ID NO: 151) | GAFSRATGIPDRFSGSGSGTDFTLTISRLEPEDFAVYYCQQYGSSPWTFG |
| | QGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQ |
| | GLSSPVTKSFNRGEC |

Activatable ipilimumab

| | |
|---|---|
| Activatable VL | CRTQLYGYNLCPYGGGSSGGSISSGLLSGRSDNHGGGSEIVLTQSPGTLS |
| YV39-2001 | LSPGERATLSCRASQSVGSSYLAWYQQKPGQAPRLLIYGAFSRATGIPDR |
| (SEQ ID NO: 152) | FSGSGSGTDFTLTISRLEPEDFAVYYCQQYGSSPWTFGQGTKVEIK |
| Activatable VL | CRTQLYGYNLCPYGGGSSGGSISSGLLSGRSDNPGGGSEIVLTQSPGTLS |
| YV39-2011 | LSPGERATLSCRASQSVGSSYLAWYQQKPGQAPRLLIYGAFSRATGIPDR |
| (SEQ ID NO: 153) | FSGSGSGTDFTLTISRLEPEDFAVYYCQQYGSSPWTFGQGTKVEIK |
| Activatable VL | CRTQLYGYNLCPYGGGSSGGSISSGLLSGRSANPGGGSEIVLTQSPGTLS |
| YV39-2012 | LSPGERATLSCRASQSVGSSYLAWYQQKPGQAPRLLIYGAFSRATGIPDR |
| (SEQ ID NO: 154) | FSGSGSGTDFTLTISRLEPEDFAVYYCQQYGSSPWTFGQGTKVEIK |
| Activatable light chain | QGQSGSCRTQLYGYNLCPYGGGSSGGSISSGLLSGRSDNPGGGSEIVLTQ |
| Spacer YV39-2011 VL | SPGTLSLSPGERATLSCRASQSVGSSYLAWYQQKPGQAPRLLIYGAFSRA |
| (SEQ ID NO: 155) | TGIPDRFSGSGSGTDFTLTISRLEPEDFAVYYCQQYGSSPWTFGQGTKVE |
| | IKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPV |
| | TKSFNRGEC |

Tremelimumab

| | |
|---|---|
| CDRH1 (SEQ ID NO: 156) | GFTFSSYG |
| CDRH2 (SEQ ID NO: 157) | AVIWYDGSNK |
| CDRH3 (SEQ ID NO: 158) | ARDPRGATLYYYYYGMDV |
| CDRL1 (SEQ ID NO: 159) | QSINSY |
| CDRL2 (SEQ ID NO: 160) | AAS |
| CDRL3 (SEQ ID NO: 161) | QQYYSTPFT |
| VH (SEQ ID NO: 162) | QVQLVESGGGVVQPGRSLRLSCAASGFTFSSYGMHWVRQAPGKGLEWVAV |
| | IWYDGSNKYYADSVKGRFTISRDNSKNTLYLQMNSLRAEDTAVYYCARDP |
| | RGATLYYYYYGMDVWGQGTTVTVSS |
| VL (SEQ ID NO: 163) | DIQMTQSPSSLSASVGDRVTITCRASQSINSYLDWYQQKPGKAPKLLIYA |
| | ASSLQSGVPSRFSGSGSGTDFTLTISSLQPEDFATYYCQQYYSTPFTFGP |
| | GTKVEIKR |
| Light chain | DIQMTQSPSSLSASVGDRVTITCRASQSINSYLDWYQQKPGKAPKLLIYA |
| (SEQ ID NO: 164) | ASSLQSGVPSRFSGSGSGTDFTLTISSLQPEDFATYYCQQYYSTPFTFGP |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC |
| Heavy chain | QVQLVESGGGVVQPGRSLRLSCAASGFTFSSYGMHWVRQAPGKGLEWVAV |
| (SEQ ID NO: 165) | IWYDGSNKYYADSVKGRFTISRDNSKNTLYLQMNSLRAEDTAVYYCARDP |
| | RGATLYYYYYGMDVWGQGTTVTVSSASTKGPSVFPLAPCSRSTSESTAAL |
| | GCLVKDYFPEPVTVSWNSGALTSGVHTFPAVLQSSGLYSLSSVVTVPSSN |
| | FGTQTYTCNVDHKPSNTKVDKTVERKCCVECPPCPAPPVAGPSVFLFPPK |
| | PKDTLMISRTPEVTCVVVDVSHEDPEVQFNWYVDGVEVHNAKTKPREEQF |
| | NSTFRVVSVLTVVHQDWLNGKEYKCKVSNKGLPAPIEKTISKTKGQPREP |
| | QVYTLPPSREEMTKNQVSLTCLVKGFYPSDIAVEWESNGQPENNYKTTPP |
| | MLDSDGSFFLYSKLTVDKSRWQQGNVFSCSVMHEALHNHYTQKSLSLSPG |
| | K |

In some aspects, the anti-CTLA-4 antibody, or antigen binding portion thereof, comprises a VH comprising a CDRH1 comprising (or consisting of) the amino acid sequence as set forth in SEQ ID NO: 156, a CDRH2 comprising (or consisting of) the amino acid sequence as set forth in SEQ ID NO: 157, and a CDRH3 comprising (or consisting of) the amino acid sequence as set forth in SEQ ID NO: 158; and a VL comprising a CDRL1 comprising (or consisting of) the amino acid sequence as set forth in SEQ ID NO: 159, a CDRL2 comprising (or consisting of) the amino acid sequence as set forth in SEQ ID NO: 160, and a CDRL3 comprising (or consisting of) the amino acid sequence as set forth in SEQ ID NO: 161.

In some aspects, the anti-CTLA-4 antibody, or antigen binding portion thereof, comprises a VH comprising (or consisting of) the amino acid sequence as set forth in SEQ ID NO: 162; and a VL comprising (or consisting of) the amino acid sequence as set forth in SEQ ID NO: 163.

In some aspects, the anti-CTLA-4 antibody comprises a heavy chain comprising (or consisting of) the amino acid sequence as set forth in SEQ ID NO: 164 and a light chain comprising (or consisting of) the amino acid sequence as set forth in SEQ ID NO: 165.

In some aspects, the anti-CTLA-4 antibody, or antigen binding portion thereof, comprises a VH comprising a CDRH1 comprising (or consisting of) the amino acid sequence as set forth in SEQ ID NO: 141, a CDRH2 comprising (or consisting of) the amino acid sequence as set forth in SEQ ID NO: 142, and a CDRH3 comprising (or consisting of) the amino acid sequence as set forth in SEQ ID NO: 143; and a VL comprising a CDRL1 comprising (or consisting of) the amino acid sequence as set forth in SEQ ID NO: 144, a CDRL2 comprising (or consisting of) the amino acid sequence as set forth in SEQ ID NO: 145, and a CDRL3 comprising (or consisting of) the amino acid sequence as set forth in SEQ ID NO: 146.

In some aspects, the anti-CTLA-4 antibody, or antigen binding portion thereof, comprises a VH comprising (or consisting of) the amino acid sequence as set forth in SEQ ID NO: 147; and a VL comprising (or consisting of) the amino acid sequence as set forth in SEQ ID NO: 148.

In some aspects, the anti-CTLA-4 antibody comprises a heavy chain comprising the amino acid sequence as set forth in SEQ ID NO: 149 or 150 and a light chain comprising the amino acid sequence as set forth in SEQ ID NO: 151.

In some aspects, the anti-CTLA-4 antibody, or antigen binding portion thereof, comprises a VH comprising (or consisting of) the amino acid sequence as set forth in SEQ ID NO: 148 and a VL comprising (or consisting of) any of the amino acid sequences as set forth in SEQ ID NO: 152-155. In some aspects, the VL further comprises a spacer as set forth in SEQ ID NO: 168 at the amino-terminus.

In some aspects, the anti-CTLA-4 antibody comprises a heavy chain comprising (or consisting of) the amino acid sequence as set forth in SEQ ID NO: 149 or 150 and a light chain comprising (or consisting of) the amino acid sequence as set forth in SEQ ID NO: 155.

In some aspects, the anti-CTLA-4 antibody is tremelimumab, ipilimumab, botensilimab, BMS-986218, BMS-986288, BMS-986249, IBI310, MK-1308 (quavonlimab), AGEN-1884 (zalifrelimab), ONC-392, ADG116, or CS1002.

In some aspects, the anti-CTLA-4 antibody is MK-1308. MK-1308 is also known as quavonlimab. See, for example, Perets et al. (2021) Ann. Oncol. 32 (3): 395-403.

In some aspects, the anti-CTLA-4 antibody is AGEN-1884. AGEN-1884 is also known as zalifrelimab. See, for example, WO 2016/196237.

In some aspects, the anti-CTLA-4 antibody is tremelimumab or ipilimumab. Ipilimumab and tremelimumab were selected to inhibit CTLA-4-B7 interactions (Keler et al. (2003) J. Immunol. 171:6251; Ribas et al. (2007) Oncologist. 12:873) and have been tested in a variety of clinical trials for multiple malignancies. Hoos et al. (2010) Semin. Oncol. 37:533; Ascierto et al. (2011) J. Transl. Med. 9:196.

In some aspects, the anti-CTLA-4 antibody is tremelimumab. Tremelimumab, sold under the brand name IMJUDO®, is a fully human monoclonal antibody used for the treatment of hepatocellular carcinoma and non-small cell lung cancer. Tremelimumab (AstraZeneca) is also known as ticilimumab, CP-675,206; see WO 2000/037504 and Ribas, A. (2007) Update Cancer Ther. 2 (3): 133-39).

In some aspects, the anti-CTLA-4 antibody is ipilimumab. Ipilimumab (sold under the brand name YERVOY®; light chain amino acid sequence set forth in SEQ ID NO: 151 and heavy chain amino acid sequence as set forth in SEQ ID NO: 150), which was first approved for the treatment of metastatic melanoma, has since been approved for use in other cancers. Hoos et al. (2010) Semin. Oncol. 37:533; Hodi et al. (2010) N. Engl. J. Med. 363:711; Pardoll, D. M. (2012) Nat. Immunol. 13 (12): 1129. Ipilimumab, a human antibody which has an IgG1 constant region, was approved in 2011 in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed. Ipilimumab is also known as MDX-010 and 10D1. See U.S. Pat. No. 6,984,720.

In some aspects, the anti-CTLA-4 antibody is an activatable anti-CTLA-4 antibody, such as an activatable anti-CTLA-4 antibody wherein the light chains of the antibody comprise a cleavable moiety and a masking moiety at the amino termini. The masking moiety interferes with binding to CTLA-4, but is preferentially released in the tumor microenvironment after cleavage of the cleavable moiety by proteases that are more prevalent and/or active in tumors than in peripheral tissues (see, in particular, WO 2018/085555). Such preferential cleavage in the tumor microenvironment enables full CTLA-4 blocking, promoting anti-tumor immune response, while minimizing CTLA-4 blockade in normal tissue, thereby reducing the risk of potential systemic toxicity of an anti-CTLA-4 antibody. In some aspects, the activatable anti-CTLA-4 antibody is an activatable form of ipilimumab, such as an antibody comprising light chains modified to comprise a masking moiety and a cleavable moiety, as disclosed, for example, in WO 2018/085555. An example of an activatable anti-CTLA-4 antibody that has entered human clinical trials is BMS-986249 (NCT03369223: “A Study of BMS-986249 Alone and in Combination with Nivolumab in Advanced Solid Tumors”). In some aspects, the anti-CTLA-4 antibody is BMS-986249.

In some aspects, the anti-CTLA-4 antibody shows an enhanced Fcγ receptor (CD16) binding. Whether an anti-CTLA-4 antibody shows an enhanced Fcγ receptor binding is assessed by comparison with the Fcγ receptor binding of ipilimumab. Anti-CTLA-4 antibodies with enhanced Fcγ receptor (CD16) binding have been proposed as therapeutic agents for treatment of cancer through depletion of Treg cells. See, in particular, WO 2014/089113. In some aspects, the anti-CTLA-4 antibody shows an Fcγ receptor (CD16) binding that is at least a two-fold enhanced when compared to the Fcγ receptor binding of ipilimumab.

Examples of anti-CTLA-4 antibodies that show enhanced Fcγ receptor (i.e., FcγRIIIA or CD16) binding include nonfucosylated anti-CTLA-4 antibodies. In some aspects, the anti-CTLA-4 antibody is a nonfucosylated anti-CTLA-4 antibody. Non-fucosylated anti-CTLA-4 antibodies lack fucose residues in its N-linked glycans. In some aspects, the non-fucosylated anti-CTLA-4 antibody is produced by expressing the chains of the antibody in a mammalian cell under conditions that prevent fucosylation, including but not limited to use of mammalian cells with genetic modifications preventing fucosylation, or growth of the cells expressing the antibody in medium containing one or more chemical compounds that inhibit fucosylation. In some aspects, the genetic modification that prevents fucosylation is inactivation, e.g. knock-out, of the FUT8 gene. In some aspects, the anti-CTLA-4 antibody is a hypofucosylated anti-CTLA-4 antibody.

An exemplary nonfucosylated anti-CTLA-4 antibody that has entered human clinical trials is BMS-986218 (e.g., NCT03110107: “First-In-Human Study of Monoclonal Antibody BMS-986218 by Itself and in Combination with Nivolumab in Participants with Advanced Solid Tumors”). BMS-986218 is a nonfucosylated antibody developed to increase the effects of CTLA-4 blockade by enhancing binding to Fcγ receptor, thus promoting APC-mediated T cell priming. In some aspects, the anti-CTLA-4 antibody is BMS-986218. See, for example, PCT/US18/19868.

In some aspects, the Fc region of the anti-CTLA-4 antibody contains amino acid substitutions in the antibody constant region to enhance binding to activating Fcγ receptors. Exemplary substitutions are G236A, S239D, A330L and I332E (all residue numbering per the EU numbering system). In some aspects, the anti-CTLA-4 antibody comprises a human IgG1 constant domain with S239D, A330L and I332E mutations.

In some aspects, the anti-CTLA-4 antibody is an activatable and nonfucosylated anti-CTLA-4 antibody. Activatable anti-CTLA-4 antibodies can also be produced as nonfucosylated antibodies having both the increased efficacy of an enhanced Fcγ receptor binding antibody, with the added safety of being masked in peripheral tissues. An exemplary non-fucosylated, activatable anti-CTLA-4 antibody that has entered human clinical trials is BMS-986288 (NCT03994601: “An Investigational Immunotherapy Study of BMS-986288 Alone and in Combination with Nivolumab in Advanced Solid Cancers”). In some aspects, the anti-CTLA-4 antibody is BMS-986288.

Human monoclonal antibodies that bind specifically to CTLA-4 with high affinity that are suitable for the methods of the present disclosure have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In some aspects, the anti-CTLA-4 antibody cross-competes with an anti-CTLA-4 antibody described herein, e.g., ipilimumab and/or tremelimumab, for binding to CTLA-4. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223). In some aspects, the Treg-depleting agent comprises an antigen binding portion of the anti-CTLA-4 antibody (such as an anti-human-CTLA-4 antibody) that cross-competes for the binding to CTLA-4 (such as human CTLA-4) with any anti-CTLA-4 antibody described herein, e.g., ipilimumab and/or tremelimumab. In some aspects, the antibody described in this paragraph is a monocloncal antibody. In some aspects, the antibody described in this paragraph is a chimeric, humanized, or human antibody.

In some aspects, the anti-CTLA-4 antibody binds the same epitope as an anti-CTLA-4 antibody described herein, e.g., ipilimumab and/or tremelimumab. In some aspects, the antibody described in this paragraph is a monocloncal antibody. In some aspects, the antibody described in this paragraph is a chimeric, humanized, or human antibody.

In some aspects, the anti-CTLA-4 antibody is a multispecific antibody, e.g., a bispecific antibody.

In some aspects, the Treg cell depleting agent is a means for specifically binding to CTLA-4 (wherein the means are described herein).

### c) Anti-GITR Antibody, or Antigen Binding Fragment Thereof

In some aspects, the Treg-depleting agent comprises an anti-GITR antibody (i.e., an antibody capable of specifically binding to GITR), or an antigen binding portion thereof.

Anti-GITR antibodies specifically bind to GITR (such as human GITR) and activate the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells. Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells.

Examples of anti-GITR antibodies have been disclosed in Int'l Publ. Nos. WO/2015/031667, WO2015/184,099, WO2015/026,684, WO11/028683 and WO/2006/105021, WO2015/187835, WO2017/087678, WO2018/213297; U.S. Pat. Nos. 7,812,135 and 8,388,967; and U.S. Publ. Nos. 2009/0136494, 2014/0220002, 2013/0183321, and 2014/0348841.

In some aspects, the anti-GITR antibody is MK-4166, TRX518, AGEN-1876, ASP1951, GWN323, BMS-986156, or REGN6569.

In some aspects, the anti-GITR antibody is TRX518 (described in, for example, Schaer et al. (2012) Curr. Opin. Immunol. 24 (2): 217-224, and WO/2006/105021).

In some aspects, the anti-GITR antibody is MK4166, MK1248, or antibodies described in WO11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO11/028683 or U.S. Pat. No. 8,709,424). In some aspects, the anti-GITR antibody is MK4166.

In some aspects, the anti-GITR antibody is an anti-GITR antibody disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667.

In some aspects, the anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979).

In some aspects, the anti-GITR antibody is an anti-GITR antibody disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune).

In some aspects, the anti-GITR antibody is an anti-GITR antibody that is described in PCT/US2015/033991 (e.g., an antibody comprising the variable regions of 28F3, 18E10 or 19D3).

In some aspects, the anti-GITR antibody cross-competes with an anti-GITR antibody described herein, e.g., TRX518, MK4166. In some aspects, the anti-GITR antibody binds the same epitope as an anti-GITR antibody described herein, e.g., TRX518, MK4166. In some aspects, the anti-GITR antibody comprises the six CDRs, the VH and the VL, and/or the heavy and light chain of an anti-GITR antibody as described herein, e.g., of TRX518, MK4166.

In some aspects, the anti-GITR antibody is a multispecific antibody, e.g., a bispecific antibody.

In some aspects, the Treg cell depleting agent is a means for specifically binding to GITR (wherein the means are described herein).

### d) Anti-TIM3 Antibody, or Antigen Binding Fragment Thereof

In some aspects, the Treg-depleting agent comprises an anti-TIM3 antibody (i.e., an antibody capable of specifically binding to TIM3), or an antigen binding portion thereof.

In some aspects, the anti-TIM3 antibody is cobolimab, sabatolimab, BGB-A425, INACGN02390, LY3321367, Sym023, or BMS-986285.

In some aspects, the anti-TIM3 antibody comprises an anti-TIM3 antibody disclosed in Int'l Publ. Nos. WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802, WO2016068803, WO2016/111947, WO/2017/031242, or WO/2019/046321.

In some aspects, the anti-TIM3 antibody is a multispecific antibody, e.g., a bispecific antibody.

In some aspects, the Treg cell depleting agent is a means for specifically binding to TIM3 (wherein the means are described herein).

### e) Anti-OX40 Antibody, or Antigen Binding Fragment Thereof

In some aspects, the Treg-depleting agent comprises an anti-OX40 antibody (i.e., an antibody capable of specifically binding to OX40), or an antigen binding portion thereof. OX40 is also known as CD134, TNFRSF4, ACT35 and/or TXGP1L.

In some aspects, the anti-OX40 antibody is BMS-986178, MEDI6469, MEDI0562, ES102, GSK3174998, INCAGN01949, KHK4083, MEDI0562, or PF-04518600.

In some aspects, the anti-OX40 antibody is BMS-986178 (Bristol-Myers Squibb Company), described in Int'l Publ. No. WO20160196228.

In some aspects, the anti-OX40 antibody is an anti-OX40 antibody described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, or WO 17096182.

In some aspects, the anti-OX40 antibody is a multispecific antibody, e.g., a bispecific antibody.

In some aspects, the Treg cell depleting agent is a means for specifically binding to OX40 (wherein the means are described herein).

### f) Anti-ICOS Antibody, or Antigen Binding Fragment Thereof

In some aspects, the Treg-depleting agent comprises an anti-ICOS antibody (i.e., an antibody capable of specifically binding to ICOS), or an antigen binding portion thereof.

ICOS is an immune checkpoint protein that is a member of the CD28-superfamily. ICOS is a 55-60 kDa type I transmembrane protein that is expressed on T cells after T cell activation and co-stimulates T-cell activation after binding its ligand, ICOS-L (B7H2). ICOS is also known as inducible T-cell co-stimulator, CVID1, AILIM, inducible costimulator, CD278, activation-inducible lymphocyte immunomediatory molecule, and CD278 antigen.

In some aspects, the anti-ICOS antibody is BMS-986226, vopratelimab, feladilimab, MEDI-570, or KY1044.

In some aspects, the anti-ICOS antibody is BMS-986226, a humanized IgG monoclonal antibody that binds to and stimulates human ICOS.

In some aspects, the anti-ICOS antibody is an anti-ICOS antibody described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), or WO 2017/220988 (Kymab Limited).

In some aspects, the anti-ICOS antibody is a multispecific antibody, e.g., a bispecific antibody.

In some aspects, the Treg cell depleting agent is a means for specifically binding to ICOS (wherein the means are described herein).

### g) Anti-TIGIT Antibody, or Antigen Binding Fragment Thereof

In some aspects, the Treg-depleting agent comprises an anti-TIGIT antibody (i.e., an antibody capable of specifically binding to TIGIT), or an antigen binding portion thereof.

In some aspects, the anti-TIGIT antibody is BGB-A1217, COM902, BMS-986207, COM701, domvanalimab, etigilimab, JS006, ociperlimab, SE-TGT, tiragolumab, vibostolimab, or IBI939.

In some aspects, the anti-TIGIT antibody is BMS-986207.

In some aspects, the anti-TIGIT antibody is clone 22G2, as described in WO 2016/106302.

In some aspects, the anti-TIGIT antibody is MTIG7192A/RG6058/RO7092284, or clone 4.1D3, as described in WO 2017/053748.

In some aspects, the anti-TIGIT antibody is an anti-TIGIT antibody described in, for example, WO 2016/106302 (Bristol-Myers Squibb Company) or WO 2017/053748 (Genentech).

In some aspects, the anti-TIGIT antibody is a multispecific antibody, e.g., a bispecific antibody.

In some aspects, the Treg cell depleting agent is a means for specifically binding to TIGIT (wherein the means are described herein).

### h) Anti-CD25 Antibody, or Antigen Binding Fragment Thereof

In some aspects, the Treg-depleting agent comprises an anti-CD25 antibody (i.e., an antibody capable of specifically binding to CD25), or an antigen binding portion thereof.

In some aspects, the anti-CD25 antibody is daclizumab.

In some aspects, the anti-CD25 antibody is an anti-CD25 antibody described in, for example, Arce Vargas et al. (2017) Immunity 46 (4): 577-86, WO2019175226A1, WO2018167104A1, WO2017174331A1, WO2014144935A2, WO2021021882A1, WO2018193104A1, WO2014145000A2, WO2020234399A1, WO2016177438A1, WO2018229218A1, WO2020248938A1, WO2020249527A1, WO2021228218A1, WO2021066840A1, WO2022106665A1, WO2022106663A1, WO2024034759A1, WO2022239993A1, WO2024213046A1, WO2024204629A1, WO2024192065A1, WO2014026053A9, WO2023016455A1, WO2022038193A1, WO2023028451A3, WO2020102591A1, or WO2022104009-A1.

In some aspects, the anti-CD25 antibody is a multispecific antibody, e.g., a bispecific antibody.

In some aspects, the Treg cell depleting agent is a means for specifically binding to CD25 (wherein the means are described herein).

### i) Anti-CCR4 Antibody, or Antigen Binding Fragment Thereof

In some aspects, the Treg-depleting agent comprises an anti-CCR4 antibody (i.e., an antibody capable of specifically binding to CCR4), or an antigen binding portion thereof.

In some aspects, the anti-CCR4 antibody is is mogamulizumab.

In some aspects, the anti-CCR4 antibody is an anti-CCR4 antibody described in, for example, Ishida et al. (2012) J. Clin. Oncol. 30:837-42, Hagemann et al. (2014) PLOS ONE 9 (7): e103776, WO-2024163530-A2, WO-2021025148-A1, WO-2018159845-A1, WO-2018101448-A1, WO-2016178779-A1, WO-2015179236-A1, WO-2013166500-A1, WO-2012076883-A1, WO-0042074-A1, or WO-2016057488-A1.

In some aspects, the anti-CCR4 antibody is a multispecific antibody, e.g., a bispecific antibody.

In some aspects, the Treg cell depleting agent is a means for specifically binding to CCR4 (wherein the means are described herein).

### 3. Additional Anti-Tumor Treatment

As outlined above, in some aspects, the methods of the present disclosure further comprise administering an additional anti-tumor treatment.

In some aspects, the method further comprises administering to the subject an additional means for anti-tumor treatment (wherein the means are described herein).

In the following, the additional anti-tumor treatment suitable for the methods of the present disclosure is further described.

In some aspects, the method may further comprise administering another Treg cell depleting agent as an additional anti-tumor treatment. For example, in aspects, wherein the method comprises administering an anti-CCR8-antibody (such as 14S15, 14S15h, or imzokitug), or antigen binding portion thereof, as Treg cell depleting agent, the method may further comprise administering an anti-CTLA-4 antibody, or an antigen binding portion thereof, as an additional anti-tumor treatment. In some aspects, the anti-CTLA-4 antibody is any anti-CTLA-4 antibody as described above in section titled “anti-CTLA-4 antibody, or antigen binding portion thereof”. In some aspects, the additional anti-tumor treatment comprises tremelimumab. In some aspects, the additional anti-tumor treatment comprises ipilimumab.

In some aspects, the additional anti-tumor treatment does not comprise another Treg cell depleting agent.

The additional anti-tumor treatment can comprise any anti-tumor treatment known in the art. In some aspects, the additional anti-tumor treatment comprises a surgery. In some aspects, the additional anti-tumor treatment comprises a radiation therapy. In some aspects, the additional anti-tumor treatment comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor (TKI) comprises sorafenib (e.g., sorafenib tosylate, also known as NEXAVAR®), lenvatinib (e.g., lenvatinib mesylate, also known as LENVIMA®), regorafenib (e.g., STIVARGA®), cabozantinib (e.g., cabozantinib S-malate, also known as CABOMETYX®), sunitinib (e.g., sunitinib malate, also known as SUTENT®), brivanib, linifanib, pemigatinib (also known as PEMAZYRE®), everolimus (also known as AFINITOR® or ZORTRESS®), gefitinib (IRESSA®, a small-molecule TKI of EGFR), imatinib (e.g., imatinib mesylate), lapatinib (e.g., lapatinib ditosylate, also known as TYKERB®), nilotinib (e.g., nilotinib hydrochloride, also known as TASIGNA®), pazopanib (e.g., pazopanib hydrochloride, also known as VOTRIENT®), temsirolimus (also known as TORISEL®), erlotinib (e.g., erlotinib hydrochloride, also known as TARCEVA®, a small-molecule TKI of EGFR), afatinib (GILOTRIF®, a small-molecule TKI of EGFR), dacomitinib (VIZIMPRO®, a small-molecule TKI of EGFR), osimeritinb (TAGRISSO®, a small-molecule TKI of EGFR), alectinib (ALECENSAR, a small-molecule TKI of ALK), ceritinib (ZYKADIA®, a small-molecule TKI of ALK and ROS-1), brigatinib (ALUNBRIG®, a small-molecule TKI of ALK), crizotinib (XALKORI®, a small-molecule TKI of ALK and ROS-1), lorlatinib (LORBRENA®, a small-molecule TKI of ALK and ROS-1), entrectinib (ROZLYTREK®, a small-molecule TKI of ROS-1 and NTRK), dabrafenib (TAFINLAR®, a small-molecule TKI of BRAF) trametinib (MEKINIST®, a small-molecule TKI of BRAF), vemurafenib (ZELBORAF®, a small-molecule TKI of BRAF), larotrectinib (VITRAKVI®, a small-molecule TKI of tropomyosin kinase receptors TrkA, TrkB, and TrkC), or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGFR, or any combination thereof. In some aspects, the anti-angiogenesis agent comprises bevacizumab (also known as AVASTIN®), ranibizumab (also known as LUCENTIS®), ramucirumab (also known as CYRAMZA®), aflibercept (also known as EYLEA® or ZALTRAP®), tanibirumab, olaratumab (also known as LARTRUVO®), nesvacumab, AMG780, MEDI3617, vanucizumab, rilotumumab, ficlatuzumab, TAK-701, onartuzumab, emibetuzumab, or any combination thereof.

In some aspects, the checkpoint inhibitor comprises a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-B) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD-1H) inhibitor, a leukocyte-associated immunoglobulin-like receptor 1 (LAIR1) inhibitor, or any combination thereof.

In some aspects, the checkpoint stimulator comprises an agonist of B7-1, B7-2, CD28, 4-1BB (CD137), 4-1BBL, GITR, inducible T cell co-stimulator (ICOS), ICOS-L, OX40, OX40L, CD70, CD27, CD40, death receptor 3 (DR3), CD28H, or any combination thereof.

In some aspects, the chemotherapeutic agent comprises an alkylating agent, an antimetabolite, an antineoplastic antibiotic, a mitotic inhibitor, a hormone or hormone modulator, a protein tyrosine kinase inhibitor, an epidermal growth factor inhibitor, a proteasome inhibitor, other neoplastic agent, or any combination thereof.

In some aspects, the immunotherapeutic agent comprises an antibody that specifically binds to EGFR (e.g., cetuximab (ERBITUX®)), ALK, ROS-1, NTRK, BRAF, ICOS, CD137 (4-1BB), CD134 (OX40), NKG2A, CD27, CD96, GITR, Herpes Virus Entry Mediator (HVEM), PD-1, PD-L1, CTLA-4, BTLA, TIM-3, A2aR, Killer cell Lectin-like Receptor G1 (KLRG-1), Natural Killer Cell Receptor 2B4 (CD244), CD160, TIGIT, VISTA, KIR, TGFB, IL-10, IL-8, B7-H4, Fas ligand, CSF1R, CXCR4, mesothelin, CEACAM-1, CD52, HER2, MICA, MICB, or any combination thereof. “Immunotheray,” in general, refers to the treatment of a subject afflicted with, or at risk of contracting or suffering a recurrence of, a disease by a method comprising inducing, enhancing, suppressing or otherwise modifying an immune response.

In some aspects, the platinum agent comprises cisplatin, carboplatin, oxaliplatin, satraplatin, picoplatin, nedaplatin, triplatin (e.g., triplatin tetranitrate), lipoplatin, phenanthriplatin, or any combination thereof.

In some aspects, the alkylating agent comprises altretamine, bendamustine, busulfan, carboplatin, carmustine, chlorambucil, cisplatin, cyclophosphamide, dacarbazine, ifosfamide, lomustine, mechlorethamine, melphalan, oxaliplatin, procarbazine, streptozocin, temozolomide, thiotepa, or any combination thereof.

In some aspects, the taxane comprises paclitaxel, albumin-bound paclitaxel, docetaxel, cabazitaxel, or any combination thereof.

In some aspects, the nucleoside analog comprises cytarabine, gemcitabine, lamivudine, entecavir, telbivudine, or any combination thereof.

In some aspects, the antimetabolite comprises capecitabine, cladribine, clofarabine, cytarabine, floxuridine, fludarabine, fluorouracil, gemcitabine, mercaptopurine, methotrexate, pemetrexed, pentostatin, pralatrexate, thioguanine, or any combination thereof.

In some aspects, the topoisomerase inhibitor comprises etoposide, mitoxantrone, doxorubicin, irinotecan, topotecan, camptothecin, or any combination thereof.

In some aspects, the anthracycline is doxorubicin, daunorubicin, epirubicin, idarubicin, or any combination thereof.

In some aspects, the vinca alkaloid is vinblastine, vincristine, vinorelbine, vindesine, vincaminol, vineridine, vinburnine, or any combination thereof.

In some aspects, the additional anti-tumor treatment comprises an antibody, or an antigen binding portion thereof. In some aspects, the antibody specifically binds CTLA-4, TIGIT, TIM3, NKG2a, OX40, ICOS, MICA, CD137, KIR, TGFB, IL-10, IL-8, B7-H4, Fas ligand, CXCR4, mesothelin, CD27, GITR, or any combination thereof.

In some aspects, the additional anti-tumor treatment comprises IL-2 (e.g., bempegaldesleukin). In some aspects, the additional anti-tumor treatment comprises IL12-Fc (e.g., BMS-986415).

In some aspects, the additional anti-tumor treatment comprises an anti-CD137 antibody. Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669. In some aspects, the anti-CD137 antibody is urelumab (BMS-663513; described, for example, in U.S. Pat. No. 7,288,638 (20H4.9-IgG4 [10C7 or BMS-663513])). In some aspects, the anti-CD137 antibody is BMS-663031 (20H4.9-IgG1; described, for example, in U.S. Pat. No. 7,288,638). In some aspects, the anti-CD137 antibody is 4E9 or BMS-554271 (described, for example, in U.S. Pat. No. 6,887,673). In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer's PF-05082566 (PF-2566). In some aspects, an anti-CD137 antibody useful for the methods of the present disclosure cross-competes with the anti-CD137 antibodies as described herein. In some aspects, an anti-CD137 antibody binds to the same epitope as the anti-CD137 antibody as described herein. In some aspects, an anti-CD137 antibody useful in the disclosure comprises the six CDRs, the VH and VL, and/or the heavy and light chain of any of the anti-CD137 antibodies as described herein. In some aspects, the anti-CD137 antibody is a multispecific antibody, e.g., a bispecific antibody

In some aspects, the additional anti-tumor treatment comprises an anti-KIR antibody. The anti-KIR antibody comprises any antibody that binds KIR and inhibits its activity. Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448. One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In some aspects, the anti-KIR antibody cross competes for binding to KIR with lirilumab or I-7F9. In some aspects, the anti-KIR antibody binds to the same epitope as lirilumab or I-7F9. In some aspects, the anti-KIR antibody comprises the six CDRs, the VH and VL, and/or the heavy and light chain of lirilumab or I-7F9. In some aspects, the anti-KIR antibody is a multispecific antibody, e.g., a bispecific antibody. In some aspects, the additional anti-tumor treatment comprises an anti-CD96 antibody.

In some aspects, the additional anti-tumor treatment comprises an anti-IL8 antibody, such as HUMAX®-IL8 (BMS-986253).

In some aspects, the additional anti-tumor treatment comprises an anti-TGFß antibody.

In some aspects, the the additional anti-tumor treatment comprises an anti-B7-H4 antibody. In certain aspects, the anti-B7-H4 antibody is an anti-B7-H4 disclosed in Int'l Publ. No. WO/2009/073533.

In some aspects, the additional anti-tumor treatment comprises an anti-CD96 antibody.

In some aspects, the additional anti-tumor treatment comprises an anti-TIM3 antibody. In some aspects, the additional anti-tumor treatment comprises an anti-VISTA antibody.

In some aspects, the additional therapeutic agent comprises an anti-NKG2A antibody. NKG2A is a member of the C-type lectin receptor family that is expressed on natural killer (NK) cells and a subset of T lymphocytes. Specifically, NKG2A primarily expressed on tumor infiltrating innate immune effector NK cells, as well as on some CD8⁺ T cells. Its natural ligand human leukocyte antigen E (HLA-E) is expressed on solid and hematologic tumors. NKG2A is an inhibitory receptor that blinds HLA-E. In some aspects, the anti-NKG2A antibody is BMS-986315, a human monoclonal antibody that blocks the interaction of NKG2A to its ligand HLA-E, thus allowing activation of an anti-tumor immune response. In some aspects, the anti-NKG2A antibody is a checkpoint inhibitor that activates T cells, NK cells, and/or tumor-infiltrating immune cells. In some aspects, the anti-NKG2A antibody is an anti-NKG2A antibody described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Pat. No. 9,447,185 (Innate Pharma S/A; Novo Nordisk A/S; University of Genova); WO 2008/009545 (Novo Nordisk A/S); U.S. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945 (Academisch Ziekenhuis Leiden H.O.D.N. LUMC); WO 2016/041947 (Innate Pharma); or WO 2016/041945 (Innate Pharma). In some aspects, the anti-NKG2A antibody is a multispecific antibody, e.g., a bispecific antibody.

In some aspects, the additional anti-tumor treatment comprises an anti-ICOS antibody.

In some aspects, the additional anti-tumor treatment comprises an anti-OX40 antibody.

In some aspects, the additional anti-tumor treatment comprises an anti-TIGIT antibody.

In some aspects, the additional anti-tumor treatment comprises an anti-Fas ligand antibody. In some aspects, the anti-Fas ligand antibody is an anti-Fas ligand disclosed in Int'l Publ. No. WO/2009/073533.

In some aspects, the additional anti-tumor treatment comprises an anti-CXCR4 antibody. In certain aspects, the anti-CXCR4 antibody is an anti-CXCR4 disclosed in U.S. Publ. No. 2014/0322208 (e.g., Ulocuplumab (BMS-936564)).

In some aspects, the additional anti-tumor treatment comprises an anti-mesothelin antibody. In some aspects, the anti-mesothelin antibody is an anti-mesothelin disclosed in U.S. Pat. No. 8,399,623.

In some aspects the additional anti-tumor treatment comprises an anti-HER2 antibody. In some aspects, the anti-HER2 antibody is Herceptin (U.S. Pat. No. 5,821,337), trastuzumab, or ado-trastuzumab emtansine (Kadcyla, e.g., WO/2001/000244).

In some aspects, the additional anti-tumor treatment comprises an anti-CD27 antibody. In some aspects, the anti-CD-27 antibody is Varlilumab (also known as “CDX-1127” and “1F5”), which is a human IgG1 antibody that is an agonist for human CD27, as disclosed in, for example, U.S. Pat. No. 9,169,325.

In some aspects, the additional anti-tumor treatment comprises an anti-CD73 antibody. In some aspects, the anti-CD73 antibody is CD73.4.IgG2C219S.IgG1.If.

In some aspects, the additional anti-tumor treatment comprises an anti-MICA/B antibody. In some aspects, the anti-MICA/B antibody is any antibody that specifically binds human MICA/B, including but not limited to, any anti-MICA/B antibody disclosed in International Publication No. WO 2019/183551, which is incorporated by reference herein in its entirety.

In some aspects, the additional anti-tumor treatment comprises an anti-IL-10 antibody. In some aspects, the additional anti-tumor treatment comprises a long-acting IL-10 molecule. In some aspects, the long-acting IL-10 molecule comprises an IL-10-Fc fusion molecule. In some aspects, the long-acting IL-10 molecule comprises a Pegylated IL-10, such as AM0010 (ARMO BioSciences).

In some aspects, the additional anti-tumor treatment comprises an anti-IL-2 antibody. In some aspects, the additional anti-tumor treatment comprises a long-acting IL-2 molecule. In some aspects, the long-acting IL-2 comprises a Pegylated IL-2, such as NKTR-214 (Nektar; see U.S. Pat. No. 8,252,275, WO12/065086 and WO15/125159).

In some aspects, the additional anti-tumor treatment comprises an anti-IL-12 antibody. In some aspects, the anti-IL-12 antibody is a multispecific antibody, e.g., a bispecific antibody.

In some aspects, the additional anti-tumor treatment comprises an anti-IL-13 antibody. In some aspects, the anti-IL-13 antibody is a multispecific antibody, e.g., a bispecific antibody.

In some aspects, the additional anti-tumor treatment comprises an anti-IL-15 antibody. In some aspects, the anti-IL-15 antibody is a multispecific antibody, e.g., a bispecific antibody.

In some aspects, the additional anti-tumor treatment comprises an anti-SIRPalpha antibody. In some aspects, the anti-SIRPalpha antibody is a multispecific antibody, e.g., a bispecific antibody.

In some aspects, the additional anti-tumor treatment comprises an anti-CD47 antibody. In some aspects, the anti-CD47 antibody is a multispecific antibody, e.g., a bispecific antibody.

In some aspects, the additional anti-tumor treatment comprises an anti-MICA antibody. In some aspects, the anti-MICA antibody is a multispecific antibody, e.g., a bispecific antibody.

In some aspects, the additional anti-tumor treatment comprises an anti-ILT4 antibody. In some aspects, the anti-ILT4 antibody is a multispecific antibody, e.g., a bispecific antibody.

In some aspects, the additional anti-tumor treatment comprises an antibody that specifically binds PD-L1, CTLA-4, TIGIT, TIM3, NKG2a, OX40, ICOS, MICA, CD137, KIR, TGFB, IL-10, IL-8, B7-H4, Fas ligand, CXCR4, mesothelin, CD27, GITR, CCR8, ILT4, or any combination thereof.

In some aspects, the additional anti-tumor treatment, e.g., an antibody, or an antigen binding portion thereof, is administered by parenteral administration (i.e., systemically), such as by intravenous administration.

In some aspects, the additional anti-tumor treatment, e.g., an antibody, or an antigen binding portion thereof, is administered every week, every two weeks, every three weeks, every four weeks, every five weeks, or every six weeks. In some aspects, the additional anti-tumor treatment, e.g., antibody or antigen binding portion thereof, is administered every week. In some aspects, the additional anti-tumor treatment, e.g., antibody or antigen binding portion thereof, is administered every two weeks. In some aspects, the additional anti-tumor treatment, e.g., antibody or antigen binding portion thereof, is administered every three weeks.

In some aspects, the additional anti-tumor treatment, e.g., an antibody, or an antigen binding portion thereof, is administered every 5 days, every 6 days, every 7 days, every 8 days, every 9 days, every 10 days, every 11 days, every 12 days, every 13 days, every 14 days, every 15 days, every 16 days, every 17 days, every 18 days, every 19 days, every 20 days, every 21 days, every 22 days, every 23 days, every 24 days, every 25 days, every 26 days, every 27 days, or every 28 days.

In some aspects, the KRAS inhibitor, the additional anti-tumor treatment, and the Treg-depleting agent are administered separately. In some aspects, the KRAS inhibitor, the additional anti-tumor treatment, and the Treg-depleting agent are administered sequentially. In some aspects, the KRAS inhibitor, the additional anti-tumor treatment, and the Treg-depleting agent are administered concurrently.

In some aspects, the KRAS inhibitor is administered prior to the Treg-depleting agent and the additional anti-tumor treatment. In some aspects, the KRAS inhibitor and the additional anti-tumor treatment are administered prior to the Treg-depleting agent. In some aspects, the KRAS inhibitor and the Treg-depleting agent are administered prior to the additional anti-tumor treatment. In some aspects, the additional anti-tumor treatment is administered prior to the KRAS inhibitor and the Treg-depleting agent. In some aspects, the additional anti-tumor treatment and the KRAS inhibitor are administered prior to the Treg-depleting agent. In some aspects, the additional anti-tumor treatment and the Treg-depleting agent are administered prior to the KRAS inhibitor. In some aspects, the Treg-depleting agent is administered prior to the KRAS inhibitor and the additional anti-tumor treatment.

### a) PD-1 Axis-Blocking Agent

In some aspects, the additional anti-tumor treatment comprises a PD-1 axis-blocking agent. In some aspects, the PD-1 axis-blocking agent comprises a PD-L1 antagonist or a PD-1 antagonist. In some aspects, the PD-1 axis-blocking agent comprises an anti-PD-L1 antibody, an anti-PD-1 antibody, or an antigen binding portion thereof.

In some aspects, the additional anti-tumor treatment comprises a PD-1 axis-blocking agent and further comprises an anti-LAG-3 antibody or an antigen binding portion thereof. Suitable anti-LAG-3 antibodies of the present disclosure are described herein, in particular, in section titled “LAG-3 antagonist”.

In some aspects, the PD-1 axis-blocking agent comprises a protein, a peptide, or a small molecule. In some aspects, the PD-1 axis-blocking agent is BMS-986189 (see, e.g., Kim, J. et al. (2023) Mol. Imaging Biol. 26 (2): 301-309). In some aspects, the PD-1 axis blocking agent is AUNP12 (see, e.g., Guzik, K. et al. (2019) Molecules. 24 (11): 2071). In some aspects, the PD-1 axis-blocking agent is CA-170 (see, e.g., Sasikumar, P. G. et al. (2021) Commun. Biol. 4 (1): 699).

In some aspects, the PD-1 axis-blocking agent comprises an antibody or an antigen binding portion thereof. In some aspects, the PD-1 axis-blocking agent comprises an antibody. In some aspects, the antibody is a chimeric antibody. In some aspects, the antibody is a human antibody. In some aspects, the antibody is a humanized antibody.

### i) PD-1

In some aspects, the PD-1 axis-blocking agent comprises a PD-1 antagonist. In some aspects, the PD-1 axis-blocking agent comprises a human PD-1 antagonist. In some aspects, the PD-1 antagonist comprises an anti-PD-1 antibody (i.e., an antibody that specifically binds to PD-1) or an antigen binding portion thereof, an immunoadhesin, an oligopeptide, or a nucleic acid molecule.

In some aspects, the PD-1 axis-blocking agent comprises an antigen binding portion of an anti-PD-1 antibody (such as a F(ab′)₂ fragment, a Fab′ fragment, a Fab fragment, a Fv fragment, a scFv fragment, a dsFv fragment, or a dAb fragment). In some aspects, the PD-1 axis-blocking agent comprises an anti-human-PD-1 antibody or antigen binding portion thereof.

In some aspects, the anti-PD-1 antibody (such as an anti-human-PD-1 antibody) is a human, a humanized, or a chimeric antibody. The antibody might be a multispecific antibody (such as a bispecific antibody). In some aspects, the multispecific antibody is a DART, a DVD-Ig, or bispecific antibody.

In some aspects, the anti-PD-1 antibody is a monospecific antibody (specifically binding to PD-1). In some aspects, the anti-PD-1 antibody is a multispecific antibody, such as a bispecific or trispecific antibody. In some aspects, the anti-PD-1 antibody is a bispecific antibody that specifically binds to (human) PD-1 and (human) LAG-3. In some aspects, the anti-PD-1 antibody is a bispecific antibody that specifically binds to (human) PD-1 and (human) PD-L1. In some aspects, the anti-PD-1 antibody is a trispecific antibody that specifically binds to (human) PD-L1, (human) LAG-3, and (human) PD-1.

Anti-PD-1 antibodies suitable for use in the methods of the present disclosure are antibodies that bind to PD-1 with high specificity and affinity, block the binding of PD-L1 and or PD-L2, and inhibit the immunosuppressive effect of the PD-1 signaling pathway.

In some aspects, the anti-PD-1 antibody or antigen binding portion thereof comprises a heavy chain constant region which is of a human IgG1, IgG2, IgG3 or IgG4 isotype. In some aspects, the anti-PD-1 antibody or antigen binding portion thereof comprises a heavy chain constant region which is of a human IgG4 isotype. In some aspects, the anti-PD-1 antibody or antigen binding portion thereof is of a human IgG1 isotype. In some aspects, the IgG4 heavy chain constant region of the anti-PD-1 antibody or antigen binding portion thereof contains an S228P mutation (numbered using the Kabat system) which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies. In some aspects, the anti-PD-1 antibody or antigen binding portion thereof comprises a light chain constant region which is a human kappa or lambda constant region.

Any anti-PD-1 antibody can be used in the presently described methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10-7 M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five or all, of the preceding characteristics.

Other anti-PD-1 monoclonal antibodies suitable for the methods of the present disclosure have been described in, for example, U.S. Pat. Nos. 6,808,710, 7,488,802, 8,168,757, 8,354,509, and 9,205,148, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Exemplary sequences of anti-PD-1 antibodies suitable for the methods of the present disclosure are given in Table 11.

TABLE 11
Exemplary sequences of anti-PD-1 antibodies suitable
for the methods of the present disclosure.
Anti-PD-1 antibody nivolumab

| | |
|---|---|
| CDRH1 (SEQ ID NO: 57) | NSGMH |
| CDRH2 (SEQ ID NO: 58) | VIWYDGSKRYYADSVKG |
| CDRH3 (SEQ ID NO: 59) | NDDY |
| CDRL1 (SEQ ID NO: 60) | RASQSVSSYLA |
| CDRL2 (SEQ ID NO: 61) | DASNRAT |
| CDRL3 (SEQ ID NO: 62) | QQSSNWPRT |
| VH (SEQ ID NO: 63) | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSS |
| VL (SEQ ID NO: 64) | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIK |
| Heavy chain | QVQLVESGGGVVQPGRSLRLDCKASGITFSNSGMHWVRQAPGKGLEWVAVI |
| (SEQ ID NO: 65) | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVESKYGPPCPPCPAPEFLGGPSVFLFPPKPKDTLMISRTPEVTCV |
| | VVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKS |
| | RWQEGNVFSCSVMHEALHNHYTQKSLSLSLGK |
| Light chain | EIVLTQSPATLSLSPGERATLSCRASQSVSSYLAWYQQKPGQAPRLLIYDA |
| (SEQ ID NO: 66) | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLLSKADYEKHKVYACEVTHQGLSSPV |
| | TKSFNRGEC |

Anti-PD-1 antibody pembrolizumab

| | |
|---|---|
| CDRH1 (SEQ ID NO: 67) | NYYMY |
| CDRH2 (SEQ ID NO: 68) | GINPSNGGTNFNEKFKN |
| CDRH3 (SEQ ID NO: 69) | RDYRFDMGFDY |
| CDRL1 (SEQ ID NO: 70) | RASKGVSTSGYSYLH |
| CDRL2 (SEQ ID NO: 71) | LASYLES |
| CDRL3 (SEQ ID NO: 72) | QHSRDLPLT |
| VH (SEQ ID NO: 73) | QVQLVQSGVEVKKPGASVKVSCKASGYTFTNYYMYWVRQAPGQGLEWMGGI |
| | NPSNGGTNFNEKFKNRVTLTTDSSTTTAYMELKSLQFDDTAVYYCARRDYR |
| | FDMGFDYWGQGTTVTVSS |
| VL (SEQ ID NO: 74) | EIVLTQSPATLSLSPGERATLSCRASKGVSTSGYSYLHWYQQKPGQAPRLL |
| | IYLASYLESGVPARFSGSGSGTDFTLTISSLEPEDFAVYYCQHSRDLPLTF |
| | GGGTKVEIK |
| Heavy chain | QVQLVQSGVEVKKPGASVKVSCKASGYTFTNYYMYWVRQAPGQGLEWMGGI |
| (SEQ ID NO: 75) | NPSNGGTNFNEKFKNRVTLTTDSSTTTAYMELKSLQFDDTAVYYCARRDYR |
| | FDMGFDYWGQGTTVTVSSASTKGPSVFPLAPCSRSTSESTAALGCLVKDYF |
| | PEPVTVSWNSGALTSGVHTFPAVLQSSGLYSLSSVVTVPSSSLGTKTYTCN |
| | VDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVSVL |
| | TVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYS |
| | RLTVDKSRWQEGNVFSCSVMHEALHNHYTQKSLSLSLGK |
| Light chain | EIVLTQSPATLSLSPGERATLSCRASKGVSTSGYSYLHWYQQKPGQAPRLL |
| (SEQ ID NO: 76) | IYLASYLESGVPARFSGSGSGTDFTLTISSLEPEDFAVYYCQHSRDLPLTF |
| | GGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC |

Anti-PD-1 antibody cemiplimab

| | |
|---|---|
| CDRH1 (SEQ ID NO: 77) | GFTFSNFG |
| CDRH2 (SEQ ID NO: 78) | ISGGGRDT |
| CDRH3 (SEQ ID NO: 79) | VKWGNIYFDY |
| CDRL1 (SEQ ID NO: 80) | LSINTF |
| CDRL2 (SEQ ID NO: 81) | AAS |
| CDRL3 (SEQ ID NO: 82) | QQSSNTPFT |
| VH (SEQ ID NO: 83) | EVQLLESGGVLVQPGGSLRLSCAASGFTFSNFGMTWVRQAPGKGLEWVSGI |
| | SGGGRDTYFADSVKGRFTISRDNSKNTLYLQMNSLKGEDTAVYYCVKWGNI |
| | YFDYWGQGTLVTVSS |
| VL (SEQ ID NO: 84) | DIQMTQSPSSLSASVGDSITITCRASLSINTFLNWYQQKPGKAPNLLIYAA |
| | SSLHGGVPSRFSGSGSGTDFTLTIRTLQPEDFATYYCQQSSNTPFTFGPGT |
| | VVDFR |
| Heavy chain | EVQLLESGGVLVQPGGSLRLSCAASGFTFSNFGMTWVRQAPGKGLEWVSGI |
| (SEQ ID NO: 85) | SGGGRDTYFADSVKGRFTISRDNSKNTLYLQMNSLKGEDTAVYYCVKWGNI |
| | YFDYWGQGTLVTVSSASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPPCPAPEFLGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGK |
| Light chain | DIQMTQSPSSLSASVGDSITITCRASLSINTFLNWYQQKPGKAPNLLIYAA |
| (SEQ ID NO: 86) | SSLHGGVPSRFSGSGSGTDFTLTIRTLQPEDFATYYCQQSSNTPFTFGPGT |
| | VVDFRRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC |

Anti-PD-1 antibody spartalizumab

| | |
|---|---|
| CDRH1 (SEQ ID NO: 87) | TYWMH |
| CDRH2 (SEQ ID NO: 88) | NIYPGTGGSNFDEKFKN |
| CDRH3 (SEQ ID NO: 89) | WTTGTGAY |
| CDRL1 (SEQ ID NO: 90) | KSSQSLLDSGNQKNFLT |
| CDRL2 (SEQ ID NO: 91) | WASTRES |
| CDRL3 (SEQ ID NO: 92) | QNDYSYPYT |
| VH (SEQ ID NO: 93) | EVQLVQSGAEVKKPGESLRISCKGSGYTFTTYWMHWVRQATGQGLEWMGNI |
| | YPGTGGSNFDEKFKNRVTITADKSTSTAYMELSSLRSEDTAVYYCTRWTTG |
| | TGAYWGQGTTVTVSS |
| VL (SEQ ID NO: 94) | EIVLTQSPATLSLSPGERATLSCKSSQSLLDSGNQKNFLTWYQQKPGQAPR |
| | LLIYWASTRESGVPSRFSGSGSGTDFTFTISSLEAEDAATYYCQNDYSYPY |
| | TFGQGTKVEIK |
| Heavy chain | EVQLVQSGAEVKKPGESLRISCKGSGYTFTTYWMHWVRQATGQGLEWMGNI |
| (SEQ ID NO: 95) | YPGTGGSNFDEKFKNRVTITADKSTSTAYMELSSLRSEDTAVYYCTRWTTG |
| | TGAYWGQGTTVTVSSASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPPCPAPEFLGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLG |
| Light chain | EIVLTQSPATLSLSPGERATLSCKSSQSLLDSGNQKNFLTWYQQKPGQAPR |
| (SEQ ID NO: 96) | LLIYWASTRESGVPSRFSGSGSGTDFTFTISSLEAEDAATYYCQNDYSYPY |
| | TFGQGTKVEIKRTVAAPSVFIFPPSDEQLKSGTASVVCLLNNFYPREAKVQ |
| | WKVDNALQSGNSQESVTEQDSKDSTYSLSSTLTLSKADYEKHKVYACEVTH |
| | QGLSSPVTKSENRGEC |

In some aspects, the anti-PD-1 antibody, antigen binding portion thereof, comprises a VH comprising a CDRH1 comprising (or consisting of) the amino acid sequence set forth in SEQ ID NO: 57, a CDRH2 comprising (or consisting of) the amino acid sequence set forth in SEQ ID NO: 58, and a CDRH3 comprising (or consisting of) the amino acid sequence set forth in SEQ ID NO: 59; and a VL comprising a CDRL1 comprising (or consisting of) the amino acid sequence set forth in SEQ ID NO: 60, a CDRL2 comprising (or consisting of) the amino acid sequence set forth in SEQ ID NO: 61, and a CDRL3 comprising (or consisting of) the amino acid sequence set forth in SEQ ID NO: 62. In some aspects, the antibody is a chimeric, humanized, or human antibody.

In some aspects, the anti-PD-1 antibody, antigen binding portion thereof, comprises a VH comprising (or consisting of) the amino acid sequence set forth in SEQ ID NO: 63, and a VL comprising (or consisting of) the amino acid sequence set forth in SEQ ID NO: 64.

In some aspects, the anti-PD-1 antibody, antigen binding portion thereof, comprises a heavy chain comprising (or consisting of) the amino acid sequence as set forth in SEQ ID NO: 65 and a light chain comprising (or consisting of) the amino acid sequence as set forth in SEQ ID NO: 66.

In some aspects, the anti-PD-1 antibody, antigen binding portion thereof, comprises a VH comprising a CDRH1 comprising (or consisting of) the amino acid sequence set forth in SEQ ID NO: 67, a CDRH2 comprising (or consisting of) the amino acid sequence set forth in SEQ ID NO: 68, and a CDRH3 comprising (or consisting of) the amino acid sequence set forth in SEQ ID NO: 69; and a VL comprising a CDRL1 comprising (or consisting of) the amino acid sequence set forth in SEQ ID NO: 70, a CDRL2 comprising (or consisting of) the amino acid sequence set forth in SEQ ID NO: 71, and a CDRL3 comprising (or consisting of) the amino acid sequence set forth in SEQ ID NO: 72. In some aspects, the antibody is a chimeric, humanized, or human antibody.

In some aspects, the anti-PD-1 antibody, antigen binding portion thereof, comprises a VH comprising (or consisting of) the amino acid sequence set forth in SEQ ID NO: 73, and a VL comprising (or consisting of) the amino acid sequence set forth in SEQ ID NO: 74.

In some aspects, the anti-PD-1 antibody, antigen binding portion thereof, comprises a heavy chain comprising (or consisting of) the amino acid sequence as set forth in SEQ ID NO: 75 and a light chain comprising (or consisting of) the amino acid sequence as set forth in SEQ ID NO: 76.

In some aspects, the anti-PD-1 antibody, antigen binding portion thereof, comprises a VH comprising a CDRH1 comprising (or consisting of) the amino acid sequence set forth in SEQ ID NO: 77, a CDRH2 comprising (or consisting of) the amino acid sequence set forth in SEQ ID NO: 78, and a CDRH3 comprising (or consisting of) the amino acid sequence set forth in SEQ ID NO: 79; and a VL comprising a CDRL1 comprising (or consisting of) the amino acid sequence set forth in SEQ ID NO: 80, a CDRL2 comprising (or consisting of) the amino acid sequence set forth in SEQ ID NO: 81, and a CDRL3 comprising (or consisting of) the amino acid sequence set forth in SEQ ID NO: 82. In some aspects, the antibody is a chimeric, humanized, or human antibody.

In some aspects, the anti-PD-1 antibody, antigen binding portion thereof, comprises a VH comprising (or consisting of) the amino acid sequence set forth in SEQ ID NO: 83, and a VL comprising (or consisting of) the amino acid sequence set forth in SEQ ID NO: 84.

In some aspects, the anti-PD-1 antibody comprises a heavy chain comprising (or consisting of) the amino acid sequence as set forth in SEQ ID NO: 85 and a light chain comprising (or consisting of) the amino acid sequence as set forth in SEQ ID NO: 86.

In some aspects, the anti-PD-1 antibody, antigen binding portion thereof, comprises a VH comprising a CDRH1 comprising (or consisting of) the amino acid sequence set forth in SEQ ID NO: 87, a CDRH2 comprising (or consisting of) the amino acid sequence set forth in SEQ ID NO: 88, and a CDRH3 comprising (or consisting of) the amino acid sequence set forth in SEQ ID NO: 89; and a VL comprising a CDRL1 comprising (or consisting of) the amino acid sequence set forth in SEQ ID NO: 90, a CDRL2 comprising (or consisting of) the amino acid sequence set forth in SEQ ID NO: 91, and a CDRL3 comprising (or consisting of) the amino acid sequence set forth in SEQ ID NO: 92. In some aspects, the antibody is a chimeric, humanized, or human antibody.

In some aspects, the anti-PD-1 antibody, antigen binding portion thereof, comprises a VH comprising (or consisting of) the amino acid sequence set forth in SEQ ID NO: 93, and a VL comprising (or consisting of) the amino acid sequence set forth in SEQ ID NO: 94.

In some aspects, the anti-PD-1 antibody comprises a heavy chain comprising (or consisting of) the amino acid sequence as set forth in SEQ ID NO: 95 and a light chain comprising (or consisting of) the amino acid sequence as set forth in SEQ ID NO: 96.

In some aspects, the anti-PD-1 antibody is pembrolizumab, nivolumab, cemiplimab, spartalizumab, camrelizumab, sintilimab, tislelizumab, toripalimab, dostarlimab, retifanlimab, pimivalimab, dostarlimab, serplulimab, zimberelimab, acrixolimab, MEDI-0680, AM-0001, STI-1110, AGEN2034, BCD-100, sasanlimab, BI 754091, or SSI-361.

In some aspects, the anti-PD-1 antibody comprises the CDRH1, CDRH2, CDRH3, CDRL1, CDRL2, CDRL3, the VH and the VL, and/or the heavy and light chains of any of pembrolizumab, nivolumab, cemiplimab, spartalizumab, camrelizumab, sintilimab, tislelizumab, toripalimab, dostarlimab, retifanlimab, pimivalimab, MEDI-0680, GLS-010, AM-0001, STI-1110, AGEN2034, BCD-100, sasanlimab, BI 754091, or SSI-361.

In some aspects, the anti-PD-1 antibody is selected from the group consisting of nivolumab (OPDIVO®; formerly designated 5C4, BMS-936558, MDX-1106, or ONO-4538), pembrolizumab (KEYTRUDA®; formerly designated lambrolizumab and MK-3475; see WO 2008/156712A1), PDR001 (see WO 2015/112900), MEDI-0680 (formerly designated AMP-514; see WO 2012/145493), REGN-2810 see WO 2015/112800), JS001 (see Liu and Wu, 2017), BGB-A317 (see WO 2015/035606 and US 2015/0079109), INCSHR1210 (SHR-1210; see WO 2015/085847; Liu and Wu, 2017), TSR-042 (ANB011; see WO 2014/179664), GLS-010 (WBP3055; see Liu and Wu, 2017), AM-0001 (see WO 2017/123557), STI-1110 (see WO 2014/194302), AGEN2034 (see WO 2017/040790), and MGD013 (see WO 2017/106061).

In some aspects, the anti-PD-1 antibody is pembrolizumab (Merck; also known as KEYTRUDA®, lambrolizumab, and MK-3475; see, for example, WO 2008/156712). Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587. In some aspects, the PD-1 axis-blocking agent comprises an antigen binding portion of pembrolizumab.

In some aspects, the anti-PD-1 antibody comprises nivolumab (also known as OPDIVO®, 5C4, BMS-936558, MDX-1106, and ONO-4538). Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (see, for example, U.S. Pat. No. 8,008,449; Wang et al., 2014 Cancer Immunol Res. 2 (9): 846-56). In some aspects, the PD-1 axis-blocking agent comprises an antigen binding portion of nivolumab.

In some aspects, the anti-PD-1 antibody is cemiplimab (Regeneron; also known as LIBTAYO or REGN-2810; see, for example, WO 2015/112800 and U.S. Pat. No. 9,987,500). In some aspects, the PD-1 axis-blocking agent comprises an antigen binding portion of cemiplimab.

In some aspects, the anti-PD-1 antibody is spartalizumab (Novartis; also known as PDR001; see, for example, WO 2015/112900 and U.S. Pat. No. 9,683,048). In some aspects, the PD-1 axis-blocking agent comprises an antigen binding portion of spartalizumab.

In some aspects, the anti-PD-1 antibody is camrelizumab (Jiangsu Hengrui Medicine; also known as SHR-1210 or INCSHR1210; see, for example, WO 2015/085847; Si-Yang Liu et al. (2017) J. Hematol. Oncol. 10:136). In some aspects, the PD-1 axis-blocking agent comprises an antigen binding portion of camrelizumab.

In some aspects, the anti-PD-1 antibody is MEDI-0680 (AstraZeneca; also known as AMP-514; see, for example, WO 2012/145493). In some aspects, the PD-1 axis-blocking agent comprises an antigen binding portion of MEDI-0680.

In some aspects, the anti-PD-1 antibody is pimivalimab (also known as JTX-4014; see, for example, Papadopoulos, et al. (2022) IOTECH 16 (Supplement 1): 100284). In some aspects, the PD-1 axis-blocking agent comprises an antigen binding portion of pimivalimab.

In some aspects, the anti-PD-1 antibody is toripalimab (TAIZHOU JUNSHI PHARMA; also known as JS001; see, for example, Si-Yang Liu et al. (2017) J. Hematol. Oncol. 10:136). In some aspects, the PD-1 axis-blocking agent comprises an antigen binding portion of toripalimab.

In some aspects, the anti-PD-1 antibody is tislelizumab (Beigene; also known as BGB-A317; see, for example, WO 2015/35606 and US 2015/0079109). In some aspects, the PD-1 axis-blocking agent comprises an antigen binding portion of tislelizumab.

In some aspects, the anti-PD-1 antibody is dostarlimab (Tesaro Biopharmaceutical; also known as ANB011 or TSR-042; see, for example, WO2014/179664). In some aspects, the PD-1 axis-blocking agent comprises an antigen binding portion of dostarlimab.

In some aspects, the anti-PD-1 antibody is GLS-010 (Wuxi/Harbin Gloria Pharmaceuticals; also known as WBP3055; see, for example, Si-Yang Liu et al. (2017) J. Hematol. Oncol. 10:136). In some aspects, the PD-1 axis-blocking agent comprises an antigen binding portion of GLS-010.

In some aspects, the anti-PD-1 antibody is AM-0001 (Armo BioSciences). In some aspects, the PD-1 axis-blocking agent comprises an antigen binding portion of AM-0001.

In some aspects, the anti-PD-1 antibody is STI-1110 (Sorrento Therapeutics; see, for example, WO 2014/194302). In some aspects, the PD-1 axis-blocking agent comprises an antigen binding portion of STI-1110.

In some aspects, the anti-PD-1 antibody is AGEN2034 (Agenus; see, for example, WO 2017/040790). In some aspects, the PD-1 axis-blocking agent comprises an antigen binding portion of AGEN2034.

In some aspects, the anti-PD-1 antibody is retifanlimab (Macrogenics, also known as MGA012, AEX-1188, and INCMGA-00012; see, for example, WO 2017/19846). In some aspects, the PD-1 axis-blocking agent comprises an antigen binding portion of retifanlimab.

In some aspects, the anti-PD-1 antibody is BCD-100 (Biocad; see, for example, Kaplon et al. (2018) mAbs 10 (2): 183-203). In some aspects, the PD-1 axis-blocking agent comprises an antigen binding portion of BCD-100.

In some aspects, the anti-PD-1 antibody is sintilimab (Innovent; also known as IBI308; see, for example, WO 2017/024465, WO 2017/025016, WO 2017/132825, and WO 2017/133540). In some aspects, the PD-1 axis-blocking agent comprises an antigen binding portion of sintilimab.

In some aspects, the anti-PD-1 antibody is sasanlimab (Pfizer; also known as PF-06801591; see, for example, US 2016/0159905). In some aspects, the PD-1 axis-blocking agent comprises an antigen binding portion of sasanlimab.

In some aspects, the anti-PD-1 antibody is BI 754091 (Boehringer Ingelheim; see, for example, Zettl, M et al. (2018) Cancer. Res. 78 (13 Suppl): Abstract 4558) In some aspects, the PD-1 axis-blocking agent comprises an antigen binding portion of any of BI 754091.

In some aspects, the anti-PD-1 antibody is SSI-361 (Lyvgen Biopharma Holdings Limited, see, for example, US 2018/0346569). In some aspects, the PD-1 axis-blocking agent comprises an antigen binding portion of SSI-361.

In some aspects, the anti-PD-1 antibody is an anti-PD-1 antibody (such as anti-human-PD-1 antibody) that cross-competes for binding to PD-1 (such as human PD-1) with any anti-PD-1 antibody described herein, e.g., nivolumab (see, e.g., U.S. Pat. Nos. 8,008,449 and 8,779,105; WO 2013/173223) and/or pembrolizumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., nivolumab and/or pembrolizumab, by virtue of their binding to the same epitope region of PD-1. Cross-competing antibodies can be readily identified based on their ability to cross-compete with the anti-PD-1 antibodies described herein, such as nivolumab and/or pembrolizumab, in standard PD-1 binding assays such as Biacore analysis, ELISA assays or flow cytometry (see, e.g., WO 2013/173223). In some aspects, the PD-1 axis-blocking agent comprises an antigen binding portion of the anti-PD-1 antibody (such as an anti-human-PD-1 antibody) that cross-competes for the binding to PD-1 (such as human PD-1) with any anti-PD-1 antibody described herein, e.g., nivolumab and/or pembrolizumab. In some aspects, the antibody described in this paragraph is a monocloncal antibody. In some aspects, the antibody described in this paragraph is a chimeric, humanized, or human antibody.

In some aspects, the anti-PD-1 antibody binds the same epitope as any of the anti-PD-1 antibodies described herein, e.g., nivolumab and/or pembrolizumab. In some aspects, the antibody described in this paragraph is a monocloncal antibody. In some aspects, the antibody described in this paragraph is a chimeric, humanized, or human antibody.

Chimeric, humanized and human Abs can be prepared and isolated by methods well known in the art, e.g., as described in U.S. Pat. No. 8,008,449.

In some aspects, the PD-1 axis-blocking agent comprises an antigen binding portion of an anti-PD-1 antibody as described herein. Suitable antigen binding portions include a Fab, F(ab′)₂, Fd or Fv fragment, a sdAb, a scFv, di-scFv or bi-scFv, a diabody, a minibody or an isolated CDR (see also Hollinger and Hudson (2005) Nature Biotech. 23 (9): 1126-36; Olafsen and Wu (2010) Semin. Nucl. Med. 40 (3): 167-81, for further details).

In some aspects, the anti-PD-1 antibody (or antigen binding portion thereof) is administered at a dose ranging from about 0.1 mg/kg to about 20.0 mg/kg of the subject's body weight once every 2, 3, 4, 5, 6, 7, or 8 weeks, e.g., about 0.1 mg/kg to about 10.0 mg/kg body weight once every 2, 3, or 4 weeks. In some aspects, the anti-PD-1 antibody (or antigen binding portion thereof) is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In some aspects, the anti-PD-1 antibody (or antigen binding portion thereof) is administered at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In some aspects, the anti-PD-1 antibody (or antigen binding portion thereof) is administered at a dose of about 5 mg/kg body weight about once every 3 weeks. In some aspects, the anti-PD-1 antibody, e.g., nivolumab, (or antigen binding portion thereof) is administered at a dose of about 3 mg/kg body weight about once every 2 weeks. In some aspects, the anti-PD-1 antibody, e.g., pembrolizumab, (or antigen binding portion thereof) is administered at a dose of about 2 mg/kg body weight about once every 3 weeks.

In some aspects, the anti-PD-1 antibody (or antigen binding portion thereof) is administered at a dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In some aspects, the anti-PD-1 antibody (or antigen binding portion thereof) is administered at a dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In some aspects, the anti-PD-1 antibody (or antigen binding portion thereof) is administered at a dose of about 200 mg to about 800 mg, about 200 mg to about 700 mg, about 200 mg to about 600 mg, about 200 mg to about 500 mg, at a dosing interval of about 1, 2, 3, or 4 weeks.

In some aspects, the anti-PD-1 antibody (or antigen binding portion thereof) is administered at a dose of about 200 mg at about once every 3 weeks. In some aspects, the anti-PD-1 antibody (or antigen binding portion thereof) is administered at a dose of about 200 mg at about once every 2 weeks. In some aspects, the anti-PD-1 antibody (or antigen binding portion thereof) is administered at a dose of about 240 mg at about once every 2 weeks. In some aspects, the anti-PD-1 antibody (or antigen binding portion thereof) is administered at a dose of about 480 mg at about once every 4 weeks.

In some aspects, nivolumab is administered at a dose of about 240 mg once about every 2 weeks. In some aspects, nivolumab is administered at a dose of about 240 mg once about every 3 weeks. In some aspects, nivolumab is administered at a dose of about 360 mg once about every 3 weeks. In some aspects, nivolumab is administered at a dose of about 480 mg once about every 4 weeks. In some aspects, nivolumab is administered at a dose of about 720 mg once about every 6 weeks. In some aspects, nivolumab is administered at a dose of about 960 mg once about every 8 weeks.

In some aspects, pembrolizumab is administered at a dose of about 200 mg once about every 2 weeks. In some aspects, pembrolizumab is administered at a dose of about 200 mg once about every 3 weeks. In some aspects, pembrolizumab is administered at a dose of about 400 mg once about every 4 weeks. In some aspects, pembrolizumab is administered at a dose of about 400 mg once about every 6 weeks.

In some aspects, the additional anti-tumor treatment comprises means for PD-1 axis-blocking (wherein the means are as described herein).

### ii) PD-L1

In some aspects, the PD-1 axis-blocking agent comprises a PD-L1 antagonist. In some aspects, the PD-1 axis-blocking agent comprises a human PD-L1 antagonist. In some aspects, the PD-L1 antagonist comprises an anti-PD-L1 antibody (i.e., an antibody that specifically binds to PD-L1) or an antigen binding portion thereof, an immunoadhesin, an oligopeptide, or a nucleic acid molecule.

In some aspects, the PD-1 axis-blocking agent comprises an antigen binding portion of an anti-PD-L1 antibody (such as a F(ab′)₂ fragment, a Fab′ fragment, a Fab fragment, a Fv fragment, a scFv fragment, a dsFv fragment, or a dAb fragment). In some aspects, the PD-1 axis-blocking agent comprises an anti-human-PD-L1 antibody or antigen binding portion thereof.

The antibody might be a multispecific antibody (such as a bispecific antibody). In some aspects, the multispecific antibody is a DART, a DVD-Ig, or bispecific antibody.

In some aspects, the anti-PD-L1 antibody is a multispecific antibody, such as a bispecific or trispecific antibody. In some aspects, the anti-PD-L1 antibody is a bispecific antibody that specifically binds to (human) PD-L1 and (human) LAG-3. In some aspects, the anti-PD-L1 antibody is a bispecific antibody that specifically binds to (human) PD-L1 and (human) PD-1. In some aspects, the anti-PD-L1 antibody is a trispecific antibody that specifically binds to (human) PD-L1, (human) LAG-3, and (human) PD-1.

Anti-PD-L1 antibodies suitable for use in the methods of the present disclosure are antibodies that bind to PD-L1 with high specificity and affinity, block the binding of PD-1, and inhibit the immunosuppressive effect of the PD-1 signaling pathway.

In some aspects, the anti-PD-L1 antibody or antigen binding portion thereof comprises a heavy chain constant region which is of a human IgG1, IgG2, IgG3 or IgG4 isotype. In some aspects, the anti-PD-L1 antibody or antigen binding portion thereof comprises a heavy chain constant region which is of a human IgG4 isotype. In some aspects, the anti-PD-L1 antibody or antigen binding portion thereof is of a human IgG1 isotype. In some aspects, the IgG4 heavy chain constant region of the anti-PD-L1 antibody or antigen binding portion thereof contains an S228P mutation (numbered using the Kabat system) which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies. In some aspects, the anti-PD-L1 antibody or antigen binding portion thereof comprises a light chain constant region which is a human kappa or lambda constant region.

Examples of anti-PD-L1 antibodies useful in the methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include antibodies, such as monoclonal antibodies, that bind specifically to human PD-L1 and exhibit at least one, in some aspects, at least five, of the preceding characteristics.

Examples of anti-PD-L1 antibodies useful in the methods of the present disclosure include the antibodies disclosed in U.S. Pat. No. 7,943,743. Other anti-PD-L1 antibodies have been described in, for example, U.S. Pat. Nos. 8,217,149, 8,779,108, 9,175,082 and 9,624,298, and PCT Publication No. WO 2012/145493. The anti-PD-1 human monoclonal antibodies disclosed in U.S. Pat. No. 7,943,743 have been demonstrated to exhibit one or more of the following characteristics: (a) binding to human PD-L1 with a K_{D} of about 50 mM or lower, as determined by SPR (BIACORE®); (b) increasing T-cell proliferation, interferon-γ production and IL-2 secretion in a MLR assay; (c) stimulating Ab responses; (d) inhibiting the binding of PD-L1 to PD-1; and (e) reversing the suppressive effect of Tregs on T cell effector cells and/or dendritic cells. Anti-PD-L1 antibodies useful in the methods of the present disclosure include antibodies, such as monoclonal antibodies, that bind specifically to human PD-L1 with high affinity and exhibit at least one, in some aspects at least three or all, of the preceding characteristics.

In some aspects, the anti-PD-L1 antibody is atezolizumab, durvalumab, avelumab, envafolimab, cosibelimab, BMS-936559, STI-1014, CX-072, LY3300054, FAZ053, CS-1001, SHR-1316, CBT-502, KN035, or BGB-A333. In some aspects, the PD-1 axis-blocking agent comprises an antigen binding portion of any of the antibodies described in this paragraph.

In some aspects, the anti-PD-L1 antibody is BMS-936559 (also known as 12A4, MDX-1105; see, e.g., U.S. Pat. No. 7,943,743 and WO 2013/173223). In some aspects, the PD-1 axis-blocking agent comprises an antigen binding portion of BMS-936559.

In some aspects, the anti-PD-L1 antibody is STI-1014 (Sorrento; see, for example, WO 2013/181634). STI-104 is designated H6 in U.S. Pat. No. 9,175,082. In some aspects, the PD-1 axis-blocking agent comprises an antigen binding portion of STI-1014.

In some aspects, the anti-PD-L1 antibody is CX-072 (Cytomx; see, for example, WO 2016/149201). In some aspects, the PD-1 axis-blocking agent comprises an antigen binding portion of CX-072.

In some aspects, the anti-PD-L1 antibody is LY3300054 (Eli Lilly Co.; see, e.g., WO 2017/034916). In some aspects, the PD-1 axis-blocking agent comprises an antigen binding portion of LY3300054.

In some aspects, the anti-PD-L1 antibody is FAZ053 (Novartis). In some aspects, the PD-1 axis-blocking agent comprises an antigen binding portion of FAZ053.

In some aspects, the anti-PD-L1 antibody is CK-301 (Checkpoint Therapeutics; see, for example, Gorelik et al. (2016) AACR: Abstract 4606). CK-301 is also referred to as cosibelimab. In some aspects, the PD-1 axis-blocking agent comprises an antigen binding portion of CK-301.

In some aspects, the anti-PD-L1 antibody is CS-1001. See, for example, Zhou et al., Journal of Clinical Oncology, Meeting Abstract, 2020 ASCO Annual Meeting I, Lung Cancer-Non-Small Cell Metastatic, e21687, and Zhang et al. (2020) Cancer Res. 80 (16_Supplement): 3260. In some aspects, the PD-1 axis-blocking agent comprises an antigen binding portion of CS-1001.

In some aspects, the anti-PD-L1 antibody is SHR-1316. See, for example, Mu et al. (2021) Thorac Cancer 12 (9): 1373-1381, and Wu et al., Anals of Oncology, Abstract, Vol. 33, Supplement 2, S72, April 2022. In some aspects, the PD-1 axis-blocking agent comprises an antigen binding portion of SHR-1316.

In some aspects, the anti-PD-L1 antibody is CBT-502 (also known as TQB2450; see, for example, Wei et al. (2018) Mol. Cancer Ther. 17 (1_Supplement): A200). In some aspects, the PD-1 axis-blocking agent comprises an antigen binding portion of CBT-502.

In some aspects, the anti-PD-L1 antibody is KN035 (3D Med/Alphamab; also referred to as envafolimab; see, for example, Zhang et al. (2017) Cell Discov. 7:-3 and Shimizu et al. (2022) Invest. New Drugs 40 (5): 1021-1031). In some aspects, the PD-1 axis-blocking agent comprises an antigen binding portion of KN035.

In some aspects, the anti-PD-L1 antibody is BGB-A333 (BeiGene; see, for example, Desai et al. (2018) JCO 36 (15suppl): TPS3113 and Desai et al. (2023) British Journal of Cancer 128:1418-1428). In some aspects, the PD-1 axis-blocking agent comprises an antigen binding portion of BGB-A333.

In certain aspects, the PD-L1 antibody is atezolizumab. Atezolizumab is a fully humanized IgG1 monoclonal anti-PD-L1 antibody. Atezolizumab (Roche) is also known as TECENTRIQ®; MPDL3280A, RG7446. See, for example, U.S. Pat. No. 8,217,149 and Herbst et al. (2013) J. Clin. Oncol. 31 (suppl): 3000). Atezolizumab is designated YW243.55S70 in U.S. Pat. No. 8,217,149. In some aspects, the PD-1 axis-blocking agent comprises an antigen binding portion of atezolizumab.

In certain aspects, the PD-L1 antibody is durvalumab. Durvalumab is a human IgG1 kappa monoclonal anti-PD-L1 antibody. Durvalumab (AstraZeneca) is also known as IMFINZI® or MEDI-4736. Durvalumab is designated 2.14H9OPT in U.S. Pat. No. 8,779,108. See, for example, WO 2011/066389. In some aspects, the PD-1 axis-blocking agent comprises an antigen binding portion of durvalumab.

In certain aspects, the PD-L1 antibody is avelumab. Avelumab is a human IgG1 lambda monoclonal anti-PD-L1 antibody. Avelumab (Pfizer) is also known as BAVENCIOR or MSB-0010718C. Avelumab is designated A09-246-2 in U.S. Pat. No. 9,624,298. See, for example, WO 2013/079174. In some aspects, the PD-1 axis-blocking agent comprises an antigen binding portion of avelumab.

In some aspects, the anti-PD-L1 antibody is an anti-PD-L1 antibody (such as an anti-human-PD-L1 antibody) that cross-competes for the binding to PD-L1 (such as human PD-L1) with any anti-PD-L1 antibody described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of the reference antibody, e.g., atezolizumab, durvalumab, and/or avelumab, by virtue of their binding to the same epitope region of PD-L1. Cross-competing antibodies can be readily identified based on their ability to cross-compete with any of the anti-PD-L1 antibodies described herein, e.g., atezolizumab, durvalumab, and/or avelumab, in standard PD-L1 binding assays such as Biacore analysis, ELISA assays or flow cytometry (see, e.g., WO 2013/173223). In some aspects, the PD-1 axis-blocking agent comprises an antigen binding portion of the anti-PD-L1 antibody (such as an anti-human-PD-L1 antibody) that cross-competes for the binding to PD-L1 (such as human PD-L1) with any anti-PD-L1 antibody described herein, e.g., atezolizumab, durvalumab, and/or avelumab. In some aspects, the antibody described in this paragraph is a monocloncal antibody. In some aspects, the antibody described in this paragraph is a chimeric, humanized, or human antibody.

In some aspects, the anti-PD-L1 antibody binds the same epitope as any of the anti-PD-L1 antibodies described herein, e.g., atezolizumab, durvalumab, and/or avelumab. In some aspects, the antibody described in this paragraph is a monocloncal antibody. In some aspects, the antibody described in this paragraph is a chimeric, humanized, or human antibody.

Chimeric, humanized and human Abs can be prepared and isolated by methods well known in the art, e.g., as described in U.S. Pat. No. 7,943,743.

In some aspects, the PD-1 axis-blocking agent comprises an antigen binding portion of an anti-PD-L1 antibody as described herein. Suitable antigen binding portions include, but are not limited to, Fab, F(ab′)₂, Fd, Fv, and scFv, di-scFv or bi-scFv, and scFv-Fc fragments, nanobodies, diabodies, triabodies, tetrabodies, and isolated CDRs, that bind to PD-L1 and exhibit functional properties similar to those of whole antibodies in inhibiting receptor binding and up-regulating the immune system.

In some aspects, the anti-PD-L1 antibody (or antigen binding portion thereof) is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody (or antigen binding portion thereof) is administered at a dose of about 15 mg/kg body weight at about once every 3 weeks. In some aspects, the anti-PD-L1 antibody (or antigen binding portion thereof) is administered at a dose of about 10 mg/kg body weight at about once every 2 weeks.

In some aspects, the anti-PD-L1 antibody (or antigen binding portion thereof) is administered at a dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody (or antigen binding portion thereof) is administered at a dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody (or antigen binding portion thereof) is administered at a dose of about 1200 mg at about once every 3 weeks. In some aspects, the anti-PD-L1 antibody (or antigen binding portion thereof) is administered at a dose of about 800 mg at about once every 2 weeks. In other aspects, the anti-PD-L1 antibody (or antigen binding portion thereof) is administered at a dose of about 840 mg at about once every 2 weeks.

In some aspects, atezolizumab is administered at a dose of about 1200 mg once about every 3 weeks. In some aspects, atezolizumab is administered at a dose of about 800 mg once about every 2 weeks. In some aspects, atezolizumab is administered at a dose of about 840 mg once about every 2 weeks.

In some aspects, avelumab is administered at a dose of about 800 mg once about every 2 weeks.

In some aspects, durvalumab is administered at a dose of about 10 mg/kg once about every 2 weeks. In some aspects, durvalumab is administered at a dose of about 800 mg/kg once about every 2 weeks. In some aspects, durvalumab is administered at a dose of about 1200 mg/kg once about every 3 weeks.

iii) PD-L2

In some aspects, the PD-1 axis-blocking agent comprises a PD-L2 antagonist. In some aspects, the PD-1 axis-blocking agent comprises a human PD-L2 antagonist. In some aspects, the PD-L2 antagonist comprises an anti-PD-L2 antibody (i.e., an antibody that specifically binds to PD-L2) or an antigen binding portion thereof, an immunoadhesin, an oligopeptide, or a nucleic acid molecule.

In some aspects, the PD-1 axis-blocking agent comprises an antigen binding portion of an anti-PD-L2 antibody (such as a F(ab′)₂ fragment, a Fab′ fragment, a Fab fragment, a Fv fragment, a scFv fragment, a dsFv fragment, or a dAb fragment). In some aspects, the PD-1 axis-blocking agent comprises an anti-human-PD-L2 antibody or antigen binding portion thereof.

In some aspects, the antibody comprises a multispecific antibody (such as a bispecific antibody). In some aspects, the multispecific antibody is a DART, a DVD-Ig, or bispecific antibody.

In some aspects, the anti-PD-L2 antibody is a monospecific antibody (specifically binding to PD-L2). In some aspects, the anti-PD-L2 antibody is a multispecific antibody, such as a bispecific or trispecific antibody. In some aspects, the anti-PD-L2 antibody is a bispecific antibody that specifically binds to (human) PD-L2 and (human) LAG-3. In some aspects, the anti-PD-L2 antibody is a bispecific antibody that specifically binds to (human) PD-L2 and (human) PD-1. In some aspects, the anti-PD-L2 antibody is a trispecific antibody that specifically binds to (human) PD-L2, (human) LAG-3, and (human) PD-1.

In some aspects, the anti-PD-L2 antibody is rHIgM12B. rHIgM12B is, for example, described in van Keulen et al. (2006) Clin Exp Immunol. 143 (2): 314-321. rHIgM12B has been evaluated in clinical trials (see clinicaltrails.gov, identifier NCT00658892). The sequences of the VH and VL (amino acid and nucleotide sequences) have been deposited in GenBank under the accession numbers DQ146928 and DQ146929, respectively. In some aspects, the PD-1 axis-blocking agent is an antigen binding portion of rHIgM12B.

In some aspects, the anti-PD-L2 antibody binds the same epitope as rHIgM12B.

### b) LAG-3 Antagonist

In some aspects, the additional anti-tumor treatment comprises a LAG-3 antagonist.

In some aspects, the LAG-3 antagonist comprises a human LAG-3 antagonist. In some aspects, the LAG-3 antagonist comprises an anti-LAG-3 antibody (i.e., an antibody that specifically binds to LAG-3) or an antigen binding portion thereof, an immunoadhesin, an oligopeptide, or a nucleic acid molecule.

In some aspects, the LAG-3 antagonist comprises an antigen binding portion of an anti-LAG-3 antibody (such as a F(ab′)₂ fragment, a Fab′ fragment, a Fab fragment, a Fv fragment, a scFv fragment, a dsFv fragment, or a dAb fragment). In some aspects, the LAG-3 antagonist comprises an anti-human-LAG-3 antibody or antigen binding portion thereof.

In some aspects, the anti-LAG-3 antibody (such as an anti-human-LAG-3 antibody) comprises a multispecific antibody (such as a bispecific antibody). In some aspects, the multispecific antibody is a dual-affinity re-targeting antibody (DART), a dual variable domain immunoglobulin (DVD-Ig), or bispecific antibody.

In some aspects, the anti-LAG-3 antibody is a monospecific antibody (specifically binding to LAG-3). In some aspects, the anti-LAG-3 antibody is a multispecific antibody, such as a bispecific or trispecific antibody.

Exemplary sequences of anti-LAG-3 antibodies suitable for the methods of the present disclosure are given in Table 12.

TABLE 12
Exemplary sequences of anti-LAG-3 antibodies suitable
for the methods of the present disclosure.
Anti-LAG-3 antibody relatlimab

| | |
|---|---|
| CDRH1 (SEQ ID NO: 97) | DYYWN |
| CDRH2 (SEQ ID NO: 98) | EINHRGSTNSNPSLKS |
| CDRH3 (SEQ ID NO: 99) | GYSDYEYNWFDP |
| CDRL1 (SEQ ID NO: 100) | RASQSISSYLA |
| CDRL2 (SEQ ID NO: 101) | DASNRAT |
| CDRL3 (SEQ ID NO: 102) | QQRSNWPLT |
| VH (SEQ ID NO: 103) | QVQLQQWGAGLLKPSETLSLTCAVYGGSFSDYYWNWIRQPPGKGLEWIGE |
| | INHRGSTNSNPSLKSRVTLSLDTSKNQFSLKLRSVTAADTAVYYCAFGYS |
| | DYEYNWFDPWGQGTLVTVSS |
| VL (SEQ ID NO: 104) | EIVLTQSPATLSLSPGERATLSCRASQSISSYLAWYQQKPGQAPRLLIYD |
| | ASNRATGIPARFSGSGSGTDFTLTISSLEPEDFAVYYCQQRSNWPLTFGQ |
| | GTNLEIK |
| Heavy chain | QVQLQQWGAGLLKPSETLSLTCAVYGGSFSDYYWNWIRQPPGKGLEWIGE |
| (SEQ ID NO: 105) | INHRGSTNSNPSLKSRVTLSLDTSKNQFSLKLRSVTAADTAVYYCAFGYS |
| | DYEYNWFDPWGQGTLVTVSSASTKGPSVFPLAPCSRSTSESTAALGCLVK |
| | DYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDT |
| | LMISRTPEVTCVVVDVSQEDPEVQFNWYVDGVEVHNAKTKPREEQFNSTY |
| | RVVSVLTVLHQDWLNGKEYKCKVSNKGLPSSIEKTISKAKGQPREPQVYT |
| | LPPSQEEMTKNQVSLTCLVKGFYPSDIAVEWESNGQPENNYKTTPPVLDS |
| | DGSFFLYSRLTVDKSRWQEGNVFSCSVMHEALHNHYTQKSLSLSLGK |
| Light chain | EIVLTQSPATLSLSPGERATLSCRASQSISSYLAWYQQKPGQAPRLLIYD |
| (SEQ ID NO: 106) | ASNRATGIPARFSGSGSGTDFTLTISSLEPEDFAVYYCQQRSNWPLTFGQ |
| | GTNLEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC |

Anti-LAG-3 antibody fianlimab (REGN3767)

| | |
|---|---|
| CDRH1 (SEQ ID NO: 107) | GFTFSSYG |
| CDRH2 (SEQ ID NO: 108) | IWYDGSNK |
| CDRH3 (SEQ ID NO: 109) | ASVATSGDFDYYGMDV |
| CDRL1 (SEQ ID NO: 110) | QRISTY |
| CDRL2 (SEQ ID NO: 111) | DAS |
| CDRL3 (SEQ ID NO: 112) | QQRSNWPLT |
| VH (SEQ ID NO: 113) | QVQLVESGGGVVQPGRSLRLSCVASGFTFSSYGMHWVRQAPGKGLEWVAI |
| | IWYDGSNKYYADSVKGRFTISRDNSKNTQYLQMNSLRAEDTAVYYCASVA |
| | TSGDFDYYGMDVWGQGTTVTVSS |
| VL (SEQ ID NO: 114) | EIVLTQSPATLSLSPGERTTLSCRASQRISTYLAWYQQKPGQAPRLLIYD |
| | ASKRATGIPARFSGSGSGTGFTLTISSLEPEDFAVYYCQQRSNWPLTFGG |
| | GTKVEIK |
| Heavy chain | QVQLVESGGGVVQPGRSLRLSCVASGFTFSSYGMHWVRQAPGKGLEWVAI |
| (SEQ ID NO: 115) | IWYDGSNKYYADSVKGRFTISRDNSKNTQYLQMNSLRAEDTAVYYCASVA |
| | TSGDFDYYGMDVWGQGTTVTVSSASTKGPSVFPLAPCSRSTSESTAALGC |
| | LVKDYFPEPVTVSWNSGALTSGVHTFPAVLQSSGLYSLSSVVTVPSSSLG |
| | TKTYTCNVDHKPSNTKVDKRVESKYGPPCPPCPAPPVAGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGK |
| Light chain | EIVLTQSPATLSLSPGERTTLSCRASQRISTYLAWYQQKPGQAPRLLIYD |
| (SEQ ID NO: 116) | ASKRATGIPARFSGSGSGTGFTLTISSLEPEDFAVYYCQQRSNWPLTFGG |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC |

Anti-LAG-3 antibody ieramilimab (LAG525)

| | |
|---|---|
| CDRH1 (SEQ ID NO: 117) | NYGMN |
| CDRH2 (SEQ ID NO: 118) | WINTDTGEPTYADDFKG |
| CDRH3 (SEQ ID NO: 119) | NPPYYYGTNNAEAMDY |
| CDRL1 (SEQ ID NO: 120) | SSSQDISNYLN |
| CDRL2 (SEQ ID NO: 121) | YTSTLHL |
| CDRL3 (SEQ ID NO: 122) | QQYYNLPWT |
| VH (SEQ ID NO: 123) | QVQLVQSGAEVKKPGASVKVSCKASGFTLTNYGMNWVRQARGQRLEWIGW |
| | INTDTGEPTYADDFKGRFVFSLDTSVSTAYLQISSLKAEDTAVYYCARNP |
| | PYYYGTNNAEAMDYWGQGTTVTVSS |
| VL (SEQ ID NO: 124) | DIQMTQSPSSLSASVGDRVTITCSSSQDISNYLNWYLQKPGQSPQLLIYY |
| | TSTLHLGVPSRFSGSGSGTEFTLTISSLQPDDFATYYCQQYYNLPWTFGQ |
| | GTKVEIK |
| VH′ (SEQ ID NO: 125) | QVQLVQSGAEVKKPGASVKVSCKASGFTLTNYGMNWVRQAPGQGLEWMGW |
| | INTDTGEPTYADDFKGRFVFSLDTSVSTAYLQISSLKAEDTAVYYCARNP |
| | PYYYGTNNAEAMDYWGQGTTVTVSS |
| VL′ (SEQ ID NO: 126) | DIQMTQSPSSLSASVGDRVTITCSSSQDISNYLNWYQQKPGKAPKLLIYY |
| | TSTLHLGIPPRFSGSGYGTDFTLTINNIESEDAAYYFCQQYYNLPWTFGQ |
| | GTKVEIK |
| Heavy chain | QVQLVQSGAEVKKPGASVKVSCKASGFTLTNYGMNWVRQARGQRLEWIGW |
| (SEQ ID NO: 127) | INTDTGEPTYADDFKGRFVFSLDTSVSTAYLQISSLKAEDTAVYYCARNP |
| | PYYYGTNNAEAMDYWGQGTTVTVSSASTKGPSVFPLAPCSRSTSESTAAL |
| | GCLVKDYFPEPVTVSWNSGALTSGVHTFPAVLQSSGLYSLSSVVTVPSSS |
| | LGTKTYTCNVDHKPSNTKVDKRVESKYGPPCPPCPAPEFLGGPSVFLFPP |
| | KPKDTLMISRTPEVTCVVVDVSQEDPEVQFNWYVDGVEVHNAKTKPREEQ |
| | FNSTYRVVSVLTVLHQDWLNGKEYKCKVSNKGLPSSIEKTISKAKGQPRE |
| | PQVYTLPPSQEEMTKNQVSLTCLVKGFYPSDIAVEWESNGQPENNYKTTP |
| | PVLDSDGSFFLYSRLTVDKSRWQEGNVFSCSVMHEALHNHYTQKSLSLSL |
| | G |
| Light chain | DIQMTQSPSSLSASVGDRVTITCSSSQDISNYLNWYLQKPGQSPQLLIYY |
| (SEQ ID NO: 128) | TSTLHLGVPSRFSGSGSGTEFTLTISSLQPDDFATYYCQQYYNLPWTFGQ |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSENRGEC |
| Heavy chain′ | QVQLVQSGAEVKKPGASVKVSCKASGFTLTNYGMNWVRQAPGQGLEWMGW |
| (SEQ ID NO: 129) | INTDTGEPTYADDFKGRFVFSLDTSVSTAYLQISSLKAEDTAVYYCARNP |
| | PYYYGTNNAEAMDYWGQGTTVTVSSASTKGPSVFPLAPCSRSTSESTAAL |
| | GCLVKDYFPEPVTVSWNSGALTSGVHTFPAVLQSSGLYSLSSVVTVPSSS |
| | LGTKTYTCNVDHKPSNTKVDKRVESKYGPPCPPCPAPEFLGGPSVFLFPP |
| | KPKDTLMISRTPEVTCVVVDVSQEDPEVQFNWYVDGVEVHNAKTKPREEQ |
| | FNSTYRVVSVLTVLHQDWLNGKEYKCKVSNKGLPSSIEKTISKAKGQPRE |
| | PQVYTLPPSQEEMTKNQVSLTCLVKGFYPSDIAVEWESNGQPENNYKTTP |
| | PVLDSDGSFFLYSRLTVDKSRWQEGNVFSCSVMHEALHNHYTQKSLSLSL |
| | G |
| Light chain′ | DIQMTQSPSSLSASVGDRVTITCSSSQDISNYLNWYQQKPGKAPKLLIYY |
| (SEQ ID NO: 130) | TSTLHLGIPPRFSGSGYGTDFTLTINNIESEDAAYYFCQQYYNLPWTFGQ |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC |

Anti-LAG-3 antibody favezelimab (MK4280)

| | |
|---|---|
| CDRH1 (SEQ ID NO: 131) | DYNVD |
| CDRH2 (SEQ ID NO: 132) | DINPNDGGTIYAQKFQE |
| CDRH3 (SEQ ID NO: 133) | NYRWFGAMDH |
| CDRL1 (SEQ ID NO: 134) | KASQSLDYEGDSDMN |
| CDRL2 (SEQ ID NO: 135) | GASNLES |
| CDRL3 (SEQ ID NO: 136) | QQSTEDPRT |
| VH (SEQ ID NO: 137) | QMQLVQSGPEVKKPGTSVKVSCKASGYTFTDYNVDWVRQARGQRLEWIGD |
| | INPNDGGTIYAQKFQERVTITVDKSTSTAYMELSSLRSEDTAVYYCARNY |
| | RWFGAMDHWGQGTTVTVSS |
| VL (SEQ ID NO: 138) | DIVMTQTPLSLSVTPGQPASISCKASQSLDYEGDSDMNWYLQKPGQPPQL |
| | LIYGASNLESGVPDRFSGSGSGTDFTLKISRVEAEDVGVYYCQQSTEDPR |
| | TFGGGTKVEIK |
| Heavy chain | QMQLVQSGPEVKKPGTSVKVSCKASGYTFTDYNVDWVRQARGQRLEWIGD |
| (SEQ ID NO: 139) | INPNDGGTIYAQKFQERVTITVDKSTSTAYMELSSLRSEDTAVYYCARNY |
| | RWFGAMDHWGQGTTVTVSSASTKGPSVFPLAPCSRSTSESTAALGCLVKD |
| | YFPEPVTVSWNSGALTSGVHTFPAVLQSSGLYSLSSVVTVPSSSLGTKTY |
| | TCNVDHKPSNTKVDKRVESKYGPPCPPCPAPEFLGGPSVFLFPPKPKDTL |
| | MISRTPEVTCVVVDVSQEDPEVQFNWYVDGVEVHNAKTKPREEQFNSTYR |
| | VVSVLTVLHQDWLNGKEYKCKVSNKGLPSSIEKTISKAKGQPREPQVYTL |
| | PPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGK |
| Light chain | DIVMTQTPLSLSVTPGQPASISCKASQSLDYEGDSDMNWYLQKPGQPPQL |
| (SEQ ID NO: 140) | LIYGASNLESGVPDRFSGSGSGTDFTLKISRVEAEDVGVYYCQQSTEDPR |
| | TFGGGTKVEIKRTVAAPSVFIFPPSDEQLKSGTASVVCLLNNFYPREAKV |
| | QWKVDNALQSGNSQESVTEQDSKDSTYSLSSTLTLSKADYEKHKVYACEV |
| | THQGLSSPVTKSFNRGEC |

In some aspects, the anti-LAG-3 antibody, antigen binding portion thereof, comprises a VH comprising a CDRH1 comprising (or consisting of) the amino acid sequence set forth in SEQ ID NO: 97, a CDRH2 comprising (or consisting of) the amino acid sequence set forth in SEQ ID NO: 98, and a CDRH3 comprising (or consisting of) the amino acid sequence set forth in SEQ ID NO: 99; and a VL comprising a CDRL1 comprising (or consisting of) the amino acid sequence set forth in SEQ ID NO: 100, a CDRL2 comprising (or consisting of) the amino acid sequence set forth in SEQ ID NO: 101, and a CDRL3 comprising (or consisting of) the amino acid sequence set forth in SEQ ID NO: 102.

In some aspects, the anti-LAG-3 antibody, antigen binding portion thereof, comprises a VH comprising (or consisting of) the amino acid sequence set forth in SEQ ID NO: 103, and a VL comprising (or consisting of) the amino acid sequence set forth in SEQ ID NO: 104.

In some aspects, the anti-LAG-3 antibody comprises a heavy chain comprising (or consisting of) the amino acid sequence as set forth in SEQ ID NO: 105 and a light chain comprising (or consisting of) the amino acid sequence as set forth in SEQ ID NO: 106.

In some aspects, the anti-LAG-3 antibody, antigen binding portion thereof, comprises a VH comprising a CDRH1 comprising (or consisting of) the amino acid sequence set forth in SEQ ID NO: 107, a CDRH2 comprising (or consisting of) the amino acid sequence set forth in SEQ ID NO: 108, and a CDRH3 comprising (or consisting of) the amino acid sequence set forth in SEQ ID NO: 109; and a VL comprising a CDRL1 comprising (or consisting of) the amino acid sequence set forth in SEQ ID NO: 110, a CDRL2 comprising (or consisting of) the amino acid sequence set forth in SEQ ID NO: 111, and a CDRL3 comprising (or consisting of) the amino acid sequence set forth in SEQ ID NO: 112.

In some aspects, the anti-LAG-3 antibody, antigen binding portion thereof, comprises a VH comprising (or consisting of) the amino acid sequence set forth in SEQ ID NO: 113, and a VL comprising (or consisting of) the amino acid sequence set forth in SEQ ID NO: 114.

In some aspects, the anti-LAG-3 antibody comprises a heavy chain comprising (or consisting of) the amino acid sequence as set forth in SEQ ID NO: 115 and a light chain comprising (or consisting of) the amino acid sequence as set forth in SEQ ID NO: 116.

In some aspects, the anti-LAG-3 antibody, antigen binding portion thereof, comprises a VH comprising a CDRH1 comprising (or consisting of) the amino acid sequence set forth in SEQ ID NO: 117, a CDRH2 comprising (or consisting of) the amino acid sequence set forth in SEQ ID NO: 118, and a CDRH3 comprising (or consisting of) the amino acid sequence set forth in SEQ ID NO: 119; and a VL comprising a CDRL1 comprising (or consisting of) the amino acid sequence set forth in SEQ ID NO: 120, a CDRL2 comprising (or consisting of) the amino acid sequence set forth in SEQ ID NO: 121, and a CDRL3 comprising (or consisting of) the amino acid sequence set forth in SEQ ID NO: 122.

In some aspects, the anti-LAG-3 antibody, antigen binding portion thereof, comprises a VH comprising (or consisting of) the amino acid sequence set forth in SEQ ID NO: 123, and a VL comprising (or consisting of) the amino acid sequence set forth in SEQ ID NO: 124.

In some aspects, the anti-LAG-3 antibody, antigen binding portion thereof, comprises a VH comprising (or consisting of) the amino acid sequence set forth in SEQ ID NO: 125, and a VL comprising (or consisting of) the amino acid sequence set forth in SEQ ID NO: 126.

In some aspects, the anti-LAG-3 antibody comprises a heavy chain comprising (or consisting of) the amino acid sequence as set forth in SEQ ID NO: 127 and a light chain comprising (or consisting of) the amino acid sequence as set forth in SEQ ID NO: 128.

In some aspects, the anti-LAG-3 antibody comprises a heavy chain comprising (or consisting of) the amino acid sequence as set forth in SEQ ID NO: 129 and a light chain comprising (or consisting of) the amino acid sequence as set forth in SEQ ID NO: 130.

In some aspects, the anti-LAG-3 antibody, antigen binding portion thereof, comprises a VH comprising a CDRH1 comprising (or consisting of) the amino acid sequence set forth in SEQ ID NO: 131, a CDRH2 comprising (or consisting of) the amino acid sequence set forth in SEQ ID NO: 132, and a CDRH3 comprising (or consisting of) the amino acid sequence set forth in SEQ ID NO: 133; and a VL comprising a CDRL1 comprising (or consisting of) the amino acid sequence set forth in SEQ ID NO: 134, a CDRL2 comprising (or consisting of) the amino acid sequence set forth in SEQ ID NO: 135, and a CDRL3 comprising (or consisting of) the amino acid sequence set forth in SEQ ID NO: 136.

In some aspects, the anti-LAG-3 antibody, antigen binding portion thereof, comprises a VH comprising (or consisting of) the amino acid sequence set forth in SEQ ID NO: 137, and a VL comprising (or consisting of) the amino acid sequence set forth in SEQ ID NO: 138.

In some aspects, the anti-LAG-3 antibody comprises a heavy chain comprising (or consisting of) the amino acid sequence as set forth in SEQ ID NO: 139 and a light chain comprising (or consisting of) the amino acid sequence as set forth in SEQ ID NO: 140.

In some aspects, the anti-LAG-3 antibody is relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3 (0414), aLAG-3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501

In some aspects, the anti-LAG-3 antibody comprises the CDRH1, CDRH2, CDRH3, CDRL1, CDRL2, CDRL3, the VH and the VL, and/or the heavy and light chains of any of relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3 (0414), aLAG-3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501.

In some aspects, the anti-LAG-3 antibody comprises relatlimab (BMS-986016). In some aspects, the anti-LAG-3 antibody comprises IMP731 (H5L7BW). In some aspects, the anti-LAG-3 antibody comprises MK4280 (28G-10, favezelimab). MK-4280 (28G-10, favezelimab) described in WO2016028672 and U.S. Publication No. 2020/0055938. In some aspects, the anti-LAG-3 antibody comprises REGN3767 (fianlimab). REGN3767 (fianlimab) is described, for example, in Burova E, et al. (2016) J. Immunother. Cancer 4 (Supp. 1): P195 and U.S. Pat. No. 10,358,495. In some aspects, the anti-LAG-3 antibody comprises GSK2831781. In some aspects, the anti-LAG-3 antibody comprises humanized BAP050. Humanized BAP050 is described, for example, in WO2017/019894. In some aspects, the anti-LAG-3 antibody comprises IMP-701 (LAG525, ieramilimab) IMP-701 (LAG525; ieramilimab) is described, for example, in U.S. Pat. No. 10,711,060 and U.S. Publ. No. 2020/0172617. In some aspects, the anti-LAG-3 antibody comprises aLAG-3 (0414). In some aspects, the anti-LAG-3 antibody comprises aLAG-3 (0416). In some aspects, the anti-LAG-3 antibody comprises Sym022. In some aspects, the anti-LAG-3 antibody comprises TSR-033. In some aspects, the anti-LAG-3 antibody comprises TSR-075. In some aspects, the anti-LAG-3 antibody comprises XmAb841 (XmAb22841). In some aspects, the anti-LAG-3 antibody comprises MGD013 (tebotelimab). In some aspects, the anti-LAG-3 antibody comprises BI754111. In some aspects, the anti-LAG-3 antibody comprises FS118. In some aspects, the anti-LAG-3 antibody comprises P 13B02-30. In some aspects, the anti-LAG-3 antibody comprises AVA-017. In some aspects, the anti-LAG-3 antibody comprises 25F7. 25F7 is described, for example, in U.S. Publ. No. 2011/0150892. In some aspects, the anti-LAG-3 antibody comprises AGEN1746. In some aspects, the anti-LAG-3 antibody comprises RO7247669. In some aspects, the anti-LAG-3 antibody comprises INCAGN02385. In some aspects, the anti-LAG-3 antibody comprises IBI-110. In some aspects, the anti-LAG-3 antibody comprises EMB-02. In some aspects, the anti-LAG-3 comprises IBI-323. In some aspects, the anti-LAG-3 antibody comprises LBL-007. In some aspects, the anti-LAG-3 antibody comprises ABL501.

In general, any anti-LAG-3 antibody can be used in the methods of the present disclosure. Antibodies that bind to LAG-3 have been disclosed in Int'l Publ. No. WO/2015/042246 and U.S. Publ. Nos. 2014/0093511 and 2011/0150892, each of which is incorporated by reference herein in its entirety. Disclosure relating to the anti-LAG-3 antibodies described herein and other anti-LAG-3 antibodies useful in the methods of the present disclosure can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

In some aspects, the anti-LAG-3 antibody is MGD013 (tebotelimab), which is a bispecific PD-1×LAG-3 DART. In some aspects, the anti-LAG-3 antibody comprises the CDRH1, CDRH2, CDRH3, CDRL1, CDRL2, CDRL3, the VH and the VL, and/or the heavy and light chains of tebotelimab.

In some aspects, the anti-LAG-3 antibody is an anti-LAG-3 antibody (such as anti-human-LAG-3 antibody) that cross-competes for binding to LAG-3 (such as human LAG-3) with any anti-LAG-3 antibody described herein, e.g., relatlimab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of the reference antibody, e.g., relatlimab, by virtue of their binding to the same epitope region of LAG-3. Cross-competing antibodies can be readily identified based on their ability to cross-compete with the anti-LAG-3 antibodies described herein, such as relatlimab, in standard LAG-3 binding assays such as Biacore analysis, ELISA assays or flow cytometry (see, e.g., WO 2013/173223). In some aspects, the LAG-3 axis-blocking agent comprises an antigen binding portion of the anti-LAG-3 antibody (such as an anti-human-LAG-3 antibody) that cross-competes for the binding to LAG-3 (such as human LAG-3) with any anti-LAG-3 antibody described herein, e.g., relatlimab. In some aspects, the antibody described in this paragraph is a monocloncal antibody. In some aspects, the antibody described in this paragraph is a chimeric, humanized, or human antibody.

In some aspects, the anti-LAG-3 antibody binds the same epitope as any of the anti-LAG-3 antibodies described herein, e.g., relatlimab. In some aspects, the antibody described in this paragraph is a monocloncal antibody. In some aspects, the antibody described in this paragraph is a chimeric, humanized, or human antibody.

In some aspects, the additional anti-tumor treatment comprises means for antagonizing LAG-3 (wherein the means are as described herein).

As outlined above, the present disclosure is, in some aspects, related to methods of treating a tumor in a subject in need thereof.

In some aspects, the tumor is a malignant (i.e., cancerous) tumor.

In some aspects, the tumor is a liquid tumor. In some aspects, the tumor is a malignant liquid tumor. A liquid tumor may also be referred to as a hematological malignancy herein. Hematological malignancies include liquid tumors derived from either of the two major blood cell lineages, i.e., the myeloid cell line (which produces granulocytes, erythrocytes, thrombocytes, macrophages and mast cells) or the lymphoid cell line (which produces B, T, NK and plasma cells), including all types of leukemias, lymphomas, and myelomas. Hematological malignancies that may be treated using the present therapy methods include, for example, acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

In some aspects, the tumor is a solid tumor, such as a lung cancer, a liver cancer, or a colorectal cancer. In some aspects, the tumor is a malignant solid tumor.

In some aspects, the tumor is a malignant tumor that is replased, metastatic, unresectable, (locally) advanced, and/or refractory.

In some aspects, the tumor is relapsed, or recurrent. Tumor relapse, or cancer recurrence, describes the return of cancer after treatment has initially successfully cleared signs and symptoms of cancer in the patient. A tumor most likely relapses as a few of the original cancer cells survived the initial treatment.

In some aspects, the tumor is metastatic. In some aspects, the tumor is non-metastatic.

In some aspects, the tumor is unresectable. In some aspects, the tumor is advanced. In some aspects, the tumor is locally advanced.

In some aspects, the tumor is refractory. A refractory is a tumor that is not responding to a treatment, wherein the treatment may be the most recent treatment that was administered to the subject.

In some aspects, the tumor is refractory to at least one (prior) anti-tumor therapy, wherein the at least one (prior) anti-tumor therapy is not a combination therapy of a KRAS inhibitor and a Treg-depleting agent. In some aspects, the tumor is refractory to one, two, three, or four (prior) anti-tumor therapies.

In some aspects, the tumor is refractory to a monotherapy with a KRAS inhibitor.

In some aspects, the tumor is refractory to a combination therapy of a KRAS inhibitor and an additional anti-tumor treatment. In some aspects, the additional anti-tumor treatment does not comprise a Treg cell-depleting agent. In some aspects, the additional anti-tumor treatment is an anti-tumor treatment as described above in the section titled “Additional anti-tumor treatment”. In some aspects, the additional anti-tumor treatment comprises a PD-1 axis blocking agent, such as an anti-PD-1 or an anti-PD-L1 antibody. In some aspects, the additional anti-tumor treatment comprises a PD-1 axis blocking agent (such as an anti-PD-1 or an anti-PD-L1 antibody) and a LAG-3 antagonist (such as an anti-LAG-3 antibody). In some aspects, the tumor is refractory to a combination therapy of a KRAS inhibitor (such as a KRAS G12C inhibitor, for example, adagrasib, sotorasib, or MRTX1257) and an anti-PD-1 antibody (such as nivolumab or pembrolizumab), and optionally an anti-LAG-3 antibody (such as relatlimab). In some aspects, the tumor is refractory to a combination therapy of a KRAS inhibitor (such as a KRAS G12C inhibitor, for example, adagrasib, sotorasib, or MRTX1257) and an anti-PD-L1 antibody (such as atezolizumab, durvalumab, or avelumab), and optionally an anti-LAG-3 antibody (such as relatlimab).

In some aspects, the tumor is refractory to a (prior) anti-tumor therapy comprising a PD-1 axis-blocking agent. In some aspects, the tumor is refractory to a monotherapy with a PD-1 axis-blocking agent. In some aspects, the tumor is refractory to a combination therapy with a PD-1 axis-blocking agent and an anti-CTLA-4 antibody (such as ipilimumab or tremelimumab). As regards the PD-1 axis-blocking agent, reference is generally made to the section titled “PD-1 axis-blocking agent” above. In some aspects, the PD-1 axis-blocking agent is an anti-PD-1 antibody (such as nivolumab or pembrolizumab). In some aspects, the PD-1 axis-blocking agent is an anti-PD-L1 antibody (such as atezolizumab, durvalumab, or avelumab).

In some aspects, the method is a first line (IL) therapy.

In some aspects, the method is a second line (2L) therapy.

In some aspects, the method is a third line (3L) therapy.

In aspects of the present disclosure, wherein the tumor is described to be a certain type of cancer (such as a tumor being a NSCLC), reference is made to a tumor which is derived from that certain type of cancer. A cancerous solid tumor, for example, is the result of cancer cells forming a lump or growth.

In some aspects, the tumor is advanced melanoma, advanced non-small cell lung cancer, metastatic renal cell carcinoma, classical Hodgkin lymphoma, advanced squamous cell carcinoma of the head and neck, metastatic urothelial carcinoma, MSI-H or dMMR metastatic colorectal cancer, hepatocellular carcinoma, small cell lung cancer, and esophageal squamous cell carcinoma.

In some aspects, the tumor is selected from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, cancers of viral origin, advanced cancer, unresectable cancer, metastatic cancer, refractory cancer, recurrent cancer, and any combination thereof. In some aspects, the tumor is an advanced, unresectable, metastatic, refractory cancer, and/or recurrent.

In some aspects, the tumor is head and neck squamous cell carcinoma (HNSC), lung adenocarcinoma (LUAD), stomach adenocarcinoma (STAD), lung squamous cell carcinoma (LUSC), pancreatic adenocarcinoma (PAAD), rectum adenocarcinoma (READ), esophageal carcinoma (ESCA), breast invasive carcinoma (BRCA), colon adenocarcinoma (COAD), cervical squamous cell carcinoma and endocervical adenocarcinoma (CESC), follicular lymphoma, acute lymphocytic leukemia, lymphoma, or any combination thereof. In some aspects, the tumor is head and neck squamous cell carcinoma (HNSCC), colorectal cancer, non-small cell lung cancer-squamous cell carcinoma (NSCLC-SCC), NSCLC-adenocarcinoma (NSCLC-ADC), cervical cancer, pancreatic cancer, gastric cancer, bladder cancer, breast cancer, or any combination thereof.

In some aspects, the tumor is selected from the group consisting of squamous cell carcinoma, small-cell lung cancer (SCLC), non-small cell lung cancer (NSCLC), squamous NSCLC, nonsquamous NSCLC, glioma, gastrointestinal cancer, renal cancer, clear cell carcinoma, ovarian cancer, liver cancer, colorectal cancer, endometrial cancer, kidney cancer, renal cell carcinoma (RCC), prostate cancer, hormone refractory prostate adenocarcinoma, thyroid cancer, neuroblastoma, pancreatic cancer, glioblastoma, glioblastoma multiforme, cervical cancer, stomach cancer, bladder cancer, hepatoma, breast cancer, colon carcinoma, head and neck cancer, gastric cancer, germ cell tumor, pediatric sarcoma, sinonasal natural killer, melanoma, bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, rectal cancer, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, environmentally-induced cancers including those induced by asbestos, virus-related cancers or cancers of viral origin (e.g., human papilloma virus (HPV-related or -originating tumors)), and any combination thereof.

In some aspects, the tumor is selected from the group consisting of pancreatic adenocarcinoma, colon adenocarcinoma, colorectal adenocarcinoma, lung adenocarcinoma, non-small cell lung cancer (NSCLC), cholangiocarcinoma, uterine endometrial carcinoma, testicular germ cell cancer, ampullary carcinoma, cervical squamous cell carcinoma, appendiceal adenocarcinoma, bladder adenocarcinoma, small intestinal carcinoma, ovarian cancer, and myelodysplastic syndromes.

In some aspects, the subject suffers from tumors derived from two or more cancers described in this paragraph.

In some aspects, the ovarian cancer is an endometrioid ovarian cancer, a low grade serous ovarian cancer, or a mucinous ovarian cancer.

In some aspects, the tumor is a lung cancer, a colorectal cancer, or a pancreatic cancer.

In some aspects, the tumor is a lung cancer. In some aspects, the lung cancer is a NSCLC. In some aspects, the NSCLC is a lung (invasive) adenocarcinoma. In some aspects, the NSCLC is a squamous cell carcinoma. In some aspects, the NSCLC is locally advanced or metastatic.

In some aspects, the tumor is a colorectal cancer. In some aspects, the colorectal cancer is a colorectal adenocarcinoma.

In some aspects, the tumor is a pancreatic cancer. In some aspects, the pancreatic cancer is a pancreatic adenocarcinoma.

In some aspects, the tumor comprises a KRAS mutation. In some aspects, the tumor comprises one or more of the following mutations: KRAS G12A, KRAS G12C, KRAS G12D, KRAS G12R, KRAS G12S, KRAS G12V, KRAS G12R, KRAS G12F, KRAS G13C, KRAS G13D, KRAS Q61L, KRAS Q61H, and KRAS Q61K. In some aspects, the tumor comprises one or more of the following mutations: KRAS G12C, KRAS G12D, and KRAS G12V. In some aspects, the tumor comprises a KRAS G12C mutation. In some aspects, the tumor comprises a KRAS G12D mutation. In some aspects, the tumor comprises a KRAS G12V mutation.

In some aspects, the tumor is a pancreatic cancer comprising a KRAS G12D mutation.

In some aspects, the tumor is a colorectal cancer (such as a colorectal adenocarcinoma) comprising a KRAS G12D mutation. In some aspects, the tumor is a lung cancer (such as a NSCLC) comprising a KRAS G12D mutation.

In some aspects, the tumor is a pancreatic cancer comprising a KRAS G12V mutation.

In some aspects, the tumor is a colorectal cancer (such as a colorectal adenocarcinoma) comprising a G12V mutation. In some aspects, the tumor is a lung cancer (such as NSCLC) comprising a G12V mutation.

In some aspects, the tumor is a lung cancer (such as NSCLC) comprising a KRAS G12C mutation. In some aspects, the tumor is a pancreatic cancer (such as a pancreatic adenocarcinoma) comprising a KRAS G12C mutation. In some aspects, the tumor is a colorectal cancer (such as a colorectal adenocarcinoma) comprising a G12C mutation.

In some aspects, the tumor is a NSCLC comprising a KRAS G12C, G12V, and/or G12D mutation. In some aspects, the tumor is a NSCLC comprising a KRAS G12C mutation.

In some aspects, the tumor is a lung (invasive) adenocarcinoma comprising a KRAS G12C, G12V, and/or G12D mutation. In some aspects, the tumor is a lung (invasive) adenocarcinoma comprising a KRAS G12C mutation.

In some aspects, the tumor is a squamous cell carcinoma comprising a KRAS G12C, G12V, and/or G12D mutation. In some aspects, the tumor is a squamous cell carcinoma comprising a KRAS G12C mutation.

### 5. Subject

In some aspects, the subject is a mammal. Examples of mammals are dog, cat, mouse, rat, horse, pig, primate (such as nonhuman primate), and human.

In some aspects, the subject is a human. In some aspects, the human is 16 years of age or older. In some aspects, the human is 18 years of age or older. In some aspects, the human is 20 years of age or older. In some aspects, the human is 25 years of age or older.

In some aspects, the subject (such as a human) has previously received at least one anti-tumor therapy. In some aspects, the at least one anti-tumor therapy is not a combination therapy of a KRAS inhibitor and a Treg cell-depleting agent. In some aspects, the at least one anti-tumor therapy is a systemic anti-tumor therapy.

In some aspects, the subject has previously received one, two, three, four, five or more anti-tumor therapies.

In some aspects, the subject has previously received a surgery, a radiation therapy, a chemotherapy, an immunotherapy, or any combination thereof.

In some aspects, the subject has previously received a therapy with a PD-1 axis-blocking agent. In some aspects, the subject has previously received a monotherapy with a PD-1 axis-blocking agent. In some aspects, the subject has previously received a combination therapy with a PD-1 axis-blocking agent and an anti-CTLA-4 antibody (such as ipilimumab or tremelimumab). As regards the PD-1 axis-blocking agent, reference is generally made to the section titled “PD-1 axis-blocking agent” above. In some aspects, the PD-1 axis-blocking agent is an anti-PD-1 antibody (such as nivolumab or pembrolizumab). In some aspects, the PD-1 axis-blocking agent is an anti-PD-L1 antibody (such as atezolizumab, durvalumab, or avelumab).

In some aspects, the subject has previously received a therapy with a KRAS inhibitor, such as a KRAS G12C inhibitor, for example, adagrasib, sotorasib, or MRTX1257. In some aspects, the subject has previously received a monotherapy with a KRAS inhibitor (such as a KRAS G12C inhibitor, for example, adagrasib, sotorasib, or MRTX1257). In some aspects, the subject has previously received a combination therapy of a KRAS inhibitor (such as a KRAS G12C inhibitor, for example, adagrasib, sotorasib, or MRTX1257) and an additional anti-tumor treatment that does not comprise a Treg cell-depleting agent (for example, a PD-1 axis-blocking agent, such as an anti-PD-1 or an anti-PD-L1 antibody).

In some aspects, the subject is determined to carry a KRAS mutation prior to administering the KRAS inhibitor. In some aspects, the subject is determined to carry a KRAS mutation by analyzing a plasma sample or tumor tissue sample from the subject.

In some aspects, the KRAS inhibitor that is administered to the subject with the Treg-depleting agent is a KRAS G12C inhibitor (such as adagrasib, sotorasib, or MRTX1257) and the subject is determined to carry a KRAS G12C mutation prior to administering the KRAS G12C inhibitor. In some aspects, the subject is determined to carry a KRAS G12C mutation by analyzing a plasma sample or tumor tissue sample from the subject.

In some aspects, the KRAS inhibitor that is administered to the subject with the Treg-depleting agent is a KRAS G12D inhibitor and the subject is determined to carry a KRAS G12D mutation prior to administering the KRAS G12D inhibitor. In some aspects, the subject is determined to carry a KRAS G12D mutation by analyzing a plasma sample or tumor tissue sample from the subject.

In some aspects, the subject is determined to express CCR8 in the tumor microenvironment. In some aspects, the subject is determined to express CTLA-4 in the tumor microenvironment. In some aspects, the subject is determined to express PD-1 in the tumor microenvironment. In some aspects, the subject is determined to express PD-L1 in the tumor microenvironment. In some aspects, the subject is determined to express LAG-3 in the tumor microenvironment. In some aspects, expression is determined from a tumor tissue sample from the subject. In some aspects, expression is determined in tissue samples taken at multiple time points, for example, before treatment, during treatment, and/or after treatment. In some aspects, the tissue samples are from different locations in the subject, for example, from a primary tumor and from a metastasis.

In some aspects, the subject has not previously received an anti-tumor therapy (i.e., the subject is treatment-naïve). In some aspects, the subject has not previously received a systemic anti-tumor therapy.

In some aspects, the subject is identified as having an increased number of Tregs in the TME. In some aspects, the subject is identified as having an increased number of Tregs in the TME and the number of Tregs is increased when compared to a reference level from one or more other subjects that are responsive to an anti-tumor therapy that was previously administered to the subject (such as a an anti-tumor therapy comprising a KRAS inhibitor). In some aspects, the number of Tregs is determined in a tumor tissue sample from the subject. In some aspects, the tumor tissue sample is a tumor tissue slice. In some aspects, the number of Tregs is determined in a spatial cellular community comprising CD4⁺ T cells, CD8⁺ T cells, Tregs, and DCs. In some aspects, the spatial cellular community is a spatial cellular community as further described herein in the section titled “Further methods of the present disclosure” below. In some aspects, the Tregs are Tregs in the neighborhood of CD8⁺ T cells. In some aspects, the CD8⁺ T cells in the neighborhood of the Tregs are CD8⁺ T cells that are further neighboring DCs and CD4⁺ T cells. In some aspects, the number of Tregs is determined by IMC.

In some aspects, the subject is identified as having a spatial cellular community comprising CD4⁺ T cells, CD8⁺ T cells, Tregs, and DCs in the TME. In some aspects, the spatial cellular community is a spatial cellular community as further described herein in the section titled “Further methods of the present disclosure” below. In some aspects, the presence or absence of the spatial cellular community is determined in a tumor tissue sample from the subject. In some aspects, the tumor tissue sample is a tumor tissue slice. In some aspects, the presence or absence of the spatial cellular community is determined by IMC.

In some aspects, the subject is identified as having an increased number of spatial cellular communities comprising CD4⁺ T cells, CD8⁺ T cells, Tregs, and DCs in the TME. In some aspects, the subject is identified as having an increased number of spatial cellular communities comprising CD4⁺ T cells, CD8⁺ T cells, Tregs, and DCs in the TME when compared to a reference level from one or more other subjects that are responsive to an anti-tumor therapy that was previously administered to the subject (such as a an anti-tumor therapy comprising a KRAS inhibitor). In some aspects, the spatial cellular community is a spatial cellular community as further described herein in the section titled “Further methods of the present disclosure” below. In some aspects, the number of spatial cellular communities is determined in a tumor tissue sample from the subject. In some aspects, the tumor tissue sample is a tumor tissue slice. In some aspects, the number of the spatial cellular communities is determined by IMC.

### 6. Medical Uses

In some aspects, the present disclosure also relates to the use of a KRAS inhibitor and a Treg-depleting agent and optionally an additional anti-tumor treatment as described herein in the manufacture of a medicament for a method of treating a tumor in a subject as described herein. In some aspects, the KRAS inhibitor is a KRAS G12C inhibitor, such as adagrasib, sotorasib, or MTRX-1257, the Treg-depleting agent is an anti-CTLA-4 antibody (such as tremelimumab or ipilimumab) or an antigen binding portion thereof, and the subject is a human. In some aspects, the KRAS inhibitor is a KRAS G12C inhibitor, such as adagrasib, sotorasib, or MTRX-1257, the Treg-depleting agent is an anti-CCR8 antibody (such as 14S15, 14S15h, or imzokitug) or an antigen binding portion thereof, and the subject is a human. In some aspects, the tumor is a lung cancer, such as NSCLC. In some aspects, the additional anti-tumor treatment is an anti-PD-1 antibody (such as nivolumab or pembrolizumab). In some aspects, the additional anti-tumor treatment is an anti-PD-L1 antibody (such as atezolizumab, durvalumab, or avelumab).

In some aspects, the present disclosure also relates to the KRAS inhibitor and the Treg-depleting agent and optionally the additional anti-tumor treatment as described herein for use in a method of treating a tumor in a subject as described herein. In some aspects, the KRAS inhibitor is a KRAS G12C inhibitor, such as adagrasib, sotorasib, or MTRX-1257, the Treg-depleting agent is an anti-CTLA-4 antibody (such as tremelimumab or ipilimumab) or an antigen binding portion thereof, and the subject is a human. In some aspects, the KRAS inhibitor is a KRAS G12C inhibitor, such as adagrasib, sotorasib, or MTRX-1257, the Treg-depleting agent is an anti-CCR8 antibody (such as 14S15, 14S15h, or imzokitug) or an antigen binding portion thereof, and the subject is a human. In some aspects, the tumor is a lung cancer, such as NSCLC. In some aspects, the additional anti-tumor treatment is an anti-PD-1 antibody (such as nivolumab or pembrolizumab). In some aspects, the additional anti-tumor treatment is an anti-PD-L1 antibody (such as atezolizumab, durvalumab, or avelumab).

### III. Kits

In some aspects, the present disclosure further relates to kits comprising the KRAS inhibitor and the Treg-depleting agent, and optionally an additional anti-tumor treatment as described herein.

In some aspects, the present disclosure relates to a kit comprising (a) one or more doses of a KRAS inhibitor; (b) one or more doses of a Treg-depleting agent; (c) optionally one or more doses of an additional anti-tumor treatment; and (d) instructions for use of the KRAS inhibitor, the Treg-depleting agent, and optionally the additional anti-tumor treatment in a method of treating a tumor.

In some aspects, the kit comprises (a) one or more daily doses of a KRAS inhibitor ranging from about 500 mg to about 2000 mg (such as from about 900 mg to about 1300 mg, for example, 960 mg or about 1200 mg); (b) one or more doses of a Treg-depleting agent ranging from about 100 mg to about 1000 mg; (c) optionally one or more doses of an additional anti-tumor treatment; and (d) instructions for use of the KRAS inhibitor, the Treg-depleting agent, and optionally the additional anti-tumor treatment in a method of treating tumor.

In some aspects, the instructions for use are instructions for use in a method of treating a tumor as described herein, in particular as described in the section titled “Method of treating a tumor comprising administering a KRAS inhibitor and a Treg-depleting agent”.

Regarding suitable doses of the KRAS inhibitor, the Treg-depleting agent, and optionally the additional anti-tumor treatment, reference is also made to corresponding disclosure on dosing in the section titled “Method of treating a tumor comprising administering a KRAS inhibitor and a Treg-depleting agent” above.

In some aspects, the tumor is a lung cancer, such as NSCLC.

In some aspects, the kit comprises one or more (daily) doses of a KRAS G12C inhibitor, such as sotorasib, adagrasib, or MRTX1257.

In some aspects, the Treg-depleting agent in the kit comprises an anti-CTLA-4 antibody (such as ipilimumab or tremelimumab) or an antigen binding portion thereof. In some aspects, the Treg-depleting agent in the kit comprises an anti-CCR8 antibody (such as 14S15, 14S15h, or imzokitug) or an antigen binding portion thereof.

In some aspects, the additional anti-tumor treatment in the kit comprises an anti-PD-L1 antibody or an antigen binding portion thereof. In some aspects, the additional anti-tumor treatment in the kit comprises an anti-PD-1 antibody or an antigen binding portion thereof. In some aspects, the additional anti-tumor treatment in the kit comprises an anti-PD-1 or an anti-PD-L1 antibody and further comprises an anti-LAG-3 antibody or an antigen binding portion thereof. In some aspects, the anti-LAG-3 antibody is relatlimab. In some aspects, the anti-PD-L1 antibody is atezolizumab, avelumab, or durvalumab. In some aspects, the anti-PD-1 antibody is nivolumab or pembrolizumab. In some aspects, the one or more doses of the additional anti-tumor treatment range from about 100 mg to about 1000 mg.

### IV. Further Methods of the Present Disclosure

In the following, further methods of the present disclosure are described.

### 1. Method of Reducing the Number of Tregs in the TME

In some aspects, the present disclosure is further directed to a method of reducing the number of Tregs in the TME in a subject who receives a therapy with a KRAS inhibitor, the method comprising administering a Treg-depleting agent to the subject.

In some aspects, the subject exhibits an increased number of Tregs in the TME compared to the number of Tregs in the TME before the therapy with the KRAS inhibitor. In some aspects, the subject exhibits an increased number of Tregs in the TME compared to a reference level from one or more other subjects having a tumor who are responsive to therapy with the KRAS inhibitor.

In some aspects, after the administering, the number of Tregs is reduced in a spatial cellular community in the TME comprising CD4⁺ T cells, CD8⁺ T cells, Tregs, and DCs. In some aspects, the spatial cellular community is further described below in the section titled “Spatial cellular community”.

In some aspects, the Tregs that are reduced after the administering are Tregs in the neighborhood of CD8⁺ T cells. In some aspects, the CD8⁺ T cells in the neighborhood of the Tregs are CD8⁺ T cells that are further neighboring DCs and CD4⁺ T cells.

In some aspects, the number of Tregs in the TME is determined in a tumor tissue sample from the subject. In some aspects, the tumor tissue sample is a tumor tissue slice.

In some aspects, the number of Tregs in the TME is determined by imaging mass cytometry (IMC).

In some aspects, administering the Treg-depleting agent reduces tumor growth compared to growth of the tumor prior to administering the Treg-depleting agent. In some aspects, administering the Treg-depleting agent reduces tumor volume compared to the volume of the tumor prior to administering the Treg-depleting agent. In some aspects, administering the Treg-depleting agent increases survival probability of the subject compared to the probability of survival prior to administering the Treg-depleting agent. In some aspects, administering the Treg-depleting agent enhances anti-tumor immunity compared to the anti-tumor immunity prior to administering the Treg-depleting agent. In some aspects, enhancing the anti-tumor immunity comprises increasing CD8⁺ T cell infiltration compared to the CD8⁺ T cell infiltration prior to administering the Treg-depleting agent. In some aspects, enhancing the anti-tumor immunity comprises increasing the number of activated CD8⁺ T cells compared to the number of activated CD8⁺ T cells prior to administering the Treg-depleting agent. In some aspects, administering the Treg-depleting agent enhances the systemic immune response compared to the systemic immune response prior to administering the Treg-depleting agent.

In some aspects, the therapy with the KRAS inhibitor is a monotherapy with the KRAS inhibitor.

In some aspects, the therapy with the KRAS inhibitor is a combination therapy with the KRAS inhibitor and an additional anti-tumor treatment. In some aspects, the additional anti-tumor treatment does not comprise a Treg cell depleting agent. In some aspects, the the additional anti-tumor treatment comprises a PD-1 axis-blocking agent, such as an anti-PD-1 antibody or an anti-PD-L1 antibody.

As regards further aspects of the method of reducing the number of Tregs as described in this section, including suitable tumors, subjects, KRAS inhibitors, administration modes, and/or

Treg-depleting agents for the method of reducing the number of Tregs as described in this section, reference is in particular made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 2. Method of Treating a Tumor in a Subject Identified as Having an Increased Number of Tregs

In some aspects, the present disclosure is further directed to a method of treating a tumor in a subject in need thereof comprising administering a Treg-depleting agent to the subject, wherein the subject is identified as having an increased number of Tregs in the TME after a therapy with a KRAS inhibitor.

In some aspects, the number of Tregs in the TME is increased compared to the number of Tregs in the TME before the therapy with the KRAS inhibitor. In some aspects, the number of Tregs in the TME is increased compared to a reference level from one or more other subjects having a tumor who are responsive to therapy with a KRAS inhibitor.

In some aspects, the Tregs in the TME further show an increased PD-1 and/or Ki67 expression compared to the Tregs in the TME before the therapy with the KRAS inhibitor.

In some aspects, the number of Tregs is determined in a tumor tissue sample from the subject. In some aspects, the tumor tissue sample is a tumor tissue slice. In some aspects, the number of Tregs is determined by imaging mass cytometry (IMC).

In some aspects, the Tregs are Tregs in the neighborhood of CD8⁺ T cells. In some aspects, the CD8⁺ T cells in the neighborhood of the Tregs are CD8⁺ T cells that are further neighboring DCs and CD4⁺ T cells.

In some aspects, the number of Tregs in the TME is determined in a spatial cellular community in the TME comprising CD4⁺ T cells, CD8⁺ T cells, Tregs, and DCs. In some aspects, the spatial cellular community is further described below in the section titled “Spatial cellular community”.

In some aspects, administering the Treg-depleting agent reduces tumor growth compared to growth of the tumor prior to administering the Treg-depleting agent. In some aspects, administering the Treg-depleting agent reduces tumor volume compared to the volume of the tumor prior to administering the Treg-depleting agent. In some aspects, administering the Treg-depleting agent increases survival probability of the subject compared to the probability of survival prior to administering the Treg-depleting agent. In some aspects, administering the Treg-depleting agent enhances anti-tumor immunity compared to the anti-tumor immunity prior to administering the Treg-depleting agent. In some aspects, enhancing the anti-tumor immunity comprises increasing CD8⁺ T cell infiltration compared to the CD8⁺ T cell infiltration prior to administering the Treg-depleting agent. In some aspects, enhancing the anti-tumor immunity comprises increasing the number of activated CD8⁺ T cells compared to the number of activated CD8⁺ T cells prior to administering the Treg-depleting agent. In some aspects, administering the Treg-depleting agent enhances the systemic immune response compared to the systemic immune response prior to administering the Treg-depleting agent.

In some aspects, the therapy with the KRAS inhibitor is a monotherapy with the KRAS inhibitor.

In some aspects, the therapy with the KRAS inhibitor is a combination therapy with the KRAS inhibitor and an additional anti-tumor treatment. In some aspects, the additional anti-tumor treatment does not comprise a Treg cell depleting agent. In some aspects, the the additional anti-tumor treatment comprises a PD-1 axis-blocking agent, such as an anti-PD-1 antibody or an anti-PD-L1 antibody.

As regards further aspects of the method of treating as described in this section, including suitable tumors, subjects, KRAS inhibitors, administration modes, and/or Treg-depleting agents for the method of treating as described in this section, reference is in particular made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 3. Method of Identifying and Method of Treating a Subject with an Increased Number of Tregs in a TME after Therapy with a KRAS Inhibitor

In some aspects, the present disclosure is further directed to a method of identifying a subject with an increased number of Tregs in a TME in the subject after a therapy with a KRAS inhibitor comprising measuring the number of Tregs in a spatial cellular community in the TME.

In some aspects, the number of Tregs in the spatial cellular community is increased upon treatment with the KRAS inhibitor compared to the number of Tregs in the spatial cellular community before the therapy with the KRAS inhibitor. In some aspects, the number of Tregs in the spatial cellular community is increased compared to a reference level from one or more other subjects having a tumor who are responsive to therapy with the KRAS inhibitor.

In some aspects, the method further comprises comparing the number of Tregs in the spatial cellular community upon treatment with the KRAS inhibitor to the number of Tregs in the spatial cellular community before the therapy with the KRAS inhibitor. In some aspects, the method further comprises comparing the number of Tregs in the spatial cellular community to a reference level from one or more other subjects having a tumor who are responsive to therapy with the KRAS inhibitor.

In some aspects, the Tregs in the spatial cellular community further show an increased PD-1 and/or Ki67 expression upon treatment with the KRAS inhibitor compared to the Tregs in the spatial cellular community before the therapy with the KRAS inhibitor.

In some aspects, the spatial cellular community comprises CD4⁺ T cells, CD8⁺ T cells, Tregs, and DCs.

In some aspects, the number of Tregs is determined in a tumor tissue sample from the subject. In some aspects, the tumor tissue sample is a tumor tissue slice. In some aspects, the number of Tregs is determined by imaging mass cytometry (IMC).

In some aspects, the Tregs are Tregs in the neighborhood of CD8⁺ T cells. In some aspects, the CD8⁺ T cells in the neighborhood of the Tregs are CD8⁺ T cells that are further neighboring DCs and CD4⁺ T cells.

In some aspects, the spatial cellular community further comprises tumor cells expressing cleaved-caspase-3 (c-casp3⁺) and wherein the number of CD8⁺ T cells in the neighborhood of c-casp3⁺ tumor cells in the spatial cellular community is increased upon treatment with the KRAS inhibitor compared to the number of CD8⁺ T cells in the neighborhood of c-casp3⁺ tumor cells in the spatial cellular community before the therapy with the KRAS inhibitor.

In some aspects, the number of CD8⁺ T cells in the neighborhood of CD4⁺ T cells, DCs, and/or CD103⁺ DCs in the spatial cellular community is increased upon treatment with the KRAS inhibitor compared to the number of CD8⁺ T cells in the neighborhood of CD4⁺ T cells, DCs, and/or CD103⁺ DCs in the spatial cellular community before the therapy with the KRAS inhibitor.

In some aspects, the CD8⁺ T cells in the spatial cellular community show an increased PD-1, CD39, LAG-3, and/or Ki67 expression upon treatment with the KRAS inhibitor compared to the CD8⁺ T cells in the spatial cellular community before the therapy with the KRAS inhibitor.

In some aspects, the CD4⁺ T cells in the spatial cellular community show an increased PD-1 and/or Ki67 expression upon treatment with the KRAS inhibitor compared to the CD4⁺ T cells in the spatial cellular community before the therapy with the KRAS inhibitor.

In some aspects, the DCs in the spatial cellular community show an increased CXCL9, PD-L1, and/or CD86 expression upon treatment with the KRAS inhibitor compared to the DCs in the spatial cellular community before the therapy with the KRAS inhibitor.

In some aspects, the minimum distance of DCs and/or CD103⁺ DCs having a mean CXCL9 expression of at least 0.5 as measured by ICM to PD-1⁺ CD8⁺ T cells, PD-1⁺ CD4⁺ T cells, and/or PD-1⁺ Tregs in the spatial cellular community is decreased upon treatment with the KRAS inhibitor compared to the minimum distance of said DCs and/or CD103⁺ DCs in the spatial community before the therapy with the KRAS inhibitor.

The spatial cellular community is further described below in section 8 (“Spatial cellular community”).

In some aspects, the present disclosure is further directed to a method of treating a tumor in a subject in need thereof comprising administering a Treg-depleting agent to a subject identified by the method as described herein as having an increased number of Tregs in the TME after a therapy with a KRAS inhibitor.

In some aspects, administering the Treg-depleting agent reduces tumor growth compared to growth of the tumor prior to administering the Treg-depleting agent. In some aspects, administering the Treg-depleting agent reduces tumor volume compared to the volume of the tumor prior to administering the Treg-depleting agent. In some aspects, administering the Treg-depleting agent increases survival probability of the subject compared to the probability of survival prior to administering the Treg-depleting agent. In some aspects, administering the Treg-depleting agent enhances anti-tumor immunity compared to the anti-tumor immunity prior to administering the Treg-depleting agent. In some aspects, enhancing the anti-tumor immunity comprises increasing CD8⁺ T cell infiltration compared to the CD8⁺ T cell infiltration prior to administering the Treg-depleting agent. In some aspects, enhancing the anti-tumor immunity comprises increasing the number of activated CD8⁺ T cells compared to the number of activated CD8⁺ T cells prior to administering the Treg-depleting agent. In some aspects, administering the Treg-depleting agent enhances the systemic immune response compared to the systemic immune response prior to administering the Treg-depleting agent.

In some aspects, the therapy with the KRAS inhibitor is a monotherapy with the KRAS inhibitor.

In some aspects, the therapy with the KRAS inhibitor is a combination therapy with the KRAS inhibitor and an additional anti-tumor treatment. In some aspects, the additional anti-tumor treatment does not comprise a Treg cell depleting agent. In some aspects, the additional anti-tumor treatment comprises a PD-1 axis-blocking agent, such as an anti-PD-1 antibody or an anti-PD-L1 antibody.

As regards further aspects of the method of identifying and the method of treating as described in this section, including suitable tumors, subjects, KRAS inhibitors, administration modes, and/or Treg-depleting agents for the method of identifying and the method of treating as described in this section, reference is in particular made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 4. Number of Treg Cells in a TME

In some aspects, the present disclosure is further directed to a method of predicting the responsiveness of a subject having a tumor to an anti-tumor therapy, e.g., an KRAS inhibitor therapy, an immunotherapy, or any combination thereof, the method comprising determining, or ordering the determination of, the number of Tregs in the TME, wherein the number of Tregs is predictive of the responsiveness of the subject to the anti-tumor therapy.

In some aspects, the subject is scheduled to be administered the anti-tumor therapy if the subject is predicted to be responsive to the anti-tumor therapy. In some aspects, the subject is scheduled to be administered the anti-tumor therapy and a Treg-depleting agent if the subject is predicted to not be responsive to the anti-tumor therapy.

In some aspects, the method further comprises comparing, or ordering the comparison of, the number of Tregs in the TME to a reference level from one or more other subjects having a tumor who are not responsive to the anti-tumor therapy and wherein a lower number of Tregs compared to the reference level is predictive that the subject will be responsive to the anti-tumor therapy.

In some aspects, the method further comprises comparing, or ordering the comparison of, the number of Tregs in the TME to a reference level from one or more other subjects having a tumor who are responsive to the anti-tumor therapy and wherein a lower number of Tregs or the same or substantially the same number of Tregs compared to the reference level is predictive that the subject will be responsive to the anti-tumor therapy.

In some aspects, the present disclosure is further directed to a method of selecting a subject having a tumor for an anti-tumor therapy, the method comprising:
determining, or ordering the determination of, the number of Tregs in the TME, wherein the number of Tregs is predictive of the responsiveness of the subject to the anti-tumor therapy; and
selecting the subject for the anti-tumor therapy on the basis that the subject is predicted to be responsive to the anti-tumor therapy.

In some aspects, the method further comprises comparing, or ordering the comparison of, the number of Tregs in the TME to a reference level from one or more other subjects having a tumor who are not responsive to the anti-tumor therapy and wherein a lower number of Tregs compared to the reference level is predictive that the subject will be responsive to the anti-tumor therapy.

In some aspects, the method further comprises comparing, or ordering the comparison of, the number of Tregs in the TME to a reference level from one or more other subjects having a tumor who are responsive to the anti-tumor therapy and wherein a lower number of Tregs or the same or substantially the same number of Tregs compared to the reference level is predictive that the subject will be responsive to the anti-tumor therapy.

In some aspects, the present disclosure is further directed to a method of treating a tumor in a subject in need thereof comprising administering an anti-tumor therapy to the subject, wherein the subject is identified as not having an increased number of Tregs in the TME prior to administering the anti-tumor therapy to the subject. In some aspects, the subject is identified as not having an increased number of Tregs in the TME compared to a reference level from one or more other subjects having a tumor who are responsive to the anti-tumor therapy.

In some aspects, the present disclosure is further directed to a method of treating a tumor in a subject in need thereof comprising administering an anti-tumor therapy and a Treg-depleting agent to the subject, wherein the subject is identified as having an increased number of Tregs in the TME prior to administering the anti-tumor therapy and the Treg-depleting agent to the subject. In some aspects, the subject is identified as having an increased number of Tregs in the TME compared to a reference level from one or more other subjects having a tumor who are responsive to the anti-tumor therapy.

In some aspects, the present disclosure is further directed to a method of treating a tumor in a subject in need thereof comprising administering an anti-tumor therapy to the subject, wherein the subject is identified as a subject that will be responsive to the anti-tumor therapy by determining the number of Tregs in the TME prior to administering the anti-tumor therapy to the subject. In some aspects, the subject is identified as a subject that will be responsive to the anti-tumor therapy by determining that the number of Tregs in the TME is lower than a reference level from one or more other subjects having a tumor who are not responsive to the anti-tumor therapy. In some aspects, the subject is identified as a subject that will be responsive to the anti-tumor therapy by determining that the number of Tregs in the TME is not increased compared to a reference level from one or more other subjects having a tumor who are responsive to the anti-tumor therapy.

In some aspects, the present disclosure is further directed to a method of treating a tumor in a subject in need thereof comprising administering an anti-tumor therapy and a Treg-depleting agent to the subject, wherein the subject is identified as a subject that will not be responsive to the anti-tumor therapy by determining the number of Tregs in the TME prior to administering the anti-tumor therapy and the Treg-depleting agent to the subject. In some aspects, the subject is identified as a subject that will not be responsive to the anti-tumor therapy by determining that the number of Tregs in the TME is higher than a reference level from one or more other subjects having a tumor who are responsive to the anti-tumor therapy. In some aspects, the subject is identified as a subject that will not be responsive to the anti-tumor therapy by determining that the number of Tregs in the TME is not decreased when compared to a reference level from one or more other subjects having a tumor who are not responsive to the anti-tumor therapy.

In some aspects, the number of Tregs is determined in a tumor tissue sample from the subject. In some aspects, the tumor tissue sample is a tumor tissue slice.

In some aspects, the number of Tregs is determined by IMC.

In some aspects, the number of Tregs is determined in a spatial cellular community in the TME comprising CD4⁺ T cells, CD8⁺ T cells, Tregs, and DCs. In some aspects, the spatial cellular community is further described below in the section titled “Spatial cellular community”.

In some aspects, the number of Tregs determined are Tregs in the neighborhood of CD8⁺ T cells. In some aspects, the CD8⁺ T cells in the neighborhood of the Tregs are CD8⁺ T cells that are further neighboring DCs and CD4⁺ T cells.

In some aspects, the subject was not previously treated with the anti-tumor therapy.

In some aspects, the subject was previously treated with the anti-tumor therapy.

In some aspects, the anti-tumor therapy does not comprise a Treg-depleting agent. In some aspects, the Treg-depleting agent comprises an anti-CCR8 antibody (such as 14S15, 14S15h, or imzokitug) or an antigen binding portion thereof. In some aspects, the Treg-depleting agent comprises an anti-CTLA-4 antibody (such as ipilimumab or tremelimumab) or an antigen binding portion thereof.

In some aspects, the anti-tumor therapy comprises a KRAS inhibitor. In some aspects, the anti-tumor therapy is a monotherapy with a KRAS inhibitor. In some aspects, the anti-tumor therapy is a combination therapy with a KRAS inhibitor and an additional anti-tumor treatment. In some aspects, the additional anti-tumor treatment comprises a PD-1 axis-blocking agent. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. In some aspects, the additional anti-tumor treatment further comprises an anti-LAG-3 antibody (such as relatlimab) or an antigen binding portion thereof. As regards further aspects of the additional anti-tumor treatment, reference is also made to the section titled “Additional anti-tumor treatment” above.

In some aspects, the anti-tumor therapy comprises a programmed cell death protein 1 (PD-1) axis-blocking agent. In some aspects, the anti-tumor therapy is a monotherapy with a PD-1 axis-blocking agent. In some aspects, the anti-tumor therapy is a combination therapy with a PD-1 axis-blocking agent and an anti-CTLA-4 antibody (such as ipilimumab or tremelimumab) or an antigen binding portion thereof. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. As regards further aspects of the PD-1 axis-blocking agent and/or the anti-CTLA-4 antibody or antigen binding portion thereof, reference is also made to the section titled “PD-1 axis blocking agent” and the section titled “anti-CTLA-4 antibody, or antigen binding portion thereof” above.

As regards further aspects of the methods as described in this section, including suitable tumors, subjects, KRAS inhibitors, administration modes, and/or Treg-depleting agents for the methods as described in this section, reference is generally also made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the KRAS inhibitor is administered orally, such as in the form of a tablet. In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 5. Presence/Absence of a Spatial Cellular Community

In some aspects, the present disclosure is further directed to a method of predicting the responsiveness of a subject having a tumor to an anti-tumor therapy, e.g., a KRAS inhibitor therapy, an immunotherapy, or any combination thereof, the method comprising determining, or ordering the determination of, the absence of a spatial cellular community comprising CD4⁺ T cells, CD8⁺ T cells, Tregs, and DCs in the TME, wherein the absence of the spatial cellular community is predictive that the subject will be responsive to the anti-tumor therapy.

In some aspects, the subject is scheduled to be administered the anti-tumor therapy if the subject is predicted to be responsive to the anti-tumor therapy. In some aspects, the subject is scheduled to be administered the anti-tumor therapy and a Treg-depleting agent if the subject is predicted to not be responsive to the anti-tumor therapy.

In some aspects, the present disclosure is further directed to a method of selecting a subject having a tumor for an anti-tumor therapy, the method comprising:
determining, or ordering the determination of, the absence of a spatial cellular community comprising CD4⁺ T cells, CD8⁺ T cells, Tregs, and DCs in the TME; and
selecting the subject for the anti-tumor therapy on the basis that the spatial cellular community is absent in the TME.

In some aspects, the present disclosure is further directed to a method of treating a tumor in a subject in need thereof comprising administering an anti-tumor therapy to the subject, wherein the subject is identified as not having a spatial cellular community comprising CD4⁺ T cells, CD8⁺ T cells, Tregs, and DCs in the TME prior to administering the anti-tumor therapy to the subject.

In some aspects, the present disclosure is further directed to a method of treating a tumor in a subject in need thereof comprising administering an anti-tumor therapy and a Treg-depleting agent to the subject, wherein the subject is identified as having a spatial cellular community comprising CD4⁺ T cells, CD8⁺ T cells, Tregs, and DCs in the TME prior to administering the anti-tumor therapy to the subject.

In some aspects, the absence of the spatial cellular community is determined in a tumor tissue sample from the subject. In some aspects, the tumor tissue sample is a tumor tissue slice.

In some aspects, the absence of the spatial cellular community is determined by IMC.

In some aspects, the spatial cellular community is further defined below in the section titled “Spatial cellular community”.

In some aspects, the subject was not previously treated with the anti-tumor therapy.

In some aspects, the subject was previously treated with the anti-tumor therapy.

In some aspects, the anti-tumor therapy does not comprise a Treg-depleting agent. In some aspects, the Treg-depleting agent comprises an anti-CCR8 antibody (such as 14S15, 14S15h, or imzokitug) or an antigen binding portion thereof. In some aspects, the Treg-depleting agent comprises an anti-CTLA-4 antibody (such as ipilimumab or tremelimumab) or an antigen binding portion thereof.

In some aspects, the anti-tumor therapy comprises a KRAS inhibitor. In some aspects, the anti-tumor therapy is a monotherapy with a KRAS inhibitor. In some aspects, the anti-tumor therapy is a combination therapy with a KRAS inhibitor and an additional anti-tumor treatment. In some aspects, the additional anti-tumor treatment comprises a PD-1 axis-blocking agent. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. In some aspects, the additional anti-tumor treatment further comprises an anti-LAG-3 antibody (such as relatlimab) or an antigen binding portion thereof. As regards further aspects of the additional anti-tumor treatment, reference is also made to the section titled “Additional anti-tumor treatment” above.

In some aspects, the anti-tumor therapy comprises a programmed cell death protein 1 (PD-1) axis-blocking agent. In some aspects, the anti-tumor therapy is a monotherapy with a PD-1 axis-blocking agent. In some aspects, the anti-tumor therapy is a combination therapy with a PD-1 axis-blocking agent and an anti-CTLA-4 antibody (such as ipilimumab or tremelimumab) or an antigen binding portion thereof. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. As regards further aspects of the PD-1 axis-blocking agent and/or the anti-CTLA-4 antibody or antigen binding portion thereof, reference is also made to the section titled “PD-1 axis blocking agent” and the section titled “anti-CTLA-4 antibody, or antigen binding portion thereof” above.

As regards further aspects of the methods as described in this section, including suitable tumors, subjects, KRAS inhibitors, administration modes, and/or Treg-depleting agents for the methods as described in this section, reference is generally also made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the KRAS inhibitor is administered orally, such as in the form of a tablet. In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 6. Number of Spatial Cellular Communities

In some aspects, the present disclosure is further directed to a method of predicting the responsiveness of a subject having a tumor to an anti-tumor therapy, the method comprising determining, or ordering the determination of, the number of spatial cellular communities comprising CD4⁺ T cells, CD8⁺ T cells, Tregs, and DCs in the TME, wherein the number of spatial cellular communities is predictive of the responsiveness of the subject to the anti-tumor therapy.

In some aspects, the subject is scheduled to be administered the anti-tumor therapy if the subject is predicted to be responsive to the anti-tumor therapy. In some aspects, the subject is scheduled to be administered the anti-tumor therapy and a Treg-depleting agent if the subject is predicted to not be responsive to the anti-tumor therapy.

In some aspects, the method further comprises comparing, or ordering the comparison of, the number of spatial cellular communities in the TME to a reference level from one or more other subjects having a tumor who are not responsive to the anti-tumor therapy and wherein a decrease in the number of the spatial cellular communities compared to the reference level is predictive that the subject will be responsive to the anti-tumor therapy.

In some aspects, the method further comprises comparing, or ordering the comparison of, the number of the spatial cellular communities in the TME to a reference level from one or more other subjects having a tumor who are responsive to the anti-tumor therapy and wherein a decrease in the number of the spatial cellular communities, or maintenance of the same or substantially the same number of spatial cellular communities, compared to the reference level is predictive that the subject will be responsive to the anti-tumor therapy.

A method of selecting a subject having a tumor for an anti-tumor therapy, the method comprising:
determining, or ordering the determination of, the number of spatial cellular communities comprising CD4⁺ T cells, CD8⁺ T cells, Tregs, and DCs in the TME, wherein the number of spatial cellular communities is predictive of a responsiveness of the subject to the anti-tumor therapy; and
selecting the subject for the anti-tumor therapy on the basis that the subject is predicted to be responsive to the anti-tumor therapy.

In some aspects, the method further comprises comparing, or ordering the comparison of, the number of spatial cellular communities in the TME to a reference level from one or more other subjects having a tumor who are not responsive to the anti-tumor therapy and wherein a decrease in the number of the spatial cellular communities compared to the reference level is predictive that the subject will be responsive to the anti-tumor therapy.

In some aspects, the method further comprises comparing, or ordering the comparison of, the number of the spatial cellular communities in the TME to a reference level from one or more other subjects having a tumor who are responsive to the anti-tumor therapy and wherein a decrease in the number of the spatial cellular communities, or maintenance of the same or substantially the same number of spatial cellular communities, compared to the reference level is predictive that the subject will be responsive to the anti-tumor therapy.

In some aspects, the present disclosure is further directed to a method of treating a tumor in a subject in need thereof comprising administering an anti-tumor therapy to the subject, wherein the subject is identified as a subject that will be responsive to the anti-tumor therapy by determining the number of spatial cellular communities comprising CD4⁺ T cells, CD8⁺ T cells, Tregs, and DCs in the TME prior to administering the anti-tumor therapy to the subject.

In some aspects, the subject is identified as a subject that will be responsive to the anti-tumor therapy by determining that the number of spatial cellular communities in the TME is decreased compared to a reference level from one or more other subjects having a tumor who are not responsive to the anti-tumor therapy.

In some aspects, the subject is identified as a subject that will be responsive to the anti-tumor therapy by determining the number of spatial cellular communities in the TME to be not increased compared to a reference level from one or more other subjects having a tumor who are responsive to the anti-tumor therapy.

In some aspects, the present disclosure is further directed to a method of treating a tumor in a subject in need thereof comprising administering an anti-tumor therapy and a Treg-depleting agent to the subject, wherein the subject is identified as a subject that will not be responsive to the anti-tumor therapy by determining the number of spatial cellular communities comprising CD4⁺ T cells, CD8⁺ T cells, Tregs, and DCs in the TME prior to administering the anti-tumor therapy and the Treg-depleting agent to the subject.

In some aspects, the subject is identified as a subject that will not be responsive to the anti-tumor therapy by determining that the number of spatial cellular communities in the TME is increased compared to a reference level from one or more other subjects having a tumor who are responsive to the anti-tumor therapy.

In some aspects, the subject is identified as a subject that will not be responsive to the anti-tumor therapy by determining that the number of spatial cellular communities in the TME is not decreased when compared to a reference level from one or more other subjects having a tumor who are not responsive to the anti-tumor therapy.

In some aspects, the number of spatial cellular communities is determined in a tumor tissue sample from the subject. In some aspects, the tumor tissue sample is a tumor tissue slice.

In some aspects, the number of spatial cellular communities is determined by IMC.

In some aspects, the spatial cellular communities are further defined below in the section titled “Spatial cellular community”.

In some aspects, the subject was not previously treated with the anti-tumor therapy. In some aspects, the subject was previously treated with the anti-tumor therapy.

In some aspects, the anti-tumor therapy does not comprise a Treg-depleting agent. In some aspects, the Treg-depleting agent comprises an anti-CCR8 antibody (such as 14S15, 14S15h, or imzokitug) or an antigen binding portion thereof. In some aspects, the Treg-depleting agent comprises an anti-CTLA-4 antibody (such as ipilimumab or tremelimumab) or an antigen binding portion thereof.

In some aspects, the anti-tumor therapy comprises a KRAS inhibitor. In some aspects, the anti-tumor therapy is a monotherapy with a KRAS inhibitor. In some aspects, the anti-tumor therapy is a combination therapy with a KRAS inhibitor and an additional anti-tumor treatment. In some aspects, the additional anti-tumor treatment comprises a PD-1 axis-blocking agent. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. In some aspects, the additional anti-tumor treatment further comprises an anti-LAG-3 antibody (such as relatlimab) or an antigen binding portion thereof. As regards further aspects of the additional anti-tumor treatment, reference is also made to the section titled “Additional anti-tumor treatment” above.

In some aspects, the anti-tumor therapy comprises a programmed cell death protein 1 (PD-1) axis-blocking agent. In some aspects, the anti-tumor therapy is a monotherapy with a PD-1 axis-blocking agent. In some aspects, the anti-tumor therapy is a combination therapy with a PD-1 axis-blocking agent and an anti-CTLA-4 antibody (such as ipilimumab or tremelimumab) or an antigen binding portion thereof. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof. As regards further aspects of the PD-1 axis-blocking agent and/or the anti-CTLA-4 antibody or antigen binding portion thereof, reference is also made to the section titled PD-1 axis blocking agent” and the section titled (“anti-CTLA-4 antibody, or antigen binding portion thereof”) above.

As regards further aspects of the methods as described in this section, including suitable tumors, subjects, KRAS inhibitors, administration modes, and/or Treg-depleting agents for the methods as described in this section, reference is generally also made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the KRAS inhibitor is administered orally, such as in the form of a tablet. In some aspects, the Treg-depleting agent comprises an anti-CTLA-4 antibody (such as ipilimumab or tremelimumab) or an antigen binding portion thereof. In some aspects, the Treg-depleting agent comprises an anti-CCR8 antibody (such as 14S15, 14S15h, or imzokitug) or an antigen binding portion thereof. In some aspects, the Treg-depleting agent is administered parenterally, such as intravenously.

### 7. Method for Identifying a Subject Who is not Suitable for Treatment with a KRAS Inhibitor and a PD-1 Axis-Blocking Agent

In some aspects, the present disclosure is further directed to a method for identifying a subject having a tumor who is not suitable for treatment with a KRAS inhibitor and a PD-1 axis-blocking agent comprising determining whether the subject possesses spatial cellular communities in the TME, wherein the spatial cellular communities are rich in Tregs.

In some aspects, the present disclosure is further directed to a method for identifying a subject having a tumor who is not suitable for treatment with a KRAS inhibitor and a PD-1 axis-blocking agent comprising determining whether the subject possesses spatial cellular communities in the TME, wherein the spatial cellular communities comprise about 0.5% to about 2% Tregs.

In some aspects, the spatial cellular communities further comprise CD4⁺ T cells, CD8⁺ T cells, and DCs. In some aspects, the spatial cellular communities are further defined below in the section titled “Spatial cellular community”.

As regards further aspects of the methods as described in this section, including suitable tumors, subjects, KRAS inhibitors, and/or PD-1 axis-blocking agents for the methods as described in this section, reference is generally also made to the section titled “Method of treating a tumor comprising administering a KRAS inhibitor and a Treg-depleting agent” above. In some aspects, the tumor is a lung cancer, such as NSCLC. In some aspects, the subject is a human. In some aspects, the KRAS inhibitor is a KRAS G12C inhibitor (such as adagrasib, sotorasib, or MRTX1257). In some aspects, the KRAS inhibitor is administered orally, such as in the form of a tablet. In some aspects, the PD-1 axis-blocking agent is an anti-PD-1 antibody (such as nivolumab or pembrolizumab) or an antigen binding portion thereof. In some aspects, the PD-1 axis-blocking agent is an anti-PD-L1 antibody (such as atezolizumab, durvalumab, avelumab) or an antigen binding portion thereof.

### 8. Spatial Cellular Community

In the following, further aspects of the spatial cellular community of the present disclosure are described. Reference to “the” or “a” “spatial cellular community” (in singular form) below includes also a reference to the corresponding plural form (“spatial cellular communities”).

In some aspects, the spatial cellular community comprises Tregs that are in the neighborhood of CD8⁺ T cells. In some aspects, the CD8⁺ T cells in the neighborhood of the Tregs are CD8⁺ T cells neighboring DCs and CD4⁺ T cells.

In some aspects, the spatial cellular community further comprises CD103⁺ DCs.

In some aspects, the spatial cellular community comprises DCs and/or CD103⁺ DCs that express CXCL9.

In some aspects, DCs and/or CD103⁺ DCs with a mean CXCL9 expression of at least 0.5 as measured by ICM have a minimum distance of about 50 to about 300 pixels to PD-1⁺ CD8⁺ T cells in the spatial community, a minimum distance of about 20 to about 130 pixels to PD-1⁺ CD4⁺ T cells in the spatial community, and/or a minimum distance of about 50 to about 300 pixels to PD-1⁺ Treg cells in the spatial community as determined by ICM.

In some aspects, the spatial cellular community comprises DCs that express PD-L1, CD86, and/or MHC-II.

In some aspects, the spatial cellular community comprises activated CD8⁺ T cells.

In some aspects, the spatial cellular community comprises CD8⁺ T cells that express PD-1, Ki67, and/or LAG-3.

In some aspects, about 30% to about 40% of the CD8⁺ T cells in the spatial cellular community express LAG-3.

In some aspects, the spatial cellular community comprises CD4⁺ T cells that express PD-1 and/or Ki67.

In some aspects, the CD4⁺ T cells and/or the CD8⁺ T cells in the spatial cellular community express TIM3, GITR, CTLA-4, and ICOS.

In some aspects, the spatial cellular community comprises Tregs that express PD-1 and/or Ki67.

In some aspects, the spatial cellular community comprises Tregs that inhibit CD4⁺ T cells and/or CD8⁺ T cells in the spatial cellular community. In some aspects, the Tregs inhibit the CD8⁺ T cells by secreting T cell suppressive cytokines, such as IL-10, IL-35, and TGF-beta, by scavenging IL-2, and/or by generation of extracellular adenosine.

In some aspects, the spatial cellular community comprises Tregs that compete with the CD8⁺ T cells and/or CD4⁺ T cells in the spatial cellular community for co-stimulatory molecules on the DCs and/or CD103⁺ DCs in the spatial cellular community.

In some aspects, the spatial community further comprises tumor cells expressing Ki67 and tumor cells expressing cleaved-caspase-3 (c-casp3).

In some aspects, the spatial cellular community comprises CD8⁺ T cells in the neighborhood of c-casp3⁺ tumor cells.

In some aspects, the spatial cellular community comprises about 0.5% to about 2% Tregs. In some aspects, the spatial cellular community comprises about 5% to about 12% CD4⁺ T cells, about 1% to about 4% CD8⁺ T cells, about 0.5% to about 2% Tregs, and about 10% to about 20% DCs, and optionally about 20% to about 45% tumor cells, and optionally about 2% to about 10% CD103⁺ DCs.

In some aspects, the spatial cellular community comprises neighborhoods with and without Tregs. In some aspects, the neighborhoods with Tregs have a higher proportion of DCs and CD4⁺ T cells compared to the neighborhoods without Tregs. In some aspects, the interactions between CD8⁺ T cells with tumor cells, DCs and/or CD103⁺ DCs in the neighborhoods with Tregs are decreased compared to the neighborhoods without Tregs. In some aspects, the neighborhoods with Tregs comprise about 20% to about 30% tumor cells, about 5% to about 10% Tregs, about 1% to about 6% CD8⁺ T cells, about 15% to about 25% CD4⁺ T cells, about 8% to about 14% Type 2 macrophages, about 1% to about 6% CD103⁺ DCs, and about 15% to about 25% DCs. In some aspects, the neighborhoods with Treg cells comprise about 25% to about 27% tumor cells, about 6.5% to about 8.5% Treg cells, about 2% to about 5% CD8⁺ T cells, about 17.5% to about 20% CD4⁺ T cells, about 10% to about 12% Type 2 macrophages, about 2% to about 5% CD103⁺ DCs, and about 17.5% to about 20% DCs.

In some aspects, the spatial cellular community is localized peritumorally.

In some aspects, the spatial cellular community is localized intratumorally.

In some aspects, the spatial cellular community comprises at least about 25 cells/mm², at least about 30 cells/mm², at least about 35 cells/mm², at least about 40 cells/mm², at least about 45 cells/mm², at least about 50 cells/mm², at least about 55 cells/mm², at least about 60 cells/mm², at least about 65 cells/mm², at least about 70 cells/mm², at least about 75 cells/mm², at least about 80 cells/mm², at least about 85 cells/mm², at least about 90 cells/mm², at least about 95 cells/mm², or at least about 100 cells/mm². In some aspects, the spatial cellular community comprises at least about 25 cells/mm².

### EXAMPLES

Having generally described this disclosure above, a further understanding can be obtained by reference to the examples provided herein below. These examples are, however, for purposes of illustration only and are not intended to be limiting.

Although KRAS-G12C inhibition was previously shown to synergise with anti-PD-1 treatment, this synergism was shown to only be the case in tumor models that are at least already moderately responsive to immune checkpoint blockade at baseline. In the present disclosure, to investigate mechanisms that restrain immunotherapy sensitivity in non-responsive tumors, multiplex imaging mass cytometry (IMC) was applied to explore spatial patterns in the tumor microenvironment (TME) of the highly immune evasive KRAS mutant murine Lewis Lung Cancer model. Clustering of close neighbor information per cell allowed characterization of spatial patterns or ‘communities’ in the tissue. A community harbouring features of localized T-cell activation, where CD4⁺ and CD8⁺ T cells and dendritic cells were gathered together, was identified. KRAS-G12C inhibition led to increased expression of PD-1 on T cells, CXCL9 expression by dendritic cells, together with increased proliferation and potential cytotoxicity of CD8⁺ T cells, indicating an effector response. However, this cellular community was also observed to comprise a high incidence of Tregs which had frequent contact with effector T cells, suggesting that Treg cells were dampening anti-tumoral immune responses following KRAS-G12C inhibition (see, in particular, Examples 1 to 6). Similar communities were detected in lung adenocarcinoma samples from treatment-naïve human patients (see, in particular, Example 7). Depleting Treg cells in vivo with a Treg cell depleting agent (in particular, with an anti-CTLA-4 or anti-CCR8 antibody) was shown to rescue the anti-tumor immune response and to lead to enhanced tumor control when applied in combination with anti-PD-1 and KRAS-G12C inhibitor therapy (see, in particular, Examples 8 and 9). See also Cole et al. (2024) Spatial multiplex analysis of lung cancer reveals that regulatory T cells attenuate KRAS-G12C inhibitor-induced immune responses. Sci. Adv. 10, ead16464, which is incorporated herein by its entirety.

### Materials and Methods

### Study Design

The goal of the study was to overcome the resistance of a KRAS-G12C inhibitor therapy in treating a tumor. A new target (i.e., a Treg population) was identified within the spatial cellular communities in the tumor microenvironment of KRAS-G12C inhibitor-treated mouse lung adenocarcinomas. Previously published data from imaging mass cytometry analysis of Lewis Lung Carcinoma treated for 7 days with KRAS-G12C inhibitors was reanalysed to investigate the presence of recurring cellular communities that could be potentially linked to therapy resistance. Relationships between cells within the communities were analysed, looking at cell-cell distances, expression of activation markers and neighbor enrichment. To validate the findings, a similar analysis was conducted in a human dataset of treatment-naïve NSCLC tumor samples. The identified suppressor cells, namely Treg cells, were then targeted in vivo using depleting antibodies to verify that the Tregs were indeed instrumental in imposing resistance to KRAS treatment (in particular, to a combination treatment with KRAS-G12C inhibitor and anti-PD-1).

### Materials and Methods for Examples 1-6

In the following, materials and experimental methods for Examples 1-6 are described.

### a) In Vivo Drug Study and Imaging Mass Cytometry (IMC)

Two datasets of IMC data generated from an in vivo experiment in which the Lewis Lung (3LL) carcinoma model was treated with the KRAS-G12C inhibitor MRTX1257 for 7 days prior to harvesting of the lungs were published previously in van Maldegem et al. (2021), Nat. Commun. 12:5906 (dataset 1) and Mugarza et al. (2022), Sci. Adv. 8: eabm8780 (dataset 2).

In brief, 10⁶ ΔNRAS 3 Lewis lung carcinoma cells (Molina-Arcas et al. (2019) Sci. Transl. Med. 11) were injected into the tail vein of 9-11 week old C57BL/6 mice. Following 3 weeks, mice were treated with either 50 mg/kg MRTX1257 or Vehicle for 7 consecutive days. Mice were sacrificed on day 8 and lungs were harvested and frozen. Tumors of 3 mice from each treatment group for dataset 1 and 4 mice from each treatment group for dataset 2 were processed for imaging mass cytometry. Tissue slices were stained with a cocktail of antibodies conjugated to heavy metal isotopes and images were obtained using a Hyperion Imaging Mass Cytometer (Standard BioTools). Different antibody panels were used for the generation of datasets 1 and 2 (FIGS. 1A and 1B). In total, 6 images were acquired from both Vehicle and MRTX1257 groups for dataset 1, and the Vehicle group for dataset 2, and 5 images were acquired from the MRTX1257 group for dataset 2.

For further details on the in vivo study and IMC, including tissue processing, antibody staining, imaging acquisition and cell typing, reference is generally made to the Materials and Methods and Supplementary Materials provided by van Maldegem et al. (2021) and Mugarza et al. (2022), which are herewith incorporated by reference.

### b) Image Segmentation

Segmentation for the image sets from cohorts 1 and 2 was carried out using CellProfiler v3.1.9, including custom modules by Bodenmiller (https://github.com/BodenmillerGroup/ImcPluginsCP) and Ilastik v1.3.3b1. For cohort 1, a sequential segmentation pipeline was run to identify individual cells from the IMC images. In brief, probability maps were created in Ilastik for the separate identification of lymphocytes, macrophages, fibroblasts, tumor cells and endothelium, while remaining cell objects were identified using a nuclei expansion of 1-pixel. This pipeline also involved domain segmentation through generation of domain probability maps, enabling normal lung tissue, tumor tissue, and the interface region between normal and tumor sections to be identified. See van Maldegem et al. (2021) for further details on the sequential segmentation strategy. For cohort 2, a segmentation pipeline was run which involved a 1-pixel expansion from the cellular nuclei to identify the cell objects. This method did not involve generation of domain information per tissue. For generation of both datasets, segmentation was run using imcyto (https://github.com/nf-core/imcyto). For details of CellProfiler modules used and project files generated for both segmentation methods see:
Dataset 1: https://hdl.handle.net/10779/crick.c.5270621.v2
Dataset 2: https://doi.org/10.25418/crick. 19590259

### c) Neighbor Identification

As described in van Maldegem et al. (2021), cellular neighbors were identified during segmentation using the CellProfiler module ‘Measure Object Neighbours’, following steps to identify individual cells. Neighbors were identified if a cell's boundary was within 15 μm (pixels) of the cell boundary of interest. Neighbor information was obtained for every cell object in each tissue.

### d) Identification of Spatial Cellular Communities—Mouse Data

Each ‘neighborhood’ was identified as a single cell and its local neighbors; defined by 15 μm object relationship output data from segmentation. For each cell, the proportion of each cell type (16 cell types for dataset 1 & 14 cell types for dataset 2) found in its neighborhood was calculated (range: 0-1), as demonstrated by the equation below:

$$proportion = \frac{no . cells of type X in neighborhood}{total no . cells in neighborhood}$$

All cells per dataset were then clustered using Rphenograph, based on the neighbor proportion values calculated. A k=250 clustering input yielded 62 neighborhood communities for dataset 1 and 254 communities for dataset 2 (data not shown). See also Cole et al. 2024 above.

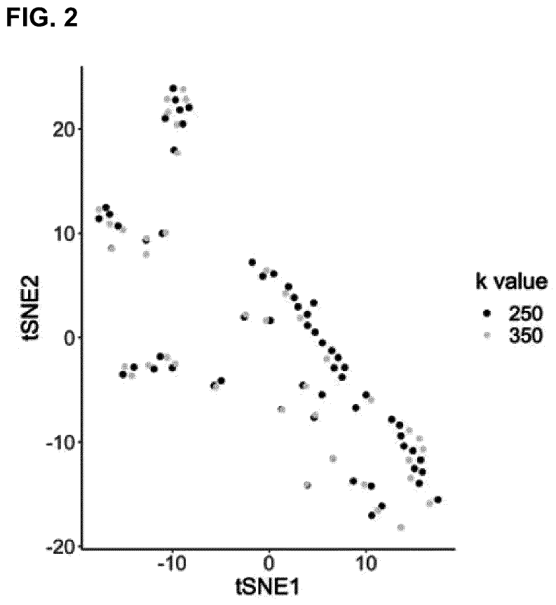

Dimensionality reduction such as the R implementation of tSNE (RtSNE) (Van der Maaten et al. (2008) J. Mach. Learn. Res. 9:2579-2605), and dendrograms were used to determine which communities were similar and could therefore be analysed together for dataset 1. See FIG. 2. Agglomerative clustering using the AgglomerativeClustering function from the sklearn.cluster package in Python 3.9 was then used to group the 62 communities into 18. The clustree package was used to represent the merging of 62 communities into 30 and subsequently 18. Agglomerative clustering was also used to group 254 communities into 18, so that analysis of communities from datasets 1 and 2 were equal and thus the datasets could be analysed in parallel (data not shown). See also Cole et al. 2024 above.

### e) Validation of Method to Identify Communities

For validation using different input parameters, clustering was run on the neighborhood proportion information for dataset 1 with a k-input value of 250 to produce 62 communities (as described in ‘Community detection’ section) and repeated for a k-input value of 350 to yield 47 communities. A tSNE was then used for dimensionality reduction of the cell type proportions contributing to each of the total 109 communities.

For validation across datasets, only the neighborhood proportion values for the cell types that were shared across datasets 1 and 2 were used, comprising B cells, Dendritic cells, Dendritic cells CD103, Fibroblasts, Macrophages type 1, Macrophages type 2, NK cells, T cells CD4, T cells CD8, T reg cells and Tumor cells. Although both datasets contained cells labelled as Unclassified, they were not included in this analysis due to not being associated with a particular cell phenotype, rather lack of, and therefore could not be directly compared across the two datasets. These neighborhood proportion values were clustered using Rphenograph with a k-input value of 250, for dataset 1 and 2 separately to yield 18 communities for both dataset 1 and dataset 2 after agglomeration. tSNE was then used for dimensionality reduction of the cell type proportions contributing to each of the total 36 communities.

### f) Normalization, Scaling, and Clustering

Expression values for each marker were normalized to the mean intensity of Xenon134. Following this, the data for each image across both treatment groups was concatenated, creating a size of 282,837 cells for dataset 1 and 626,070 cells for dataset 2. Each channel was then scaled to the 99^{th} percentile. Mean pixel intensity of 17 cellular markers for dataset 1 (αSMA, B220, CD103, CD11c, CD3, CD44, CD45, CD4, CD68, CD8, EPCAM, F480, LY6G, MHCII, NKp46, PECAM, PVR) and 14 cellular markers for dataset 2 (aSMA, B220, CD103, CD11c, CD3, CD44, CD45, CD4, CD68, CD8, F480, Foxp3, MHC-II and NKp46) were used for clustering using Rphenograph (Levine et al., 2015, Data-Driven Phenotypic Dissection of AML Reveals Progenitor-like Cells that Correlate with Prognosis. Cell 162, 184-197) with k=20 to identify 30 clusters for dataset 1 and 33 clusters for dataset 2. These clusters formed the basis to identify cell types present in the tissue, including tumor, lymphocytes, myeloid cells, fibroblasts, and endothelium. More details are available in van Maldegem et al., 2021 for dataset 1 and Mugarza et al., 2022 for dataset 2.

### g) Pearson Correlation

For comparing cell type relationships per region of interest (ROI), the proportion of each cell type contributing to each ROI was calculated. For comparing cell type relationships within communities, the proportion of each cell type contributing to each of the 18 communities was calculated. For both instances this was followed by a Pearson correlation calculation of each cell type pair across all communities. A significant correlation was depicted by a p-value below 0.05, but significance was tiered as *=p<0.05, **=p<0.01, ***=p<0.001. Cell types were clustered based on their correlations.

### h) Neighborhood Enrichment Analysis

As described in van Maldegem et al., 2021, the neighbourhood method developed by Bodenmiller (https://github.com/BodenmillerGroup/neighbourhood; Schapiro et al., 2017, histoCAT: analysis of cell phenotypes and interactions in multiplex image cytometry data. Nat Methods 14, 873-876) was used to identify the enrichment of cell types within the 15-pixel neighborhood of each cell compared to random permutations of events, with a modification of only calculating enrichment in the CD8⁺ T cell neighborhood, separated by community and using 1000 rounds of permutation. This was carried out for the community names T cell/normal adjacent community (T/NA), T cell/Type 1 macrophage community (T/M1), T cell/Dendritic cell community (T/DC), T cell/Type 2 macrophage community 1 (T/M2_1) and T cell/Type 2 macrophage community 2 (T/M2_2) communities for the MRTX1257 treatment setting.

Neighborhood enrichment analysis was also used for exploring cell pair relationships in the presence or absence of Tregs within the T/DC and T/M2_2 communities following MRTX treatment. Enrichment scores were only deemed statistically significant if the p-value when comparing real neighbors to randomized neighbors was ≤0.01.

### i) Distance Calculations

X- and Y-coordinates based on the centre of each cell, as generated through segmentation in CellProfiler, where used to calculate distances between cells. The cKDTree function from scipy.spatial package in Python 3.9 was used to compute distances of Dendritic cells and CD103⁺ Dendritic cells to CD4⁺, CD8⁺ and Tregs that were PD-1⁺ with a distance threshold of 800 pixels within the T/DC community following MRTX1257 treatment.

### Materials and Methods for Example 7

In the following, materials and experimental methods for Example 7 are described.

### a) Identification of Spatial Cellular Communities

The community identification method developed by Schurch et al. (2020) Cell 183:838) was applied to 141 non-small cell lung cancer tumor cores that were imaged with the Pan-Immune panel and 137 tumor cores that were imaged with the T cells & Stroma panel to identify groups of cells that commonly localized near one another.

Briefly, the method was as follows: a window was defined around every cell in an image and its 10 nearest neighboring cells including the centre cell. These windows were clustered by their composition with respect to the 18 cell types in the Pan-Immune panel and the 20 cell types in the T cells & Stroma panel (with at least 10 cells on average per image) using MiniBatchKMeans. The parameters of the method by Schurch et al. were optimized and 10 spatial cellular communities from the Pan-Immune panel were identified. 30 spatial cellular communities were detected using the T cells & Stroma panel. Communities were then assigned representative names based on the enrichment of cell densities within them.

Spatial community identities were mapped onto segmented cells and visualized using Cytomapper (Eling et al. (2020) Bioinformatics 36 (24): 5706-5708), which were then validated by a pathologist's assessment of serial H&E-stained tissue sections.

The cell density of spatial cellular communities was calculated by taking the number of cells assigned to a spatial cellular community divided by the total tissue area (cells/mm²).

### b) Association Between Cell Densities of Cell Subtypes (T Cells & Stroma Panel) and Spatial Cellular Communities (Pan-Immune Panel)

The cell density of stroma-localized T cell subtypes detected was correlated using the T cells & Stroma antibody panel and the cell density of ten spatial communities detected using the Pan-immune antibody panel in cores from the same tumor region. For comparisons with multiple tumor cores/regions per tumor, linear mixed effects (LME) model analysis was applied to incorporate patient ID as a random effect. The T-score and p-value of the model were reported.

### c) Co-Occurrence of Spatial Cellular Communities

The lowest quartile of the cell densities of Treg communities was approximately 25 cells/mm². Therefore, 25 cells/mm² was used as the threshold to determine if a spatial community was present or absent in a tumor core. The proportion of paired tumor cores (T cells & Stroma panel, Pan-immune panel) with at least 25 cells/mm² in p2_C1: Tumor border communities and in p1 Treg communities was reported.

### Materials and Methods for Example 8

In the following, materials and experimental methods to Example 8 are described.

3LL-ΔNRAS tumor cell lines were generated as previously described in Molina-Arcas et al. (2019) Sci. Transl. Med. 11 (510): eaaw7999 (PMID: 31534020). Cells were cultured in RPMI medium supplemented with 10% fetal calf serum, 4 mM L-glutamine (Sigma-Aldrich), penicillin (100 U/ml), and streptomycin (100 mg/ml; Sigma-Aldrich). Cell lines were tested for mycoplasma and authenticated by short-tandem repeat DNA profiling. Cells were allowed to grow for not more than 20 subculture passages.

Intravenous tail-vein injections of 106 3LL-ΔNRAS cells were carried out for orthotopic studies using 8-12 week-old C57BL/6J mice. 17-18 days after injection, presence of tumors was confirmed by CT scan and mice were randomized in treatment groups.

Mice were anesthetized by isoflurane inhalation and scanned using the Quantum GX2 micro-CT imaging system (PerkinElmer) at a 50-μm isotropic pixel size. Serial lung images were reconstructed and analysed using Analyze12 (AnalyzeDirect) as previously described in Zaw Thin et al. (2022) Nat. Protoc. 18 (3): 990-1015 (PMID: 36494493).

Mice were euthanized, with an overdose of pentobarbitone, when a humane endpoint of 15% weight loss was reached or any sign of distress was observed (i.e. hunched, piloerection, difficulty of breathing). In addition, if a mouse was observed to have a tumor burden in excess of 70% of lung volume when assessed by micro-CT scanning, they were deemed at risk of rapid deterioration in health and euthanized immediately.

MRTX849 (adagrasib) was prepared in 10% captisol and administered by oral gavage daily at 100 mg/kg for 40 days. Four doses (twice weekly for two weeks) of antibodies were administered at 200 μg/dose (per antibody) by intraperitoneal injection. The antibodies used were an anti-PD-1 antibody (clone 4H2, g1-D265A), an Fc-optimized anti-CTLA-4 antibody (clone 9D9, mlgG2a; Selby et al. (2013) Cancer Immunol. Res. 1:32-42) and an anti-CCR8 antibody (mouse IgG2a isotype, mIgG2a), as well as the mlgG1-D265A and mlgG2a isotope controls. The anti-CCR8-mlgG2a antibody used was derived from the commercial rat anti-mouse-CCR8 monoclonal antibody sold by BioLegend as Clone SA214G2, which was modified to change the rat IgG2b isotype to a mouse IgG2a isotype. Anti-CCR8-mIgG2a binds to the N-terminus of CCR8 and its mlgG2a backbone provides maximal FcγR binding for driving an ADCC response similar to a human nonfucosylated anti-CCR8 antibody. See also WO 2021/194942, and, in particular, the Examples of WO 2021/194942, herewith incorporated by reference.

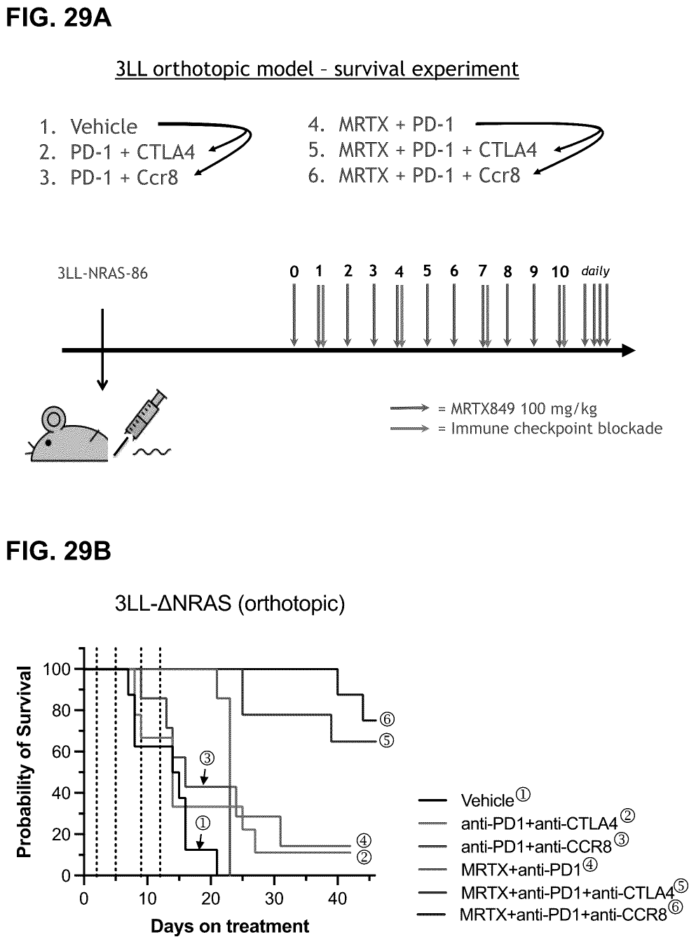

A schematic outline of how an in vivo survival experiment was performed is shown in FIG. 29A. MRTX849 and immune checkpoint blockage were administered together on Day 1, Day 4, Day 7, and Day 10 while on other days (Day 0, 2, 3, 4, 5, 6, 8, 9, or 10), only MRTX849 was administered.

### Materials and Methods for Example 9

In the following, materials and experimental methods to Example 9 are described.

3LL-ΔNRAS lung tumors were developed and treated as described for Example 8 above. Mice were treated for 6 days with adagrasib (100 mg/kg). The day after the last dose of adagrasib, mice were culled, lung tumors were isolated and tumors from the same lung were pooled together. Tumors were cut finely and incubated with digestion solution (collagenase 1 mg/ml; ThermoFisher, and DNase I 50 U/ml; Life Technologies) at 37° C. for 45 minutes. Cells were then filtered through 70 μm strainers (Falcon) and red blood cells were lysed using ACK buffer (Life Technologies). After washes in PBS, cells were stained with fixable viability dye eFluor870 (BD Horizon) for 30 minutes at 4° C. and blocked with CD16/32 antibody (BioLegend) for 10 minutes. Samples were then washed three times in FACS buffer (2 mM EDTA and 0.5% bovine serum albumin in PBS, pH 7.2) before staining of surface markers using fluorescently labelled antibody mixes (see Table 13 below). Cells were fixed with a Fix/lyse solution (eBioscience) after staining. If intracellular staining was carried out, such as for FOXP3 transcription factor, cells were instead fixed with Fix/Perm solution (eBiosciences/Invitrogen), followed by intracellular antibody staining using Perm buffer. Samples were then resuspended in FACS buffer and analysed using a FACSymphony analyser (BD). Data was analysed using FlowJo.

TABLE 13
Antibodies used for flow cytometry.

| Protein | Clone | Fluorophore | Reference | Source |
|---|---|---|---|---|
| CD3 | 17A2 | FITC | 100204 | BioLegend |
| CD45 | 30-F11 | PerCP | 103129 | BioLegend |
| Foxp3 | FJK-16s | eF660 | 50-5773-82 | eBioscience |
| CD44 | IM7 | BV421 | 103040 | BioLegend |
| CD69 | H1.2F3 | BV605 | 104529 | BioLegend |
| CD62L | MEL-14 | BV711 | 104445 | BioLegend |
| PD-1 | 29F.1A12 | BV785 | 135225 | BioLegend |
| (CD279) | | | | |
| CD8a | 53-6.7 | BUV395 | 563786 | BD Horizon |
| CD4 | GK1.5 | BUV737 | 612761 | BD Horizon |
| TIM3 | RMT3-23 | PE | 119703 | BioLegend |
| (CD366) | | | | |
| LAG-3 | C9B7W | PE-Cy7 | 125225 | BioLegend |
| (CD223) | | | | |
| CTLA4 | UC10-4B9 | BV421 | 106312 | BioLegend |
| (CD152) | | | | |
| TIM3 | RMT3-23 | BV605 | 119721 | BioLegend |
| (CD366) | | | | |
| Ki67 | B56 | BV711 | 563755 | BD Horizon |
| TCF1/TCF7 | C63D9 | PE | 14456 | Cell |
| | | | | Signaling |
| CD39 | 24DMS1 | PE-Cy7 | 25-0391-80 | eBioscience |
| MHCII | M5/114.15.2 | FITC | 107605 | BioLegend |
| (I-A/I-E) | | | | |
| CD103 | 2E7 | BV421 | 121421 | BioLegend |
| CD24 | M1/69 | BV605 | 101827 | BioLegend |
| CD86 | GL-1 | BV785 | 105043 | BioLegend |
| CD11c | HL3 | BUV395 | 564080 | BD Horizon |
| CD11b | M1/70 | BUV737 | 612801 | BD Horizon |
| PD-L1 | 10F.9G2 | PE | 124308 | BioLegend |
| (CD274) | | | | |
| FcgRI | X54-5/7.1 | PE-Cy7 | 139313 | BioLegend |
| (CD64) | | | | |

### Example 1—Introduction to Spatial Cellular Communities and Validation

A cellular community analysis was adopted to cluster cells based on the composition of their local neighborhood (adapted with modifications from Schurch et al. (2020) Cell 182:1341-1359). Analysis focused on two previously published datasets (see Mugarza et al. (2022) Sci. Adv. 8: eabm8780, and van Maldegem et al. (2021) Nat. Commun. 12:5906), both generated from an in vivo experiment in which the Lewis Lung (3LL) carcinoma model was treated with the KRAS-G12C inhibitor MRTX1257 for 7 days prior to harvesting of the lungs. The tumors were stained with two partially overlapping antibody panels to give two datasets, with the dataset 2 panel being more T-cell oriented (FIGS. 1A and 1B).

Neighbors were identified by a 15-pixel expansion of the cell boundary through segmentation in CellProfiler. A 15-pixel radius was chosen as it depicts the average size of a cell, and therefore cells identified as neighbors would be those ‘up to one cell away’. As a result, neighborhoods were not equal in cell number, but instead reflected the local density surrounding each cell. Louvain clustering using Rphenograph was then run on the neighbor proportion information per cell to identify recurring spatial patterns in the tissue, labelled ‘spatial communities’ or just ‘communities’ in short (data not shown). See also Cole et al. (2024) Spatial multiplex analysis of lung cancer reveals that regulatory T cells attenuate KRAS-G12C inhibitor-induced immune responses. Sci. Adv. 10, ead16464.

Graph building with a k-nearest neighbor input value of 250 yielded 62 communities for dataset 1, which were agglomerated to 30, and subsequently 18 communities. Agglomeration merged the communities with the lowest granularity, representing mainly small variations in the tumor cell neighbors (agglomerated community 3), while the highly granular communities, such as those with a high proportion of immune cells, remained unchanged (data not shown).

To determine whether the method to identify spatial communities was robust under altered clustering input conditions, Rphenograph clustering was also run on the neighborhood information for all cells in dataset 1 with a k-input value of 350. Dimensionality reduction using tSNE (t-distributed Stochastic Neighbor Embedding) of the 62 communities identified from k-input of 250 and the 47 communities identified from a k-input of 350 revealed very similar patterns of community phenotypes (FIG. 2). Additionally, there were multiple overlaps between communities identified using the different k-values for clustering, suggesting matching phenotypes. Moreover, the formation of separated groups could be seen, demonstrating the capture of unique spatial features between communities.

The 254 communities identified from neighbor clustering of dataset 2 were also agglomerated to 18 communities to enable parallel analysis with the communities identified in dataset 1. The communities in both datasets varied largely in size, with some containing fewer than 2,000 cells, and others comprising over 50,000 cells (data not shown). There was also differing heterogeneity of these spatial groups, with some being dominated by a single cell type, such as tumor cells in community 3 from dataset 1 and community 9 from dataset 2, whilst others comprised a mixture of various cell types in more balanced proportions (data not shown).

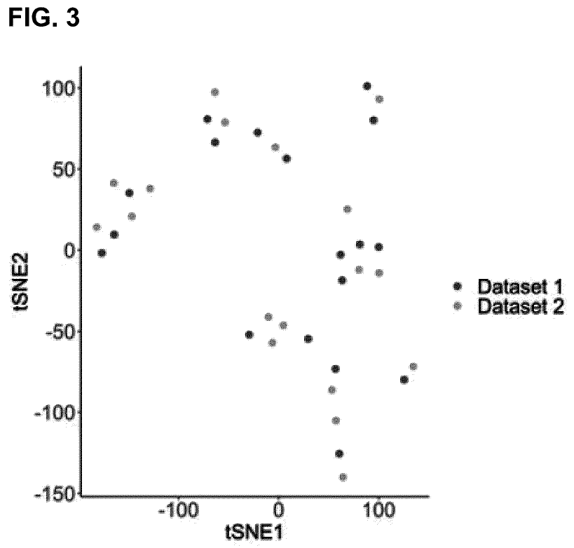

Datasets 1 and 2 were based on different antibody panels and therefore do not identify all the same cell types. For example, lack of markers EPCAM and PECAM for dataset 2 meant that endothelial and epithelial cells could not be identified and thus a large number of cells were labelled as ‘unclassified’. Nevertheless, a separate community clustering analysis on both datasets based on shared cell types only, demonstrated that the method to generate communities was stable to altered input data (FIG. 3).

### Example 2—Spatial Communities Reflect Refined Tissue Architecture and have Functional Relevance

As the spatial communities are based on local neighborhoods, but these local neighborhoods are likely to be different within different regions of the tissue, the link with tissue architecture was further explored. For dataset 1, information about the three tissue domains that each cell had been assigned to during image segmentation, i.e., tumor, normal and interface, was available. As the communities had a non-random distribution across the domains, the distribution of each community relative to the cross section through the tissue was visualized, to further expand on this spatial organization of the communities (data not shown). For example, in the Vehicle setting, community 18, with high endothelium and B cell portion was restricted to the non-tumor region as its cell count diminished going into the tumor bulk (data not shown). Additionally, community 10, with high Type 1 macrophage contribution peaked in cell count at the tumor boundary, demonstrating a clear interface region between normal tissue and the tumor bulk (data not shown). There were also further communities, number 2 and 3, that were found only in the tumor region, albeit at very different frequencies between Vehicle and MRTX conditions (data not shown).

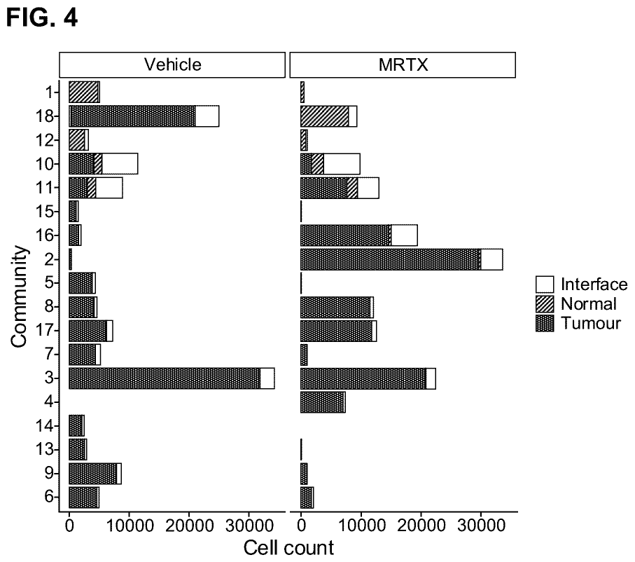

Following treatment with MRTX1257, the immune excluded phenotype of this 3LL Lewis lung carcinoma model was remodelled into a more inflammatory immune infiltrated TME (van Maldegem et al. (2021) Nat. Commun. 12:5906). This conversion was also reflected in spatial distribution of the communities, suggesting that tissue domain definition was lost following KRAS-G12C inhibition. Not only did the concentration of communities such as 10 and 18 to the interface and normal regions become less pronounced, but also spatial patterns within the tumor bulk changed following KRAS-G12C inhibition, as the frequency of many communities, such as 2 (type 2 macrophage dominant), 3 (tumor dominant) and 16 (mixed phenotype with high type 2 macrophage portion) were altered, suggesting transition to a new organization of the TME (FIG. 4).

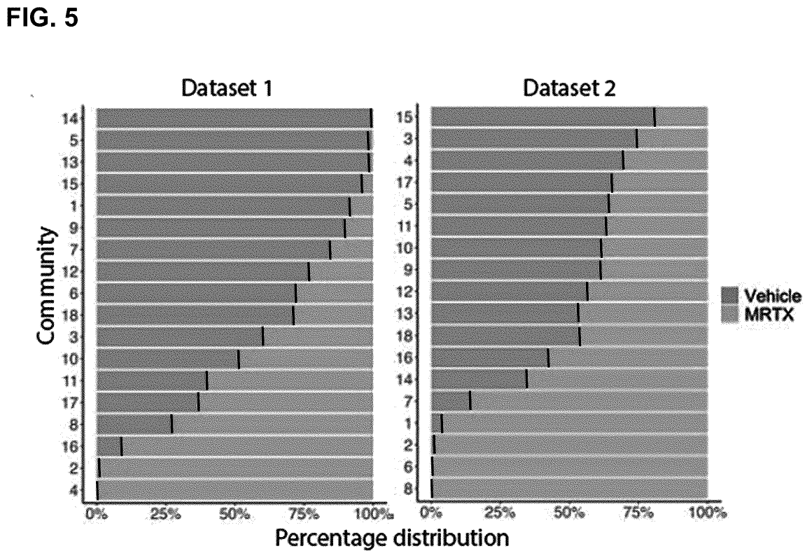

Comparing the relative contribution of each community between the Vehicle and MRTX1257 treatment groups revealed that prevalence of many spatial patterns was altered following KRAS-G12C inhibition. Some of these shifts in communities captured changes that were previously described at the single cell level (van Maldegem et al. (2021) Nat. Commun. 12:5906). For example, communities 5, 13, 14, 15 from dataset 1 were found solely in tumors treated with Vehicle (FIG. 5). These communities represent abundant interactions between tumor cells and neutrophils, which were lost or dispersed following treatment with MRTX1257 (data not shown). This is in line with a previous observation that the number of neutrophils within the tumor domain was significantly reduced following treatment (van Maldegem et al. (2021) Nat. Commun. 12:5906). Alternatively, communities 2, 4 and 16 from dataset 1 and communities 2, 6 and 8 from dataset 2 were almost exclusively found in tumors following treatment with MRTX1257. A shared feature for these communities was a neighborhood involving high numbers of Type 2 macrophages (F4/80⁺ CD206⁺), which have previously been shown to become greatly increased in number upon KRAS-G12C inhibition (van Maldegem et al. (2021) Nat. Commun. 12:5906). This suggests that the communities are reflecting relevant biological processes. Further supporting this notion was the ability to largely separate tissues into the two treatment groups when clustering the frequency of each spatial community per region of interest (ROI).

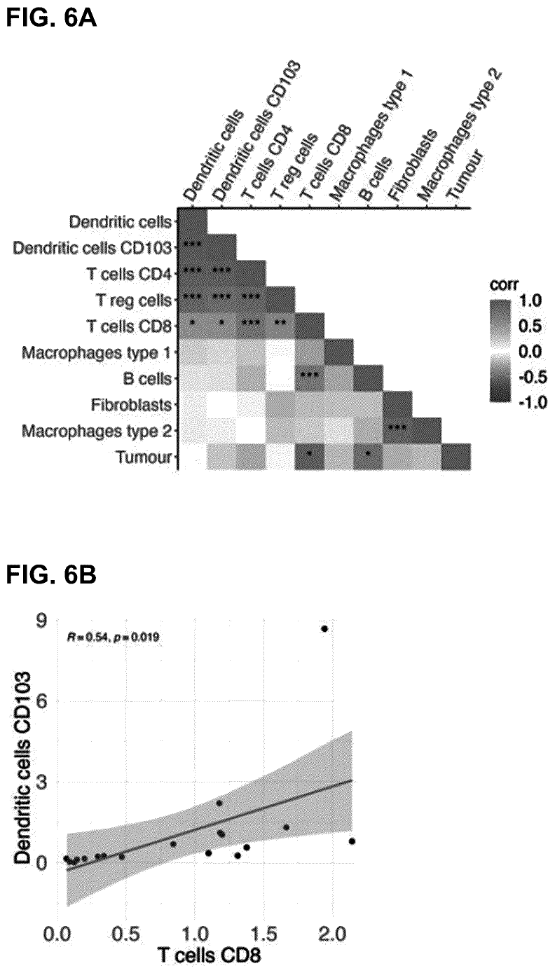

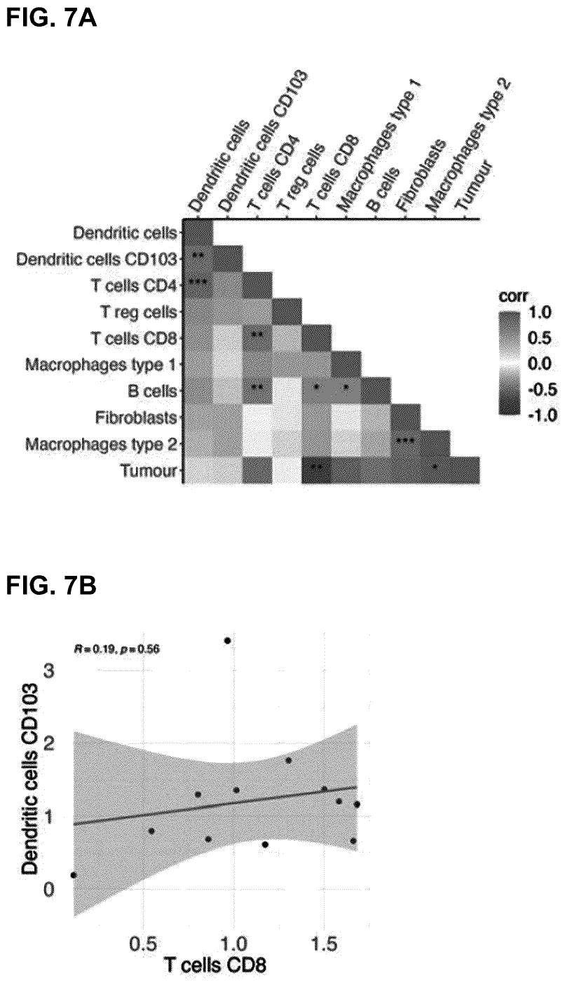

One way to infer potential cellular relationships is by correlating cell frequencies, measuring co-occurrences. Therefore, the correlation of each cell type pair within communities was calculated. Strong positive correlations were seen between certain cell types, such as T cells amongst themselves, T cells with dendritic cells, Type 2 macrophages with fibroblasts and CD8⁺ T cells with B cells when calculated per community (FIGS. 6A-6B). By contrast, a number of these, such as Tregs with CD4⁺ and CD8⁺ T cells, and most T cell-dendritic cell relationships showed lack of significance when regarded in the ROIs (FIGS. 7A-7B). This demonstrated the benefit of studying cellular relationships through the identification of localized spatial patterns, as they provide increased statistical power about the interactions occurring in the TME in comparison to measuring interaction of cells across a whole tissue.

### Example 3—Spatial Communities Abundant in CD8⁺ T Cells

Because the abundance of CD8⁺ T cells is associated with positive outcomes in relation to anti-tumoral immune response, T cell-rich communities from The 3LL ΔNRAS carcinoma tumor model were further investigated. Previous analysis revealed increased numbers of CD8⁺ T cells inside the tumor domain following KRAS-G12C inhibition in this model (van Maldegem et al. (2021) Nat. Commun. 12:5906).

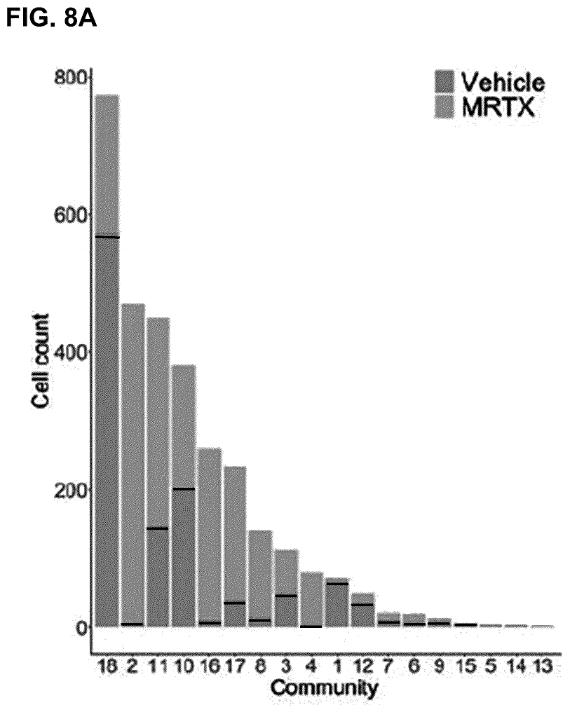

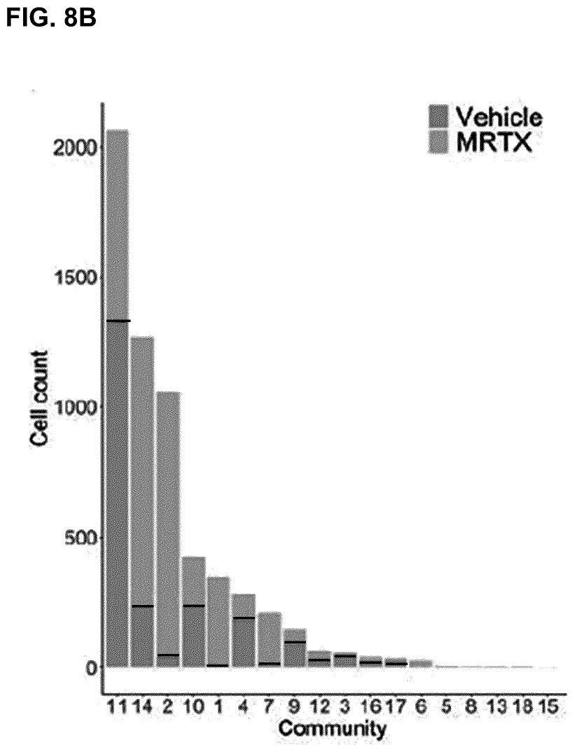

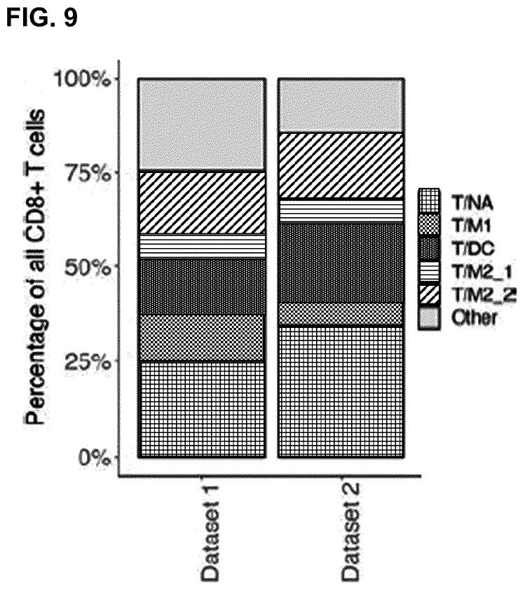

The top five communities with the highest CD8⁺ T cell count were identified from dataset 1 and dataset 2 (FIGS. 8A-8B). These five CD8⁺ T cell rich communities from both datasets paired up phenotypically, in particular when only shared cell types between datasets were considered (data not shown). Therefore, unique colours and the names T cell/normal adjacent community (T/NA), T cell/Type 1 macrophage community (T/M1), T cell/Dendritic cell community (T/DC), T cell/Type 2 macrophage community 1 (T/M2_1) and T cell/Type 2 macrophage community 2 (T/M2_2) were assigned to each pair for parallel analysis going forward. These 5 communities contained over 75% of the total T cell population from both datasets, suggesting a representative population of the overall cohort (FIG. 9). Interestingly these communities had widely different compositions, placing the T cells in very diverging spatial contexts. The T/NA community was characterized by high endothelial cell content, in the T/M1 community the Type 1 macrophages (CD11c⁺ CD68⁺; van Maldegem et al. (2021) Nat. Commun. 12:5906) were the most abundant cell type, the T/DC community was strongly enriched in various T cell subsets as well as dendritic cells, and T/M2_1 and T/M2_2 communities were dominated by tumor cells and Type 2 macrophages (F4/80⁺ CD206⁺; van Maldegem et al. (2021) Nat. Commun. 12:5906) in different ratios.

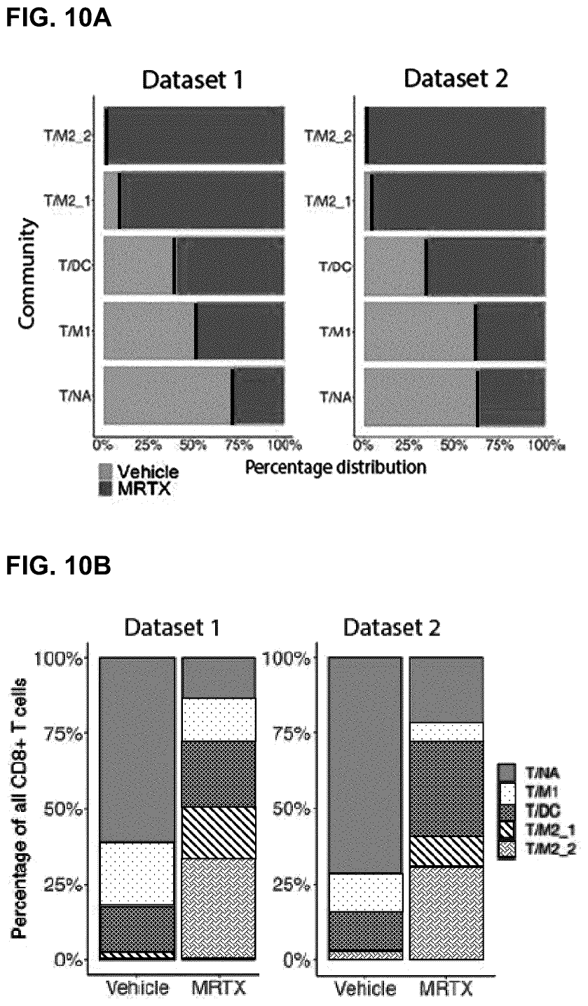

These CD8⁺ T cell rich communities also differed in frequency between treatment groups. The T/NA community was more frequently found following treatment with Vehicle, while the T/M2_2 community was almost exclusively detected in MRTX1257 treated tissues (FIG. 10A). Following from this, the majority of CD8⁺ T cells in the Vehicle treated tumors were found within T/NA and T/M1 communities, while CD8⁺ T cells in the MRTX treatment group were more likely to reside within T/DC, T/M2_1 and T/M2_2 communities (FIG. 10B).

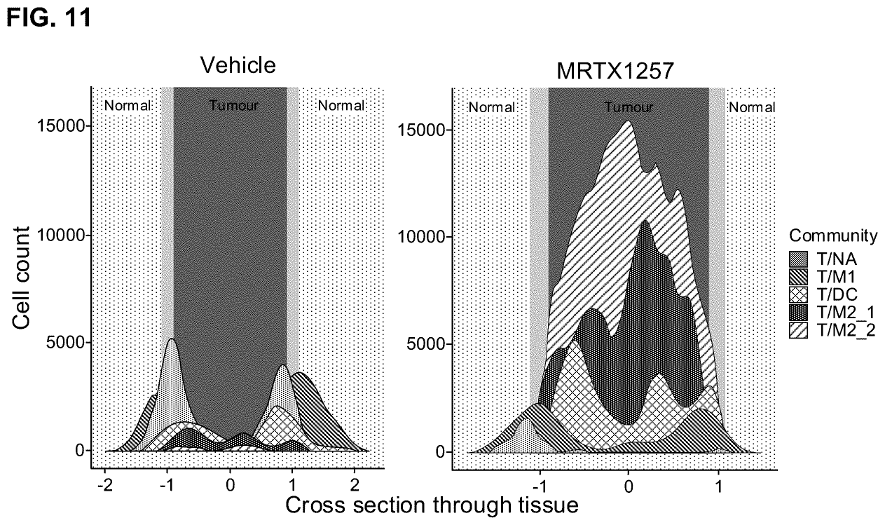

Furthermore, the spatial location of the top 5 communities also varied largely in relation to three assigned tissue domains: normal, interface and tumor (FIG. 11). In particular, in the vehicle setting, the T/NA community was situated predominantly in the ‘normal’ non-tumor region and the T/M1 community restricted to the ‘interface’, situated as a clear ring around the tumor bulk (data not shown). The T/DC community was located just inside the tumor bulk in the Vehicle setting but increased in size and its position moved towards the tumor core following treatment with MRTX1257. The T/M2_1 and T/M2_2 communities were concentrated within the tumor domain and predominantly found within the MRTX-treated tumors. Evidently, treatment with MRTX1257 led to a shift in spatial distribution and neighborhood environment of the CD8⁺ T cells.

### Example 4—T Cell Rich Communities Respond Directly to KRAS-G12C Inhibition

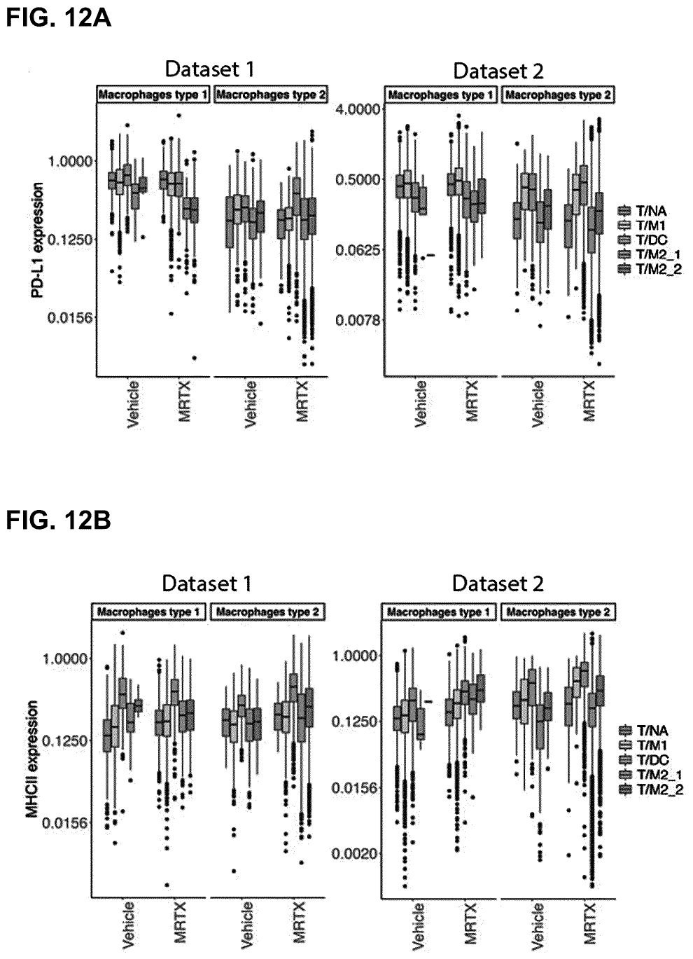

Communities were defined based on cell types using lineage markers independently of maturation or activation markers. Thus, it was next explored how cells within the communities responded to KRAS-G12C inhibition based on the maturation and activation markers included in both datasets. It was previously described how the two subsets of macrophages in this tumor model differed in spatial location and response to treatment with MRTX1257 (van Maldegem et al. (2021) Nat. Commun. 12:5906). The most notable observation was that the type 2 macrophages increased in cell number and upregulated activation markers such as PD-L1 and MHC-II. Zooming in on the communities here revealed that the upregulated expression could be largely attributed to the type 2 macrophages in the T/DC community (FIGS. 12A-12B).

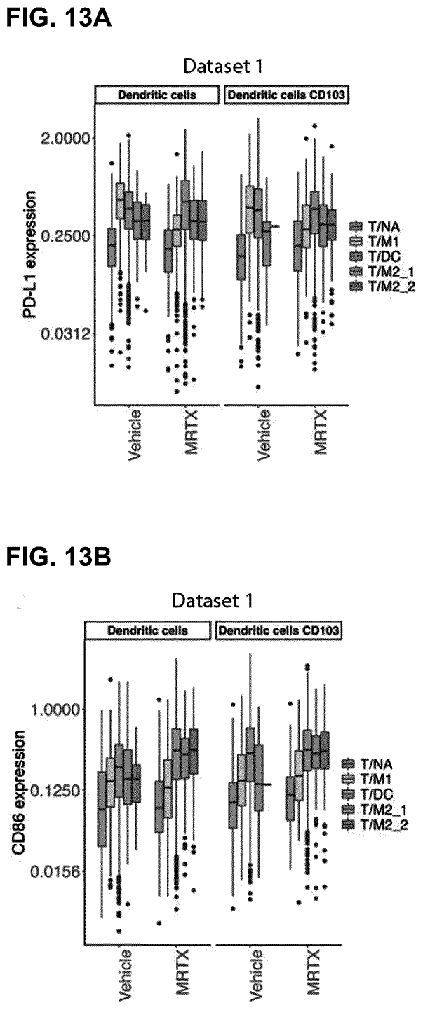

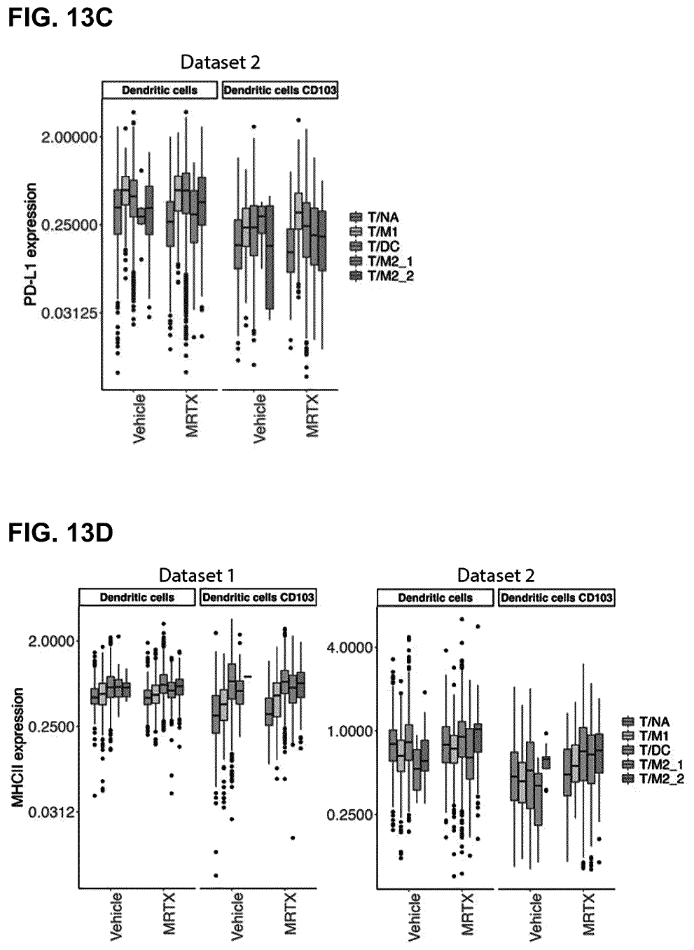

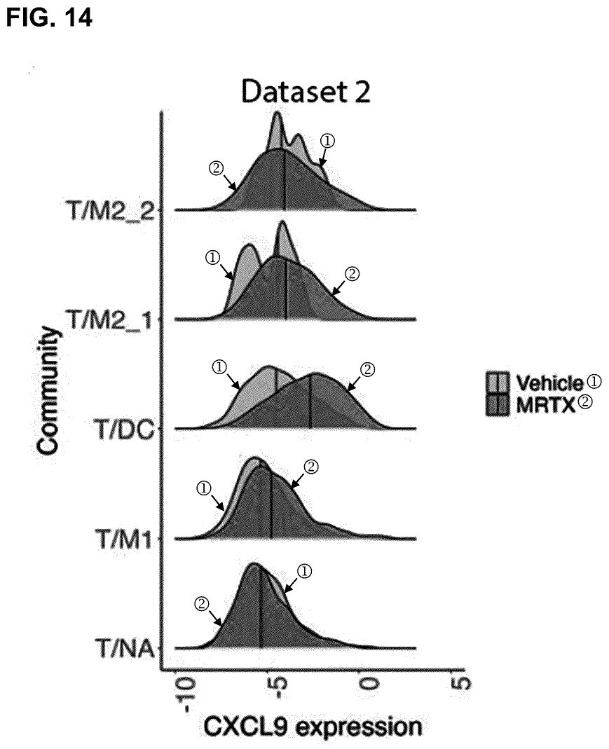

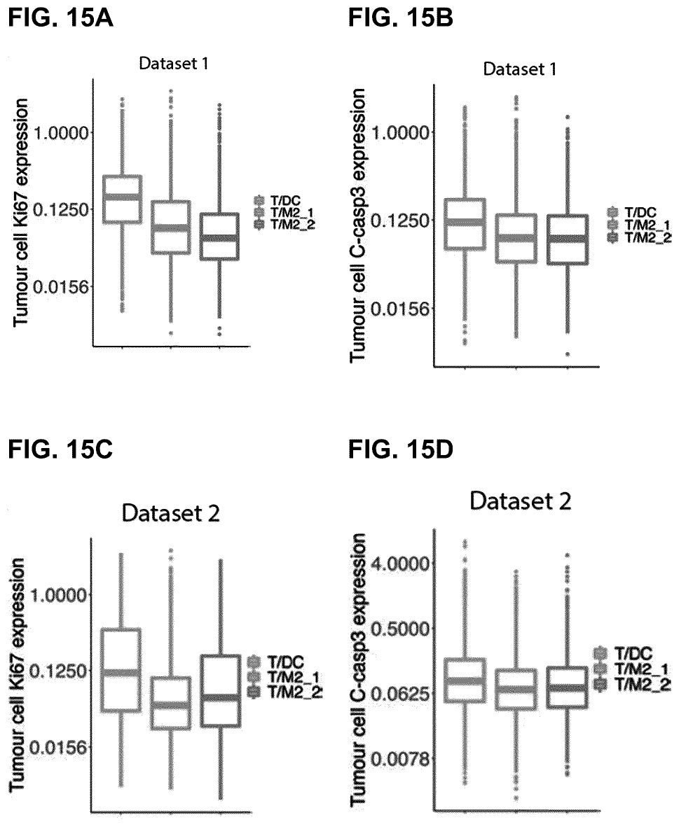

Expression of PD-L1 and co-stimulatory receptor CD86 on dendritic cells was also investigated. Differences were more noticeable across the communities than between treatment groups, suggesting dendritic cell phenotypes were more influenced by surrounding neighbors than treatment with MRTX1257 (FIGS. 13A-13C). The behaviour of expression varied slightly across these markers, with T/M1 community harbouring high PD-L1 but low CD86 expression in the Vehicle setting, but less so in inhibitor treated conditions. Such high PD-L1 and low CD86 expression is thought to be a characteristic of regulatory or migratory tolerogenic DCs, with a potential to inhibit immune responses. Alternatively, in T/DC, T/M2_1 and T/M2_2, increased PD-L1 expression on dendritic cells in the MRTX1257 treatment group was accompanied by high CD86 expression, indicative of a more activated DC phenotype making these cells better equipped to facilitate T cell activation. This differential in activation status was further supported by the expression levels of MHC-II, being highest in the T/DC community (FIG. 13D). Additionally, MRTX1257 treatment increased expression of CXCL9, with the highest overall expression in the T/DC community, suggesting most of the T cell attraction was occurring within this environment (FIG. 14). This is in agreement with the finding that the T/DC community contained the largest T cell density. Further, expression of proliferative marker Ki67 and apoptotic marker cleaved-caspase-3 (c-casp3) on tumor cells was compared in the tumor-associated communities (T/DC, T/M2_1 and T/M2_2). A higher expression of both markers was found in the T/DC community (FIGS. 15A-15B). This demonstrates the high level of activity occurring within this community, where some tumor cells were thriving whilst others were dying.

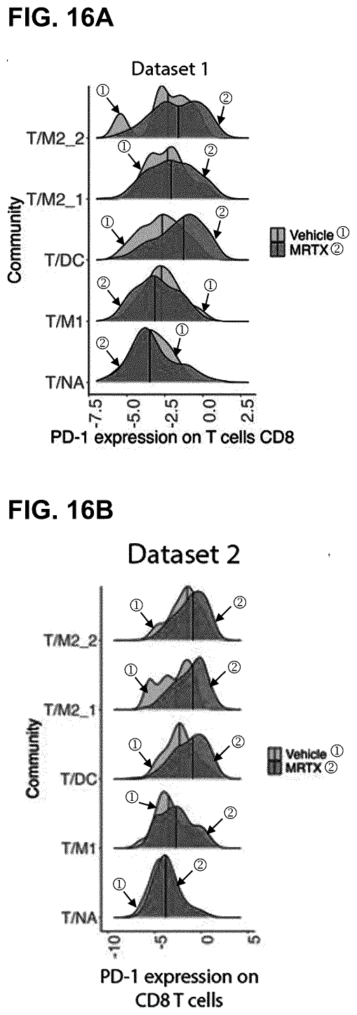

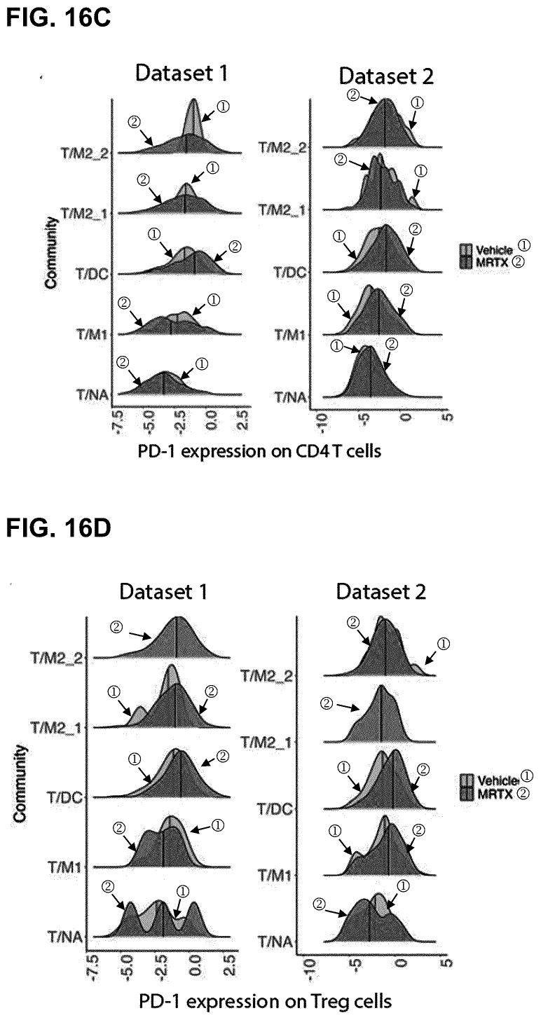

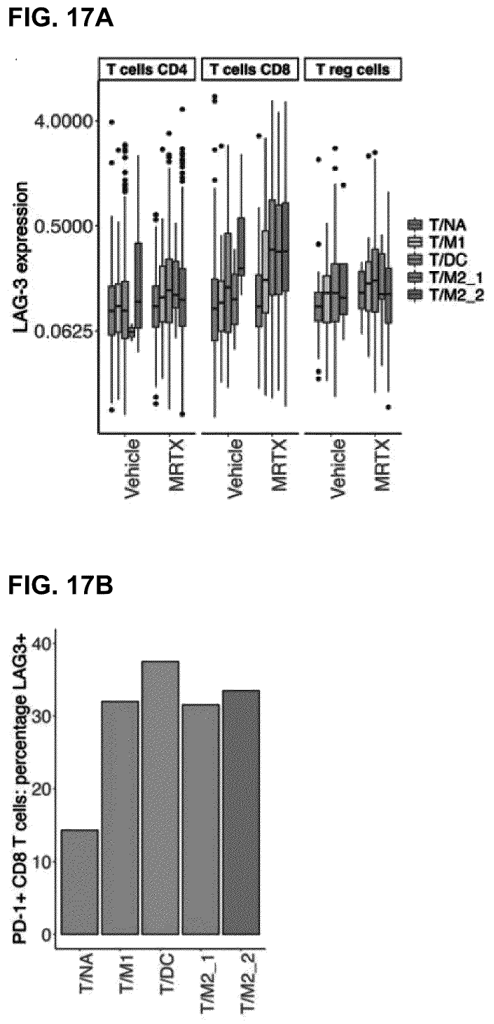

As it was expected that these different neighborhoods would also have an impact on the phenotype of the T cells, their PD-1 expression was investigated to understand how T cell activation state changed following KRAS-G12C inhibition, per community. In the T/NA and T/M1 communities, the PD-1 expression was negligible in both treatment groups, compared to the T/DC, T/M2_1 and T/M2_2 communities, where an increase occurred following treatment with MRTX1257, most pronounced in the T/DC community, indicating a switch in cell state from naïve to activated in these communities following KRAS-G12C inhibition (FIGS. 16A-16B). A similar pattern could be seen for CD4⁺ T cells, whereas the T reg cells had higher PD-1 expression mainly in the T/DC community following MRTX1257 treatment (FIGS. 16C-16D). However, expression of LAG-3 was also distinctly higher in CD8⁺ T cells, specifically in the tumor-associated communities, in which ˜30% of PD-1⁺ T cells were also LAG-3⁺ following treatment with MRTX, suggesting that activation of the T cells was in part accompanied with induction of T cell exhaustion following KRAS-G12C inhibition (FIGS. 17A-17B).

### Example 5—Positive and Negative Regulation of Anti-Tumoral Immune Responses Meet in the T/DC Community

While the T/DC, T/M2_1 and T/M2_2 communities contained most of the CD8⁺ T cells in the tumor tissue, these cells expressed significant levels of potential exhaustion markers such as PD-1 and LAG-3. Co-localization of PD-L1 expressing macrophages and PD-1⁺ CD8⁺ T cells has recently been highlighted as associated with good response to ICI (Färkkilä et al. (2020) Nat. Commun. 11:1459; Bortolomeazzi et al. (2021) Gastroenterology 161:1179-1193). However, previous attempts to reinvigorate these T cells using MRTX in combination with ICIs anti-PD-1 or anti-PD-L1 and anti-LAG-3 had failed to achieve any improved tumor control in the 3LL model (Mugarza et al. (2022) Sci. Adv. 8: eabm8780). The signals provided to the T cells prior to moving into the core of the tumor and becoming incapacitated by exhaustion were thus investigated in a next step. The high proportion of activated dendritic cells and increased expression of markers associated with T cell attraction and activation following KRAS-G12C inhibition, as well as evidence for local tumor cell death, pointed towards the T/DC community as a potential cytotoxic T cell activation hub.

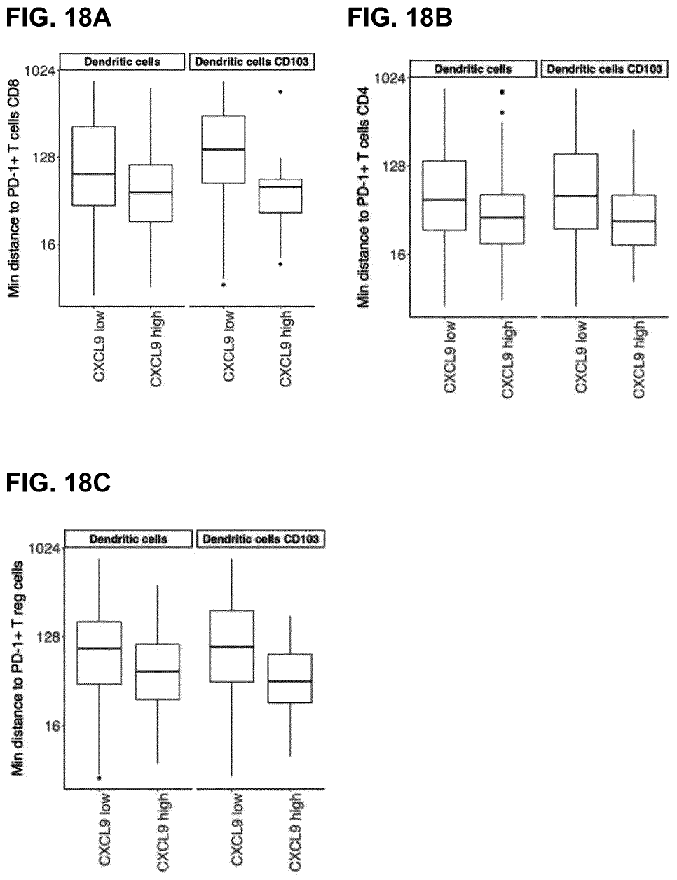

As noted, CXCL9 expression was highest within antigen presenting cells in the T/DC community (FIG. 14). Separating dendritic cells based on their CXCL9 expression into ‘CXCL9 low’ and ‘CXCL9 high’ groups showed that CXCL9 high dendritic cells had a significantly shorter distance to their nearest PD-1⁺ CD8⁺ T cell, PD-1⁺ CD4⁺ T cells and PD-1⁺ Tregs (FIGS. 18A-18C). Visual inspection indeed confirmed that CXCL9 high dendritic cells were frequently found in proximity to and interacting with PD-1⁺ CD8⁺ T cells (data not shown). CXCL9 expression could therefore be one of the key mediators driving the aggregation of the T cells and dendritic cells within the T/DC community, as part of a mechanism to draw in the activated T cells to launch an anti-tumor immune response. This would be in agreement with previous work showing that CXCL9 expression was one of the strongest predictors of ICI (Litchfield et al. (2021) Cell 184:596-614.e14).

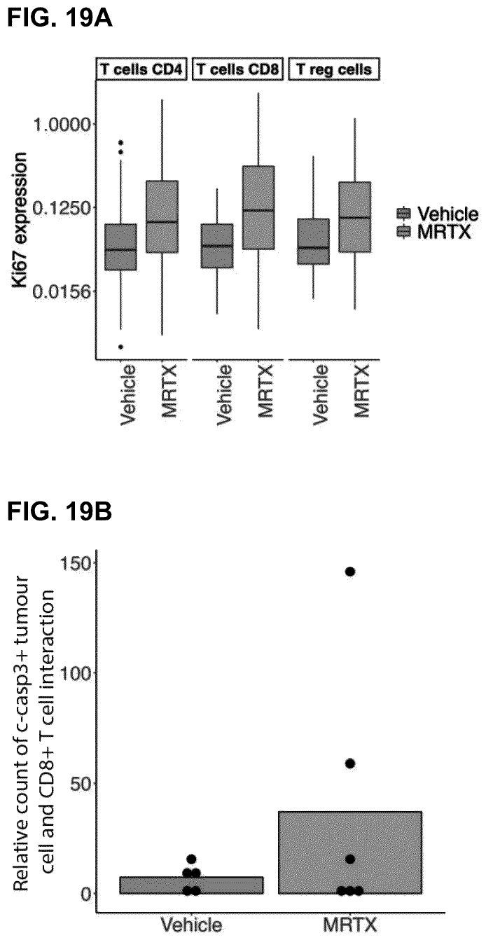

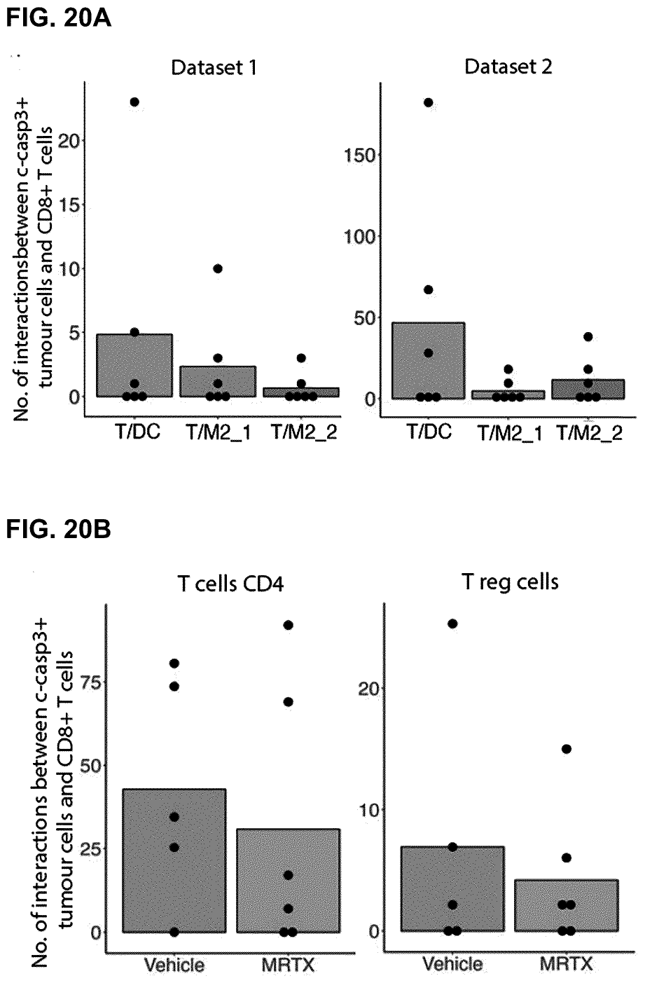

It was further sought to determine additional indication of active anti-tumoral immune response within this community. A significant increase of Ki67 expression was identified for CD4⁺ and CD8⁺ T cells, and a similar trend for Treg cells, suggesting that the T cells had increased proliferation following KRAS-G12C inhibition (FIG. 19A). Frequency of casp3⁺ tumor cells was highest in the T/DC community and the occurrences in which a c-casp3⁺ tumor cell was found in the 15-pixel neighborhood of a CD8⁺ T cell increased following treatment with MRTX1257 (FIG. 19B). These interactions were primarily found within the T/DC community (FIG. 20A). There was no such increase in spatial interactions identified for CD4⁺ or Treg cells with c-casp3⁺ tumor cells (FIG. 20B). Such increased interactions point to increased cytotoxicity of the T cells against the tumor cells following KRAS-G12C inhibition, suggesting that the neighborhood of the T/DC community was indeed likely to be able to support the effector function of the CD8⁺ T cells.

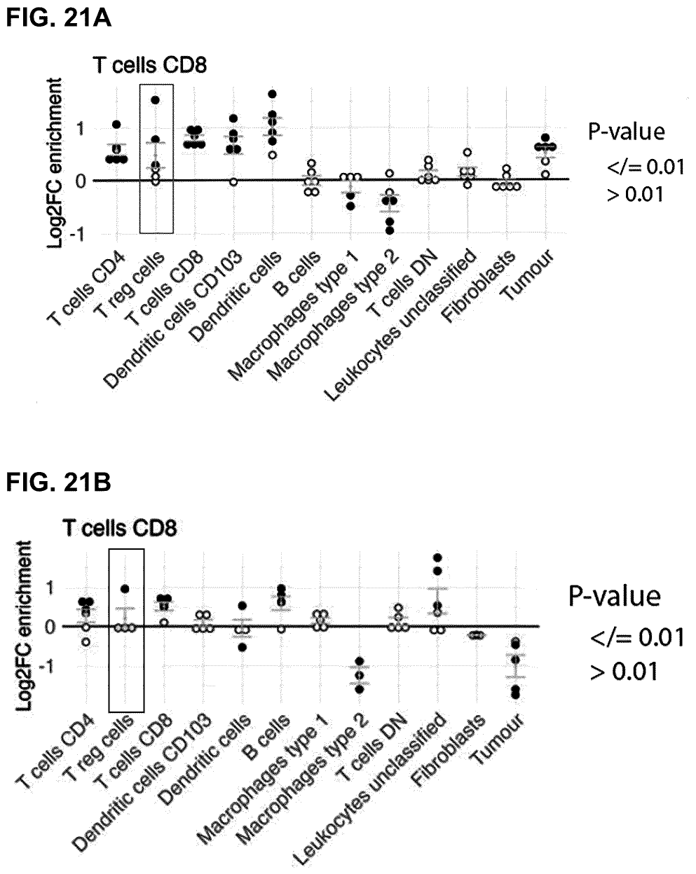

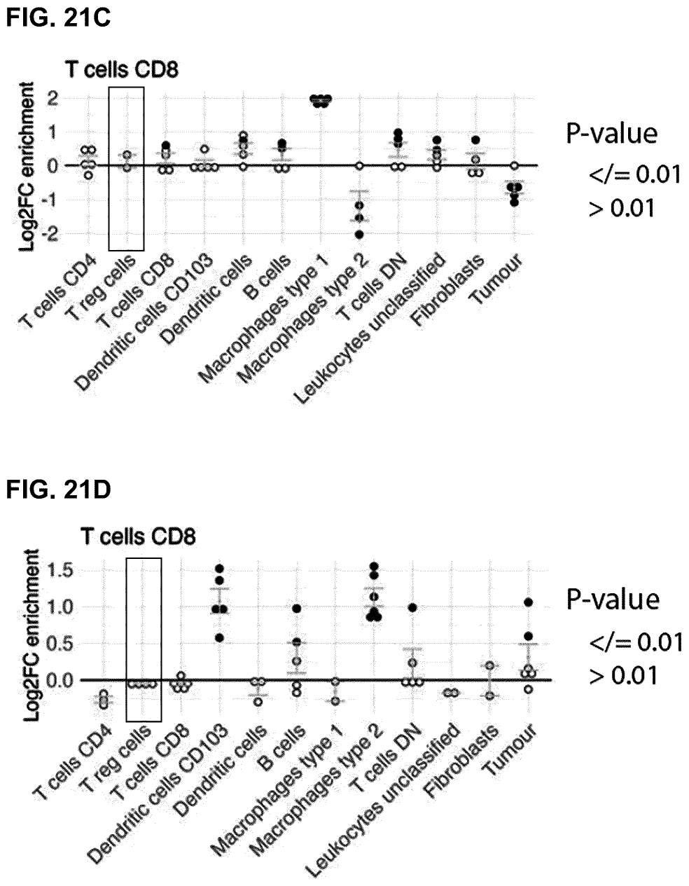

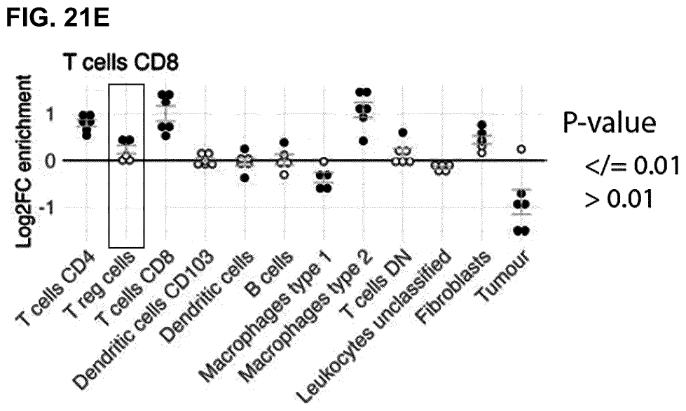

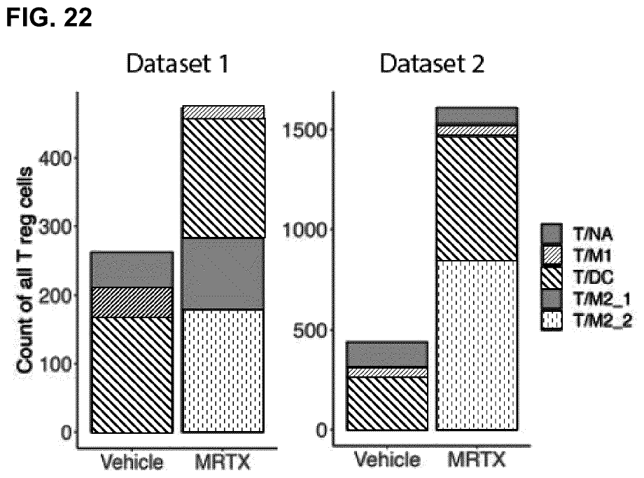

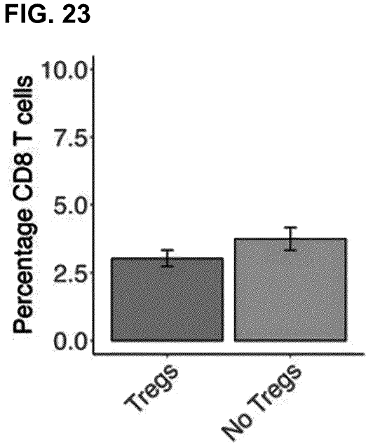

It was then investigated why such a cytotoxic response was not effective enough to mediate clinical benefit and what could be driving the induction of T cell exhaustion or dysfunction. To identify potential negative regulatory influences of the immune response, another way of interrogating spatial relationship was employed, namely by calculating enrichment scores for cells in the near neighborhood, compared to randomized data (van Maldegem et al. (2021) Nat. Commun. 12:5906; Schapiro et al. (2017) Nat. Methods 14:873-876). Following MRTX1257 treatment, it was determined that CD4⁺ T cells and dendritic cells were significantly enriched in the neighborhood of a CD8⁺ T cell within the T/DC community in at least five out of the six images, compared to random permutation (FIG. 21A). This is supportive of a microenvironment promoting anti-tumoral immune response, which was not apparent from T/NA, T/M1, T/M2_1 and T/M2_2 communities (FIGS. 21B-21E, respectively). However, Treg cells were also significantly enriched in the neighborhood of CD8⁺ T cells in 3 out of the 6 images, indicating the presence of immune suppressor cells in the vicinity where T cell activation and effector functions may be taking place (FIG. 21A). Treg cell frequencies were highest within the T/DC and T/M2_2 communities. This T cell subset also increased in size, most notably in the T/DC and T/M2_2 communities and, as reported above, was showing evidence of increased activation after MRTX1257 treatment (FIG. 22, FIG. 16D). Indeed, Treg cells were seen intermixed with effector T cells and dendritic cells, suggesting a potential role in locally dampening anti-tumoral immune responses.

### Example 6-Assigning an Important Role for Treg Cells in Dampening Anti-Tumoral Immune Responses

Upon revealing the high presence of Treg cells within the T/DC community and showing that they are enriched within the neighborhood of CD8⁺ T cells and neighboring dendritic cells and CD4⁺ T cells following treatment with MRTX1257, their role within this community in relation to anti-tumoral immune response was explored in a next step. The T/DC community was thus split up into two neighborhoods: those with presence of Treg cells, named ‘Tregs’, or those with absence of Treg cells, named ‘No Tregs’. The neighborhoods differed slightly in their composition of cell types, with the ‘Tregs’ neighborhood comprising a higher proportion of dendritic cells and CD4⁺ T cells, whereas the ‘No Tregs’ neighborhood contained a higher tumor and type 2 macrophage portion (data not shown).

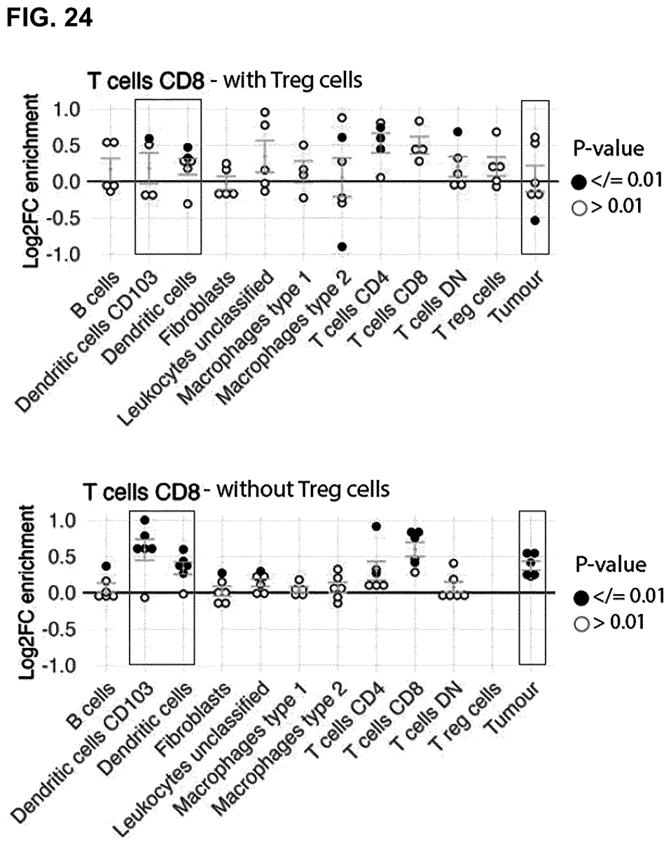

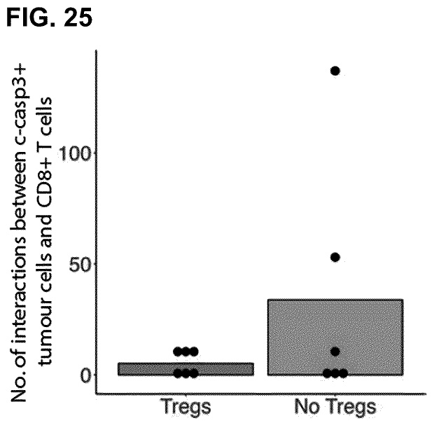

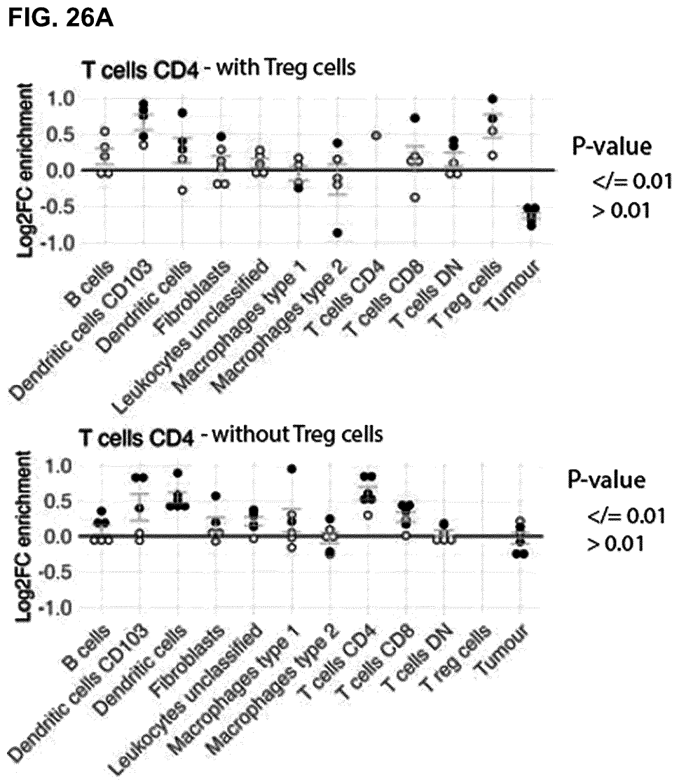

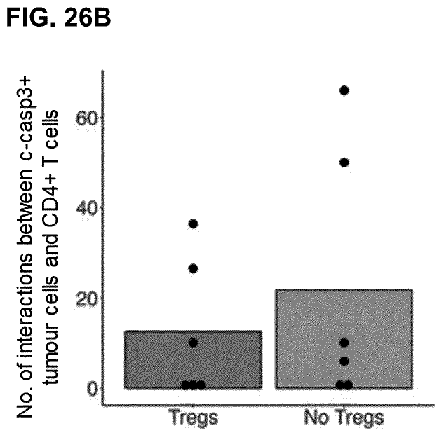

While the frequency of CD8⁺ T cells within both the ‘Tregs’ and ‘No Tregs’ neighborhoods were similar (FIG. 23), the cellular interactions for CD8⁺ T cells differed substantially between these environments. Despite the slightly higher dendritic cell frequency in the ‘Tregs’ neighborhoods, a lack of spatial enrichment between CD8⁺ T cells and both dendritic cell subsets was observed when Treg cells were present, in contrast to a positive enrichment of dendritic cells in their neighborhood when Treg cells were absent (FIG. 24). Additionally, when Treg cells were missing in the CD8⁺ T cell neighborhood, a strong enrichment of tumor cells was identified following MRTX treatment, compared to slight depletion when the Treg cells were nearby. Furthermore, the frequency of c-casp3⁺ tumor cells in the CD8⁺ T cell close neighborhood increased on MRTX treatment when Treg cells were absent (FIG. 25). Interactions with CD8⁺ T cells indicative of an active anti-tumoral immune response were thus only observed when the Treg cells were absent, suggesting a strong inhibitory role for the Treg cells. These changes in neighborhood enrichment were not observed when comparing CD4⁺ T cells with or without Treg cells in their neighborhood, indicating that Treg presence may affect CD8⁺, but not CD4⁺ T cell relationships (FIGS. 26A-26B). Also other changes to the cellular interactions were observed when sub-setting the T/DC community based on presence or absence of Treg cells, suggesting that Treg cells may be impacting the local milieu of this community in many ways, further pointing towards their potential negative influence on anti-tumoral immune response.

Overall, these analyses indicated that the T/DC community potentially could provide an activating environment for the anti-tumor cytotoxic T cell response, but the presence of Treg cells was likely imposing a strong negative influence.

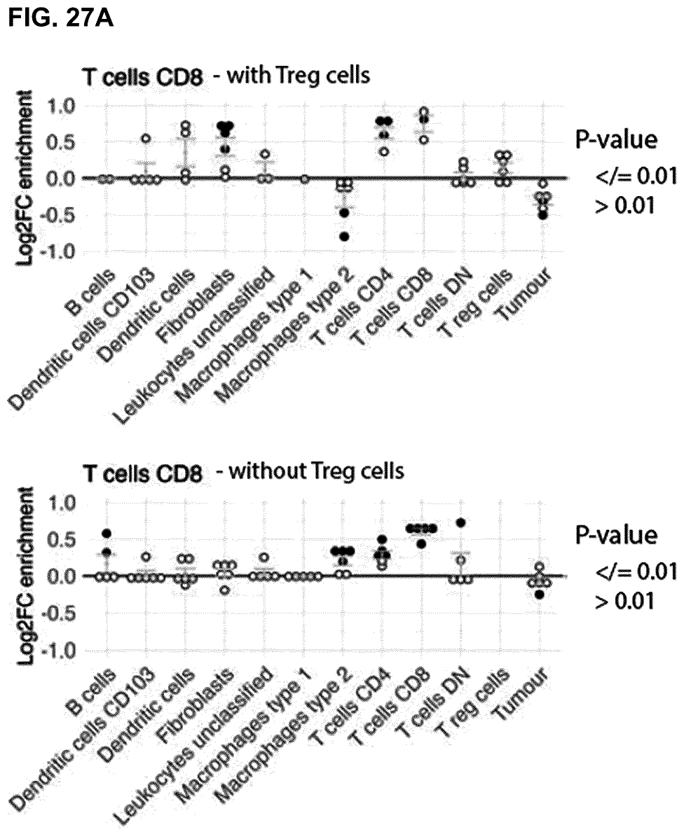

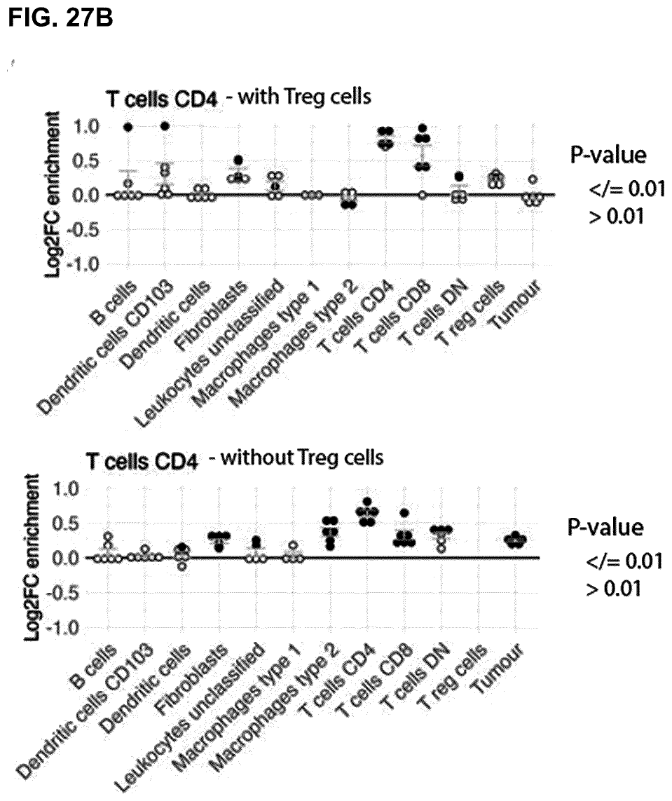

A similar analysis for the T/M2_2 community, also relatively rich in Tregs (data not shown), showed that CD8⁺ T cell and Treg interactions focused mainly on fibroblasts, while CD8⁺ T cell interactions with M2 macrophages were largely interrupted in presence of Treg cells (FIG. 27A). Likewise, CD4⁺ T cell interactions with M2 macrophages were also diminished in presence of Treg cells (FIG. 27B). This further solidifies the likely role of Treg cells suppressing T cell function through downregulating interactions with antigen presenting cells following KRAS-G12C inhibition, which occurred across multiple spatial groups.

### Example 7—Treg Cell Spatial Cellular Communities in Human Non-Small Cell Lung Cancer (NSCLC)

It was next investigated whether communities similar to the mouse communities could be identified in human lung cancer clinical samples, focusing on the communities rich in Treg cells, effector T cells and dendritic cells, like the T/DC community observed in the mice samples.

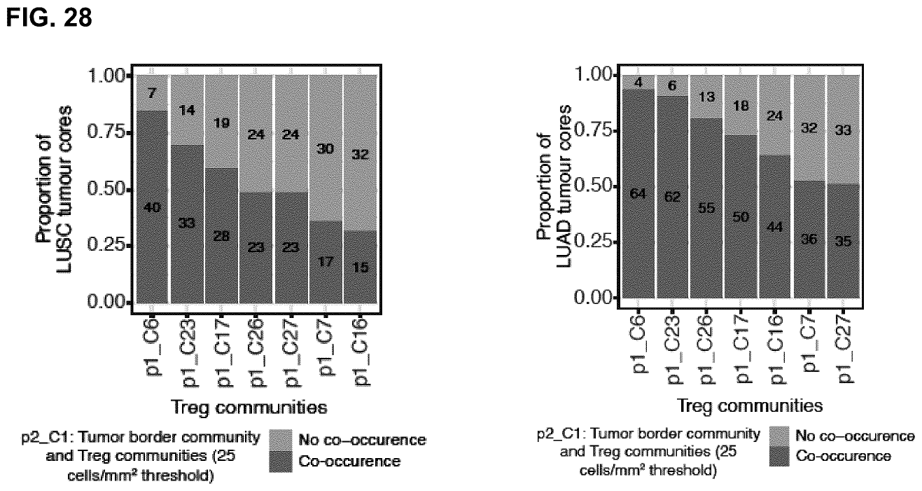

151 tumor regions from 81 untreated NSCLC patients from the TRACERx longitudinal study were analysed with imaging mass cytometry using two 35-plex antibody panels: panel 2, the pan-immune cell panel and panel 1, designed to assess the differentiation states of T cells and stromal cells in greater detail (T cell panel). The T cell panel provides the opportunity to not only match cell type compositions with the mouse data above, but also to potentially refine the T cell signatures that are associated with them. Therefore, communities were detected based on the T cell panel (panel 1) with relatively high granularity, obtaining 30 communities, including 7 that were rich in Treg cells and other T cells (data not shown). Five of these communities (p1_C7, p1_C17, p1_C23, p1_C26 and p1_C27) included mature and/or exhausted CD4 and CD8⁺ T cells, similar to the mouse T/DC community. Another characteristic feature of the mouse T/DC community was the presence of dendritic cells, as well as the peritumoral localization. Dendritic cells were not identified from the T cell panel (panel 1, p1). However, community analysis of the pan-immune (panel 2) data identified 10 communities in which p2_C1: tumor border also contained dendritic cells and was in the peritumoral region (data not shown). Interestingly, the presence of p2_C1: tumor border correlated with the density of Treg cells in lung adenocarcinoma, LUAD (data not shown), but not in lung squamous cell carcinoma, LUSC (data not shown). In a significant subset of patients, the co-occurrence of p2_C1: tumor border from panel 2 and Treg communities from panel 1 was confirmed (FIG. 28), and that in particular Treg communities p1_C7, p1_C17, p1_C23, p1_C26 and p1_C27 displayed a peritumoral distribution close to p2_C1: tumor border (data not shown). Homing in on the phenotypes of the T cells associated with these communities revealed that p1_C27 was standing out among the others. CD4⁺ and CD8⁺ T cells in p1_C27 expressed high levels of immune checkpoints (TIM3, GITR, CTLA-4, ICOS). However, in contrast to the mouse T/DC community, there was no evidence of ongoing T cell activation, with absence of proliferation marker Ki67 and cytotoxicity marker Granzyme B (GZMB) (data not shown). The Treg cells in p1_C27 expressed very high levels of all tested markers except GITR, an immune checkpoint that was recently described to mark the most immune suppressive Treg subset in NSCLC and associated with PD-1 resistance (Dykema et al., 2023, Sci Immunol 8, eadg1487). Some of the other communities expressed similar or higher levels of GITR on the Treg cells (p1_C26, p1_C23, p1_C6, p1_C17) and showed above average expression of immune checkpoints on the CD4⁺ and CD8⁺ T cells (p1_C7, p1_C23, p1_C17, p1_C26), more similar to the mouse T/DC community.

The MYSTIC trial, which tested the efficacy of durvalumab (anti-PD-L1) with or without tremelimumab (anti-CTLA-4) vs. standard chemotherapy in first-line treatment of metastatic NSCLC (Rizvi et al., 2020, JAMA Oncol 6, 661-674), demonstrated that the combination of durvalumab and tremelimumab showed improved survival compared to durvalumab alone only in patients with high tumor mutational burden, TMB (>20 mutations/Mb). Using the genomics data available for the patients of this TRACERx cohort (Jamal-Hanjani et al. (2017) N. Engl. J. Med. 376:2109-2121), it was found that p1_C7 and p1_C17 also correlated with the most increased TMB in LUAD, suggesting that these Treg communities could be targeted with anti-CTLA-4.

Overall, the data from human lung cancer samples that have not been treated with KRAS inhibitory drugs suggest that cellular communities similar to the mouse T/DC community exist at baseline, where CD4⁺ and CD8⁺ T cells, dendritic cells and Treg cells were gathered together at the tumor periphery.

### Example 8—Combination of Treg Cell Depleting Agents with a KRAS Inhibitor Controls Tumor Growth In Vivo

In a next step, it was investigated whether Treg cell depleting agents can promote immune-mediated control of orthotopic 3LL-ΔNRAS lung tumors treated with a KRAS-G12C inhibitor. The 3LL-ΔNRAS cell line can form orthotopic tumors in the lungs of C57BL/6 mice when delivered intravenously. The study outline is shown in FIG. 29A, and the probability of survival over the days of treatment for the different treatment groups is shown in FIG. 29B.

ADCC-mediated depletion of Tregs has been attempted using antibodies to target antigens expressed on Tregs such as, for example, CD25 (Arce Vargas et al. (2017) Immunity 46 (4): 577-586), CCR4 (Ishida et al. (2012) J. Clin. Oncol. 30:837-842; Hagemann et al. (2014) PLOS ONE 9 (7): e103776), CTLA-4 (Korman et al. (2017) Cancer Res. 77 (13_Suppl): Abstract SY09-01), and CCR8 (Campbell et al. (2021) Cancer Res. 81 (11): 2983-2994). However, attempts to treat tumors by agents specifically depleting Tregs from the TME have given mixed results. The administration of anti-CD25 antibody against established tumors failed to delay tumor growth, which was attributed, in part, to potential depletion of activated effector CD8⁺ and CD4⁺ T cells that also express high levels of CD25 (Onizuka et al. (1999) Cancer Res. 59:3128-3133). Despite documented anti-CTLA-4 mediated Treg depletion in mouse tumor models (Selby et al. (2013) Cancer Immunol. Res. 1 (1): 32-42; Simpson et al. (2013) J. Exp. Med. 210 (9): 1695-1710), evidence for Treg depletion by ipilimumab or tremelimumab (anti-human CTLA-4 antibodies) in human cancer is unclear (Sharma et al. (2019) Clin. Cancer Res. 25 (4): 1233-1238; Sharma et al. (2019) Clin. Cancer Res. 25 (11): 3469-3470). Treg depletion was achieved with the nonfucosylated anti-human CCR4 antibody, mogamulizumab, but significant depletion of conventional CD4⁺ T cells and modest reductions in CD8⁺ T cell numbers were also observed (Kurose et al. (2015) Clin. Cancer Res. 21 (19): 4327-4336), limiting its utility in the treatment of solid tumors. CCR8 expression is selectively upregulated in tumor-infiltrating Tregs (Plitas et al. (2016) Immunity 5:1122-1134; De Simone et al. (2016) Immunity 5:1135-1147), and several groups have demonstrated using mouse tumor models that anti-CCR8 antibodies are effective in treating tumors by depleting Tregs (see, e.g., WO2020138489; WO2021142002; WO2021152186; WO2021163064; WO2021194942; WO2023010054; WO2023288241). Thus, anti-CCR8 antibody is a promising immunotherapeutic candidate for depleting Tregs in the TME and treating cancer. In the present study, anti-CTLA-4 and anti-CCR8 were tested as Treg-depleting agents in combination with other anti-tumor agents.

The probability of survival over the days of treatment for the different treatment groups is shown in FIG. 29B. Treatment with an anti-PD-1 antibody in combination with either an anti-CTLA-4 or anti-CCR8 antibody failed to significantly control growth of 3LL-ΔNRAS tumors and to extend survival of mice compared to the treatment with a Vehicle (FIG. 29B). In addition, treatment with a combination of MRTX849 and anti-PD-1 led to short-term tumor control after one week on treatment and extended survival during the first 3 weeks of treatment, but tumors eventually relapsed and nearly all mice reached their endpoint within 5-6 weeks (FIG. 29B). Thus, administration of anti-PD-1 antibodies did not improve the response to MRTX in this mouse 3LL-ΔNRAS lung tumor model. See Mugarza et al., Sci. Adv. 8, eabm8780 (2022).

In contrast, addition of anti-CTLA-4 or anti-CCR8 antibodies, which are believed to mediate the depletion of tumor-infiltrating Tregs, extended the survival of mice treated with adagrasib+ anti-PD-1 (FIG. 29B). As shown in FIG. 29B, the probability of survival did not decline for about the first 25 days of treatment with the triple combination of MRTX, anti-PD-1, and anti-CTLA4, and there was an approximately 65% probability of survival after 6 weeks of treatment. Anti-CCR8 was even more effective than anti-CTLA-4 in extending survival: the substitution of anti-CCR8 for anti-CTLA-4 in the triple combination with MRTX and anti-PD-1 led to no decline in the probability of survival for about the first 40 days of treatment, and the probability of survival after 6 weeks of treatment was about 85%.

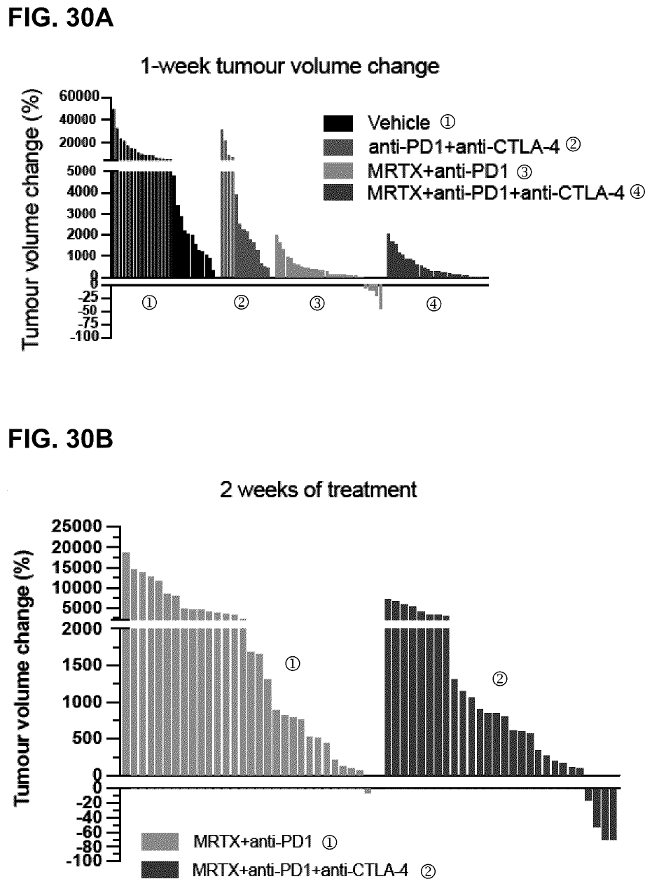

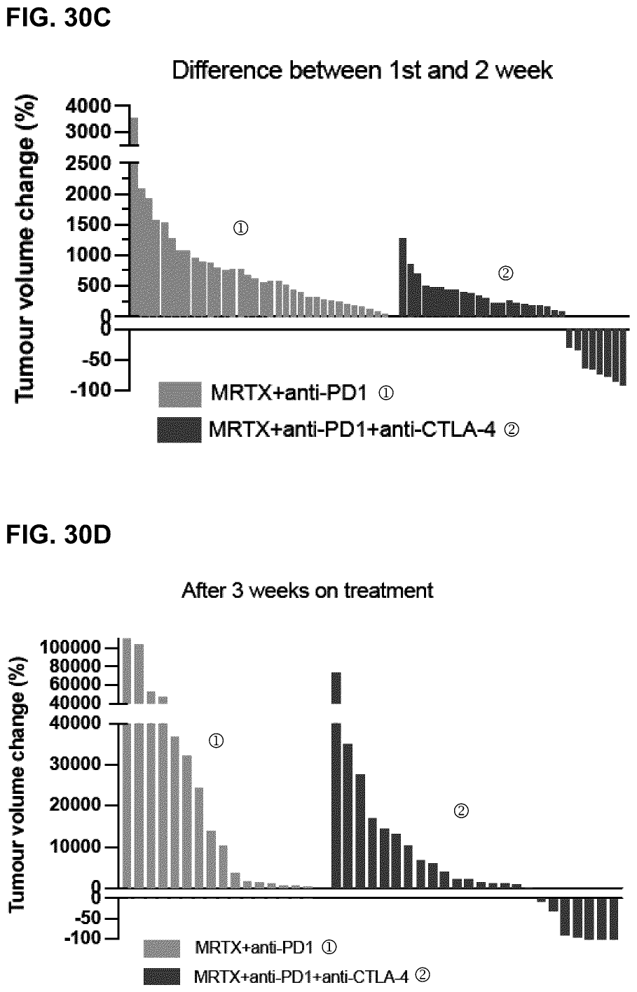

Although after one week of treatment, a combination of MRTX and anti-PD-1 with Treg-depleting anti-CTLA-4 therapy did not significantly impact tumor growth (FIG. 30A), after two weeks, tumor volume assessment revealed most tumors from the MRTX+ anti-PD-1 group had relapsed in contrast to triple combination with anti-CTLA-4 where tumors kept regressing (FIGS. 30B and 30C). This response pattern continued after 3 weeks of treatment (FIG. 30D).

At the end of the treatment period (40 days), 1 out of 8 mice on the triple combination therapy with anti-CTLA-4 (MRTX+ anti-PD-1+ anti-CTLA-4), and 2 out of 8 mice on the triple combination with anti-CCR8 (MRTX+ anti-PD-1+ anti-CCR8), were completely tumor free (as measured by CT scan). These mice remained tumor free after withdrawal of treatment for 30 days.

In summary, it was shown that the triple combination of MRTX+ anti-PD-1+a Treg cell depleting agent resulted in significant tumor control and extended the survivial compared to double combination therapy with MRTX and anti-PD-1. Anti-CCR8, which is more effective than anti-CTLA-4 in depleting tumor-infiltrating Tregs, exhibited greater in vivo anti-tumor efficacy than anti-CTLA-4 when included in triple combinations with MRTX and anti-PD-1, evidenced by increased tumor control and extension of survival in the orthotopic 3LL mouse lung tumor model.

### Example 9—Combination of Treg Cell Depleting Agents with a KRAS Inhibitor Depletes Tumor-Infiltrating Treg Cells In Vivo as Shown by FACS

It was further investigated whether Treg cell depleting agents can induce depletion of Treg cells in orthotopic 3LL-ΔNRAS lung tumors treated with a KRAS-G12C inhibitor.

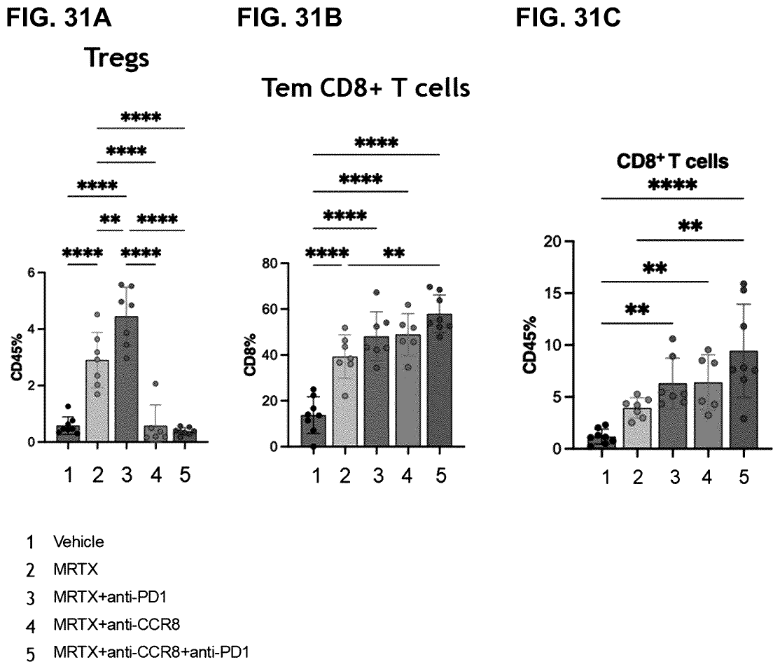

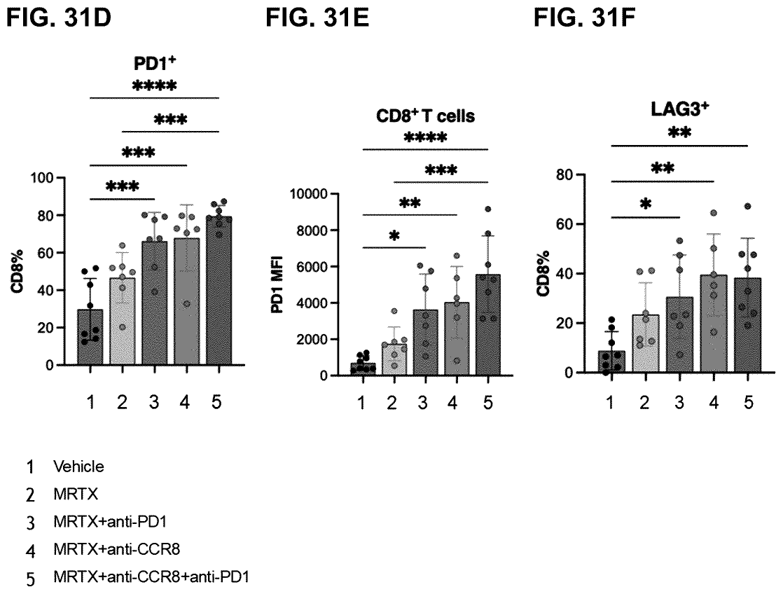

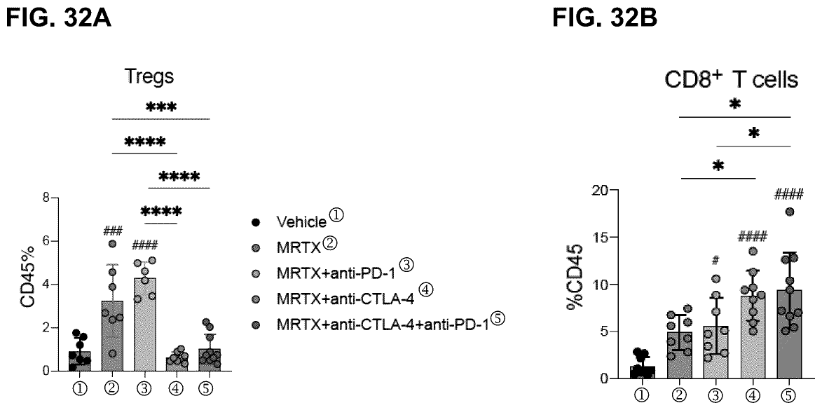

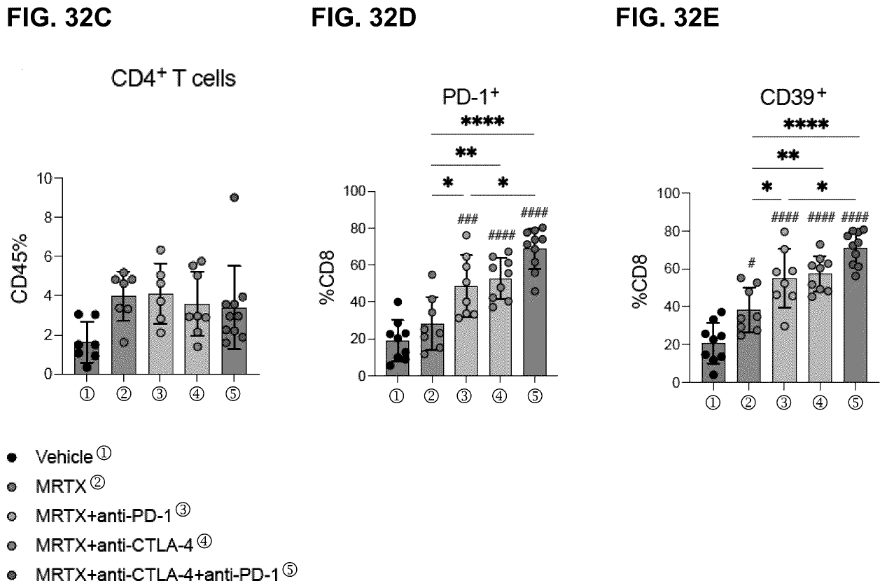

FACS analysis demonstrated increased Treg (CD4⁺ FOXP3⁺) infiltration upon adagrasib treatment, which was exacerbated with the addition of anti-PD-1 (FIGS. 31A and 32A). The majority of infiltrating Tregs were characterized by the expression of CCR8.

The addition of anti-CCR8 therapy (adagrasib+ anti-CCR8, and adagrasib+ anti-PD-1+ anti-CCR8) enabled depletion of Treg levels (FIG. 31A). Moreover, addition of anti-CCR8 (adagrasib+ anti-CCR8, and in particular adagrasib+ anti-PD-1+ anti-CCR8) increased the infiltration and activation of CD8⁺ T cells (FIG. 31B-31F).

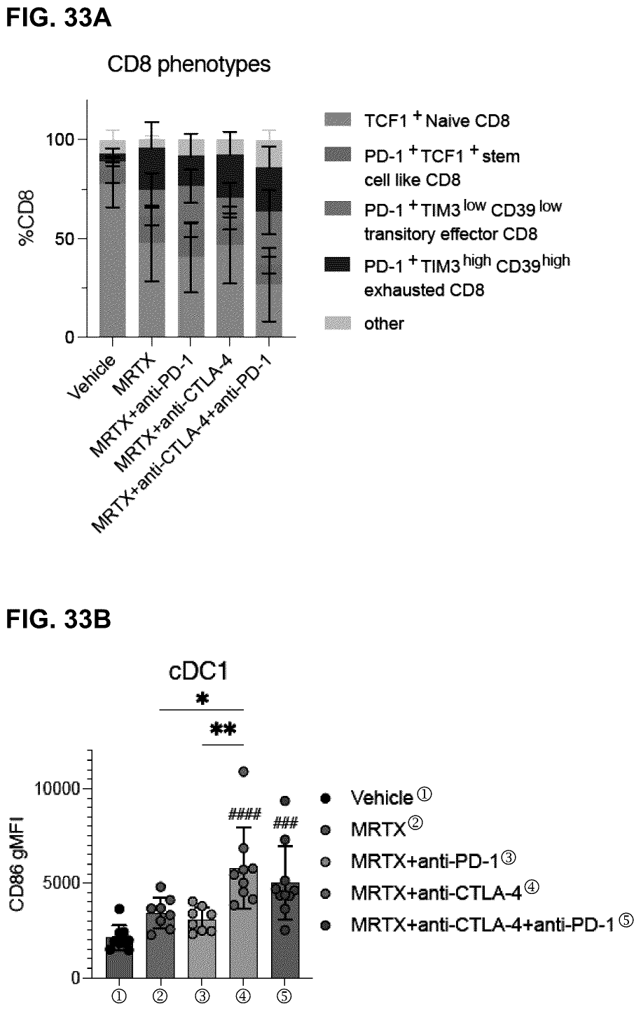

Similarly to anti-CCR8 treatment, anti-CTLA-4 was also able to deplete Tregs and prevent the increase in Treg infiltration observed after adagrasib and adagrasib+ anti-PD-1 treatment (FIG. 32A). This occurred with an increased CD8⁺ T cell infiltration, beyond what was observed with MRTX alone or in the MRTX+ anti-PD-1 combination (FIG. 32B), while total CD4⁺ T cell numbers remained stable between all conditions (FIG. 32C). CD8⁺ T cells in the tumor upregulated activation markers PD-1 and CD39, suggestive of increased tumor-reactive T cell rates following Treg depletion (FIGS. 32D and 32E), and progressively converted from predominantly naïve in the vehicle control setting to effector and exhausted CD8⁺ T cells upon addition of the three therapeutics (MRTX, anti-PD-1 and anti-CTLA-4; FIG. 33A). Moreover, upon combination treatment with MRTX+ anti-PD-1+ anti-CTLA-4, and also MRTX+ anti-CTLA-4, an elevated CD86 expression on CD103⁺ Type 1 Dendritic cells (cDC1s) was observed, an indication of increased antigen presentation and explainable by reduced transendocytosis in absence of Treg cells (FIG. 33B).

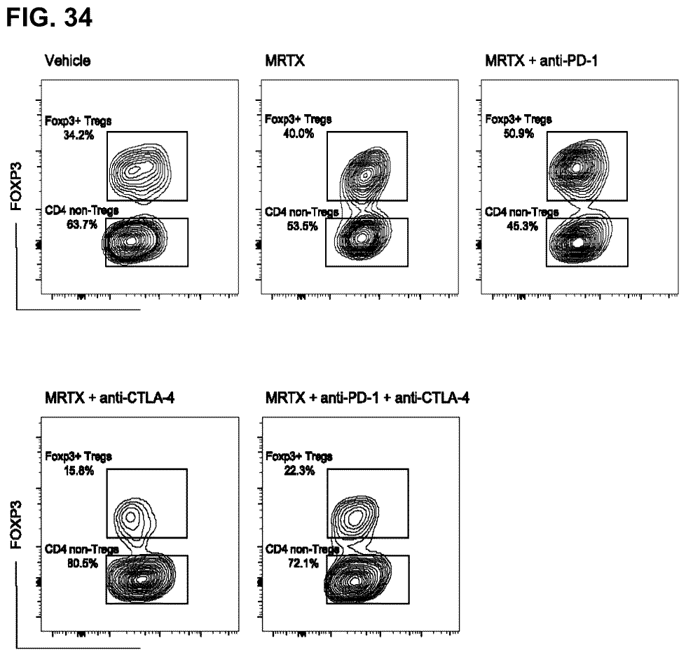

Representative flow cytometry contour plots for CD4⁺ FOXP3⁺ Treg cells are shown in FIG. 34.

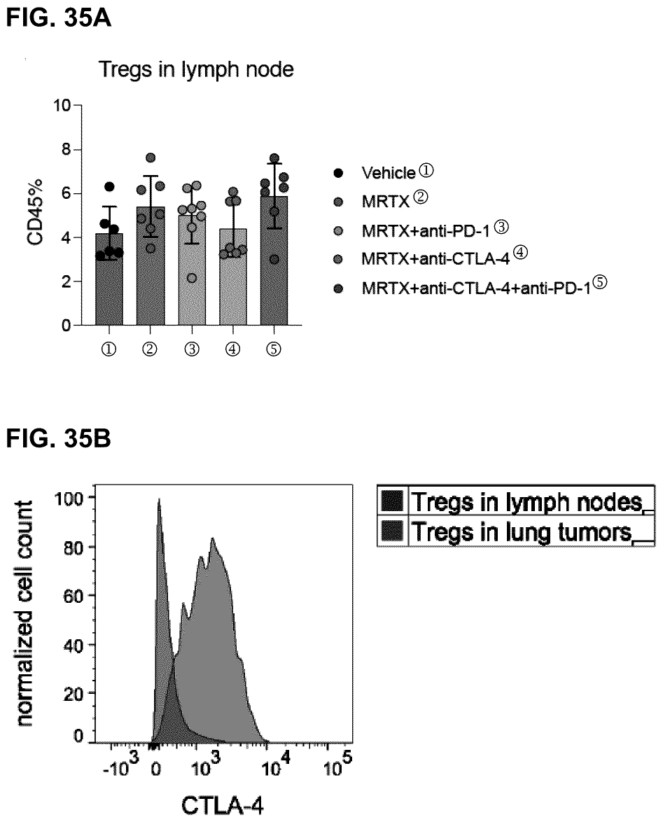

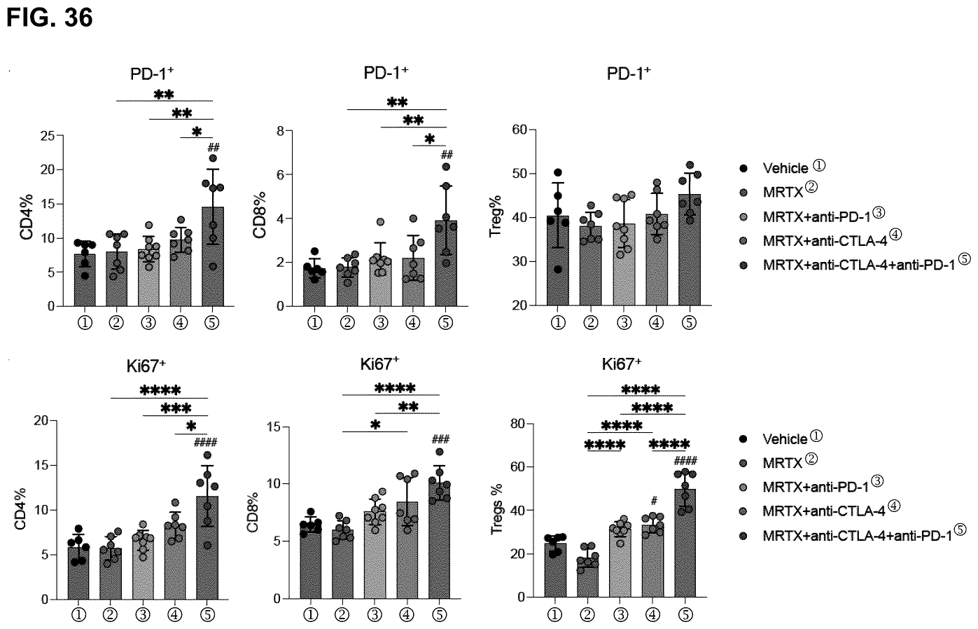

Interestingly, in the pulmonary lymph nodes, Treg cells were not depleted by the addition of anti-CTLA-4 (FIG. 35A). It was found that expression of surface CTLA-4 on Treg cells was higher in tumors compard to lymph nodes (FIG. 35B). Despite this, the lymph nodes showed pronounced T cell activation, evidenced by increased expression of PD-1 and proliferation marker Ki67 in particular in the triple combination treatment group (adagrasib+ anti-PD-1+ anti-CTLA-4), indicative of the priming of a systemic immune response (FIG. 36).

Overall, the results demonstrated that combining adagrasib with a Treg cell depleting agent (such as anti-CTLA-4 or anti-CCR8) upregulates T-cell mediated anti-tumoral immune response in the tumor, leading to reduced tumor volume and better survival in mice.

### CONCLUSION ON EXAMPLES

Despite objective response rates of roughly 30-40%, the licensed KRAS-G12C inhibitors sotorasib (de Langen et al. (2023) Lancet 401:733-746; Hong et al. (2020) N. Engl. J. Med. 383:1207-1217; Skoulidis et al. (2021) N. Engl. J. Med. 384:2371-2381) and more recently adagrasib (Jänne et al. (2022) N. Engl. J. Med. 387:120-131; Sabari et al. (2021) J. Clin. Oncol. 39: TPS146-TPS146) have so far achieved only a modest improvement in progression free survival of about one month and no improvement in overall survival. This very limited clinical benefit has been attributed to intrinsic and acquired resistance mechanisms (Suzuki et al. (2021) Clin. Cancer Res. 27:5697-5707; Vaclova et al. (2022) Sci. Rep. 12:2699; Tanaka et al. (2021) Cancer Discov. 11:1913-1922; Awad et al. (2021) N. Engl. J. Med. 384:2382-2393).

Combination therapies including KRAS inhibitors have been explored in an effort to overcome these resistance mechanisms. Besides combinatorial targeting of multiple tumor cell intrinsic pathways (Khan et al. (2022) Cancer Res. Commun. 2:342-352; Khan et al. (2022) J. Hematol. Oncol. 15:23; Tammaccaro et al. (2023) Pharmaceuticals (Basel) 16; Molina-Arcas et al. (2019) Sci. Transl. Med. 11), early pre-clinical experiments also suggested a potential synergy with immune therapy (Canon et al. (2019) Nature 575:217-223; Briere et al. (2021) Mol. Cancer Ther. 20 (6): 975-985). Inhibiting KRAS-G12C was able to turn a cold tumor into a hot pro-inflammatory environment, potentially supporting anti-tumor immune responses in combination with immune checkpoint inhibition (van Maldegem et al. (2021) Nat. Commun. 12:5906).

However, pre-clinical work demonstrated that despite the presence of TME changes, anti-PD-1 refractory tumors did not become responsive to immune checkpoint inhibition (ICI) as a result of KRAS-G12C inhibition (Mugarza et al. (2022), Sci. Adv. 8: eabm8780). Similarly, first reports of the safety and efficacy of sotorasib in combination with pembrolizumab or atezolizumab in advanced KRAS-G12C NSCLC have suggested poor response rates in patients that previously progressed on immune checkpoint inhibition, at least in part due to severe combination toxicities (Chour et al. (2023) J. Thorac. Oncol. 18 (10): 1408-1415).

To evaluate this persisting resistance to therapy, in the present disclosure an immune refractory KRAS-G12C mutant NSCLC model was investigated. The 3LL Lewis lung carcinoma model has a history of repeated passaging through immunocompetent mice and as a result has developed a complex mixture of immune resistance mechanisms (Mayo (1972) Cancer Chemother. Rep. 2 (3): 325-330). As shown previosuly, treating orthotopic 3LL lung tumors with KRAS-G12C inhibitors only provided temporary tumor control, and combinations with anti-PD-1 or anti-PD-L1 and anti-LAG-3 failed to give additional benefit (Mugarza et al. (2022) Sci. Adv. 8: eabm8780).

The present disclosure describes the investigation of spatial cellular communities in the therapy-resistant 3LL model for evidence of immune suppressive patterns. A T cell rich cellular community (T/DC) with evidence of T cell activation, proliferation, exhaustion and inhibition was identified. In this cellular community, abundant interactions between CD4⁺ and CD8⁺ T cells and mature antigen presenting DCs were observed, similar to interactions normally seen within the T cell zones of lymph nodes but not resembling tertiary lymphoid structures as B cell involvement was sparse. Furthermore, evidence of anti-tumor cytotoxicity within this community was found, with enrichment for interactions between CD8⁺ PD-1⁺ T cells and cleaved-caspase 3 expressing tumor cells, suggestive of T cell-induced apoptosis. After treatment with a KRAS G12C inhibitory drug, increased CXCL9 expression, which is involved in T cell recruitment, potentially from the periphery, was observed. Tumor draining lymph nodes (tdLNs) are considered to play a key role in facilitating the anti-tumor immune response, with the priming of new waves of antitumor T cells by both migratory and resident DC in tumor and tdLN, which are subsequently attracted to the tumor by chemokine expressing DC in the TME (van Pul et al. (2021) Front. Immunol. 12:643291). It was shown that patients responding to ICI harbored more shared TCR clones between tumor and tdLNs, but also provided evidence for local expansion and proliferation of CD8⁺ T cells in the TME (Magen et al. (2023) Nat. Med. 29:1389-1399). To what extent the make-up of spatial cellular communities in the TME are a direct reflection of processes occurring in the tdLNs remains to be determined.

While Treg cells were generally quite scarce in the data, they were found strongly enriched within the T/DC community. Treg cells have direct and indirect mechanisms to inhibit T cell function. Firstly, Tregs can inhibit CD8⁺ T cells directly by secreting or exposing T cell suppressive cytokines, such as IL-10, IL-35 and TGF-beta, by scavenging IL-2, and by the generation of extracellular adenosine (Schmidt et al. (2012) Front. Immunol. 3:51). Secondly, Treg cells can compete with CD8⁺ and CD4⁺ T cells for the co-stimulatory molecules on the antigen presenting cells, primarily by transendocytosis or trogocytosis of CD80/CD86 through the interaction with CTLA-4 (Tekguc et al. (2021) Proc. Natl. Acad. Sci. USA 118; Kennedy et al. (2022) Nat. Immunol. 23:1365-1378). CD8/DC interactions were indeed less frequently observed in the neighborhoods containing Treg cells, potentially reducing the opportunity for local activation of CD8⁺ T cells. Furthermore, depletion of Tregs using anti-CTLA-4, in combination with MRTX and PD-1, led to increased expression of activation markers, and most importantly, the CTLA-4 ligand CD86 on the DCs, and enabled tumor control by enhanced immunity in this otherwise strongly immune evasive model.

The data suggest that the presence of a spatial cellular community that is rich in activated T cells and DCs but also Treg cells can predict resistance to a combined therapy of KRAS-G12C inhibition and PD-1 blockade. Spatial investigation of cellular communities in pre-treatment biopsies would thus allow for correlation with clinical outcome. This is highly relevant as, for example, initial results in the CodeBreak 100/101 clinical trials, showed that the combination of sotorasib with PD-1 or PD-L1 led to unexpected liver toxicity (Li et al. Presentation at the 2022 World Conference on Lung Cancer, Vienna, Austria, Aug. 6-9, 2022). Hence, identifying and excluding patients that will likely be resistant to such combination therapies due to Treg cell activity would help to prevent unnecessary ineffective treatments that in turn have the potential to increase toxicity risks. Being able to predict the outcome of such anti-tumor therapies is thus of utmost value. Furthermore, the findings of the present disclosure open up the possibility of using anti-CTLA-4, anti-CCR8, or other Treg-depleting therapies in combination with KRAS inhibitors such as sotorasib in selected patients based on a spatial Treg cell community biomarker.

But, as the data show, Treg cell-rich communities are not unique to the KRAS inhibitor treated conditions, as the T/DC community was present at similar frequencies in the vehicle setting. Likewise, a deeper analysis of the RUBICON cohort demonstrated that various Treg cell-rich communities were also present in a significant proportion of treatment-naïve human patients. These observations suggest that DC, T cell and Treg cell-rich communities are a recurring feature, in particular, in lung cancer and could have the potential to predict response in a broader therapeutic context.

For example, spatial analysis of pre-treatment biopsies from cohorts such as ARCTIC (Planchard et al. (2020) Ann. Oncol. 31:609-618), MYSTIC (Garon et al. (2021) Clin. Lung Cancer 22:301-312.e8), and CHECKMATE 227 (Hellmann et al. (2019) N. Engl. J. Med. 381:2020-2031) comparing anti-PD-L1/PD-1 as monotherapy or in combination with CTLA-4, could help to better define the Treg communities that confer resistance to blocking the PD-1/PD-L1 axis alone, yet may be responsive to the addition of Treg targeting.

Further, clinical trials with NSCLC patients that combine nivolumab or durvalumab (anti-PD-(L)1) with ipilimumab or tremelimumab (anti-CTLA-4) have shown no or only modest additional clinical benefit compared to monotherapy PD-(L)1 blockade. This may be due to lack of statistical power, by absence of a biomarker to select the patients that may benefit from additional CTLA-4 blockade. Interestingly, the MYSTIC trial showed that the combination of durvalumab and tremelimumab versus durvalumab monotherapy was performing worse across the whole patient population, but showed improved survival in a select patient group with high tumor mutational burden (TMB) (Rizvi et al. (2020) JAMA Oncol. 6:661-674). In line with this, two spatial cellular communities with strong similarities to the spatial cellular community that was identified in mice were observed. These spatial cellular communities correlated with increased TMB. The spatial cellular communities identified in the present disclosure thus possibly also have the potential to be applied as a biomarker for predicting resistance to combination therapies of PD-1-(L)1 blockade with Treg-targeting agents, such as CTLA-4 blockade. Patients predicted to be resistant could then be excluded from most likely unsuccessful therapies.

In conclusion, spatial cellular community analysis was used to investigate the nature of resistance to combined KRAS-G12C and PD-1 inhibition in a strongly immune evasive mouse model for NSCLC. A cellular community that is rich in T cells and DCs, with evidence of local T cell activation and cytotoxicity, but that is inhibited by Treg-mediated suppression, was identified. Depletion of the Tregs led to a profound reduction in tumor growth, longer survival and enhanced, and in some cases sustained, anti-tumor immunity. Thus, this disclosure is in certain aspects directed to methods of treating a tumor in a subject in need thereof comprising administering to the subject a KRAS-G12C inhibitor (such as adagrasib, sotorasib, or MRTX1257), a Treg-depleting agent such as an anti-CCR8 antibody (such as imzokitug, 14S15 or 14S15h), and optionally a PD-1 axis-blocking agent, such as an anti-PD-1 antibody (such as nivolumab or pembrolizumab) or an anti-PD-L1 antibody (such as atezolizumab, durvalumab, or avelumab). Analysis of treatment-naïve patients showed that similar communities rich in CD4, CD8⁺ and Treg cells were found to co-occur with DCs in the peri-tumor space and correlated with increased TMB. A detailed spatial analysis of Treg rich communities in clinical samples from patients treated with KRAS-inhibitors, anti-PD-1/PDL1 or anti-CTLA-4 may thus provide the foundations for a very specific predictive biomarker.

1. (canceled)

2. A method of treating a tumor in a subject in need thereof comprising:
(a) administering a KRAS inhibitor to the subject;
(b) measuring the number of regulatory T cells (“Tregs”) in the tumor microenvironment (“TME); and
(c) administering a regulatory T cell (“Treg”)-depleting agent to the subject.

3. A method of treating a tumor in a subject in need thereof comprising administering a Treg-depleting agent to the subject, wherein the subject is identified as having an increased number of Tregs in the tumor microenvironment (TME) after administration of a KRAS inhibitor.

4. A method of treating a tumor in a subject in need thereof comprising administering a KRAS inhibitor and a Treg-depleting agent to the subject, wherein:
(a) the KRAS inhibitor and the Treg-depleting agent are administered separately; or
(b) wherein the KRAS inhibitor and the Treg-depleting agent are administered concurrently.

5-7. (canceled)

8. The method of claim 4, further comprising administering to the subject an additional anti-tumor treatment.

9-11. (canceled)

12. The method of claim 3, wherein the Tregs are located in a spatial cellular community in the TME.

13-17. (canceled)

18. The method of claim 12, wherein the spatial cellular community comprises:
(a) Tregs that are in the neighborhood of CD8⁺ T cells, wherein the CD8⁺ T cells in the neighborhood of the Tregs are CD8⁺ T cells neighboring DCs and CD4⁺ T cells;
(b) activated CD8⁺ T cells;
(c) Tregs that inhibit CD4⁺ T cells and/or CD8⁺ T cells in the spatial cellular community;
(d) tumor cells expressing Ki67 and tumor cells expressing cleaved-caspase-3 (c-casp3);
(e) CD8+ T cells in the neighborhood of c-casp3+ tumor cells; or
(f) any combination of (a)-(e).

19-24. (canceled)

25. The method of claim 12, wherein the spatial cellular community comprises about 5% to about 12% CD4⁺ T cells, about 1% to about 4% CD8⁺ T cells, about 0.5% to about 2% Tregs, and about 10% to about 20% DCs, and optionally about 20% to about 45% tumor cells, and/or optionally about 2% to about 10% CD103⁺ DCs.

26-27. (canceled)

28. The method of claim 8, wherein the additional anti-tumor treatment comprises an immunotherapy.

29-32. (canceled)

33. The method of claim 3, wherein the tumor is comprises a lung cancer, a colorectal cancer, a pancreatic cancer, pancreatic adenocarcinoma, colon adenocarcinoma, colorectal adenocarcinoma, lung adenocarcinoma, non-small cell lung cancer (NSCLC), cholangiocarcinoma, uterine endometrial carcinoma, testicular germ cell cancer, ampullary carcinoma, cervical squamous cell carcinoma, appendiceal adenocarcinoma, bladder adenocarcinoma, small intestinal carcinoma, ovarian cancer, and or myelodysplastic syndromes.

34-38. (canceled)

39. The method of claim 3, wherein the Treg-depleting agent is capable of specifically binding to CCR8, CTLA-4, CCR4, CD25, TIM-3, VISTA, GITR, 4-1BB, OX-40, CD27, ICOS, CD15s (sialyl Lewis x), MALT1, TNFR2, TGF-β receptor, and/or TIGIT.

40-46. (canceled)

47. The method of claim 39 46, wherein the Treg-depleting agent comprises is an anti-CTLA-4 antibody or an anti-CCR8 antibody.

48. The method of claim 47, wherein the anti-CTLA-4 antibody comprises:
i) a heavy chain variable domain (VH) comprising a heavy chain complementarity determining region 1 (CDRH1) comprising the amino acid sequence as set forth in SEQ ID NO: 156, a heavy chain complementarity determining region 2 (CDRH2) comprising the amino acid sequence as set forth in SEQ ID NO: 157, and a heavy chain complementarity determining region 3 (CDRH3) comprising the amino acid sequence as set forth in SEQ ID NO: 158; and a light chain variable domain (VL) comprising a light chain complementarity determining region 1 (CDRL1) comprising the amino acid sequence as set forth in SEQ ID NO: 159, a light chain complementarity determining region 2 (CDRL2) comprising the amino acid sequence as set forth in SEQ ID NO: 160, and a light chain complementarity determining region 3 (CDRL3) comprising the amino acid sequence as set forth in SEQ ID NO: 161; or
ii) a VH comprising a CDRH1 comprising the amino acid sequence as set forth in SEQ ID NO: 141, a CDRH2 comprising the amino acid sequence as set forth in SEQ ID NO: 142, and a CDRH3 comprising the amino acid sequence as set forth in SEQ ID NO: 143; and a VL comprising a CDRL1 comprising the amino acid sequence as set forth in SEQ ID NO: 144, a CDRL2 comprising the amino acid sequence as set forth in SEQ ID NO: 145, and a CDRL3 comprising the amino acid sequence as set forth in SEQ ID NO: 146.

49-52. (canceled)

53. The method of claim 47, wherein the anti-CCR8 antibody comprises:
i) a VH comprising a CDRH1 comprising the amino acid sequence as set forth in SEQ ID NO: 1, a CDRH2 comprising the amino acid sequence as set forth in SEQ ID NO: 2, and a CDRH3 comprising the amino acid sequence as set forth in SEQ ID NO: 3, and a VL comprising a CDRL1 comprising the amino acid sequence as set forth in SEQ ID NO: 4, a CDRL2 comprising the amino acid sequence as set forth in SEQ ID NO: 5, and a CDRL3 comprising the amino acid sequence as set forth in SEQ ID NO: 6; or
ii) a VH comprising a CDRH1 comprising the amino acid sequence as set forth in SEQ ID NO: 21, a CDRH2 comprising the amino acid sequence as set forth in SEQ ID NO: 22, and a CDRH3 comprising the amino acid sequence as set forth in SEQ ID NO: 23, and a VL comprising a CDRL1 comprising the amino acid sequence as set forth in SEQ ID NO: 24, a CDRL2 comprising the amino acid sequence as set forth in SEQ ID NO: 25, and a CDRL3 comprising the amino acid sequence as set forth in SEQ ID NO: 26; or
iii) a VH comprising a CDRH1 comprising the amino acid sequence as set forth in SEQ ID NO: 47, a CDRH2 comprising the amino acid sequence as set forth in SEQ ID NO: 48, and a CDRH3 comprising the amino acid sequence as set forth in SEQ ID NO: 49, and a VL comprising a CDRL1 comprising the amino acid sequence as set forth in SEQ ID NO: 50, a CDRL2 comprising the amino acid sequence as set forth in SEQ ID NO: 51, and a CDRL3 comprising the amino acid sequence as set forth in SEQ ID NO: 52.

54-67. (canceled)

68. The method of claim 3, wherein the KRAS inhibitor is selected from comprises sotorasib, adagrasib, MRTX-1257, ARS-853, ARS-1620, MRTX-EX185, MRTX-1133, ASP2453, RMC-6291, RMC-6236, RMC-036, RMC-037, BBO-8520, BI 1701963, ERAS-3490, JAB-21822, JNJ-74699157, GFH925, YL-15293, YL-17231, SY-5933, and JDQ443.

69-83. (canceled)

84. The method of claim 8, wherein the additional anti-tumor treatment comprises a PD-1 axis-blocking agent.

85-87. (canceled)

88. The method of claim 84, wherein the PD-1 axis-blocking agent comprises nivolumab, pembrolizumab, cemiplimab, spartalizumab, camrelizumab, sintilimab, tislelizumab, toripalimab, dostarlimab, retifanlimab, pimivalimab, serplulimab, zimberelimab, acrixolimab, sasanlimab, MEDI-0680, AM-0001, STI-1110, AGEN2034, BCD-100, BI 754091, SSI-361, atezolizumab, durvalumab, avelumab, envafolimab, cosibelimab, BMS-936559, STI-1014, pacmilimab, lodapolimab, FAZ053, CS-1001, SHR-1316, CBT-502, KN035, or BGB-A333.

89-98. (canceled)

99. The method of claim 8, wherein the additional anti-tumor treatment comprises relatlimab, IMP731, GSK2831781, humanized BAP050, LAG-525, MK-4280, REGN3767, aLAG-3 (0414), aLAG-3 (0416), TSR-033, TSR-075, Sym022, FS-118, XmAb841, MGD013, BI754111, P 13B02-30, AVA-017, 25F7, AGEN1746, RO7247669, INCAGN02385, IBI-110, EMB-02, IBI-323, LBL-007, or ABL501.

100-102. (canceled)

103. A kit comprising (a) a KRAS inhibitor; (b) a Treg-depleting agent; (c) optionally an additional anti-tumor treatment; and (d) instructions for use of the KRAS inhibitor and the Treg-depleting agent, according to the method of claim 3.

104-108. (canceled)

109. The kit of claim 103, further comprising an additional anti-tumor agent, which comprises an immunotherapy.

110. A composition comprising a KRAS inhibitor for the method according to claim 2.
